(19) United States
(12) Patent Application Publication
(10) Pub. No.: US 2023/0403931 A1
(43) Pub. Date: Dec. 14, 2023

(54) ORGANIC MOLECULES FOR OPTOELECTRONIC DEVICES

(71) Applicant: Samsung Display Co., Ltd., Yongin-si, Gyeonggi-do (KR)

(72) Inventors: Michael DANZ, Eggenstein-Leopoldshafen (DE); Damien THIRION, Karlsdorf-Neuthard (DE); Daniel ZINK, Graben-Neudorf (DE)

(21) Appl. No.: 18/248,032

(22) PCT Filed: Oct. 7, 2021
(86) PCT No.: PCT/EP2021/077699

§ 371 Date: Apr. 5, 2023

(30) Foreign Application Priority Data
Oct. 9, 2020 (EP) 20201178.9

(51) Int. Cl. H10K 85/60; C07D 403/14; C09K 11/06; C07D 487/04; C07D 405/14

(52) U.S. Cl. CPC H10K 85/6572; C07D 403/14; C09K 11/06; H10K 85/654; C07D 487/04; C07D 405/14; H10K 85/6574; H10K 50/12

(57) ABSTRACT

Embodiments of the present disclosure relate to a light emitting organic molecule, for application in optoelectronic devices. According to the invention, the organic molecule has a first chemical moiety having a structure of formula I-a or formula I-b, and a second chemical moiety having a structure of formula II, wherein the first chemical moiety is linked to the second chemical moiety via a single bond.

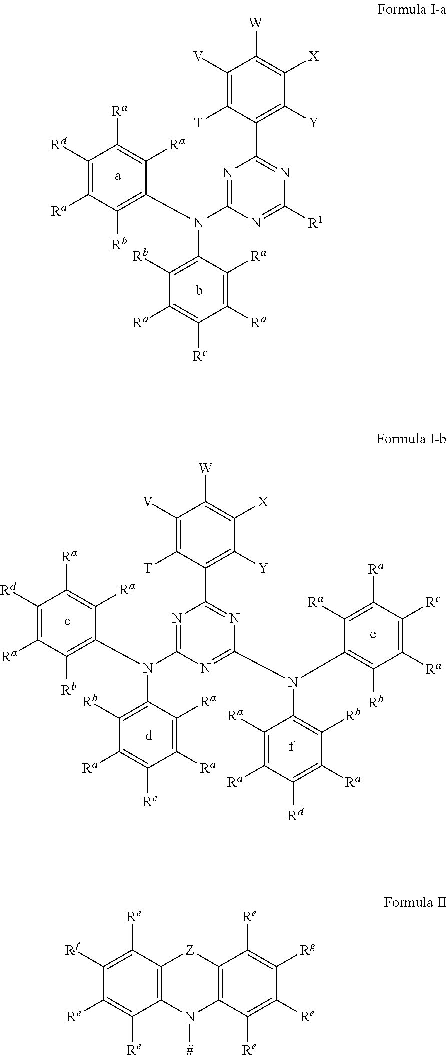

### CROSS-REFERENCE TO RELATED APPLICATIONS

This application is a U.S. National Phase Patent Application of International Patent Application Number PCT/EP2021/077699, filed on Oct. 7, 2021, which claims priority to European Patent Application Number 20201178.9, filed on Oct. 9, 2020, the entire content of all of which is incorporated herein by reference.

### BACKGROUND

Embodiments of the present disclosure relates to light-emitting organic molecules and their use in organic light-emitting diodes (OLEDs) and in other optoelectronic devices.

### BRIEF DESCRIPTION OF THE FIGURES

The accompanying drawings are included to provide a further understanding of the subject matter of the present disclosure, and are incorporated in and constitute a part of this specification. The drawings illustrate exemplary embodiments of the present disclosure and, together with the description, serve to explain principles of the present disclosure. In the drawings:

FIG. 1 depicts an emission spectrum of example 1 (10% by weight) in PMMA.

FIG. 2 depicts an emission spectrum of example 2 (10% by weight) in PMMA.

FIG. 3 depicts an emission spectrum of example 3 (10% by weight) in PMMA.

FIG. 4 depicts an emission spectrum of example 4 (10% by weight) in PMMA.

FIG. 5 depicts an emission spectrum of example 5 (10% by weight) in PMMA.

FIG. 6 depicts an emission spectrum of example 6 (10% by weight) in PMMA.

FIG. 7 depicts an emission spectrum of example 7 (10% by weight) in PMMA.

FIG. 8 depicts an emission spectrum of example 8 (10% by weight) in PMMA.

FIG. 9 depicts an emission spectrum of example 9 (10% by weight) in PMMA.

FIG. 10 depicts an emission spectrum of example 10 (10% by weight) in PMMA.

FIG. 11 depicts an emission spectrum of example 11 (10% by weight) in PMMA.

FIG. 12 depicts an emission spectrum of example 12 (10% by weight) in PMMA.

FIG. 13 depicts an emission spectrum of example 13 (10% by weight) in PMMA.

### DESCRIPTION

The object of embodiments of the present disclosure is to provide molecules which are suitable for use in optoelectronic devices.

This object is achieved by embodiments of the present disclosure which provide a new class of organic molecules.

The organic molecules of embodiments of the present disclosure are purely organic molecules, e.g., they do not contain any metal ions in contrast to metal complexes used in optoelectronic devices.

The organic molecules according to embodiments of the present disclosure exhibit emission maxima in the deep blue, sky blue, green or yellow spectral range, for example, in the deep blue, sky blue, and green spectral range, and, for example, in the deep blue or green spectral range. The organic molecules exhibit emission maxima between 420 and 580 nm, between 440 and 560 nm, between 440 and 480 nm or between 500 and 550 nm, and, for example, between 450 and 470 nm or between 520 and 540 nm. The photoluminescence quantum yields of the organic molecules according to embodiments of the present disclosure are equal to or higher than 10%, equal to or higher than 20%, equal to or higher than 30%, equal to or higher than 40%, and, for example, equal to or higher than 50%. The molecules of embodiments of the present disclosure exhibit thermally activated delayed fluorescence (TADF). The use of the molecules according to embodiments of the present disclosure in an optoelectronic device, for example, an organic light-emitting diode (OLED), leads to higher efficiencies of the device. Corresponding OLEDs have a higher stability than OLEDs including other emitter materials and comparable color and/or by employing the molecules according to embodiments of the present disclosure in an OLED display, a more accurate reproduction of visible colors in nature, e.g., a higher resolution in the displayed image, is achieved. In some embodiments, the molecules can be used in combination with a fluorescence emitter to enable so-called hyper-fluorescence.

The organic molecules according to embodiments of the present disclosure comprise or consist of:
a first chemical moiety comprising or consisting of a structure of formula I-a or formula I-b:

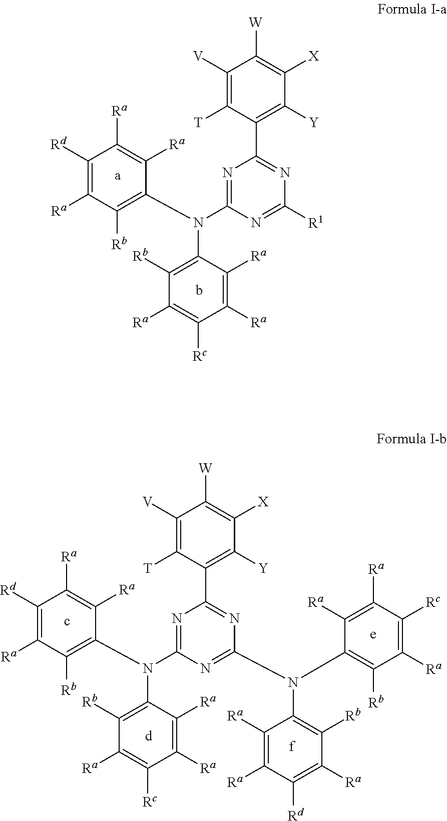

and
a second chemical moiety, comprising or consisting of a structure of formula II:

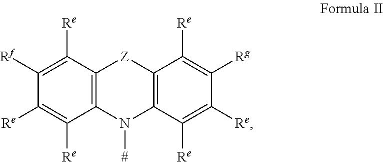

wherein the first chemical moiety is linked to the second chemical moiety via a single bond.

T is the binding site of a single bond linking the first chemical moiety to the second chemical moiety, or is selected from the group consisting of R² and R^{X}.

V is the binding site of a single bond linking the first chemical moiety to the second chemical moiety, or is hydrogen (H).

W is the binding site of a single bond linking the first chemical moiety to the second chemical moiety, or is selected from the group consisting of R² and R^{X}.

X is selected from the group consisting of R² and R^{X}.

Y is selected from the group consisting of R² and R^{X}.

R^{X} is selected from CN and CF₃ or R^{X} comprises or consists of a structure of formula BN-I,

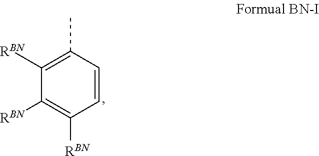

which is bonded to the structure of formula I-a or I-b via a single bond indicated by the dashed line and wherein exactly one R^{BN} group is CN while the other two R^{BN} groups are both hydrogen (H), e.g., in a case R^{X} is represented by formula BN-I, it comprises or consists of a structure according to any of the formulas BN-I-a, BN-I-b, and BN-I-c:

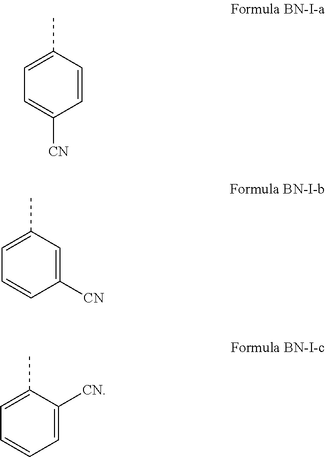

R¹ is selected from the group consisting of: hydrogen, deuterium, OR³, Si(R³)₃, B(OR³)₂, OSO₂R³, CF₃, CN, F, Cl, Br, I,

C₁-C₄₀-alkyl,

which is optionally substituted with one or more substituents R³ and

wherein one or more non-adjacent CH₂-groups are optionally substituted by R³C═CR³, C≡C, Si(R³)₂, Ge(R³)₂, Sn(R³)₂, C═O, C═S, C═Se, C═NR³, P(═O)(R³), SO, SO₂, NR³, O, S or CONR³;

C₁-C₄₀-alkoxy,

which is optionally substituted with one or more substituents R³ and

wherein one or more non-adjacent CH₂-groups are optionally substituted by R³C═CR³, C≡C, Si(R³)₂, Ge(R³)₂, Sn(R³)₂, C═O, C═S, C═Se, C═NR³, P(═O)(R³), SO, SO₂, NR³, O, S or CONR³;

C₁-C₄₀-thioalkoxy,

which is optionally substituted with one or more substituents R³ and

wherein one or more non-adjacent CH₂-groups are optionally substituted by R³C═CR³, C≡C, Si(R³)₂, Ge(R³)₂, Sn(R³)₂, C═O, C═S, C═Se, C═NR³, P(═O)(R³), SO, SO₂, NR³, O, S or CONR³;

C₂-C₄₀-alkenyl,

which is optionally substituted with one or more substituents R³ and

wherein one or more non-adjacent CH₂-groups are optionally substituted by R³C═CR³, C≡C, Si(R³)₂, Ge(R³)₂, Sn(R³)₂, C═O, C═S, C═Se, C═NR³, P(═O)(R³), SO, SO₂, NR³, O, S or CONR³;

C₂-C₄₀-alkynyl,

which is optionally substituted with one or more substituents R³ and

wherein one or more non-adjacent CH₂-groups are optionally substituted by R³C═CR³, C≡C, Si(R³)₂, Ge(R³)₂, Sn(R³)₂, C═O, C═S, C═Se, C═NR⁷, P(═O)(R³), SO, SO₂, NR³, O, S or CONR³;

C₆-C₆₀-aryl,

which is optionally substituted with one or more substituents R³.

R³ is at each occurrence independently of each other selected from the group consisting of: hydrogen, deuterium, N(R⁴)₂, OR⁴, Si(R⁴)₃, B(OR⁴)₂, OSO₂R⁴, CF₃, CN, F, Cl, Br, I,

C₁-C₄₀-alkyl,

which is optionally substituted with one or more substituents R⁴ and

wherein one or more non-adjacent CH₂-groups are optionally substituted by R⁴C═CR⁴, C≡C, Si(R⁴)₂, Ge(R⁴)₂, Sn(R⁴)₂, C═O, C═S, C═Se, C═NR⁴, P(═O)(R⁴), SO, SO₂, NR⁴, O, S or CONR⁴;

C₁-C₄₀-alkoxy,

which is optionally substituted with one or more substituents R⁴ and

wherein one or more non-adjacent CH₂-groups are optionally substituted by R⁴C═CR⁴, C≡C, Si(R⁴)₂, Ge(R⁴)₂, Sn(R⁴)₂, C═O, C═S, C═Se, C═NR⁴, P(═O)(R⁴), SO, SO₂, NR⁴, O, S or CONR⁴;

C₁-C₄₀-thioalkoxy,

which is optionally substituted with one or more substituents R⁴ and

wherein one or more non-adjacent CH₂-groups are optionally substituted by R⁴C═CR⁴, C≡C, Si(R⁴)₂, Ge(R⁴)₂, Sn(R⁴)₂, C═O, C═S, C═Se, C═NR⁴, P(═O)(R⁴), SO, SO₂, NR⁴, O, S or CONR⁴;

C₂-C₄₀-alkenyl,

which is optionally substituted with one or more substituents R⁴ and

wherein one or more non-adjacent CH₂-groups are optionally substituted by R⁴C═CR⁴, C≡C, Si(R⁴)₂, Ge(R⁴)₂, Sn(R⁴)₂, C═O, C═S, C═Se, C═NR⁴, P(═O)(R⁴), SO, SO₂, NR⁴, O, S or CONR⁴;

C₂-C₄₀-alkynyl,

which is optionally substituted with one or more substituents R⁴ and

wherein one or more non-adjacent CH₂-groups are optionally substituted by R⁴C═CR⁴, C≡C, Si(R⁴)₂, Ge(R⁴)₂, Sn(R⁴)₂, C═O, C═S, C═Se, C═NR⁴, P(═O)(R⁴), SO, SO₂, NR⁴, O, S or CONR⁴;

C₆-C₆₀-aryl,

which is optionally substituted with one or more substituents R⁴; and

C₃-C₆₀-heteroaryl,

which is optionally substituted with one or more substituents R⁴.

R² is at each occurrence independently of each other selected from the group consisting of:

hydrogen, deuterium,

C₁-C₁₀-alkyl,

wherein one or more hydrogen atoms are optionally substituted by deuterium;

C₂-C₁₀-alkenyl,

wherein one or more hydrogen atoms are optionally substituted by deuterium;

C₂-C₁₀-alkynyl,

wherein one or more hydrogen atoms are optionally substituted by deuterium;

C₅-C₁₀-aryl;

wherein one or more hydrogen atoms are optionally substituted by a group R⁵.

Optionally, the two moieties R^{b} comprised in the first chemical moiety combine and together form a group Y, which is selected from the group consisting of a direct bond, CR⁶R⁷, C═CR⁶R⁷, C═O, C═NR⁶, NR⁶, O, SiR⁶R⁷, S, S(O) and S(O)₂.

R^{a}, R^{b}, R^{c}, R^{d}, R⁶, and R⁷ are at each occurrence independently of each other selected from the group consisting of: hydrogen, deuterium, N(R⁸)₂, OR⁸, Si(R⁸)₃, B(OR⁸)₂, OSO₂R⁸, CF₃, CN, F, Cl, Br, I,

C₁-C₄₀-alkyl,

which is optionally substituted with one or more substituents R⁶ and

wherein one or more non-adjacent CH₂-groups are optionally substituted by R⁸C═CR⁸, C≡C, Si(R⁸)₂, Ge(R⁸)₂, Sn(R⁸)₂, C═O, C═S, C═Se, C═NR⁸, P(═O)(R⁸), SO, SO₂, NR⁸, O, S or CONR⁸;

C₁-C₄₀-alkoxy,

which is optionally substituted with one or more substituents R⁸ and

wherein one or more non-adjacent CH₂-groups are optionally substituted by R⁸C═CR⁸, C≡C, Si(R⁸)₂, Ge(R⁸)₂, Sn(R⁸)₂, C═O, C═S, C═Se, C═NR⁸, P(═O)(R⁸), SO, SO₂, NR⁸, O, S or CONR⁸;

C₁-C₄₀-thioalkoxy,

which is optionally substituted with one or more substituents R⁸ and

wherein one or more non-adjacent CH₂-groups are optionally substituted by R⁸C═CR⁸, C≡C, Si(R⁸)₂, Ge(R⁸)₂, Sn(R⁸)₂, C═O, C═S, C═Se, C═NR⁸, P(═O)(R⁸), SO, SO₂, NR⁸, O, S or CONR⁸;

C₂-C₄₀-alkenyl,

which is optionally substituted with one or more substituents R⁸ and

wherein one or more non-adjacent CH₂-groups are optionally substituted by R⁸C═CR⁸, C≡C, Si(R⁸)₂, Ge(R⁸)₂, Sn(R⁸)₂, C═O, C═S, C═Se, C═NR⁸, P(═O)(R⁸), SO, SO₂, NR⁸, O, S or CONR⁸;

C₂-C₄₀-alkynyl,

which is optionally substituted with one or more substituents R⁸ and

wherein one or more non-adjacent CH₂-groups are optionally substituted by R⁸C═CR⁸, C≡C, Si(R⁸)₂, Ge(R⁸)₂, Sn(R⁸)₂, C═O, C═S, C═Se, C═NR⁸, P(═O)(R⁸), SO, SO₂, NR⁸, O, S or CONR⁸;

C₆-C₆₀-aryl,

which is optionally substituted with one or more substituents R⁸; and

C₃-C₆₀-heteroaryl,

which is optionally substituted with one or more substituents R⁸.

R⁸ is at each occurrence independently of each other selected from the group consisting of: hydrogen, deuterium, N(R⁹)₂, OR⁹, Si(R⁹)₃, B(OR⁹)₂, OSO₂R⁹, CF₃, CN, F, Cl, Br, I,

C₁-C₄₀-alkyl,

which is optionally substituted with one or more substituents R⁹ and

wherein one or more non-adjacent CH₂-groups are optionally substituted by R⁹C═CR⁹, C≡C, Si(R⁹)₂, Ge(R⁹)₂, Sn(R⁹)₂, C═O, C═S, C═Se, C═NR⁹, P(═O)(R⁹), SO, SO₂, NR⁹, O, S or CONR⁹;

C₁-C₄₀-alkoxy,

which is optionally substituted with one or more substituents R⁹ and

wherein one or more non-adjacent CH₂-groups are optionally substituted by R⁹C═CR⁹, C≡C, Si(R⁹)₂, Ge(R⁹)₂, Sn(R⁹)₂, C═O, C═S, C═Se, C═NR⁹, P(═O)(R⁹), SO, SO₂, NR⁹, O, S or CONR⁹;

C₁-C₄₀-thioalkoxy,

which is optionally substituted with one or more substituents R⁹ and

wherein one or more non-adjacent CH₂-groups are optionally substituted by R⁹C═CR⁹, C≡C, Si(R⁹)₂, Ge(R⁹)₂, Sn(R⁹)₂, C═O, C═S, C═Se, C═NR⁹, P(═O)(R⁹), SO, SO₂, NR⁹, O, S or CONR⁹;

C₂-C₄₀-alkenyl,

which is optionally substituted with one or more substituents R⁹ and

wherein one or more non-adjacent CH₂-groups are optionally substituted by R⁹C═CR⁹, C≡C, Si(R⁹)₂, Ge(R⁹)₂, Sn(R⁹)₂, C═0, C═S, C═Se, C═NR⁹, P(═O)(R⁹), SO, SO₂, NR⁹, O, S or CONR⁹;

C₂-C₄₀-alkynyl,

which is optionally substituted with one or more substituents R⁹ and

wherein one or more non-adjacent CH₂-groups are optionally substituted by R⁹C═CR⁹, C≡C, Si(R⁹)₂, Ge(R⁹)₂, Sn(R⁹)₂, C═0, C═S, C═Se, C═NR⁹, P(═O)(R⁹), SO, SO₂, NR⁹, O, S or CONR⁹;

C₆-C₆₀-aryl,

which is optionally substituted with one or more substituents R⁹; and

C₃-C₅₇-heteroaryl,

which is optionally substituted with one or more substituents R⁹.

Optionally, any of the substituents R^{a}, R^{b}, R^{c}, R^{d}, R⁶, R⁷, and R⁸ independently of each other form a mono- or polycyclic, aliphatic or aromatic, carbo- or heterocyclic and/or benzo-fused ring or ring system with one or more adjacent substituents selected from R^{a}, R^{b}, R^{c}, R^{d}, R⁶, R⁷, and R⁸; wherein the optionally so formed additional rings may optionally be substituted with one or more substituents R¹⁰.

#represents the binding site of the first chemical moiety to the second chemical moiety.

Z is selected from the group consisting of a direct bond, CR¹¹R¹², C═CR¹¹R¹², C═O, C═NR¹¹, NR¹¹, O, SiR¹¹R¹², S, S(O), and S(O)₂.

R^{e}, R^{f}, R^{g}, R¹¹, and R¹² are at each occurrence independently of each other selected from the group consisting of: hydrogen, deuterium, N(R¹³)₂, OR¹³, Si(R¹³)₃, B(OR¹³)₂, OSO₂R¹³, CF₃, CN, F, Cl, Br, I,

C₁-C₄₀-alkyl,

which is optionally substituted with one or more substituents R¹³ and

wherein one or more non-adjacent CH₂-groups are optionally substituted by R¹³C═CR¹³, C≡C, Si(R¹³)₂, Ge(R¹³)₂, Sn(R¹³)₂, C═O, C═S, C═Se, C═NR¹³, P(═O)(R¹³), SO, SO₂, NR¹³, O, S or CONR¹³;

C₁-C₄₀-alkoxy,

which is optionally substituted with one or more substituents R¹³ and

wherein one or more non-adjacent CH₂-groups are optionally substituted by R¹³C═CR¹³, C≡C, Si(R¹³)₂, Ge(R¹³)₂, Sn(R¹³)₂, C═O, C═S, C═Se, C═NR¹³, P(═O)(R¹³), SO, SO₂, NR¹³, O, S or CONR¹³;

C₁-C₄₀-thioalkoxy,

which is optionally substituted with one or more substituents R¹³ and

wherein one or more non-adjacent CH₂-groups are optionally substituted by R¹³C═CR¹³, C≡C, Si(R¹³)₂, Ge(R¹³)₂, Sn(R¹³)₂, C═O, C═S, C═Se, C═NR¹³, P(═O)(R¹³), SO, SO₂, NR¹³, O, S or CONR¹³;

C₂-C₄₀-alkenyl,

which is optionally substituted with one or more substituents R¹³ and

wherein one or more non-adjacent CH₂-groups are optionally substituted by R¹³C═CR¹³, C≡C, Si(R¹³)₂, Ge(R¹³)₂, Sn(R¹³)₂, C═O, C═S, C═Se, C═NR¹³, P(═O)(R¹³), SO, SO₂, NR¹³, O, S or CONR¹³;

C₂-C₄₀-alkynyl,

which is optionally substituted with one or more substituents R¹³ and

wherein one or more non-adjacent CH₂-groups are optionally substituted by R¹³C═CR¹³, C≡C, Si(R¹³)₂, Ge(R¹³)₂, Sn(R¹³)₂, C═O, C═S, C═Se, C═NR¹³, P(═O)(R¹³), SO, SO₂, NR¹³, O, S or CONR¹³;

C₆-C₆₀-aryl,

which is optionally substituted with one or more substituents R¹³; and

C₃-C₆₀-heteroaryl,

which is optionally substituted with one or more substituents R¹³.

R¹³ is at each occurrence independently of each other selected from the group consisting of: hydrogen, deuterium, N(R¹⁴)₂, OR¹⁴, Si(R¹⁴)₃, B(OR¹⁴)₂, OSO₂R¹⁴, CF₃, CN, F, Br, I,

C₁-C₄₀-alkyl,

which is optionally substituted with one or more substituents R¹⁴ and

wherein one or more non-adjacent CH₂-groups are optionally substituted by R¹⁴C═CR¹⁴, C≡C, Si(R¹⁴)₂, Ge(R¹⁴)₂, Sn(R¹⁴)₂, C═O, C═S, C═Se, C═NR¹⁴, P(═O)(R¹⁴), SO, SO₂, NR¹⁴, O, S or CONR¹⁴;

C₁-C₄₀-alkoxy,

which is optionally substituted with one or more substituents R¹⁴ and

wherein one or more non-adjacent CH₂-groups are optionally substituted by R¹⁴C═CR¹⁴, C≡C, Si(R¹⁴)₂, Ge(R¹⁴)₂, Sn(R¹⁴)₂, C═O, C═S, C═Se, C═NR¹⁴, P(═O)(R¹⁴), SO, SO₂, NR¹⁴, O, S or CONR¹⁴;

C₁-C₄₀-thioalkoxy,

which is optionally substituted with one or more substituents R¹⁴ and

wherein one or more non-adjacent CH₂-groups are optionally substituted by R¹⁴C═CR¹⁴, C≡C, Si(R¹⁴)₂, Ge(R¹⁴)₂, Sn(R¹⁴)₂, C═O, C═S, C═Se, C═NR¹⁴, P(═O)(R¹⁴), SO, SO₂, NR¹⁴, O, S or CONR¹⁴;

C₂-C₄₀-alkenyl,

which is optionally substituted with one or more substituents R¹⁴ and

wherein one or more non-adjacent CH₂-groups are optionally substituted by R¹⁴C═CR¹⁴, C≡C, Si(R¹⁴)₂, Ge(R¹⁴)₂, Sn(R¹⁴)₂, C═O, C═S, C═Se, C═NR¹⁴, P(═O)(R¹⁴), SO, SO₂, NR¹⁴, O, S or CONR¹⁴;

C₂-C₄₀-alkynyl,

which is optionally substituted with one or more substituents R¹⁴ and

wherein one or more non-adjacent CH₂-groups are optionally substituted by R¹⁴C═CR¹⁴, C≡C, Si(R¹⁴)₂, Ge(R¹⁴)₂, Sn(R¹⁴)₂, C═O, C═S, C═Se, C═NR¹⁴, P(═O)(R¹⁴), SO, SO₂, NR¹⁴, O, S or CONR¹⁴;

C₆-C₆₀-aryl,

which is optionally substituted with one or more substituents R¹⁴; and

C₃-C₅₇-heteroaryl,

which is optionally substituted with one or more substituents R¹⁴.

Optionally, any of the substituents R^{e}, R^{f}, R^{g}, R¹¹, R¹², and R¹³ independently of each other form a mono- or polycyclic, aliphatic or aromatic, carbo- or heterocyclic and/or benzo-fused ring or ring system with one or more adjacent substituents selected from R^{e}, R^{f}, R^{g}, R¹¹, R¹², and R¹³; wherein the optionally so formed additional rings may optionally be substituted with one or more substituents R¹⁵.

R⁴, R⁹, R¹⁰, R¹⁴, and R¹⁵ are at each occurrence independently of each other selected from the group consisting of: hydrogen, deuterium, Oph (Ph═phenyl), CF₃, CN, F,

C₁-C₅-alkyl,

wherein one or more hydrogen atoms are optionally, independently of each other substituted by deuterium, CN, CF₃, or F;

C₁-C₅-alkoxy,

wherein one or more hydrogen atoms are optionally, independently of each other substituted by deuterium, CN, CF₃, or F;

C₁-C₅-thioalkoxy,

wherein one or more hydrogen atoms are optionally, independently of each other substituted by deuterium, CN, CF₃, or F;

C₂-C₅-alkenyl,

wherein one or more hydrogen atoms are optionally, independently of each other substituted by deuterium, CN, CF₃, or F;

C₂-C₅-alkynyl,

wherein one or more hydrogen atoms are optionally, independently of each other substituted by deuterium, CN, CF₃, or F;

C₆-C₁₈-aryl,

wherein one or more hydrogen atoms are optionally, independently of each other substituted by deuterium, C₁-C₅-alkyl, Ph or CN;

C₃-C₁₅-heteroaryl,

wherein one or more hydrogen atoms are optionally, independently of each other substituted by deuterium, Ph or C₁-C₅-alkyl;

N(C₆-C₁₈-aryl)₂;

N(C₃-C₁₇-heteroaryl)₂, and

N(C₃-C₁₇-heteroaryl)(C₆-C₁₈-aryl).

R⁵ is at each occurrence independently of each other selected from the group consisting of: hydrogen, deuterium, Oph, CF₃, F,

C₁-C₅-alkyl,

wherein one or more hydrogen atoms are optionally, independently of each other substituted by deuterium, CN, CF₃, or F;

C₁-C₅-alkoxy,

wherein one or more hydrogen atoms are optionally, independently of each other substituted by deuterium, CN, CF₃, or F;

C₁-C₅-thioalkoxy,

wherein one or more hydrogen atoms are optionally, independently of each other substituted by deuterium, CN, CF₃, or F;

C₂-C₅-alkenyl,

wherein one or more hydrogen atoms are optionally, independently of each other substituted by deuterium, CN, CF₃, or F;

C₂-C₅-alkynyl,

wherein one or more hydrogen atoms are optionally, independently of each other substituted by deuterium, CN, CF₃, or F;

C₅-C₁₈-aryl,

wherein one or more hydrogen atoms are optionally, independently of each other substituted by deuterium, C₁-C₅-alkyl, Ph or CN;

C₃-C₁₅-heteroaryl,

wherein one or more hydrogen atoms are optionally, independently of each other substituted by deuterium, Ph or C₁-C₅-alkyl;

N(C₆-C₁₈-aryl)₂;

N(C₃-C₁₇-heteroaryl)₂, and

N(C₃-C₁₇-heteroaryl)(C₆-C₁₈-aryl).

According to embodiments of the present disclosure, exactly one substituent selected from the group consisting of T, W, X, and Y is R^{X}, and exactly one substituent selected from the group consisting of T, V, and W represents the binding site of a single bond linking the first chemical moiety and the second chemical moiety.

Furthermore, according to embodiments of the present disclosure, W is hydrogen (H), if T is R^{X} and V is the binding site of a single bond linking the first chemical moiety and the second chemical moiety.

Formula I-a with R¹ being

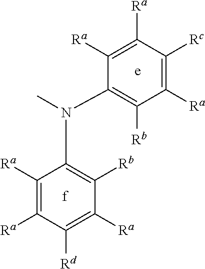

leads to formula I-b.

In certain embodiments of the present disclosure, T is the binding site of a single bond linking the first chemical moiety and the second chemical moiety and W is R^{X}.

In embodiments of the present disclosure, T is the binding site of a single bond linking the first chemical moiety and the second chemical moiety and X is R^{X}.

In certain embodiments of the present disclosure, T is the binding site of a single bond linking the first chemical moiety and the second chemical moiety and Y is R^{X}.

In certain embodiments of the present disclosure, V is the binding site of a single bond linking the first chemical moiety and the second chemical moiety and T is R^{X}.

In certain embodiments of the present disclosure, V is the binding site of a single bond linking the first chemical moiety and the second chemical moiety and W is R^{X}.

In certain embodiments of the present disclosure, V is the binding site of a single bond linking the first chemical moiety and the second chemical moiety and X is R^{X}.

In certain embodiments of the present disclosure, V is the binding site of a single bond linking the first chemical moiety and the second chemical moiety and Y is R^{X}.

In certain embodiments of the present disclosure, W is the binding site of a single bond linking the first chemical moiety and the second chemical moiety and T is R^{X}.

In embodiments of the present disclosure, W is the binding site of a single bond linking the first chemical moiety and the second chemical moiety and X is R^{X}.

In certain embodiments of the present disclosure, W is the binding site of a single bond linking the first chemical moiety and the second chemical moiety and Y is R^{X}.

In certain embodiments of the present disclosure, R^{X} comprises or consists of a structure of formula BN-I.

In certain embodiments of the present disclosure, R^{X} comprises or consists of a structure of formula BN-I-a.

In certain embodiments of the present disclosure, R^{X} comprises or consists of a structure of formula BN-I-b.

In certain embodiments of the present disclosure, R^{X} comprises or consists of a structure of formula BN-I-c.

In certain embodiments of the present disclosure, R^{X} is CF₃.

In embodiments of the present disclosure, R^{X} is CN.

In one embodiment of the present disclosure, in the first chemical moiety,

R¹ is selected from the group consisting of: hydrogen, deuterium, OR³, Si(R³)₃, CF₃, CN,

C₁-C₅-alkyl,

which is optionally substituted with one or more substituents R³;

C₆-C₁₈-aryl,

which is optionally substituted with one or more substituents R³;

R³ is at each occurrence independently of each other selected from the group consisting of: hydrogen, deuterium, N(R⁴)₂, OR⁴, Si(R⁴)₃, CF₃, CN, F,

C₁-C₅-alkyl,

which is optionally substituted with one or more substituents R⁴;

C₆-C₁₈-aryl,

which is optionally substituted with one or more substituents R⁴; and

C₃-C₁₅-heteroaryl,

which is optionally substituted with one or more substituents R⁴;

R² is at each occurrence independently of each other selected from the group consisting of:

hydrogen, deuterium,

C₁-C₅-alkyl,

wherein one or more hydrogen atoms are optionally substituted by deuterium;

C₆-C₁₈-aryl;

wherein one or more hydrogen atoms are optionally substituted by a group R⁵;

wherein the two moieties R^{b} comprised in the first chemical moiety optionally combine and together form a group Y, which is selected from the group consisting of a direct bond, CR⁶R⁷, C═CR⁶R⁷, C═O, C═NR⁶, NR⁶, O, SiR⁶R⁷, S, S(O) and S(O)₂;

R^{a}, R^{b}, R^{c}, R^{d}, R⁶, and R⁷ are at each occurrence independently of each other selected from the group consisting of: hydrogen, deuterium, N(R⁸)₂, OR⁸, Si(R⁸)₃, F, CF₃, CN,

C₁-C₅-alkyl,

which is optionally substituted with one or more substituents R⁸ and

C₆-C₁₈-aryl,

which is optionally substituted with one or more substituents R⁸; and

C₃-C₁₅-heteroaryl,

which is optionally substituted with one or more substituents R⁸;

R⁸ is at each occurrence independently of each other selected from the group consisting of: hydrogen, deuterium, N(R⁹)₂, OR⁹, Si(R⁹)₃, CF₃, CN, F,

C₁-C₅-alkyl,

which is optionally substituted with one or more substituents R⁹ and

C₆-C₁₈-aryl,

which is optionally substituted with one or more substituents R⁹; and

C₃-C₁₅-heteroaryl,

which is optionally substituted with one or more substituents R⁹;

wherein, optionally, any of the substituents R^{a}, R^{b}, R^{c}, R^{d}, R⁶, R⁷, and R⁸ independently of each other form a mono- or polycyclic, aliphatic or aromatic, carbo- or heterocyclic and/or benzo-fused ring or ring system with one or more adjacent substituents selected from R^{a}, R^{b}, R^{c}, R^{d}, R⁶, R⁷, and R⁸; wherein the optionally so formed fused ring system constructed from the benzene ring a, b, c, d, e or f of formula I-a or I-b and the additional rings formed by adjacent substituents comprises in total 9 to 30 ring atoms, out of which 1 to 3 atoms may be heteroatoms independently of each other selected from nitrogen (N), oxygen (O), and sulfur (S); and wherein the optionally so formed additional rings may optionally be substituted with one or more substituents R¹⁰;

R⁴, R⁹, and R¹⁰ are at each occurrence independently of each other selected from the group consisting of: hydrogen, deuterium, Oph, CF₃, CN, F, N(Ph)₂,

C₁-C₅-alkyl,

wherein one or more hydrogen atoms are optionally, independently of each other substituted by deuterium;

C₆-C₁₈-aryl,

wherein one or more hydrogen atoms are optionally, independently of each other substituted by deuterium, C₁-C₅-alkyl, Ph or CN;

C₃-C₁₅-heteroaryl,

wherein one or more hydrogen atoms are optionally, independently of each other substituted by deuterium, C₁-C₅-alkyl, Ph or CN;

R⁵ is at each occurrence independently of each other selected from the group consisting of: hydrogen, deuterium, Oph, CF₃, F, N(Ph)₂,

C₁-C₅-alkyl,

wherein one or more hydrogen atoms are optionally, independently of each other substituted by deuterium;

C₆-C₁₈-aryl,

wherein one or more hydrogen atoms are optionally, independently of each other substituted by deuterium, C₁-C₅-alkyl, Ph or CN;

C₃-C₁₅-heteroaryl,

wherein one or more hydrogen atoms are optionally, independently of each other substituted by deuterium, C₁-C₅-alkyl, Ph or CN.

In an embodiment of the present disclosure, in the first chemical moiety,

R¹ is selected from the group consisting of: hydrogen, deuterium,

C₁-C₅-alkyl,

which is optionally substituted with one or more substituents R³;

C₆-C₁₈-aryl,

which is optionally substituted with one or more substituents R³;

R³ is at each occurrence independently of each other selected from the group consisting of: hydrogen, deuterium, N(Ph)₂, Oph, Si(Me)₃, Si(Ph)₃, CF₃, CN, F,

C₁-C₅-alkyl,

which is optionally substituted with one or more substituents R⁴;

C₆-C₁₈-aryl,

which is optionally substituted with one or more substituents R⁴; and

C₃-C₁₅-heteroaryl,

which is optionally substituted with one or more substituents R⁴;

R² is at each occurrence independently of each other selected from the group consisting of:

hydrogen, deuterium,

C₁-C₅-alkyl,

wherein one or more hydrogen atoms are optionally substituted by deuterium;

C₆-C₁₈-aryl;

wherein one or more hydrogen atoms are optionally substituted by a group R⁵;

wherein the two moieties R^{b} comprised in the first chemical moiety optionally combine and together form a group Y, which is selected from the group consisting of a direct bond, C═O, NR⁶, O, SiR⁶R⁷, S, S(O) and S(O)₂;

R^{a}, R^{b}, R^{c}, R^{d}, R⁶, and R⁷ are at each occurrence independently of each other selected from the group consisting of: hydrogen, deuterium, Oph, Si(Me)₃, Si(Ph)₃, N(Ph)₂, CF₃, CN,

C₁-C₅-alkyl,

which is optionally substituted with one or more substituents R⁸ and

C₆-C₁₈-aryl,

which is optionally substituted with one or more substituents R⁸; and

C₃-C₁₅-heteroaryl,

which is optionally substituted with one or more substituents R⁸;

R⁸ is at each occurrence independently of each other selected from the group consisting of: hydrogen, deuterium, Oph, Si(Me)₃, Si(Ph)₃, CF₃, CN, F,

C₁-C₅-alkyl,

which is optionally substituted with one or more substituents R⁹ and

C₆-C₁₈-aryl,

which is optionally substituted with one or more substituents R⁹; and

C₃-C₁₅-heteroaryl,

which is optionally substituted with one or more substituents R⁹;

wherein, optionally, any of the substituents R^{a}, R^{b}, R^{c}, R^{d}, R⁶, R⁷, and R⁸ independently of each other form a mono- or polycyclic, aliphatic or aromatic, carbo- or heterocyclic and/or benzo-fused ring or ring system with one or more adjacent substituents selected from R^{a}, R^{b}, R^{c}, R^{d}, R⁶, R⁷, and R⁸; wherein the optionally so formed fused ring system constructed from the respective benzene ring a, b, c, d, e or f of formula I-a or I-b and the additional rings formed by adjacent substituents comprises in total 9 to 30 ring atoms, of which 1 to 3 atoms may be heteroatoms independently of each other selected from nitrogen (N), oxygen (O), and sulfur (S); and wherein the optionally so formed additional rings may optionally be substituted with one or more substituents R¹⁰;

R⁴, R⁹, and R¹⁰ are at each occurrence independently of each other selected from the group consisting of: hydrogen, deuterium, Oph, CF₃, CN, F, N(Ph)₂,

C₁-C₅-alkyl,

wherein one or more hydrogen atoms are optionally, independently of each other substituted by deuterium;

C₆-C₁₈-aryl,

wherein one or more hydrogen atoms are optionally, independently of each other substituted by deuterium, C₁-C₅-alkyl, Ph or CN;

C₃-C₁₅-heteroaryl,

wherein one or more hydrogen atoms are optionally, independently of each other substituted by deuterium, C₁-C₅-alkyl, Ph or CN;

R⁵ is at each occurrence independently of each other selected from the group consisting of: hydrogen, deuterium, Oph, CF₃, F, N(Ph)₂,

C₁-C₅-alkyl,

wherein one or more hydrogen atoms are optionally, independently of each other substituted by deuterium;

C₆-C₁₈-aryl,

wherein one or more hydrogen atoms are optionally, independently of each other substituted by deuterium, Me, ⁱPr, ^{t}Bu, Ph or CN;

C₃-C₁₅-heteroaryl,

wherein one or more hydrogen atoms are optionally, independently of each other substituted by deuterium, Me, ⁱPr, ^{t}Bu, Ph or CN.

In an embodiment of the present disclosure, in the first chemical moiety,

R¹ is selected from the group consisting of: hydrogen, deuterium,

C₁-C₅-alkyl,

which is optionally substituted with one or more substituents R³;

C₆-C₁₈-aryl,

which is optionally substituted with one or more substituents R³;

R³ is at each occurrence independently of each other selected from the group consisting of: hydrogen, deuterium, N(Ph)₂, Oph, Si(Me)₃, Si(Ph)₃, CF₃, CN, F,

C₁-C₅-alkyl,

wherein one or more hydrogen atoms are optionally, independently of each other substituted by deuterium;

C₆-C₁₈-aryl,

wherein one or more hydrogen atoms are optionally, independently of each other substituted by deuterium, Me, ⁱPr, ^{t}Bu, Ph or CN;

R² is at each occurrence independently of each other selected from the group consisting of:

hydrogen, deuterium,

C₁-C₅-alkyl,

wherein one or more hydrogen atoms are optionally substituted by deuterium;

C₆-C₁₈-aryl;

wherein one or more hydrogen atoms are optionally substituted by deuterium, Me, ⁱPr, ^{t}Bu or Ph;

wherein the two moieties R^{b} comprised in the first chemical moiety optionally combine and together form a group Y, which is at each occurrence a direct bond;

R^{a}, R^{b}, R^{c}, and R^{d} are at each occurrence independently of each other selected from the group consisting of: hydrogen, deuterium, CN, CF₃, N(Ph)₂,

C₁-C₅-alkyl,

wherein one or more hydrogen atoms are optionally, independently of each other substituted by deuterium, CN, CF₃, F or Ph;

C₆-C₁₈-aryl,

wherein one or more hydrogen atoms are optionally, independently of each other substituted by deuterium, Me, ⁱPr, ^{t}Bu, CF₃, CN or Ph;

pyridinyl, wherein one or more hydrogen atoms are optionally, independently of each other substituted by deuterium, Me, ⁱPr, ^{t}Bu, CF₃, CN or Ph;

pyrimidinyl, wherein one or more hydrogen atoms are optionally, independently of each other substituted by deuterium, Me, ⁱPr, ^{t}Bu, CF₃, CN or Ph;

carbazolyl, wherein one or more hydrogen atoms are optionally, independently of each other substituted by deuterium, Me, ⁱPr, ^{t}Bu, CF₃, CN or Ph;

triazinyl, wherein one or more hydrogen atoms are optionally, independently of each other substituted by deuterium, Me, ⁱPr, ^{t}Bu, CF₃, CN or Ph;

wherein, optionally, any of the substituents R^{a}, R^{b}, R^{c}, and R^{d} independently of each other form a mono- or polycyclic, aliphatic or aromatic, carbo- or heterocyclic and/or benzo-fused ring or ring system with one or more adjacent substituents selected from R^{a}, R^{b}, R^{c}, and R^{d}; wherein the optionally so formed fused ring system constructed from the respective benzene ring a, b, c, d, e or f of formula I-a or I-b and the additional rings formed by adjacent substituents comprises in total 9 to 30 ring atoms, of which 1 to 3 atoms may be heteroatoms independently of each other selected from nitrogen (N), oxygen (O), and sulfur (S); and wherein the optionally so formed additional rings may optionally be substituted with one or more substituents R¹⁰;

R¹⁰ is at each occurrence independently of each other selected from the group consisting of: hydrogen, deuterium, Oph, CF₃, CN, F, N(Ph)₂, Me, ⁱPr, ^{t}Bu,

C₆-C₁₈-aryl,

wherein one or more hydrogen atoms are optionally, independently of each other substituted by deuterium, Me, ⁱPr, ^{t}Bu or Ph.

In an embodiment of the present disclosure, in the first chemical moiety,

R¹ is selected from the group consisting of: hydrogen, deuterium,

C₁-C₅-alkyl,

which is optionally substituted with one or more substituents R³;

C₆-C₁₈-aryl,

which is optionally substituted with one or more substituents R³;

R³ is at each occurrence independently of each other selected from the group consisting of: hydrogen, deuterium, N(Ph)₂, Oph, Si(Me)₃, Si(Ph)₃, CF₃, CN, F,

C₁-C₅-alkyl,

wherein one or more hydrogen atoms are optionally, independently of each other substituted by deuterium;

C₆-C₁₈-aryl,

wherein one or more hydrogen atoms are optionally, independently of each other substituted by deuterium, Me, ⁱPr, ^{t}Bu, Ph or CN;

R² is at each occurrence independently of each other selected from the group consisting of:

hydrogen, deuterium,

C₁-C₅-alkyl,

wherein one or more hydrogen atoms are optionally substituted by deuterium;

C₆-C₁₈-aryl;

wherein one or more hydrogen atoms are optionally substituted by deuterium, Me, ⁱPr, ^{t}Bu or Ph;

wherein the two moieties R^{b} comprised in the first chemical moiety optionally combine and together form a group Y, which is at each occurrence a direct bond;

R^{a}, R^{b}, R^{c}, and R^{d} are at each occurrence independently of each other selected from the group consisting of: hydrogen, deuterium, CN, CF₃, N(Ph)₂,

C₁-C₅-alkyl,

wherein one or more hydrogen atoms are optionally, independently of each other substituted by deuterium, CN, CF₃, F or Ph;

Ph, wherein one or more hydrogen atoms are optionally, independently of each other substituted by deuterium, Me, ⁱPr, ^{t}Bu, CF₃, CN or Ph;

carbazolyl, wherein one or more hydrogen atoms are optionally, independently of each other substituted by deuterium, Me, ⁱPr, ^{t}Bu, CF₃, CN or Ph;

triazinyl, wherein one or more hydrogen atoms are optionally, independently of each other substituted by deuterium, Me, ⁱPr, ^{t}Bu, CF₃, CN or Ph;

wherein, optionally, any of the substituents R^{a}, R^{b}, R^{c}, and R^{d} independently of each other form a mono- or polycyclic, aliphatic or aromatic, carbo- or heterocyclic and/or benzo-fused ring or ring system with one or more adjacent substituents selected from R^{a}, R^{b}, R^{c}, and R^{d}; wherein the optionally so formed fused ring system constructed from the respective benzene ring a, b, c, d, e or f of formula I-a or I-b and the additional rings formed by adjacent substituents comprises in total 9 to 30 ring atoms, of which 1 to 3 atoms may be heteroatoms independently of each other selected from nitrogen (N), oxygen (O), and sulfur (S); and wherein the optionally so formed additional rings may optionally be substituted with one or more substituents R¹⁰;

R¹⁰ is at each occurrence independently of each other selected from the group consisting of: hydrogen, deuterium, Oph, CF₃, CN, F, N(Ph)₂, Me, ⁱPr, ^{t}Bu,

Ph, wherein one or more hydrogen atoms are optionally, independently of each other substituted by deuterium, Me, ⁱPr, ^{t}Bu or Ph.

In an embodiment of the present disclosure, in the first chemical moiety,

R¹ is selected from the group consisting of: hydrogen, deuterium, Me, ⁱPr, ^{t}Bu, and

Ph, which is optionally substituted with one or more substituents R³;

R³ is at each occurrence independently of each other selected from the group consisting of: hydrogen, deuterium, CF₃, CN, Me, ⁱPr, ^{t}Bu, and

Ph, wherein one or more hydrogen atoms are optionally, independently of each other substituted by deuterium, Me, ⁱPr, ^{t}Bu, Ph or CN;

R² is at each occurrence independently of each other selected from the group consisting of:

hydrogen, deuterium, Me, ⁱPr, ^{t}Bu, and

Ph, wherein one or more hydrogen atoms are optionally substituted by deuterium, Me, ⁱPr, ^{t}Bu or Ph;

wherein the two moieties R^{b} comprised in the first chemical moiety optionally combine and together form a group Y, which is at each occurrence a direct bond;

R^{a}, R^{b}, R^{c}, and R^{d} are at each occurrence independently of each other selected from the group consisting of: hydrogen, deuterium, CN, CF₃, N(Ph)₂, Me, ⁱPr, ^{t}Bu,

Ph, wherein one or more hydrogen atoms are optionally, independently of each other substituted by deuterium, Me, ⁱPr, ^{t}Bu, CF₃, CN or Ph;

carbazolyl, wherein one or more hydrogen atoms are optionally, independently of each other substituted by deuterium, Me, ⁱPr, ^{t}Bu, CF₃, CN or Ph;

wherein, optionally, any of the substituents R^{a}, R^{b}, R^{c}, and R^{d} independently of each other form a mono- or polycyclic, aliphatic or aromatic, carbo- or heterocyclic and/or benzo-fused ring or ring system with one or more adjacent substituents selected from R^{a}, R^{b}, R^{c}, and R^{d}; wherein the optionally so formed fused ring system constructed from the respective benzene ring a, b, c, d, e or f of formula I-a or I-b and the additional rings formed by adjacent substituents comprises in total 9 to 30 ring atoms, out of which 1 to 3 atoms may be heteroatoms independently of each other selected from nitrogen (N), oxygen (O), and sulfur (S); and wherein the optionally so formed additional rings may optionally be substituted with one or more substituents R¹⁰; and

R¹⁰ is at each occurrence independently of each other selected from the group consisting of: hydrogen, deuterium, CF₃, CN, Me, ⁱPr, ^{t}Bu,

Ph, wherein one or more hydrogen atoms are optionally, independently of each other substituted by deuterium, Me, ⁱPr, ^{t}Bu or Ph.

In an embodiment of the present disclosure, in the first chemical moiety,

R¹ is selected from the group consisting of: hydrogen, deuterium, Me, ⁱPr, ^{t}Bu, and

Ph, wherein one or more hydrogen atoms are optionally, independently of each other substituted by deuterium, Me, ⁱPr, ^{t}Bu, Ph or CN;

R² is at each occurrence independently of each other selected from the group consisting of:

hydrogen, deuterium, Me, ⁱPr, ^{t}Bu, and

Ph, wherein one or more hydrogen atoms are optionally substituted by deuterium, Me, ⁱPr, ^{t}Bu or Ph;

wherein the two moieties R^{b} comprised in the first chemical moiety optionally combine and together form a group Y, which is at each occurrence a direct bond;

R^{a}, R^{b}, R^{c}, and R^{d} are at each occurrence independently of each other selected from the group consisting of: hydrogen, deuterium, CN, Me, ⁱPr, ^{t}Bu,

Ph, wherein one or more hydrogen atoms are optionally, independently of each other substituted by deuterium, Me, ⁱPr, ^{t}Bu, CN or Ph;

wherein, optionally, any of the substituents R^{a}, R^{b}, R^{c}, and R^{d} independently of each other form a mono- or polycyclic, aliphatic or aromatic, carbo- or heterocyclic and/or benzo-fused ring or ring system with one or more adjacent substituents selected from R^{a}, R^{b}, R^{c}, and R^{d}; wherein the optionally so formed fused ring system constructed from the respective benzene ring a, b, c, d, e or f of formula I-a or I-b and the additional rings formed by adjacent substituents comprises in total 9 to 30 ring atoms, out of which 1 to 3 atoms may be heteroatoms independently of each other selected from nitrogen (N), oxygen (O), and sulfur (S); and wherein the optionally so formed additional rings may optionally be substituted with one or more substituents R¹⁰; and

R¹⁰ is at each occurrence independently of each other selected from the group consisting of: hydrogen, deuterium, CN, Me, ⁱPr, ^{t}Bu,

Ph, wherein one or more hydrogen atoms are optionally, independently of each other substituted by deuterium, Me, ⁱPr, ^{t}Bu or Ph.

In an embodiment of the present disclosure, in the first chemical moiety,

R¹ is selected from the group consisting of: hydrogen, deuterium, Me, ⁱPr, ^{t}Bu, and

Ph, wherein one or more hydrogen atoms are optionally, independently of each other substituted by deuterium, Me, ⁱPr, ^{t}Bu, Ph or CN;

R² is at each occurrence independently of each other selected from hydrogen and deuterium;

wherein the two moieties R^{b} comprised in the first chemical moiety optionally combine and together form a group Y, which is at each occurrence a direct bond;

R^{a}, R^{b}, R^{c}, and R^{d} are at each occurrence independently of each other selected from the group consisting of: hydrogen, deuterium, CN, Me, ⁱPr, ^{t}Bu,

Ph, wherein one or more hydrogen atoms are optionally, independently of each other substituted by deuterium, Me, ⁱPr, ^{t}Bu, CN or Ph;

wherein, optionally, any of the substituents R^{a}, R^{b}, R^{c}, and R^{d} independently of each other form a mono- or polycyclic, aliphatic or aromatic, carbo- or heterocyclic and/or benzo-fused ring or ring system with one or more adjacent substituents selected from R^{a}, R^{b}, R^{c}, and R^{d}; wherein the optionally so formed fused ring system constructed from the respective benzene ring a, b, c, d, e or f of formula I-a or I-b and the additional rings formed by adjacent substituents comprises in total 9 to 30 ring atoms, of which 1 to 3 atoms may be heteroatoms independently of each other selected from nitrogen (N), oxygen (O), and sulfur (S); and wherein the optionally so formed additional rings may optionally be substituted with one or more substituents R¹⁰; and

R¹⁰ is at each occurrence independently of each other selected from the group consisting of: hydrogen, deuterium, Me, ⁱPr, ^{t}Bu,

Ph, wherein one or more hydrogen atoms are optionally, independently of each other substituted by deuterium, Me, ⁱPr, ^{t}Bu or Ph.

In one embodiment of the present disclosure, none of the substituents selected from R^{a}, R^{b}, R^{c}, R^{d}, R⁶, R⁷, and R⁸ form an additional ring or ring system with any adjacent substituents selected from R^{a}, R^{b}, R^{c}, R^{d}, R⁶, R⁷, and R⁸.

In one embodiment of the present disclosure, R^{a}, R^{b}, R^{c}, and R^{d} are at each occurrence hydrogen.

In one embodiment of the present disclosure, R^{a}, R^{c}, and R^{d} are at each occurrence hydrogen and the two groups R^{b} combine and together form a group Y, which is at each occurrence a direct bond.

In one embodiment of the present disclosure, the first chemical moiety comprises or consists of a structure according to any of formulas I-a-1, I-b-1, I-a-2, and I-b-2:

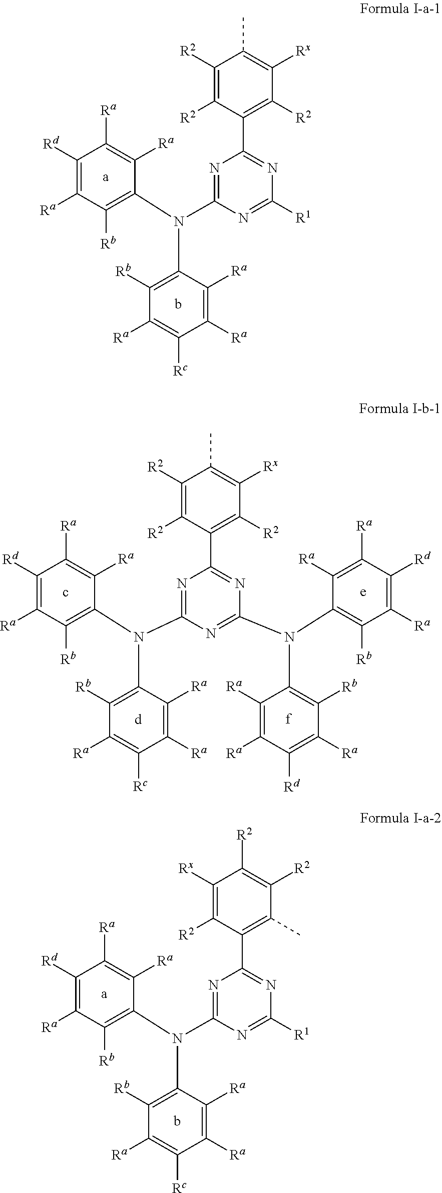

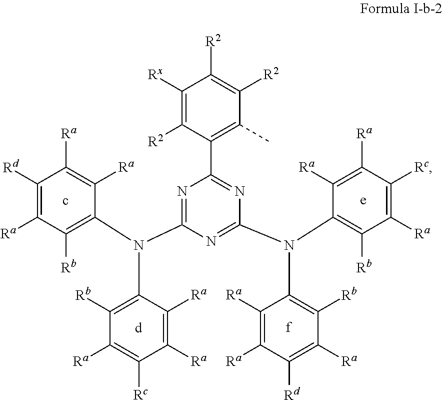

wherein the dashed line indicates the single bond linking the first chemical moiety and the second chemical moiety, and wherein apart from that the aforementioned definitions apply.

In one embodiment of the present disclosure, the first chemical moiety comprises or consists of a structure according to any of formulas I-a-1 and I-a-2, wherein the aforementioned definitions apply.

In one embodiment of the present disclosure, the first chemical moiety comprises or consists of a structure according to any of formulas I-b-1 and I-b-2, wherein the aforementioned definitions apply.

In one embodiment of the present disclosure, the first chemical moiety comprises or consists of a structure according to any of formulas I-a-1 and I-b-1, wherein the aforementioned definitions apply.

In one embodiment of the present disclosure, the first chemical moiety comprises or consists of a structure according to any of formulas I-a-2 and I-b-2, wherein the aforementioned definitions apply.

In an embodiment of the present disclosure, the first chemical moiety comprises or consists of a structure according to any of formulas I-a-1-1, I-b-1-1, I-a-2-1, and I-b-2-1:

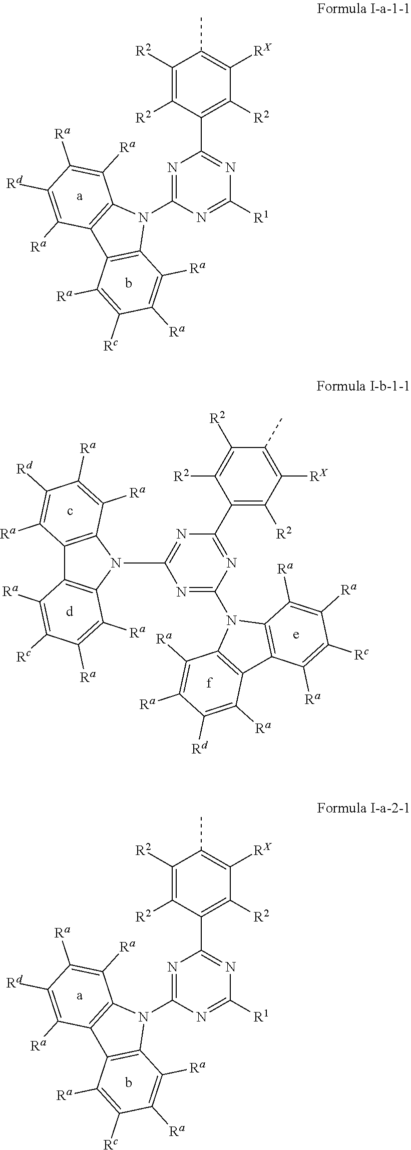

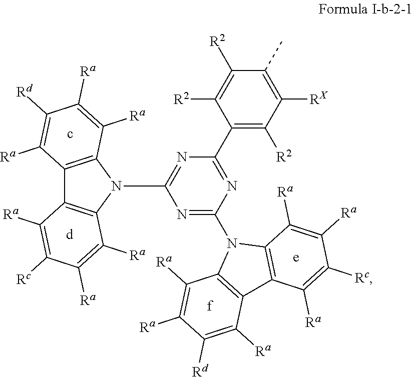

wherein the dashed line indicates the single band linking the first chemical moiety and the second chemical moiety, and wherein apart from that the aforementioned definitions apply.

In one embodiment of the present disclosure, the first chemical moiety comprises or consists of a structure according to any of formulas I-a-1-1 and I-a-2-1, wherein the aforementioned definitions apply.

In one embodiment of the present disclosure, the first chemical moiety comprises or consists of a structure according to any of formulas I-b-1-1 and I-b-2-1, wherein the aforementioned definitions apply.

In one embodiment of the present disclosure, the first chemical moiety comprises or consists of a structure according to any of formulas I-a-1-1 and I-b-1-1, wherein the aforementioned definitions apply.

In one embodiment of the present disclosure, the first chemical moiety comprises or consists of a structure according to any of formulas I-a-2-1 and I-b-2-1, wherein the aforementioned definitions apply.

In an embodiment of the present disclosure, the first chemical moiety comprises or consists of a structure according to any of formulas I-a-1-1-a, I-a-1-1-b, I-b-1-1-a, I-b-1-1-b, I-a-2-1-a, I-a-2-1-b, I-b-2-1-a, and I-b-2-1-b:

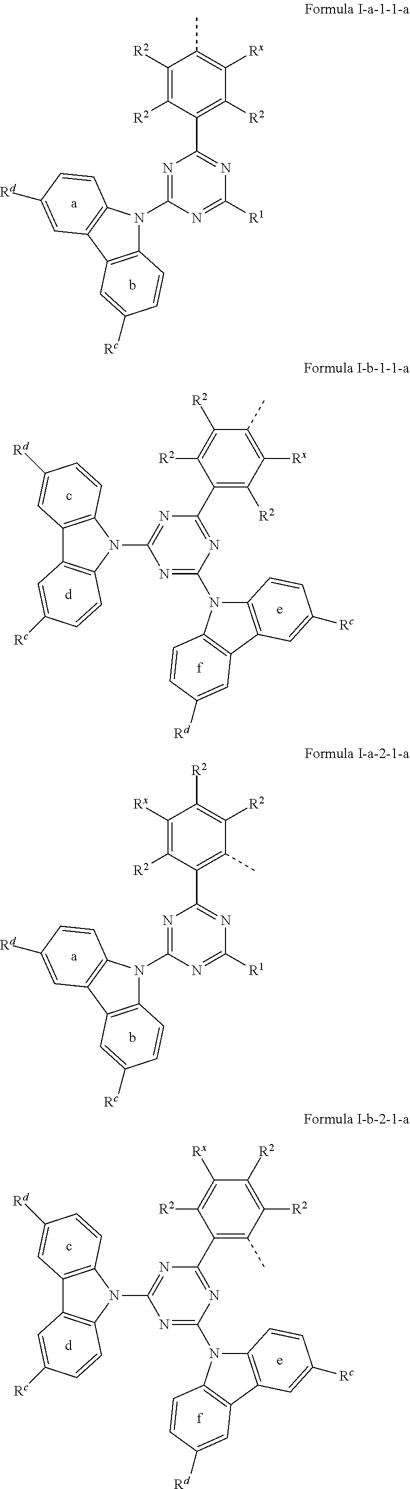

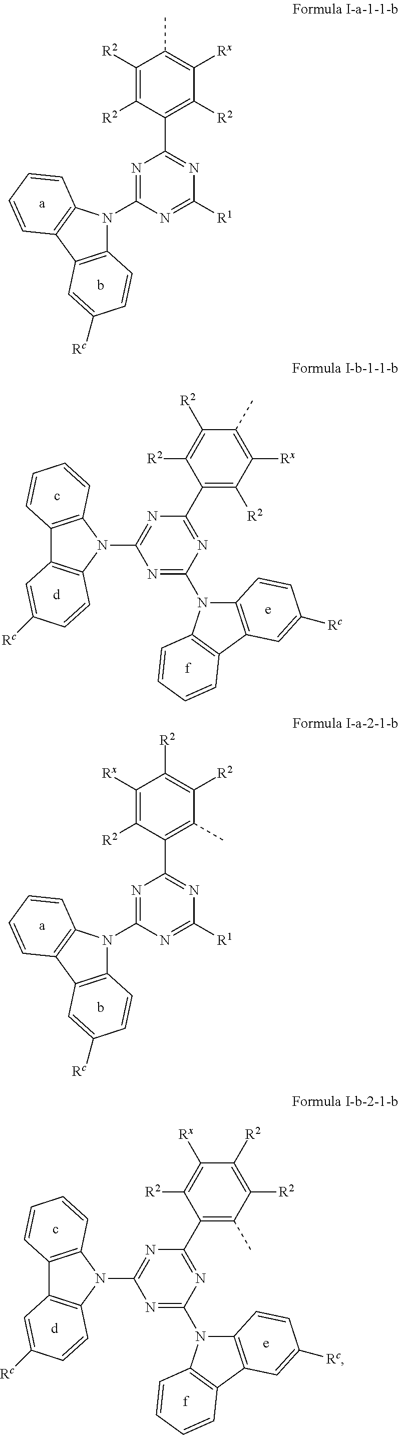

wherein the dashed line indicates the single band linking the first chemical moiety and the second chemical moiety, and wherein apart from that the aforementioned definitions apply.

In one embodiment of the present disclosure, the first chemical moiety comprises or consists of a structure according to any of formulas I-a-1-1-a and I-a-1-1-b, wherein the aforementioned definitions apply.

In one embodiment of the present disclosure, the first chemical moiety comprises or consists of a structure according to any of formulas I-b-1-1-a and I-b-1-1-b, wherein the aforementioned definitions apply.

In one embodiment of the present disclosure, the first chemical moiety comprises or consists of a structure according to any of formulas I-a-2-1-a and I-a-2-1-b, wherein the aforementioned definitions apply.

In one embodiment of the present disclosure, the first chemical moiety comprises or consists of a structure according to any of formulas I-b-2-1-a and I-b-2-1-b, wherein the aforementioned definitions apply.

In one embodiment of the present disclosure, the first chemical moiety comprises or consists of a structure according to any of formulas I-a-1-1-a and I-a-2-1-a, wherein the aforementioned definitions apply.

In one embodiment of the present disclosure, the first chemical moiety comprises or consists of a structure according to any of formulas I-b-1-1-a and I-b-2-1-a, wherein the aforementioned definitions apply.

In one embodiment of the present disclosure, the first chemical moiety comprises or consists of a structure according to any of formulas I-a-1-1-a and I-b-1-1-a, wherein the aforementioned definitions apply.

In one embodiment of the present disclosure, the first chemical moiety comprises or consists of a structure according to any of formulas I-a-2-1-a and I-b-2-1-a, wherein the aforementioned definitions apply.

In one embodiment of the present disclosure, the first chemical moiety comprises or consists of a structure according to any of formulas I-a-1-1-b and I-a-2-1-b, wherein the aforementioned definitions apply.

In one embodiment of the present disclosure, the first chemical moiety comprises or consists of a structure according to any of formulas I-b-1-1-b and I-b-2-1-b, wherein the aforementioned definitions apply.

In one embodiment of the present disclosure, the first chemical moiety comprises or consists of a structure according to any of formulas I-a-1-1-b and I-b-1-1-b, wherein the aforementioned definitions apply.

In one embodiment of the present disclosure, the first chemical moiety comprises or consists of a structure according to any of formulas I-a-2-1-b and I-b-2-1-b, wherein the aforementioned definitions apply.

In an embodiment of the present disclosure, the first chemical moiety comprises or consists of a structure according to any of formulas I-a-1-1-a, I-b-1-1-a, I-a-1-1-b, and I-b-1-1-b, wherein the aforementioned definitions apply.

In an embodiment of the present disclosure, the first chemical moiety comprises or consists of a structure according to any of formulas I-a-1-2-a, I-b-1-2-a, I-a-2-1-b, and I-b-2-1-b, wherein the aforementioned definitions apply.

In one embodiment of the present disclosure, the first chemical moiety comprises or consists of a structure according to any of formulas I-a-1-1-a, I-a-1-1-b, I-a-2-1-a, and I-a-2-1-b, wherein the aforementioned definitions apply.

In one embodiment of the present disclosure, the first chemical moiety comprises or consists of a structure according to any of formulas I-b-1-1-a, I-b-1-1-b, I-b-2-1-a, and I-b-2-1-b, wherein the aforementioned definitions apply.

In one embodiment of the present disclosure, the first chemical moiety comprises or consists of a structure according to any of formulas I-a-1-1-a, I-b-1-1-a, I-a-2-1-a, and I-b-2-1-a, wherein the aforementioned definitions apply.

In one embodiment of the present disclosure, the first chemical moiety comprises or consists of a structure according to any of formulas I-a-1-1-b, I-b-1-1-b, I-a-2-1-b, and I-b-2-1-b, wherein the aforementioned definitions apply.

In one embodiment of the present disclosure, R^{e}, R^{f}, R^{g}, R¹¹, and R¹² of the second chemical moiety are at each occurrence independently of each other selected from the group consisting of: hydrogen, deuterium, N(R¹³)₂, OR¹³, Si(R¹³)₃, F, CF₃, CN,

C₁-C₅-alkyl,

which is optionally substituted with one or more substituents R¹³ and

C₆-C₁₈-aryl,

which is optionally substituted with one or more substituents R¹³; and

C₃-C₁₅-heteroaryl,

which is optionally substituted with one or more substituents R¹³;

R¹³ is at each occurrence independently of each other selected from the group consisting of: hydrogen, deuterium, N(R¹⁴)₂, OR¹⁴, Si(R¹⁴)₃, CF₃, CN, F,

C₁-C₅-alkyl,

which is optionally substituted with one or more substituents R¹⁴ and

C₆-C₁₈-aryl,

which is optionally substituted with one or more substituents R¹⁴; and

C₃-C₁₅-heteroaryl,

which is optionally substituted with one or more substituents R¹⁴;

wherein, optionally, any of the substituents R^{e}, R^{f}, R^{g}, R¹¹, R¹², and R¹³ independently of each other form a mono- or polycyclic, aliphatic or aromatic, carbo- or heterocyclic and/or benzo-fused ring or ring system with one or more adjacent substituents selected from R^{e}, R^{f}, R^{g}, R¹¹, R¹², and R¹³; wherein the optionally so formed fused ring system constructed from the structure according to formula II (counting as 13 or 14 ring atoms in total, depending on the nature of Z) and the additional rings formed by adjacent substituents comprises in total 16 to 30 ring atoms, out of which 1 to 3 atoms may be heteroatoms independently of each other selected from nitrogen (N), oxygen (O), and sulfur (S); and wherein the optionally so formed additional rings may optionally be substituted with one or more substituents R¹⁵;

R¹⁴, and R¹⁵ are at each occurrence independently of each other selected from the group consisting of: hydrogen, deuterium, Oph, CF₃, CN, F, N(Ph)₂,

C₁-C₅-alkyl,

wherein one or more hydrogen atoms are optionally, independently of each other substituted by deuterium;

C₆-C₁₈-aryl,

wherein one or more hydrogen atoms are optionally, independently of each other substituted by deuterium, C₁-C₅-alkyl, Ph or CN;

C₃-C₁₅-heteroaryl,

wherein one or more hydrogen atoms are optionally, independently of each other substituted by deuterium, C₁-C₅-alkyl, Ph or CN.

In one embodiment of the present disclosure, R^{e}, R^{f}, R^{g}, R¹¹, and R¹² of the second chemical moiety are at each occurrence independently of each other selected from the group consisting of: hydrogen, deuterium, N(R⁸)₂, OR⁸, Si(R⁸)₃, F, CF₃, CN,

C₁-C₅-alkyl,

which is optionally substituted with one or more substituents R¹³ and

C₆-C₁₈-aryl,

which is optionally substituted with one or more substituents R¹³; and

C₃-C₁₅-heteroaryl,

which is optionally substituted with one or more substituents R¹³;

R¹³ is at each occurrence independently of each other selected from the group consisting of: hydrogen, deuterium, Oph, CF₃, CN, F, N(Ph)₂,

C₁-C₅-alkyl,

wherein one or more hydrogen atoms are optionally, independently of each other substituted by deuterium;

C₆-C₁₈-aryl,

wherein one or more hydrogen atoms are optionally, independently of each other substituted by deuterium, C₁-C₅-alkyl, Ph or CN;

C₃-C₁₅-heteroaryl,

wherein one or more hydrogen atoms are optionally, independently of each other substituted by deuterium, C₁-C₅-alkyl, Ph or CN;

wherein, optionally, any of the substituents R^{e}, R^{f}, R^{g}, R¹¹ and R¹² independently of each other form a mono- or polycyclic, aliphatic or aromatic, carbo- or heterocyclic and/or benzo-fused ring or ring system with one or more adjacent substituents selected from R^{e}, R^{f}, R^{g}, R¹¹, and R¹²; wherein the optionally so formed fused ring system constructed from the structure according to formula II (counting as 13 or 14 ring atoms in total, depending on the nature of Z) and the additional rings formed by adjacent substituents comprises in total 16 to 30 ring atoms, out of which 1 to 3 atoms may be heteroatoms independently of each other selected from nitrogen (N), oxygen (O), and sulfur (S); and wherein the optionally so formed additional rings may optionally be substituted with one or more substituents R¹⁵;

R¹⁵ is at each occurrence independently of each other selected from the group consisting of: hydrogen, deuterium, Oph, CF₃, CN, F, N(Ph)₂,

C₁-C₅-alkyl,

wherein one or more hydrogen atoms are optionally, independently of each other substituted by deuterium;

C₆-C₁₈-aryl,

wherein one or more hydrogen atoms are optionally, independently of each other substituted by deuterium, C₁-C₅-alkyl, Ph or CN.

In one embodiment of the present disclosure, R^{e}, R^{f}, R^{g}, R¹¹, and R¹² of the second chemical moiety are at each occurrence independently of each other selected from the group consisting of: hydrogen, deuterium, N(R⁸)₂, OR⁸, Si(R⁸)₃, F, CF₃, CN,

C₁-C₅-alkyl,

which is optionally substituted with one or more substituents R¹³ and

C₆-C₁₈-aryl,

which is optionally substituted with one or more substituents R¹³; and

C₃-C₁₅-heteroaryl,

which is optionally substituted with one or more substituents R¹³;

R¹³ is at each occurrence independently of each other selected from the group consisting of: hydrogen, deuterium, Me, ⁱPr, ^{t}Bu, CF₃, CN, F, N(Ph)₂,

C₆-C₁₈-aryl,

wherein one or more hydrogen atoms are optionally, independently of each other substituted by deuterium, C₁-C₅-alkyl, Ph or CN;

wherein, optionally, any of the substituents R^{e}, R^{f}, R^{g}, R¹¹ and R¹² independently of each other form a mono- or polycyclic, aliphatic or aromatic, carbo- or heterocyclic and/or benzo-fused ring or ring system with one or more adjacent substituents selected from R^{e}, R^{f}, R^{g}, R¹¹, and R¹²; wherein the optionally so formed fused ring system constructed from the structure according to formula II (counting as 13 or 14 ring atoms in total, depending on the nature of Z) and the additional rings formed by adjacent substituents comprises in total 16 to 30 ring atoms, out of which 1 to 3 atoms may be heteroatoms independently of each other selected from nitrogen (N), oxygen (O), and sulfur (S); and wherein the optionally so formed additional rings may optionally be substituted with one or more substituents R¹⁵;

R¹⁵ is at each occurrence independently of each other selected from the group consisting of: hydrogen, deuterium, Oph, CF₃, CN, F, N(Ph)₂,

C₁-C₅-alkyl,

wherein one or more hydrogen atoms are optionally, independently of each other substituted by deuterium;

C₆-C₁₈-aryl,

wherein one or more hydrogen atoms are optionally, independently of each other substituted by deuterium, Me, ⁱPr, ^{t}Bu, Ph or CN.

In an embodiment of the present disclosure, R^{e}, R^{f}, R^{g}, R¹¹, and R¹² of the second chemical moiety are at each occurrence independently of each other selected from the group consisting of: hydrogen, deuterium, N(Ph)₂, Oph, Si(Me)₃, Si(Ph)₃, F, CF₃, CN,

C₆-C₁₈-aryl,

wherein one or more hydrogen atoms are optionally, independently of each other substituted by deuterium, Me, ⁱPr, ^{t}Bu, CF₃, CN or Ph;

C₃-C₁₅-heteroaryl,

wherein one or more hydrogen atoms are optionally, independently of each other substituted by deuterium, Me, ⁱPr, ^{t}Bu, CF₃, CN or Ph;

wherein, optionally, any of the substituents R^{e}, R^{f}, R^{g}, R¹¹ and R¹² independently of each other form a mono- or polycyclic, aliphatic or aromatic, carbo- or heterocyclic and/or benzo-fused ring or ring system with one or more adjacent substituents selected from R^{e}, R^{f}, R^{g}, R¹¹, and R¹²; wherein the optionally so formed fused ring system constructed from the structure according to formula II (counting as 13 or 14 ring atoms in total, depending on the nature of Z) and the additional rings formed by adjacent substituents comprises in total 16 to 30 ring atoms, out of which 1 to 3 atoms may be heteroatoms independently of each other selected from nitrogen (N), oxygen (O), and sulfur (S); and wherein the optionally so formed additional rings may optionally be substituted with one or more substituents R¹⁵;

R¹⁵ is at each occurrence independently of each other selected from the group consisting of: hydrogen, deuterium, CN, Me, ⁱPr, ^{t}Bu, and

Ph, wherein one or more hydrogen atoms are optionally, independently of each other substituted by deuterium, Me, ⁱPr, ^{t}Bu, Ph or CN.

In an embodiment of the present disclosure, R^{e}, R^{f}, R^{g}, R¹¹, and R¹² of the second chemical moiety are at each occurrence independently of each other selected from the group consisting of: hydrogen, deuterium, Me, ⁱPr, ^{t}Bu, CN, N(Ph)₂,

Ph; wherein one or more hydrogen atoms are optionally substituted by deuterium, Me, ⁱPr, ^{t}Bu, CN, CF₃, and Ph;

pyridinyl, wherein one or more hydrogen atoms are optionally substituted by deuterium, Me, ⁱPr, ^{t}Bu, CN, CF₃, and Ph,

pyrimidinyl, wherein one or more hydrogen atoms are optionally substituted by deuterium, Me, ⁱPr, ^{t}Bu, CN, CF₃, and Ph

carbazolyl, wherein one or more hydrogen atoms are optionally substituted by deuterium, Me, ⁱPr, ^{t}Bu, CN, CF₃, and Ph

triazinyl, wherein one or more hydrogen atoms are optionally substituted by deuterium, Me, ⁱPr, ^{t}Bu, CN, CF₃, and Ph;

wherein, optionally, any of the substituents R^{e}, R^{f}, R^{g}, R¹¹ and R¹² independently of each other form a mono- or polycyclic, aliphatic or aromatic, carbo- or heterocyclic and/or benzo-fused ring or ring system with one or more adjacent substituents selected from R^{e}, R^{f}, R^{g}, R¹¹, and R¹²; wherein the optionally so formed fused ring system constructed from the structure according to formula II (counting as 13 or 14 ring atoms in total, depending on the nature of Z) and the additional rings formed by adjacent substituents comprises in total 16 to 30 ring atoms, out of which 1 to 3 atoms may be heteroatoms independently of each other selected from nitrogen (N), oxygen (O), and sulfur (S); and wherein the optionally so formed additional rings may optionally be substituted with one or more substituents R¹⁵;

R¹⁵ is at each occurrence independently of each other selected from the group consisting of: hydrogen, deuterium, CN, Me, ⁱPr, ^{t}Bu, and

Ph, wherein one or more hydrogen atoms are optionally, independently of each other substituted by deuterium, Me, ⁱPr, ^{t}Bu, Ph or CN.

In an embodiment of the present disclosure, R^{e}, R^{f}, R^{g}, R¹¹, and R¹² of the second chemical moiety are at each occurrence independently of each other selected from the group consisting of: hydrogen, deuterium, Me, ⁱPr, ^{t}Bu, CN, CF₃, N(Ph)₂,

Ph; wherein one or more hydrogen atoms are optionally substituted by deuterium, Me, ⁱPr, ^{t}Bu, CN, CF₃, and Ph;

carbazolyl, wherein one or more hydrogen atoms are optionally substituted by deuterium, Me, ⁱPr, ^{t}Bu, CN, CF₃, and Ph;

triazinyl, wherein one or more hydrogen atoms are optionally substituted by deuterium, Me, ⁱPr, ^{t}Bu, CN, CF₃, and Ph;

wherein, optionally, any of the substituents R^{e}, R^{f}, R^{g}, R¹¹ and R¹² independently of each other form a mono- or polycyclic, aliphatic or aromatic, carbo- or heterocyclic and/or benzo-fused ring or ring system with one or more adjacent substituents selected from R^{e}, R^{f}, R^{g}, R¹¹, and R¹²; wherein the optionally so formed fused ring system constructed from the structure according to formula II (counting as 13 or 14 ring atoms in total, depending on the nature of Z) and the additional rings formed by adjacent substituents comprises in total 16 to 30 ring atoms, out of which 1 to 3 atoms may be heteroatoms independently of each other selected from nitrogen (N), oxygen (O), and sulfur (S); and wherein the optionally so formed additional rings may optionally be substituted with one or more substituents R¹⁵;

R¹⁵ is at each occurrence independently of each other selected from the group consisting of: hydrogen, deuterium, Me, ⁱPr, ^{t}Bu, and

Ph, wherein one or more hydrogen atoms are optionally, independently of each other substituted by deuterium, Me, ⁱPr, ^{t}Bu or Ph.

In an embodiment of the present disclosure, R^{e}, R^{f}, R^{g}, R¹¹, and R¹² of the second chemical moiety are at each occurrence independently of each other selected from the group consisting of: hydrogen, deuterium, Me, ⁱPr, ^{t}Bu, CN, N(Ph)₂,

Ph; wherein one or more hydrogen atoms are optionally substituted by deuterium, Me, ⁱPr, ^{t}Bu, CN, and Ph;

carbazolyl, wherein one or more hydrogen atoms are optionally substituted by deuterium, Me, ⁱPr, ^{t}Bu, CN, and Ph;

wherein, optionally, any of the substituents R^{e}, R^{f}, R^{g}, R¹¹ and R¹² independently of each other form a mono- or polycyclic, aliphatic or aromatic, carbo- or heterocyclic and/or benzo-fused ring or ring system with one or more adjacent substituents selected from R^{e}, R^{f}, R^{g}, R¹¹, and R¹²; wherein the optionally so formed fused ring system constructed from the structure according to formula II (counting as 13 or 14 ring atoms in total, depending on the nature of Z) and the additional rings formed by adjacent substituents comprises in total 16 to 30 ring atoms, out of which 1 to 3 atoms may be heteroatoms independently of each other selected from nitrogen (N), oxygen (O), and sulfur (S); and wherein the optionally so formed additional rings may optionally be substituted with one or more substituents R¹⁵;

R¹⁵ is at each occurrence independently of each other selected from the group consisting of: hydrogen, deuterium, Me, ⁱPr, ^{t}Bu, and

Ph, wherein one or more hydrogen atoms are optionally, independently of each other substituted by deuterium, Me, ⁱPr, ^{t}Bu or Ph.

In an embodiment of the present disclosure, R^{e}, R^{f}, R^{g}, R¹¹, and R¹² of the second chemical moiety are at each occurrence independently of each other selected from the group consisting of: hydrogen, deuterium, Me, ⁱPr, ^{t}Bu, CN, and

Ph; wherein one or more hydrogen atoms are optionally substituted by deuterium, Me, ⁱPr, ^{t}Bu, CN, and Ph;

wherein, optionally, any of the substituents R^{e}, R^{f}, R^{g}, R¹¹ and R¹² independently of each other form a mono- or polycyclic, aliphatic or aromatic, carbo- or heterocyclic and/or benzo-fused ring or ring system with one or more adjacent substituents selected from R^{e}, R^{f}, R^{g}, R¹¹, and R¹²; wherein the optionally so formed fused ring system constructed from the structure according to formula II (counting as 13 or 14 ring atoms in total, depending on the nature of Z) and the additional rings formed by adjacent substituents comprises in total 16 to 30 ring atoms, out of which 1 to 3 atoms may be heteroatoms independently of each other selected from nitrogen (N), oxygen (O), and sulfur (S); and wherein the optionally so formed additional rings may optionally be substituted with one or more substituents R¹⁵;

R¹⁵ is at each occurrence independently of each other selected from the group consisting of: hydrogen, deuterium, Me, ⁱPr, ^{t}Bu, and

Ph, wherein one or more hydrogen atoms are optionally, independently of each other substituted by deuterium, Me, ⁱPr, ^{t}Bu or Ph.

In some embodiments of the present disclosure, R^{e}, R^{f}, R^{g}, R¹¹, and R¹² of the second chemical moiety are at each occurrence independently of each other selected from the group consisting of: hydrogen, deuterium, Me, ⁱPr, ^{t}Bu, and

Ph; wherein one or more hydrogen atoms are optionally substituted by deuterium, Me, ⁱPr, ^{t}Bu, and Ph;

wherein, optionally, any of the substituents R^{e}, R^{f}, R^{g}, R¹¹ and R¹² independently of each other form a mono- or polycyclic, aliphatic or aromatic, carbo- or heterocyclic and/or benzo-fused ring or ring system with one or more adjacent substituents selected from R^{e}, R^{f}, R^{g}, R¹¹, and R¹²; wherein the optionally so formed fused ring system constructed from the structure according to formula II (counting as 13 or 14 ring atoms in total, depending on the nature of Z) and the additional rings formed by adjacent substituents comprises in total 16 to 30 ring atoms, out of which 1 to 3 atoms may be heteroatoms independently of each other selected from nitrogen (N), oxygen (O), and sulfur (S); and wherein the optionally so formed additional rings may optionally be substituted with one or more substituents R¹⁵;

R¹⁵ is at each occurrence independently of each other selected from the group consisting of: hydrogen, deuterium, Me, ⁱPr, ^{t}Bu, and Ph.

In one embodiment of the present disclosure, R^{e} is at each occurrence hydrogen or forms an additional ring or ring system with an adjacent substituent selected from R^{e}, R^{f}, R^{g}, R¹¹, and R¹² as stated above.

In an embodiment of the present disclosure, the second chemical moiety comprises or consists of a structure of formula II-a:

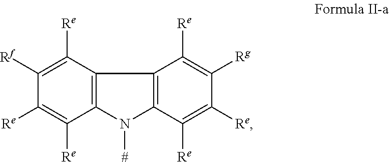

wherein the aforementioned definitions apply.

In one embodiment of the present disclosure, the second chemical moiety comprises or consists of a structure according to any of formulas II-a-1, II-a-2, II-a-3, II-a-4, II-a-5, II-a-6, II-a-7, II-a-8, II-a-9, II-a-10, II-a-11, II-a-12, II-a-13, II-a-14, and II-a-15:

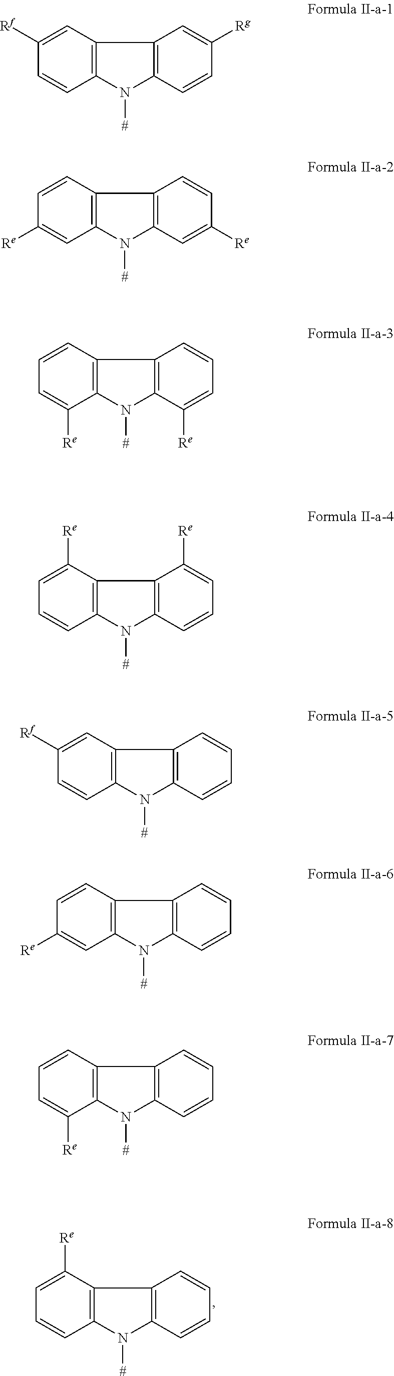

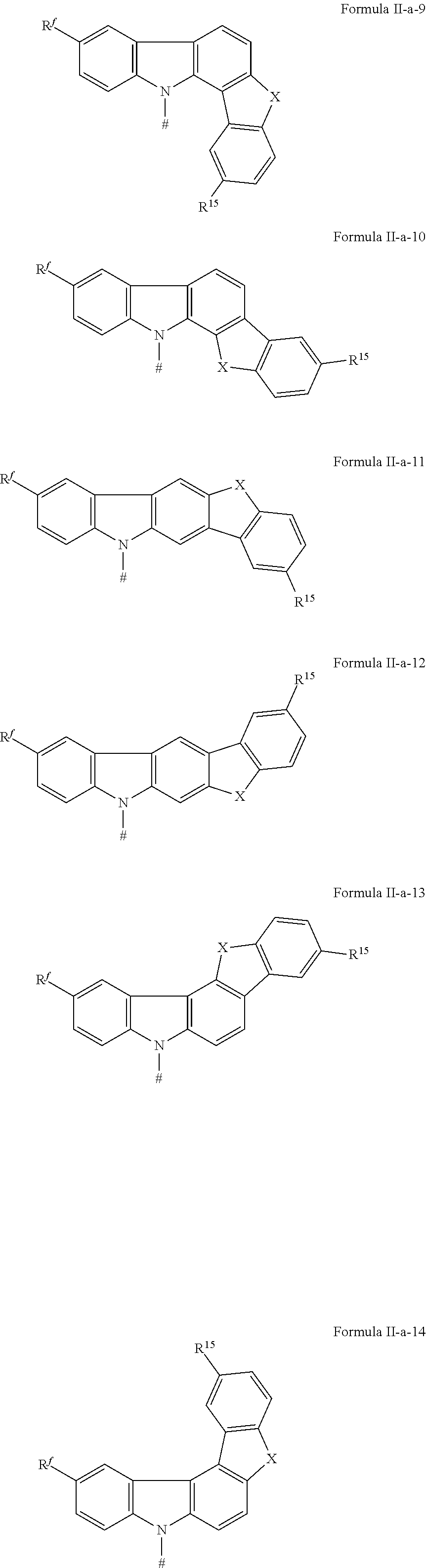

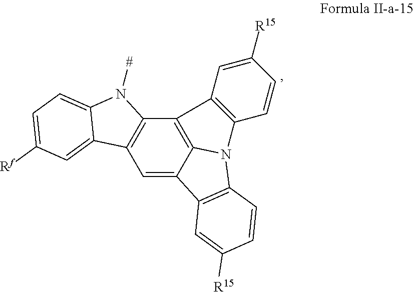

wherein X is selected from the group consisting of C(R¹⁶)₂, NR¹⁶, O, and S;

R¹⁶ is at each occurrence independently of each other selected from the group consisting of: hydrogen, deuterium, Me, ⁱPr, ^{t}Bu, and

Ph, wherein one or more hydrogen atoms are optionally substituted by deuterium, Me, ⁱPr, ^{t}Bu, and Ph.

In an embodiment of the present disclosure, the second chemical moiety comprises or consists of a structure according to any of formulas II-a-1, II-a-5, II-a-9, II-a-10, II-a-11, II-a-12, II-a-13, II-a-14, and II-a-15:

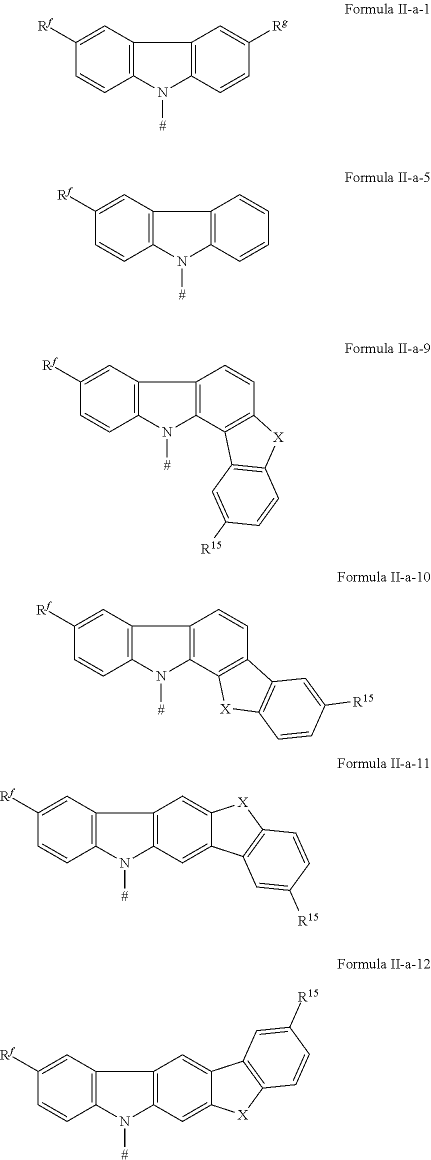

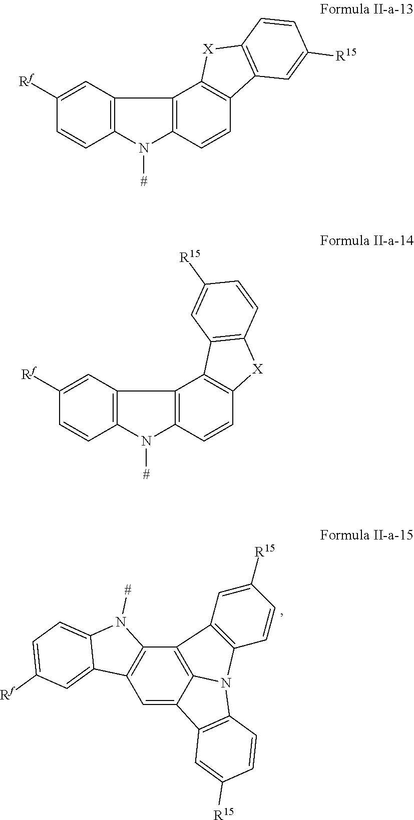

wherein X is selected from C(R¹⁶)₂, NR¹⁶, O, and S;

R¹⁶ is at each occurrence independently of each other selected from the group consisting of: hydrogen, deuterium, Me, ⁱPr, ^{t}Bu, and

Ph, wherein one or more hydrogen atoms are optionally substituted by deuterium, Me, ⁱPr, ^{t}Bu, and Ph.

In one embodiment of the present disclosure, the second chemical moiety comprises or consists of a structure according to formula II-a-1, wherein the aforementioned definitions apply.

In one embodiment of the present disclosure, the second chemical moiety comprises or consists of a structure according to formula II-a-5, wherein the aforementioned definitions apply.

In an embodiment of the present disclosure, the second chemical moiety comprises or consists of a structure according to formula II-a-1 or II-a-5, wherein the aforementioned definitions apply.

Below, examples of the second chemical moiety are shown:

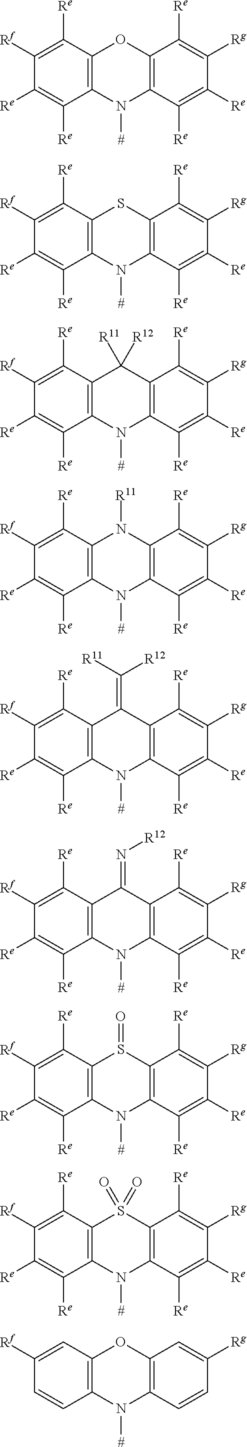

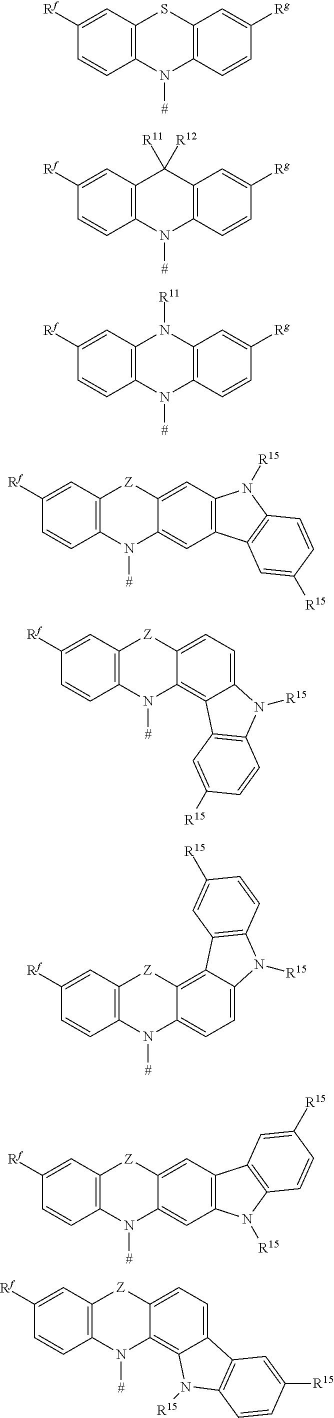

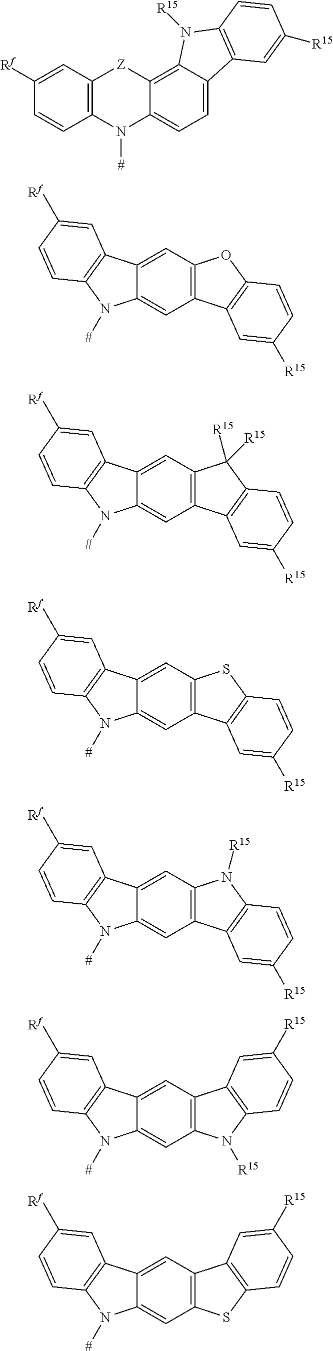

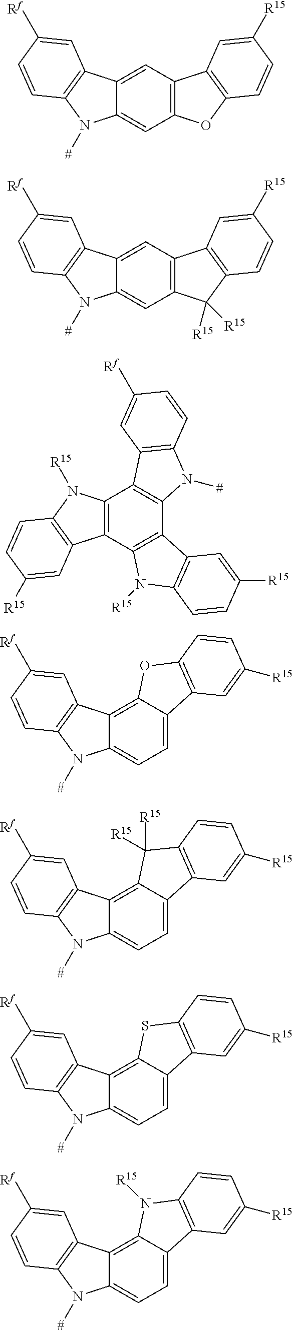

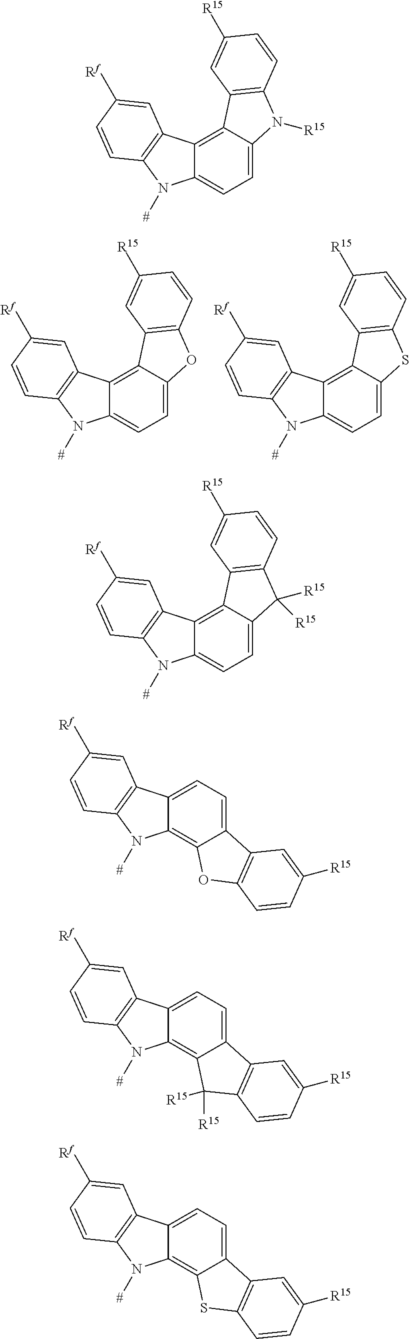

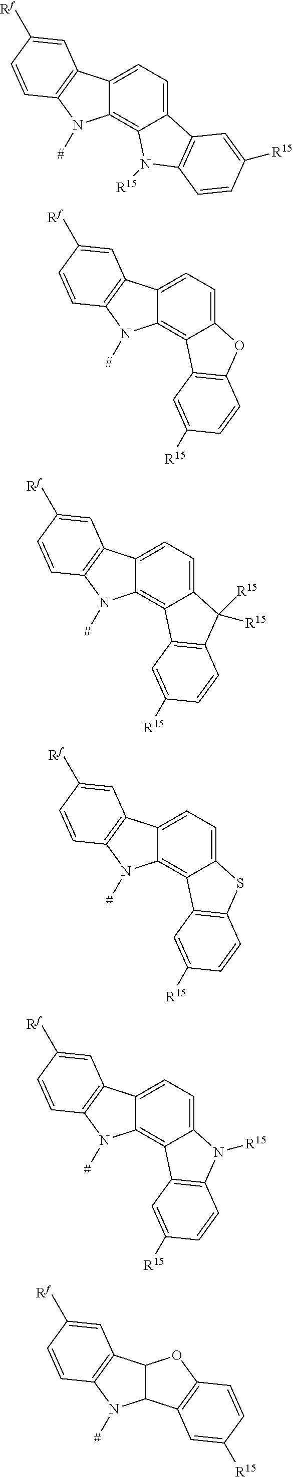

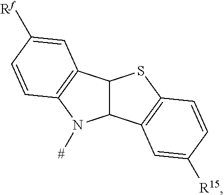

wherein this does not imply that embodiments of the present disclosure are limited to organic molecules comprising a second chemical moiety represented by any of the example structures shown above.

As used throughout the present application, the term “cyclic group” may be understood in the broadest sense as any mono-, bi- or polycyclic moieties.

As used throughout the present application, the terms “ring” and “ring system” may be understood in the broadest sense as any mono-, bi- or polycyclic moieties.

The term “ring atom” refers to any atom which is part of the cyclic core of a ring or a ring structure, and not part of a substituent optionally attached to the cyclic core.

As used throughout the present application, the term “carbocycle” may be understood in the broadest sense as any cyclic group in which the cyclic core structure comprises only carbon atoms that may of course be substituted with hydrogen or any other substituents defined in the embodiments of the present disclosure. It is understood that the term “carbocyclic” as an adjective refers to cyclic groups in which the cyclic core structure comprises only carbon atoms that may of course be substituted with hydrogen or any other substituents defined in the embodiments of the present disclosure.

As used throughout the present application, the term “heterocycle” may be understood in the broadest sense as any cyclic group in which the cyclic core structure comprises not just carbon atoms, but also at least one heteroatom. It is understood that the term “heterocyclic” as adjective refers to cyclic groups in which the cyclic core structure comprises not just carbon atoms, but also at least one heteroatom. The heteroatoms may, unless stated otherwise in specific embodiments, at each occurrence be the same or different and be individually selected from the group consisting of N, O, and S. All carbon atoms or heteroatoms comprised in a heterocycle in the context of the present disclosure may of course be substituted with hydrogen or any other substituents defined in the embodiments of the present disclosure.

As used throughout the present application, the term “aromatic ring system” may be understood in the broadest sense as any bi- or polycyclic aromatic moiety.

As used throughout the present application, the term “heteroaromatic ring system” may be understood in the broadest sense as any bi- or polycyclic heteroaromatic moiety.

As used throughout the present application, the term “fused” when referring to aromatic or heteroaromatic ring systems means that the aromatic or heteroaromatic rings that are “fused” share at least one bond that is part of both ring systems. For example naphthalene (or naphthyl when referred to as substituent) or benzothiophene (or benzothiophenyl when referred to as substituent) are considered fused aromatic ring systems in the context of embodiments of the present disclosure, in which two benzene rings (for naphthalene) or a thiophene and a benzene (for benzothiophene) share one bond. It is also understood that sharing a bond in this context includes sharing the two atoms that build up the respective bond and that fused aromatic or heteroaromatic ring systems can be understood as one aromatic or heteroaromatic system. Additionally, it is understood, that more than one bond may be shared by the aromatic or heteroaromatic rings building up a fused aromatic or heteroaromatic ring system (e.g., in pyrene). Furthermore, it will be understood that aliphatic ring systems may also be fused and that this has the same meaning as for aromatic or heteroaromatic ring systems, with the exception of course, that fused aliphatic ring systems are not aromatic.

As used throughout the present application, the terms “aryl” and “aromatic” may be understood in the broadest sense as any mono-, bi- or polycyclic aromatic moieties. Accordingly, unless specified differently in specific embodiments of the present disclosure, an aryl group contains 6 to 60 aromatic ring atoms, and a heteroaryl group contains 5 to 60 aromatic ring atoms, of which at least one is a heteroatom. Notwithstanding, throughout the application the number of aromatic ring carbon atoms may be given as a subscripted number in the definition of certain substituents. In some embodiments, the heteroaromatic ring includes one to three heteroatoms. Again, the terms “heteroaryl” and “heteroaromatic” may be understood in the broadest sense as any mono-, bi- or polycyclic hetero-aromatic moieties that include at least one heteroatom. The heteroatoms may, unless stated otherwise in specific embodiments, at each occurrence be the same or different and be individually selected from the group consisting of N, O, and S. Accordingly, the term “arylene” refers to a divalent substituent that bears two binding sites to other molecular structures and thereby serving as a linker structure. In case, a group in the example embodiments is defined differently from the definitions given here, for example, the number of aromatic ring atoms or number of heteroatoms differs from the given definition, the definition in the example embodiments is to be applied. According to embodiments of the present disclosure, a condensed (annulated) aromatic or heteroaromatic polycycle is built of two or more single aromatic or heteroaromatic cycles, which formed the polycycle via a condensation reaction.

In some embodiments, as used throughout the present application the term “aryl group” or “heteroaryl group” comprises groups which can be bound via any suitable position of the aromatic or heteroaromatic group, derived from benzene, naphthalene, anthracene, phenanthrene, pyrene, dihydropyrene, chrysene, perylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene; pyrrole, indole, isoindole, carbazole, indolocarbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthoimidazole, phenanthroimidazole, pyridoimidazole, pyrazinoimidazole, quinoxalinoimidazole, oxazole, benzoxazole, naphthooxazole, anthroxazol, phenanthroxazol, isoxazole, 1,2-thiazole, 1,3-thiazole, benzothiazole, pyridazine, benzopyridazine, pyrimidine, benzopyrimidine, 1,3,5-triazine, quinoxaline, pyrazine, phenazine, naphthyridine, carboline, benzocarboline, phenanthroline, 1,2,3-triazole, 1,2,4-triazole, benzotriazole, 1,2,3-oxadiazole, 1,2,4-oxadiazole, 1,2,5-oxadiazole, 1,2,3,4-tetrazine, purine, pteridine, indolizine and/or benzothiadiazole or combinations of the abovementioned groups.

In certain embodiments of the present disclosure, adjacent substituents bonded to an aromatic or heteroaromatic ring or ring system may together form an additional mono- or polycyclic, aliphatic or aromatic, carbocyclic or heterocyclic ring or ring system which is fused to the aromatic or heteroaromatic ring or ring system to which the substituents are bonded. It is understood that the optionally so formed fused ring system will be larger (meaning it comprises more ring atoms) than the aromatic or heteroaromatic ring or ring system to which the adjacent substituents are bonded. In these cases, the “total” amount of ring atoms comprised in the fused ring system is to be understood as the sum of ring atoms comprised in the aromatic or heteroaromatic ring or ring system to which the adjacent substituents are bonded and the ring atoms of the additional ring system formed by the adjacent substituents, wherein, however, the carbon atoms that are shared by fused rings are counted once and not twice. For example, a benzene ring may have two adjacent substituents that form another benzene ring so that a naphthalene core is built. This naphthalene core then comprises 10 ring atoms as two carbon atoms are shared by the two benzene rings and are thus only counted once and not twice. The term “adjacent substituents” in this context refers to substituents attached to the same or to neighboring atoms.

As used throughout the present application, the terms “adjacent substituents” or “adjacent groups” refer to substituents or groups bonded to either the same or to neighboring atoms.

As used throughout the present application, the term “aliphatic” when referring to ring systems may be understood in the broadest sense and means that none of the rings that build up the ring system is an aromatic or heteroaromatic ring. It is understood that such an aliphatic ring system may be fused to one or more aromatic rings so that some (but not all) carbon- or heteroatoms comprised in the core structure of the aliphatic ring system are part of an attached aromatic ring.

As used above and herein, the term “alkyl group” may be understood in the broadest sense as any linear, branched, or cyclic alkyl substituent. In some embodiments, the term alkyl comprises the substituents methyl (Me), ethyl (Et), n-propyl (ⁿPr), i-propyl (ⁱPr), cyclopropyl, n-butyl (ⁿBu), i-butyl (ⁱBu), s-butyl (^{s}Bu), t-butyl (^{t}Bu), cyclobutyl, 2-methylbutyl, n-pentyl, s-pentyl, t-pentyl, 2-pentyl, neo-pentyl, cyclopentyl, n-hexyl, s-hexyl, t-hexyl, 2-hexyl, 3-hexyl, neo-hexyl, cyclohexyl, 1-methylcyclopentyl, 2-methylpentyl, n-heptyl, 2-heptyl, 3-heptyl, 4-heptyl, cycloheptyl, 1-methylcyclohexyl, n-octyl, 2-ethylhexyl, cyclooctyl, 1-bicyclo[2,2,2]octyl, 2-bicyclo[2,2,2]-octyl, 2-(2,6-dimethyl)octyl, 3-(3,7-dimethyl)octyl, adamantyl, 2,2,2-trifluorethyl, 1,1-dimethyl-n-hex-1-yl, 1,1-dimethyl-n-hept-1-yl, 1,1-dimethyl-n-oct-1-yl, 1,1-dimethyl-n-dec-1-yl, 1,1-dimethyl-n-dodec-1-yl, 1,1-dimethyl-n-tetradec-1-yl, 1,1-dimethyl-n-hexadec-1-yl, 1,1-dimethyl-n-octadec-1-yl, 1,1-diethyl-n-hex-1-yl, 1,1-diethyl-n-hept-1-yl, 1,1-diethyl-n-oct-1-yl, 1,1-diethyl-n-dec-1-yl, 1,1-diethyl-n-dodec-1-yl, 1,1-diethyl-n-tetradec-1-yl, 1,1-diethyln-n-hexadec-1-yl, 1,1-diethyl-n-octadec-1-yl, 1-(n-propyl)-cyclohex-1-yl, 1-(n-butyl)-cyclohex-1-yl, 1-(n-hexyl)-cyclohex-1-yl, 1-(n-octyl)-cyclohex-1-yl and 1-(n-decyl)-cyclohex-1-yl.

As used above and herein, the term “alkenyl” comprises linear, branched, and cyclic alkenyl substituents. The term alkenyl group comprises, for example, the substituents ethenyl, propenyl, butenyl, pentenyl, cyclopentenyl, hexenyl, cyclohexenyl, heptenyl, cycloheptenyl, octenyl, cyclooctenyl or cyclooctadienyl.

As used above and herein, the term “alkynyl” comprises linear, branched, and cyclic alkynyl substituents. The term alkynyl group comprises, for example, ethynyl, propynyl, butynyl, pentynyl, hexynyl, heptynyl or octynyl.

As used above and herein, the term “alkoxy” comprises linear, branched, and cyclic alkoxy substituents. The term alkoxy group comprises, for example, methoxy, ethoxy, n-propoxy, i-propoxy, n-butoxy, i-butoxy, s-butoxy, t-butoxy and 2-methylbutoxy.

As used above and herein, the term “thioalkoxy” comprises linear, branched, and cyclic thioalkoxy substituents, in which the O of the example alkoxy groups is replaced by S.

As used above and herein, the terms “halogen” and “halo” may be understood in the broadest sense as being, for example, fluorine, chlorine, bromine or iodine.

It is understood that when a molecular fragment is described as being a substituent or otherwise attached to another moiety, its name may be written as if it were a fragment (e.g., naphthyl, dibenzofuryl) or as if it were the whole molecule (e.g., naphthalene, dibenzofuran). As used herein, these different ways of designating a substituent or attached fragment are considered to be equivalent.

All hydrogen atoms (H) comprised in any structure referred to herein may at each occurrence independently of each other, and without this being indicated specifically, be replaced by deuterium (D). The replacement of hydrogen by deuterium should be readily apparent to the person skilled in the art upon reviewing this disclosure.

In one embodiment of the present disclosure, the organic molecules according to embodiments of the present disclosure have an excited state lifetime of not more than 50 μs, of not more than 25 μs, of not more than 15 μs, of not more than 10 μs, of not more than 8 μs or not more than 6 μs, or of not more than 4 μs in a film of poly(methyl methacrylate) (PMMA) including 10% by weight of the organic molecule at room temperature.

In one embodiment of the present disclosure, the organic molecules according to embodiments of the present disclosure represent thermally-activated delayed fluorescence (TADF) emitters, which exhibit a ΔE_{ST} value, which corresponds to the energy difference between the first excited singlet state (S1) and the first excited triplet state (T1), of less than 5000 cm⁻¹, less than 3000 cm⁻¹, less than 1500 cm⁻¹, less than 1000 cm⁻¹ or even less than 500 cm⁻¹.

In a further embodiment of the present disclosure, the organic molecules according to embodiments of the present disclosure have an emission peak in the visible or nearest ultraviolet range, e.g., in the range of a wavelength of from 380 to 800 nm, with a full width at half maximum of less than 0.60 eV, less than 0.50 eV, less than 0.45 eV, less than 0.43 eV or even less than 0.40 eV in a film of poly(methyl methacrylate) (PMMA) including 10% by weight of the organic molecule at room temperature.

Orbital and excited state energies can be determined either by means of experimental methods or by calculations employing quantum-chemical methods, for example, density functional theory calculations. The energy of the highest occupied molecular orbital E^{HOMO} is determined by methods readily recognizable to the person skilled in the art from cyclic voltammetry measurements with an accuracy of 0.1 eV. The energy of the lowest unoccupied molecular orbital E^{LUMO} is determined as the onset of the absorption spectrum.

Absorption spectra of organic molecules according to embodiments of the present disclosure may be recorded from a film of the organic molecule according to embodiments of the present disclosure in poly(methyl methacrylate) (PMMA) with 10% by weight of organic molecule at room temperature (e.g., approx. 20° C.). In some embodiments, they may also be recorded from solutions of the respective molecules, wherein the concentration of the solution is chosen so that the maximum absorbance is in a range of 0.1 to 0.5.

The onset of an absorption spectrum is determined by computing the intersection of the tangent to the absorption spectrum with the x-axis. The tangent to the absorption spectrum is set at the low-energy side of the absorption band and at the point at half maximum of the maximum intensity of the absorption spectrum.

Unless stated otherwise, the energy of the first excited triplet state T1 is determined from the onset the phosphorescence spectrum at 77K (steady-state spectrum; film of 10% by weight of emitter in PMMA).

Unless stated otherwise, the energy of the first excited singlet state S1 is determined from the onset the fluorescence spectrum at room temperature (e.g., approx. 20° C.; steady-state spectrum; film of 10% by weight of emitter in PMMA).

The onset of an emission spectrum is determined by computing the intersection of the tangent to the emission spectrum with the x-axis. The tangent to the emission spectrum is set at the high-energy side of the emission band and at the point at half maximum of the maximum intensity of the emission spectrum.

The ΔE_{ST} value, which corresponds to the energy difference between the first excited singlet state (S1) and the first excited triplet state (T1), is determined based on the first excited singlet state energy and the first excited triplet state energy, which were determined as stated above.

A further aspect of embodiments of the present disclosure relates to the use of an organic molecule according to embodiments of the present disclosure as a luminescent emitter or as an absorber, and/or as host material and/or as electron transport material, and/or as hole injection material, and/or as hole blocking material in an optoelectronic device.

The optoelectronic device may be understood in the broadest sense as any suitable device based on organic materials that is suitable for emitting light in the visible or nearest ultraviolet (UV) range, e.g., in the range of a wavelength of from 380 to 800 nm. For example, the optoelectronic device may be able to emit light in the visible range, e.g., of from 400 to 800 nm.

In the context of such use, the optoelectronic device may be selected from the group consisting of:
organic light-emitting diodes (OLEDs),
light-emitting electrochemical cells,
OLED sensors, especially in gas and vapor sensors not hermetically externally shielded,
organic diodes,
organic solar cells,
organic transistors,
organic field-effect transistors,
organic lasers and
down-conversion elements.

A light-emitting electrochemical cell consists of three layers, namely a cathode, an anode, and an active layer, which contains the organic molecule according to embodiments of the present disclosure.

In an embodiment in the context of such use, the optoelectronic device is a device selected from the group consisting of an organic light emitting diode (OLED), a light emitting electrochemical cell (LEC), an organic laser, and a light-emitting transistor.

In one embodiment, the light-emitting layer of an organic light-emitting diode comprises not only the organic molecules according to embodiments of the present disclosure but also a host material whose triplet (T1) and singlet (S1) energy levels are energetically higher than the triplet (T1) and singlet (S1) energy levels of the organic molecule.

A further aspect of embodiments of the present disclosure relates to a composition comprising or consisting of:
(a) the organic molecule of embodiments of the present disclosure, for example, in the form of an emitter and/or a host, and
(b) one or more emitter and/or host materials, which differ from the organic molecule of embodiments of the present disclosure, and
(c) optionally, one or more dyes and/or one or more solvents.

In a further embodiment of the present disclosure, the composition has a photoluminescence quantum yield (PLQY) of more than 26%, more than 40%, more than 60%, more than 80% or even more than 90% at room temperature.

Compositions with at Least One Further Emitter

One embodiment of the present disclosure relates to a composition comprising or consisting of:
(i) 1-50% by weight, 5-40% by weight, or 10-30% by weight, of the organic molecule according to embodiments of the present disclosure;
(ii) 5-98% by weight, 30-93.9% by weight, or 40-88% by weight, of one host compound H;
(iii) 1-30% by weight, 1-20% by weight, or 1-5% by weight, of at least one further emitter molecule F having a structure differing from the structure of the molecules according to embodiments of the present disclosure; and
(iv) optionally 0-94% by weight, 0.1-65% by weight, or 1-50% by weight, of at least one further host compound D having a structure differing from the structure of the molecules according to embodiments of the present disclosure; and
(v) optionally 0-94% by weight, 0-65% by weight, or 0-50% by weight, of a solvent.

The components or the compositions are chosen such that the sum of the weight of the components add up to 100%.

In a further embodiment of the present disclosure, the composition has an emission peak in the visible or nearest ultraviolet range, e.g., in the range of a wavelength of from 380 to 800 nm.

In one embodiment of the present disclosure, the at least one further emitter molecule F is a purely organic emitter.

In one embodiment of the present disclosure, the at least one further emitter molecule F is a purely organic TADF emitter. Purely organic TADF emitters are known from the state of the art, e.g., Wong and Zysman-Colman (Purely Organic Thermally Activated Delayed Fluorescence Materials for Organic Light-Emitting Diodes.”, Adv. Mater. 2017 June; 29(22)).

In one embodiment of the present disclosure, the at least one further emitter molecule F is a fluorescence emitter, for example, a blue, a green or a red fluorescence emitter.

In a further embodiment of the present disclosure, the composition, containing the at least one further emitter molecule F shows an emission peak in the visible or nearest ultraviolet range, e.g., in the range of a wavelength of from 380 to 800 nm, with a full width at half maximum of less than 0.30 eV, less than 0.25 eV, less than 0.22 eV, less than 0.19 eV or even less than 0.17 eV at room temperature, with a lower limit of 0.05 eV.

### Light-Emitting Layer EML

In one embodiment, the light-emitting layer EML of an organic light-emitting diode of embodiments of the present disclosure comprises (or essentially consists of) a composition comprising or consisting of:
(i) 1-50% by weight, 5-40% by weight, or 10-30% by weight, of one or more organic molecules according to embodiments of the present disclosure;
(ii) 5-99% by weight, 30-94.9% by weight, or 40-89% by weight, of at least one host compound H; and
(iii) optionally 0-94% by weight, 0.1-65% by weight, or 1-50% by weight, of at least one further host compound D having a structure differing from the structure of the molecules according to embodiments of the present disclosure; and
(iv) optionally 0-94% by weight, 0-65% by weight, or 0-50% by weight, of a solvent; and
(v) optionally 0-30% by weight, 0-20% by weight, or 0-5% by weight, of at least one further emitter molecule F having a structure differing from the structure of the molecules according to embodiments of the present disclosure.

As an example, energy can be transferred from the host compound H to the one or more organic molecules of embodiments of the present disclosure, for example, transferred from the first excited triplet state T1(H) of the host compound H to the first excited triplet state T1(E) of the one or more organic molecules according to embodiments of the present disclosure and/or from the first excited singlet state S1(H) of the host compound H to the first excited singlet state S1(E) of the one or more organic molecules according to embodiments of the present disclosure.

In one embodiment, the host compound H has a highest occupied molecular orbital HOMO(H) having an energy E^{HOMO}(H) in the range of from −5 eV to −6.5 eV and one organic molecule according to embodiments of the present disclosure E has a highest occupied molecular orbital HOMO(E) having an energy E^{HOMO}(E), wherein E^{HOMO}(H)>E^{HOMO}(E).

In a further embodiment, the host compound H has a lowest unoccupied molecular orbital LUMO(H) having an energy E^{LUMO}(H) and the one organic molecule according to embodiments of the present disclosure E has a lowest unoccupied molecular orbital LUMO(E) having an energy E^{LUMO}(E), wherein E^{LUMO}(H)>E^{LUMO}(E).

### Light-Emitting Layer EML Comprising at Least One Further Host Compound D

In a further embodiment, the light-emitting layer EML of an organic light-emitting diode of embodiments of the present disclosure comprises (or essentially consists of) a composition comprising or consisting of:
(i) 1-50% by weight, 5-40% by weight, or 10-30% by weight, of one organic molecule according to embodiments of the present disclosure;
(ii) 5-99% by weight, 30-94.9% by weight, or 40-89% by weight, of one host compound H; and
(iii) 0-94% by weight, 0.1-65% by weight, or 1-50% by weight, of at least one further host compound D having a structure differing from the structure of the molecules according to embodiments of the present disclosure; and
(iv) optionally 0-94% by weight, 0-65% by weight, or 0-50% by weight, of a solvent; and
(v) optionally 0-30% by weight, 0-20% by weight, or 0-5% by weight, of at least one further emitter molecule F having a structure differing from the structure of the molecules according to embodiments of the present disclosure.

In one embodiment of the organic light-emitting diode of embodiments of the present disclosure, the host compound H has a highest occupied molecular orbital HOMO(H) having an energy E^{HOMO}(H) in the range of from −5 eV to −6.5 eV and the at least one further host compound D has a highest occupied molecular orbital HOMO(D) having an energy E^{HOMO}(D), wherein E^{HOMO}(H)>E^{HOMO}(D). The relation E^{HOMO}(H)>E^{HOMO}(D) favors an efficient hole transport.

In a further embodiment, the host compound H has a lowest unoccupied molecular orbital LUMO(H) having an energy E^{LUMO}(H) and the at least one further host compound D has a lowest unoccupied molecular orbital LUMO(D) having an energy E^{LUMO}(D), wherein E^{LUMO}(H)>E^{LUMO}(D). The relation E^{LUMO}(H)>E^{LUMO}(D) favors an efficient electron transport.

In one embodiment of the organic light-emitting diode of embodiments of the present disclosure, the host compound H has a highest occupied molecular orbital HOMO(H) having an energy E^{HOMO}(H) and a lowest unoccupied molecular orbital LUMO(H) having an energy E^{LUMO}(H), and

the at least one further host compound D has a highest occupied molecular orbital HOMO(D) having an energy E^{HOMO}(D) and a lowest unoccupied molecular orbital LUMO(D) having an energy E^{LUMO}(D),

the organic molecule E of embodiments of the present disclosure has a highest occupied molecular orbital HOMO(E) having an energy E^{HOMO}(E) and a lowest unoccupied molecular orbital LUMO(E) having an energy E^{LUMO}(E),

wherein E^{HOMO}(H)>E^{HOMO}(D) and the difference between the energy level of the highest occupied molecular orbital HOMO(E) of organic molecule according to embodiments of the present disclosure (E^{HOMO}(E)) and the energy level of the highest occupied molecular orbital HOMO(H) of the host compound H (E^{HOMO}(H)) is between −0.5 eV and 0.5 eV, between −0.3 eV and 0.3 eV, between −0.2 eV and 0.2 eV or even between −0.1 eV and 0.1 eV; and

E^{LUMO}(H)>E^{LUMO}(D) and the difference between the energy level of the lowest unoccupied molecular orbital LUMO(E) of organic molecule according to embodiments of the present disclosure (E^{LUMO}(E)) and the lowest unoccupied molecular orbital LUMO(D) of the at least one further host compound D (E^{LUMO}(D)) is between −0.5 eV and 0.5 eV, between −0.3 eV and 0.3 eV, between −0.2 eV and 0.2 eV or even between −0.1 eV and 0.1 eV.

### Light-Emitting Layer EML Comprising at Least One Further Emitter Molecule F

In a further embodiment, the light-emitting layer EML comprises (or (essentially) consists of) a composition comprising or consisting of:
(i) 1-50% by weight, 5-40% by weight, or 10-30% by weight, of one organic molecule according to embodiments of the present disclosure;
(ii) 5-98% by weight, 30-93.9% by weight, or 40-88% by weight, of one host compound H;
(iii) 1-30% by weight, 1-20% by weight, or 1-5% by weight, of at least one further emitter molecule F having a structure differing from the structure of the molecules according to embodiments of the present disclosure; and
(iv) optionally 0-94% by weight, 0.1-65% by weight, or 1-50% by weight, of at least one further host compound D having a structure differing from the structure of the molecules according to embodiments of the present disclosure; and
(v) optionally 0-94% by weight, 0-65% by weight, or 0-50% by weight, of a solvent.

In a further embodiment, the light-emitting layer EML comprises (or (essentially) consists of) a composition as described in Compositions with at least one further emitter, with the at least one further emitter molecule F as defined in Composition wherein the at least one further emitter molecule F is a blue fluorescence emitter.

In a further embodiment, the light-emitting layer EML comprises (or (essentially) consists of) a composition as described in Compositions with at least one further emitter, with the at least one further emitter molecule F as defined in Composition wherein the at least one further emitter molecule F is a triplet-triplet annihilation (TTA) fluorescence emitter.

In a further embodiment, the light-emitting layer EML comprises (or (essentially) consists of) a composition as described in Compositions with at least one further emitter, with the at least one further emitter molecule F as defined in Composition wherein the at least one further emitter molecule F is a green fluorescence emitter.

In a further embodiment, the light-emitting layer EML comprises (or (essentially) consists of) a composition as described in Compositions with at least one further emitter, with the at least one further emitter molecule F as defined in Composition wherein the at least one further emitter molecule F is a red fluorescence emitter.

In one embodiment of the light-emitting layer EML comprising at least one further emitter molecule F, energy can be transferred from the one or more organic molecules of embodiments of the present disclosure E to the at least one further emitter molecule F, for example, transferred from the first excited singlet state S1(E) of one or more organic molecules of embodiments of the present disclosure E to the first excited singlet state S1(F) of the at least one further emitter molecule F.

In one embodiment, the first excited singlet state S1(H) of one host compound H of the light-emitting layer is higher in energy than the first excited singlet state S1(E) of the one or more organic molecules of embodiments of the present disclosure E: S1(H)>S1(E), and the first excited singlet state S1(H) of one host compound H is higher in energy than the first excited singlet state S1(F) of the at least one emitter molecule F: S1(H)>S1(F).

In one embodiment, the first excited triplet state T1(H) of one host compound H is higher in energy than the first excited triplet state T1(E) of the one or more organic molecules of embodiments of the present disclosure E: T1(H)>T1(E), and the first excited triplet state T1(H) of one host compound H is higher in energy than the first excited triplet state T1(F) of the at least one emitter molecule F: T1(H)>T1(F).

In one embodiment, the first excited singlet state S1(E) of the one or more organic molecules of embodiments of the present disclosure E is higher in energy than the first excited singlet state S1(F) of the at least one emitter molecule F: S1(E)>S1(F).

In one embodiment, the first excited triplet state T1(E) of the one or more organic molecules E of embodiments of the present disclosure is higher in energy than the first excited singlet state T1(F) of the at least one emitter molecule F: T1(E)>T1(F).

In one embodiment, the first excited triplet state T1(E) of the one or more organic molecules E of embodiments of the present disclosure is higher in energy than the first excited singlet state T1(F) of the at least one emitter molecule F: T1(E)>T1(F), wherein the absolute value of the energy difference between T1(E) and T1(F) is larger than 0.3 eV, larger than 0.4 eV, or even larger than 0.5 eV.

In one embodiment, the host compound H has a highest occupied molecular orbital HOMO(H) having an energy E^{HOMO}(H) and a lowest unoccupied molecular orbital LUMO(H) having an energy E^{LUMO}(H), and

the one organic molecule according to embodiments of the present disclosure E has a highest occupied molecular orbital HOMO(E) having an energy E^{HOMO}(E) and a lowest unoccupied molecular orbital LUMO(E) having an energy E^{LUMO}(E),

the at least one further emitter molecule F has a highest occupied molecular orbital HOMO(F) having an energy E^{HOMO}(F) and a lowest unoccupied molecular orbital LUMO(E) having an energy E^{LUMO}(F),

wherein E^{HOMO}(H)>E^{HOMO}(E) and the difference between the energy level of the highest occupied molecular orbital HOMO(F) of the at least one further emitter molecule (E^{HOMO}(F)) and the energy level of the highest occupied molecular orbital HOMO(H) of the host compound H (E^{HOMO}(H)) is between −0.5 eV and 0.5 eV, between −0.3 eV and 0.3 eV, between −0.2 eV and 0.2 eV or even between −0.1 eV and 0.1 eV; and

E^{LUMO}(H)>E^{LUMO}(E) and the difference between the energy level of the lowest unoccupied molecular orbital LUMO(F) of the at least one further emitter molecule (E^{LUMO}(F)) and the lowest unoccupied molecular orbital LUMO(E) of the one organic molecule according to embodiments of the present disclosure (E^{LUMO}(E)) is between −0.5 eV and 0.5 eV, between −0.3 eV and 0.3 eV, between −0.2 eV and 0.2 eV or even between −0.1 eV and 0.1 eV.

### Optoelectronic Devices

In a further aspect, embodiments of the present disclosure relates to an optoelectronic device comprising an organic molecule or a composition as described herein, for example, in the form of a device selected from the group consisting of organic light-emitting diode (OLED), light-emitting electrochemical cell, OLED sensor, gas and vapour sensors not hermetically externally shielded, organic diode, organic solar cell, organic transistor, organic field-effect transistor, organic laser and down-conversion element.

In an embodiment, the optoelectronic device is a device selected from the group consisting of an organic light emitting diode (OLED), a light emitting electrochemical cell (LEC), and a light-emitting transistor.

In one embodiment of the optoelectronic device of the present disclosure, the organic molecule according to embodiments of the present disclosure is used as an emission material in a light-emitting layer EML.

In one embodiment of the optoelectronic device of the present disclosure, the light-emitting layer EML consists of the composition according to embodiments of the present disclosure described herein.

When the optoelectronic device is an OLED, it may, for example, exhibit the following layer structure:
1. Substrate
2. Anode layer A
3. Hole injection layer, HIL
4. Hole transport layer, HTL
5. Electron blocking layer, EBL
6. Emitting layer, EML
7. Hole blocking layer, HBL
8. Electron transport layer, ETL
9. Electron injection layer, EIL
10. Cathode layer,
wherein the OLED comprises each layer only optionally, different layers may be merged and the OLED may comprise more than one layer of each layer type (or kind) defined above.

Furthermore, the optoelectronic device may optionally comprise one or more protective layers protecting the device from damaging exposure to harmful species in the environment including, for example, moisture, vapor and/or gases.

In one embodiment of the present disclosure, the optoelectronic device is an OLED, which exhibits the following inverted layer structure:
1. Substrate
2. Cathode layer
3. Electron injection layer, EIL
4. Electron transport layer, ETL
5. Hole blocking layer, HBL
6. Emitting layer, EML
7. Electron blocking layer, EBL
8. Hole transport layer, HTL
9. Hole injection layer, HIL
10. Anode layer A
wherein the OLED with an inverted layer structure comprises each layer only optionally, different layers may be merged and the OLED may comprise more than one layer of each layer types (or kinds) defined above.

In one embodiment of the present disclosure, the optoelectronic device is an OLED, which may exhibit a stacked architecture. In this architecture, contrary to other arrangements, where the OLEDs are placed side by side, the individual units are stacked on top of each other. Blended light may be generated with OLEDs exhibiting a stacked architecture, for example, white light may be generated by stacking blue, green and red OLEDs. Furthermore, the OLED exhibiting a stacked architecture may optionally comprise a charge generation layer (CGL), which may be between two OLED subunits and may consist of a n-doped and p-doped layer with the n-doped layer of one CGL being closer to the anode layer.

In one embodiment of the present disclosure, the optoelectronic device is an OLED, which comprises two or more emission layers between anode and cathode. In some embodiments, this so-called tandem OLED comprises three emission layers, wherein one emission layer emits red light, one emission layer emits green light and one emission layer emits blue light, and optionally may comprise further layers such as charge generation layers, blocking or transporting layers between the individual emission layers. In a further embodiment, the emission layers are adjacently stacked. In a further embodiment, the tandem OLED comprises a charge generation layer between each two emission layers. In addition, adjacent emission layers or emission layers separated by a charge generation layer may be merged.

The substrate may be formed by any suitable material or composition of materials. Most frequently, glass slides are used as substrates. In some embodiments, thin metal layers (e.g., copper, gold, silver and/or aluminum films) and/or plastic films and/or slides may be used. This may allow a higher degree of flexibility. The anode layer A is mostly composed of materials allowing to obtain an (essentially) transparent film. As at least one selected from the two electrodes should be (essentially) transparent in order to allow light emission from the OLED, either the anode layer A or the cathode layer C is transparent. For example, the anode layer A comprises a large content or even consists of transparent conductive oxides (TCOs). Such anode layer A may, for example, comprise indium tin oxide, aluminum zinc oxide, fluorine doped tin oxide, indium zinc oxide, PbO, SnO, zirconium oxide, molybdenum oxide, vanadium oxide, wolfram oxide, graphite, doped Si, doped Ge, doped GaAs, doped polyaniline, doped polypyrrol and/or doped polythiophene.

In some embodiments, the anode layer A (essentially) consists of indium tin oxide (ITO) (e.g., (InO₃)0.9(SnO₂)0.1). The roughness of the anode layer A caused by the transparent conductive oxides (TCOs) may be compensated by using a hole injection layer (HIL). Further, the HIL may facilitate the injection of quasi charge carriers (e.g., holes) in that the transport of the quasi charge carriers from the TCO to the hole transport layer (HTL) is facilitated. The hole injection layer (HIL) may comprise poly-3,4-ethylenedioxy thiophene (PEDOT), polystyrene sulfonate (PSS), MoO₂, V₂O₅, CuPC and/or CuI, for example, a mixture of PEDOT and PSS. The hole injection layer (HIL) may also prevent or reduce the diffusion of metals from the anode layer A into the hole transport layer (HTL). The HIL may comprise PEDOT:PSS (poly-3,4-ethylendioxy thiophene: polystyrene sulfonate), PEDOT (poly-3,4-ethylendioxy thiophene), mMTDATA (4,4′,4″-tris[phenyl(m-tolyl)amino]triphenylamine), Spiro-TAD (2,2′,7,7′-tetrakis(n,n-diphenylamino)-9,9′-spirobifluorene), DNTPD (N1,N1′-(biphenyl-4,4′-diyl)bis(N1-phenyl-N4,N4-di-m-tolylbenzene-1,4-diamine), NPB (N,N′-bis-(1-naphthalenyl)-N,N′-bis-phenyl-(1,1′-biphenyl)-4,4′-diamine), NPNPB (N,N′-diphenyl-N,N′-di-[4-(N,N-diphenyl-amino)phenyl]benzidine), MeO-TPD (N,N,N′,N′-tetrakis(4-methoxyphenyl)benzidine), HAT-CN (1,4,5,8,9,11-hexaazatriphenylen-hexacarbonitrile) and/or Spiro-NPD (N,N′-diphenyl-N,N′-bis-(1-naphthyl)-9,9′-spirobifluorene-2,7-diamine).

Adjacent to the anode layer A or hole injection layer (HIL) a hole transport layer (HTL) may be located. Herein, any suitable hole transport compound may be used. For example, electron-rich heteroaromatic compounds such as triarylamines and/or carbazoles may be used as hole transport compound. The HTL may decrease the energy barrier between the anode layer A and the light-emitting layer EML. The hole transport layer (HTL) may also be an electron blocking layer (EBL). In some embodiments, hole transport compounds bear comparably high energy levels of their triplet states T1. For example, the hole transport layer (HTL) may comprise a star-shaped heterocycle such as tris(4-carbazoyl-9-ylphenyl)amine (TCTA), poly-TPD (poly(4-butylphenyl-diphenyl-amine)), [alpha]-NPD (poly(4-butylphenyl-diphenyl-amine)), TAPC (4,4′-cyclohexyliden-bis[N,N-bis(4-methylphenyl)benzenamine]), 2-TNATA (4,4′,4″-tris[2-naphthyl(phenyl)amino]triphenylamine), Spiro-TAD, DNTPD, NPB, NPNPB, MeO-TPD, HAT-CN and/or TrisPcz (9,9′-diphenyl-6-(9-phenyl-9H-carbazol-3-yl)-9H,9′H-3,3′-bicarbazole). In addition, the HTL may comprise a p-doped layer, which may be composed of an inorganic and/or organic dopant in an organic hole-transporting matrix. Transition metal oxides such as vanadium oxide, molybdenum oxide or tungsten oxide may be used as inorganic dopant. Tetrafluorotetracyanoquinodimethane (F4-TCNQ), copper-pentafluorobenzoate (Cu(I)pFBz) and/or transition metal complexes may be used as organic dopant.

The EBL may comprise mCP (1,3-bis(carbazol-9-yl)benzene), TCTA, 2-TNATA, mCBP (3,3-di(9H-carbazol-9-yl)biphenyl), tris-Pcz, CzSi (9-(4-tert-Butylphenyl)-3,6-bis(triphenylsilyl)-9H-carbazole), and/or DCB (N,N′-dicarbazolyl-1,4-dimethylbenzene).

Adjacent to the hole transport layer (HTL), the light-emitting layer EML may be located. The light-emitting layer EML comprises at least one light emitting molecule. In some embodiments, the EML comprises at least one light emitting molecule according to embodiments of the present disclosure. In some embodiments, the EML additionally comprises one or more host material. As an example, the host material may be selected from CBP (4,4′-Bis-(N-carbazolyl)-biphenyl), mCP, mCBP Sif87 (dibenzo[b,d]thiophen-2-yltriphenylsilane), CzSi, Sif88 (dibenzo[b,d]thiophen-2-yl)diphenylsilane), DPEPO (bis[2-(diphenylphosphino)phenyl] ether oxide), 9-[3-(dibenzofuran-2-yl)phenyl]-9H-carbazole, 9-[3-(dibenzofuran-2-yl)phenyl]-9H-carbazole, 9-[3-(dibenzothiophen-2-yl)phenyl]-9H-carbazole, 9-[3,5-bis(2-dibenzofuranyl)phenyl]-9H-carbazole, 9-[3,5-bis(2-dibenzothiophenyl)phenyl]-9H-carbazole, T2T (2,4,6-tris(biphenyl-3-yl)-1,3,5-triazine), T3T (2,4,6-tris(triphenyl-3-yl)-1,3,5-triazine) and/or TST (2,4,6-tris(9,9′-spirobifluorene-2-yl)-1,3,5-triazine). The host material may be selected to exhibit first triplet (T1) and first singlet (S1) energy levels, which are energetically higher than the first triplet (T1) and first singlet (S1) energy levels of the organic molecule.

In one embodiment of the present disclosure, the EML comprises a so-called mixed-host system with at least one hole-dominant host and one electron-dominant host. In some embodiments, the EML comprises exactly one light emitting molecule species according to embodiments of the present disclosure and a mixed-host system comprising T2T as electron-dominant host and a host selected from CBP, mCP, mCBP, 9-[3-(dibenzofuran-2-yl)phenyl]-9H-carbazole, 9-[3-(dibenzofuran-2-yl)phenyl]-9H-carbazole, 9-[3-(dibenzothiophen-2-yl)phenyl]-9H-carbazole, 9-[3,5-bis(2-dibenzofuranyl)phenyl]-9H-carbazole and 9-[3,5-bis(2-dibenzothiophenyl)phenyl]-9H-carbazole as hole-dominant host. In a further embodiment the EML comprises 50-80% by weight, or 60-75% by weight of a host selected from CBP, mCP, mCBP, 9-[3-(dibenzofuran-2-yl)phenyl]-9H-carbazole, 9-[3-(dibenzofuran-2-yl)phenyl]-9H-carbazole, 9-[3-(dibenzothiophen-2-yl)phenyl]-9H-carbazole, 9-[3,5-bis(2-dibenzofuranyl)phenyl]-9H-carbazole and 9-[3,5-bis(2-dibenzothiophenyl)phenyl]-9H-carbazole; 10-45% by weight, or 15-30% by weight of T2T and 5-40% by weight, or 10-30% by weight of a light emitting molecule according to embodiments of the present disclosure.

Adjacent to the light-emitting layer EML an electron transport layer (ETL) may be located. Herein, any suitable electron transporter may be used. In some embodiments, compounds poor of electrons such as, e.g., benzimidazoles, pyridines, triazoles, oxadiazoles (e.g., 1,3,4-oxadiazole), phosphinoxides and/or sulfone, may be used. An electron transporter may also be a star-shaped heterocycle such as 1,3,5-tri(1-phenyl-1H-benzo[d]imidazol-2-yl)phenyl (TPBi). The ETL may comprise Nbphen (2,9-bis(naphthalen-2-yl)-4,7-diphenyl-1,10-phenanthroline), Alq3 (Aluminum-tris(8-hydroxyquinoline)), TSPO1 (diphenyl-4-triphenylsilylphenyl-phosphinoxide), BpyTP2 (2,7-di(2,2′-bipyrdin-5-yl)triphenyle), Sif87 (dibenzo[b,d]thiophen-2-yltriphenylsilane), Sif88 (dibenzo[b,d]thiophen-2-yl)diphenylsilane), BmPyPhB (1,3-bis[3,5-di(pyridin-3-yl)phenyl]benzene) and/or BTB (4,4′-bis-[2-(4,6-diphenyl-1,3,5-triazinyl)]-1,1′-biphenyl). Optionally, the ETL may be doped with materials such as Liq. The electron transport layer (ETL) may also block or reduces transport of holes or a holeblocking layer (HBL) is introduced.

The HBL may, for example, comprise BCP (2,9-dimethyl-4,7-diphenyl-1,10-phenanthroline=Bathocuproine), BAIq (bis(8-hydroxy-2-methylquinoline)-(4-phenylphenoxy)aluminum), Nbphen (2,9-bis(naphthalen-2-yl)-4,7-diphenyl-1,10-phenanthroline), Alq3 (Aluminum-tris(8-hydroxyquinoline)), TSPO1 (diphenyl-4-triphenylsilylphenyl-phosphinoxide), T2T (2,4,6-tris(biphenyl-3-yl)-1,3,5-triazine), T3T (2,4,6-tris(triphenyl-3-yl)-1,3,5-triazine), TST (2,4,6-tris(9,9′-spirobifluorene-2-yl)-1,3,5-triazine), and/or TCB/TCP (1,3,5-tris(N-carbazolyl)benzol/1,3,5-tris(carbazol)-9-yl) benzene).

A cathode layer C may be located adjacent to the electron transport layer (ETL). For example, the cathode layer C may comprise or may consist of a metal (e.g., Al, Au, Ag, Pt, Cu, Zn, Ni, Fe, Pb, LiF, Ca, Ba, Mg, In, W, and/or Pd) and/or a metal alloy. For practical reasons, the cathode layer may also consist of (essentially) non-transparent metals such as Mg, Ca and/or Al. Alternatively or additionally, the cathode layer C may also comprise graphite and/or carbon nanotubes (CNTs). In one or more embodiments, the cathode layer C may also consist of nanoscalic silver wires.

An OLED may further, optionally, comprise a protection layer between the electron transport layer (ETL) and the cathode layer C (which may be designated as electron injection layer (EIL)). This layer may comprise lithium fluoride, cesium fluoride, silver, Liq (8-hydroxyquinolinolatolithium), Li₂O, BaF₂, MgO and/or NaF.

Optionally, also the electron transport layer (ETL) and/or a hole blocking layer (HBL) may comprise one or more host compounds.

In order to modify the emission spectrum and/or the absorption spectrum of the light-emitting layer EML further, the light-emitting layer EML may further comprise one or more further emitter molecule F. Such an emitter molecule F may be any suitable emitter molecule generally used in the art. Such an emitter molecule F is a molecule having a structure differing from the structure of the molecules according to embodiments of the present disclosure. The emitter molecule F may optionally be a TADF emitter. In some embodiments, the emitter molecule F may optionally be a fluorescent and/or phosphorescent emitter molecule which is able to shift the emission spectrum and/or the absorption spectrum of the light-emitting layer EML. For example, the triplet and/or singlet excitons may be transferred from the emitter molecule according to embodiments of the present disclosure to the emitter molecule F before relaxing to the ground state S0 by emitting light that may be red-shifted in comparison to the light emitted by emitter molecule E. Optionally, the emitter molecule F may also provoke two-photon effects (e.g., the absorption of two photons of half the energy of the absorption maximum).

Optionally, an optoelectronic device (e.g., an OLED) may, for example, be an essentially white optoelectronic device. For example, such white optoelectronic device may comprise at least one (deep) blue emitter molecule and one or more emitter molecules emitting green and/or red light. Then, there may also optionally be energy transmittance between two or more molecules as described above.

As used herein, if not defined more specifically in the particular context, the designation of the colors of emitted and/or absorbed light is as follows:
violet: wavelength range of greater than 380 to 420 nm;
deep blue: wavelength range of greater than 420 to 480 nm;
sky blue: wavelength range of greater than 480 to 500 nm;
green: wavelength range of greater than 500 to 560 nm;
yellow: wavelength range of greater than 560-580 nm;
orange: wavelength range of greater than 580 to 620 nm;
red: wavelength range of greater than 620 to 800 nm.

With respect to emitter molecules, such colors refer to the emission maximum. Therefore, for example, a deep blue emitter has an emission maximum in the range of from greater than 420 to 480 nm, a sky-blue emitter has an emission maximum in the range of from greater than 480 to 500 nm, a green emitter has an emission maximum in a range of from greater than 500 to 560 nm, a red emitter has an emission maximum in a range of from greater than 620 to 800 nm.

A further aspect of embodiments of the present disclosure relates to an OLED, which emits light having CIEx and CIEy color coordinates close to the CIEx (=0.131) and CIEy (=0.046) color coordinates of the primary color blue (CIEx=0.131 and CIEy=0.046) as defined by ITU-R Recommendation BT.2020 (Rec. 2020) and thus is suited for the use in Ultra High Definition (UHD) displays, e.g. UHD-TVs. Accordingly, a further aspect of embodiments of the present disclosure relates to an OLED, whose emission exhibits a CIEx color coordinate of between 0.02 and 0.30, between 0.03 and 0.25, between 0.05 and 0.20 or between 0.08 and 0.18 or even between 0.10 and 0.15 and/or a CIEy color coordinate of between 0.00 and 0.45, between 0.01 and 0.30, between 0.02 and 0.20 or between 0.03 and 0.15 or even between 0.04 and 0.10.

A further embodiment of embodiments of the present disclosure relates to an OLED, which emits light having CIEx and CIEy color coordinates close to the CIEx (=0.170) and CIEy (=0.797) color coordinates of the primary color green (CIEx=0.170 and CIEy=0.797) as defined by ITU-R Recommendation BT.2020 (Rec. 2020) and thus is suited for the use in Ultra High Definition (UHD) displays, e.g. UHD-TVs. In this context, the term “close to” refers to the ranges of CIEx and CIEy coordinates provided at the end of this paragraph. In commercial applications, top-emitting (top-electrode is transparent) devices may be used, whereas test devices as used throughout the present application represent bottom-emitting devices (bottom-electrode and substrate are transparent). Accordingly, a further aspect of embodiments of the present disclosure relates to an OLED, whose emission exhibits a CIEx color coordinate of between 0.15 and 0.45 between 0.15 and 0.35, between 0.15 and 0.30 or between 0.15 and 0.25 or even between 0.15 and 0.20 and/or a CIEy color coordinate of between 0.60 and 0.92, between 0.65 and 0.90, between 0.70 and 0.88 or between 0.75 and 0.86 or even between 0.79 and 0.84.

A further aspect of embodiments of the present disclosure relates to an OLED, which exhibits an external quantum efficiency at 14500 cd/m² of more than 10%, of more than 13%, of more than 15%, of more than 17% or even more than 20% and/or exhibits an emission maximum between 500 and 560 nm, between 510 and 550 nm, between 520 and 540 nm and/or exhibits an LT97 value at 14500 cd/m² of more than 100 h, more than 250 h, more than 50 h, more than 750 h or even more than 1000 h.

A further aspect of embodiments of the present disclosure relates to an OLED, which exhibits an external quantum efficiency at 1000 cd/m² of more than 8%, of more than 10%, of more than 13%, of more than 15% or even more than 20% and/or exhibits an emission maximum between 420 and 500 nm, between 430 and 490 nm, between 440 and 480 nm or still and/or exhibits an LT80 value at 500 cd/m2 of more than 100 h, more than 200 h, more than 400 h, more than 750 h or even more than 1000 h.

A further aspect of embodiments of the present disclosure relates to an OLED, which emits light at a distinct color point. According to embodiments of the present disclosure, the OLED emits light having a narrow emission band (small full width at half maximum (FWHM). In one aspect, the OLED according to embodiments of the present disclosure emits light having a FWHM of the main emission peak of less than 0.50 eV, less than 0.48 eV, less than 0.45 eV, less than 0.43 eV or even less than 0.40 eV.

In a further aspect, embodiments of the present disclosure relates to a method for producing an optoelectronic component. In this case an organic molecule of embodiments of the present disclosure is used.

The optoelectronic device, for example, the OLED according to embodiments of the present disclosure can be fabricated by any suitable means of vapor deposition and/or liquid processing. Accordingly, at least one layer is:
prepared by a sublimation process,
prepared by an organic vapor phase deposition process,
prepared by a carrier gas sublimation process,
solution processed and/or printed.

The methods used to fabricate the optoelectronic device, for example, the OLED according to embodiments of the present disclosure may be any suitable methods generally used in the art. The different layers are individually and successively deposited on a suitable substrate by means of subsequent deposition processes. The individual layers may be deposited using the same or differing deposition methods.

Vapor deposition processes may comprise thermal (co)evaporation, chemical vapor deposition and/or physical vapor deposition. For active matrix OLED display, an AMOLED backplane is used as a substrate. The individual layer may be processed from solutions and/or dispersions employing adequate solvents. Solution deposition processes, for example, comprise spin coating, dip coating and/or jet printing. Liquid processing may optionally be carried out in an inert atmosphere (e.g., in a nitrogen atmosphere) and/or the solvent may optionally be completely or partially removed by any suitable method generally used in the art.

### EXAMPLES

### General Synthesis Scheme I

As an example, the general synthesis scheme I provides a synthesis scheme for organic molecules M1 according to embodiments of the present disclosure, wherein the first chemical moiety has a structure according to formula I-a with T being the binding site of a single band linking the first chemical moiety to the second chemical moiety and with X being R^{X}:

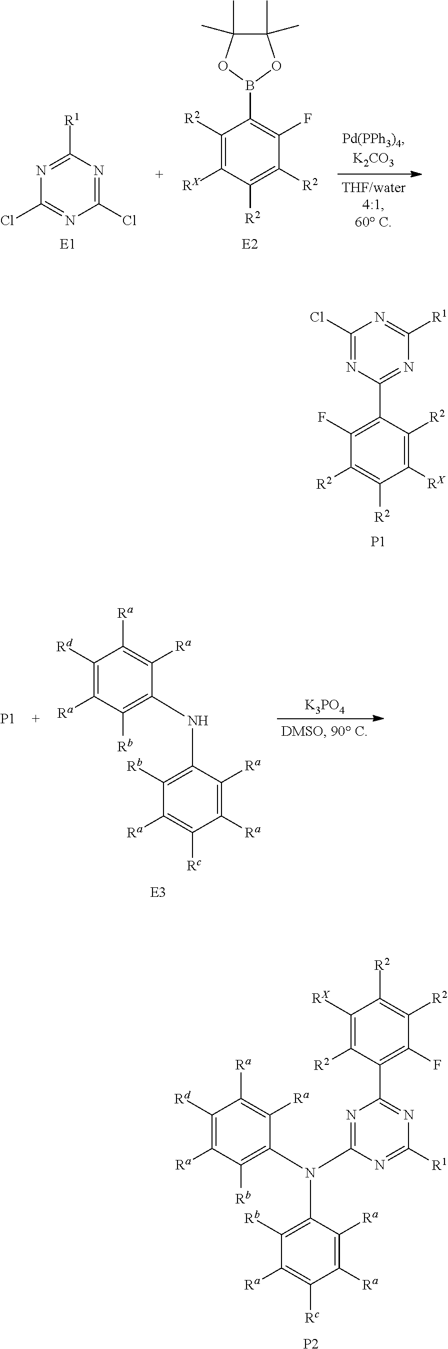

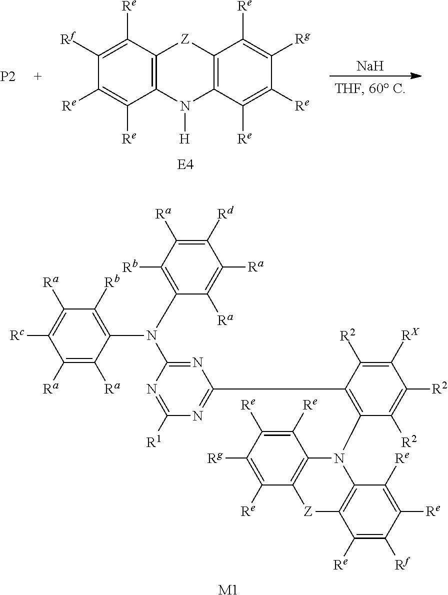

wherein, in case E3 and E4 are identical, both nucleophilic substitution reactions can be performed in a single synthetic step (in other words: M1 is obtained directly from P1). For this purpose, the reactant E3=E4 is employed in a threefold excess, as will be laid out in the synthetic procedures (procedure 4).

### General Synthesis Scheme II

As an example, the general synthesis scheme II provides a synthesis scheme for organic molecules M2 according to embodiments of the present disclosure, wherein the first chemical moiety has a structure according to formula I-b with T being the binding site of a single bond linking the first chemical moiety to the second chemical moiety and with X being R^{X}:

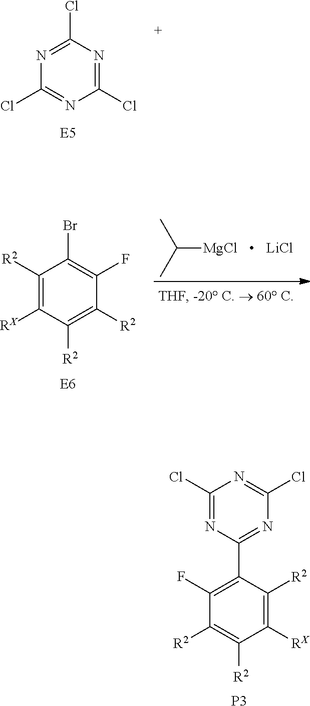

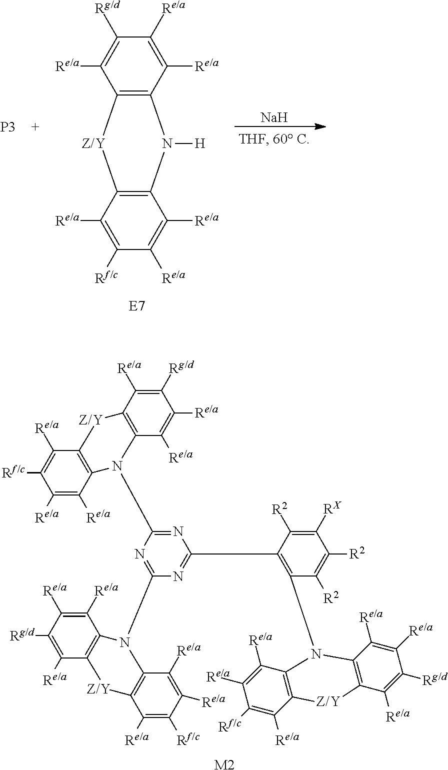

Unlike the general synthesis scheme I, general synthesis scheme II, for example, shows a two-step synthesis of compound M2, rendered feasible by the fact that all donor moieties (reactant: E7, used in excess) in M2 are, for example, chosen to be identical. As seen in the general synthesis scheme I, this is not a prerequisite. More detail can be derived from the experimental procedures.

### General Synthesis Scheme III

As an example, the general synthesis scheme Ill provides a synthesis scheme for organic molecules M3 according to embodiments of the present disclosure, wherein the first chemical moiety has a structure according to formula I-a with W being the binding site of a single band linking the first chemical moiety to the second chemical moiety and with X being R^{X}:

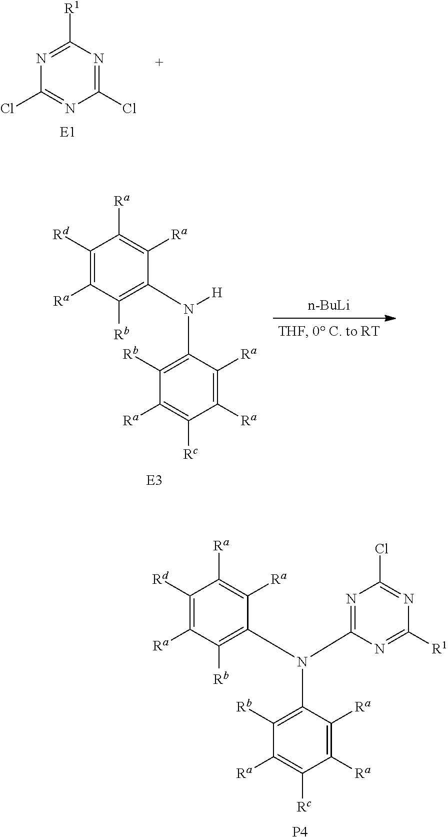

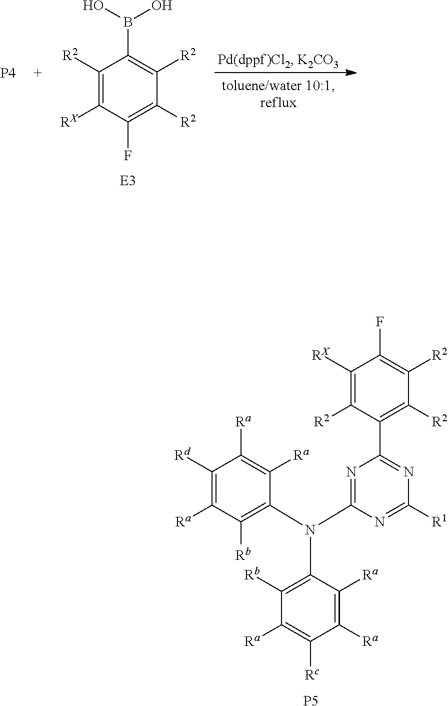

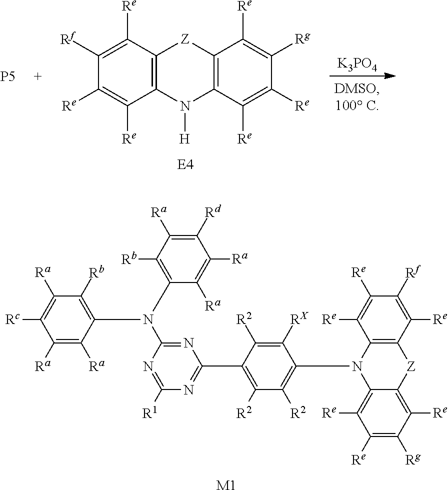

### General Synthesis Scheme IV

As an example, the general synthesis scheme IV provides a synthesis scheme for organic molecules M4 according to embodiments of the present disclosure, wherein the first chemical moiety has a structure according to formula I-b with W being the binding site of a single bond linking the first chemical moiety to the second chemical moiety and with X being R^{X}:

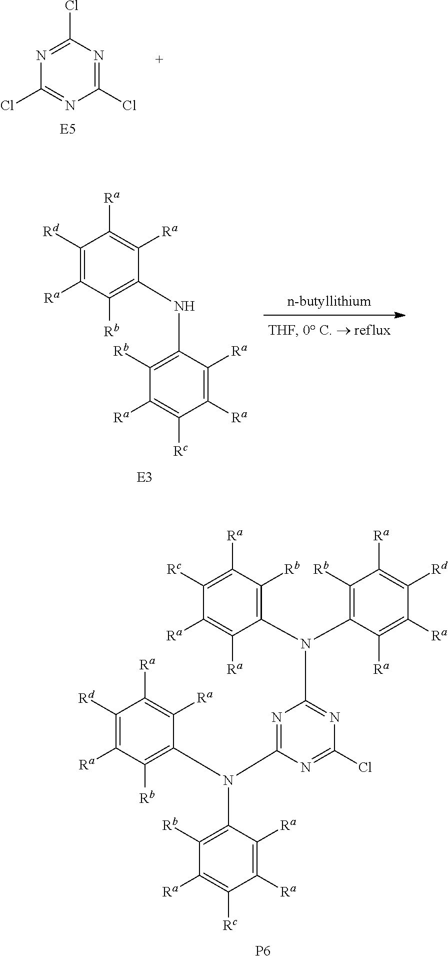

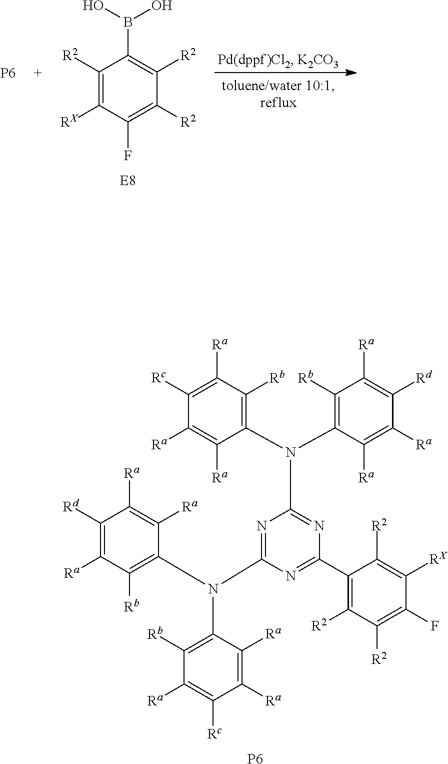

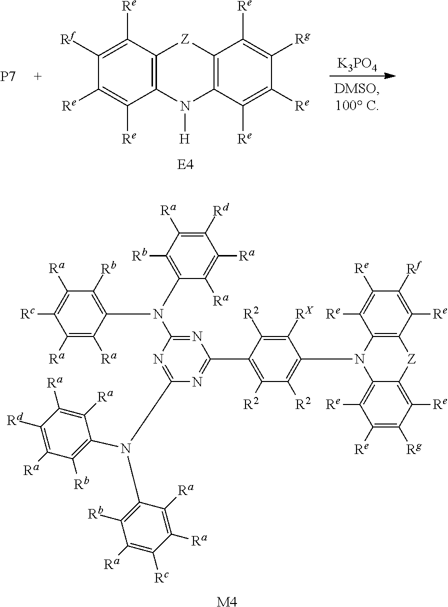

### Synthesis of E1

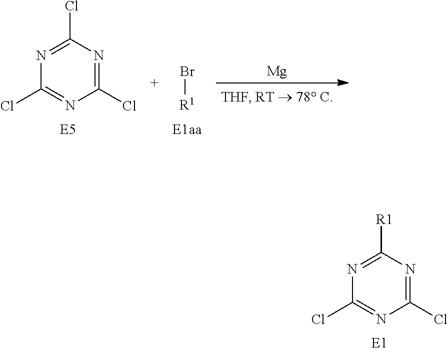

### General Synthesis Scheme V

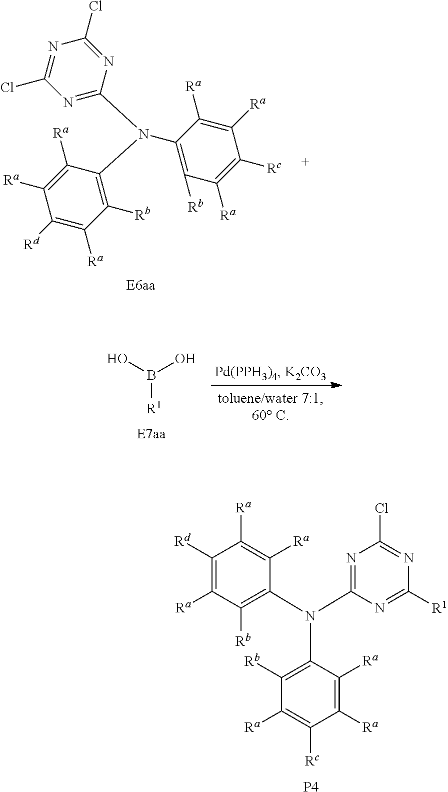

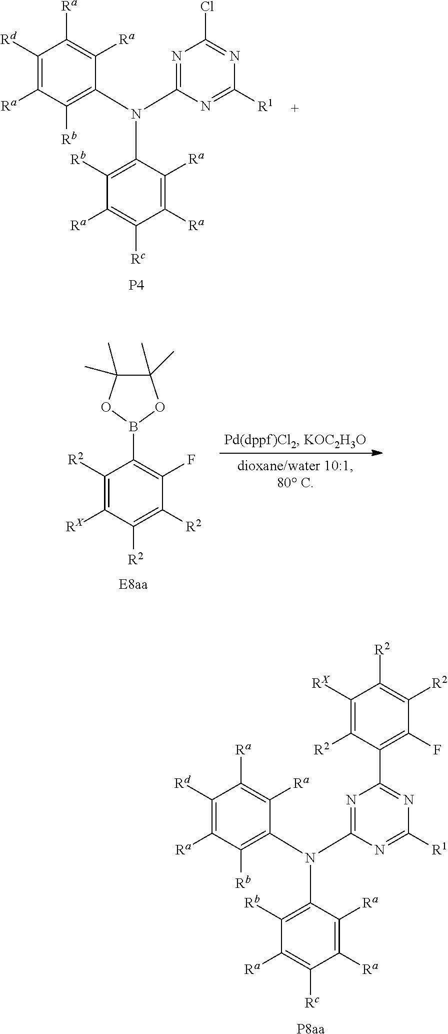

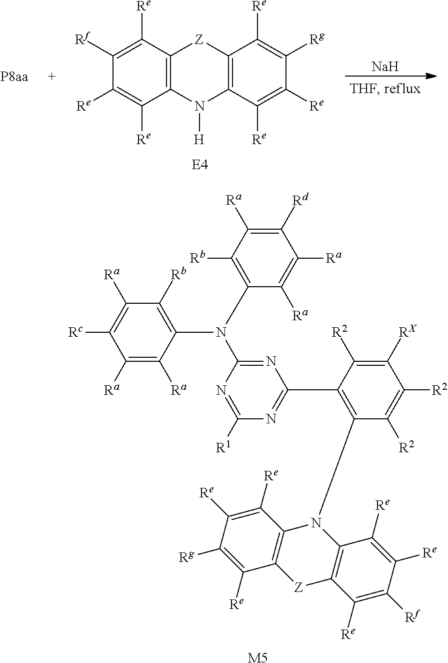

### General Synthesis Scheme VI

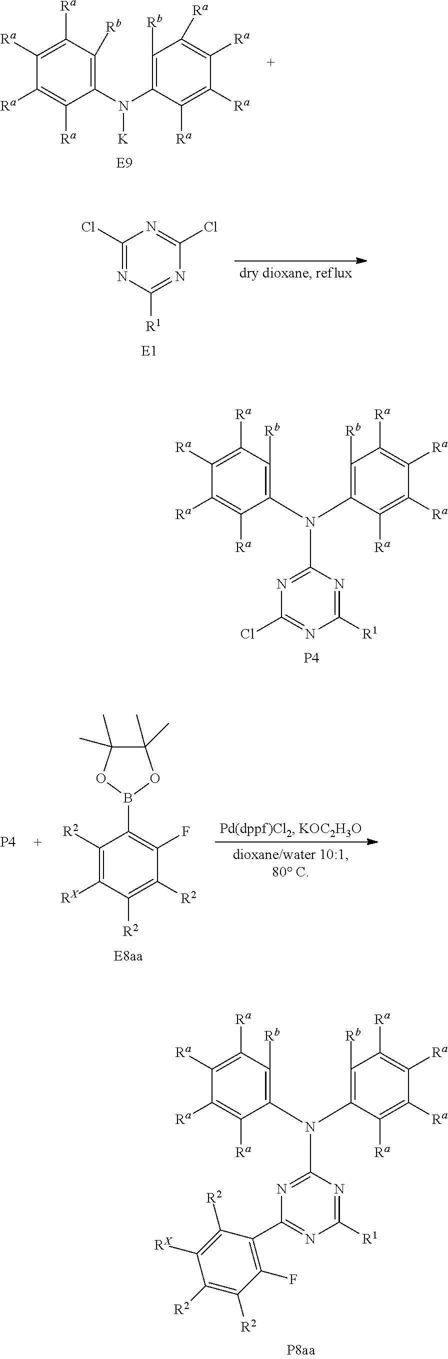

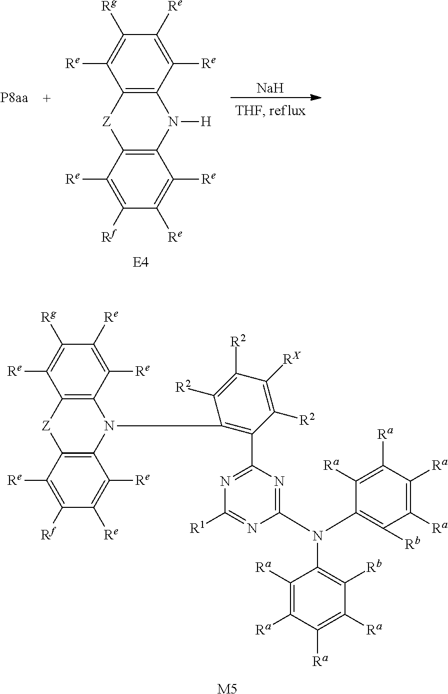

### General Synthesis Scheme VII

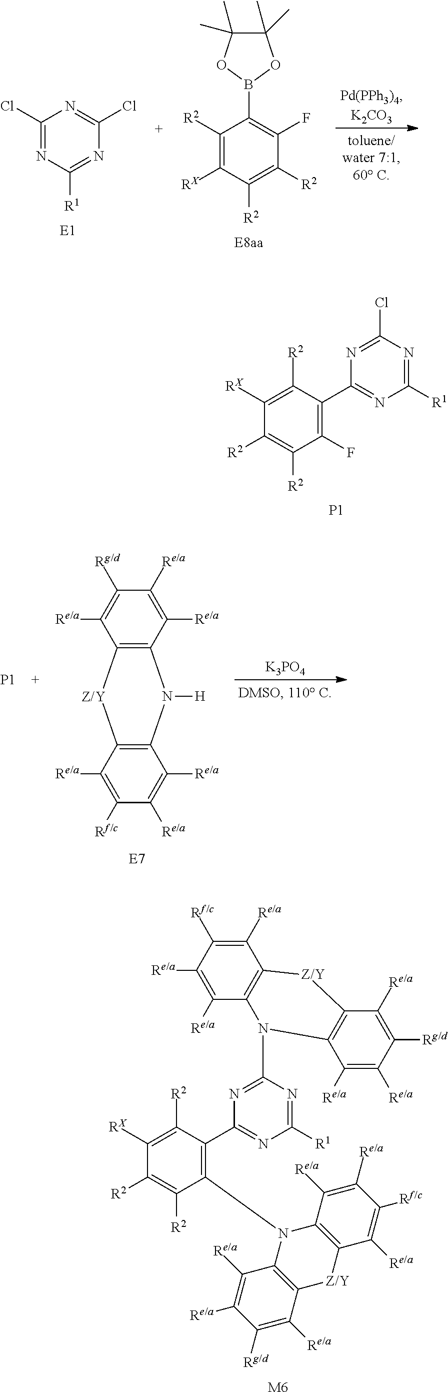

### General Procedure for Synthesis

### Procedures for Synthesis Scheme I

### Procedure 1

Under nitrogen atmosphere, a mixture of THF and water (ratio of 4:1) is added to the boronic pinacol ester E2 (1.00 equivalents), the 2,4-dichloro-1,3,5-triazine derivative (1.50 equivalents), potassium carbonate (2.00 equivalents), and tetrakis(triphenylphosphine)palladium(0) (0.03 equivalents, CAS 14221-01-3), followed by nitrogen-sparging for 10 min. The resultant reaction mixture is stirred at 60° C. until full conversion of the baronic pinacol ester E2 is reached as judged by GC/MS and TLC. After coaling to roam temperature, the reaction mixture is extracted with ethyl acetate and brine. The organic extracts are concentrated under reduced pressure. The resulting crude product is purified by column chromatography to afford P1 as a solid.

### Procedure 2

P1 (1.20 equivalents, product of procedure 1), E3 (1.00 equivalents), and tribasic potassium phosphate (2.00 equivalents) are suspended under nitrogen atmosphere in dry DMSO and stirred at 90° C. for 2 h (reaction monitored via GC/MS and TLC). Subsequently, the resultant reaction mixture is poured into a stirred mixture of water and ice. The resulting precipitate is filtered off and washed with water and ethanol. The crude product is purified by additional washing with dichloromethane to obtain P2 as a solid.

### Procedure 3

Under nitrogen atmosphere, dry THF is added to P2 (1.00 equivalents, product of procedure 2) and E4 (1.30 equivalents), followed by the addition of sodium hydride (1.30 equivalents). Once the H₂-evolution has ceased the resultant reaction mixture is heated to 60° C. under stirring. After reaction is finished based on reaction monitoring by LC/MS and TLC, the reaction mixture is carefully poured into water. The resulting precipitate is filtered off, followed by washing with water, ethanol, and hexanes. The crude product is purified by column chromatography and hot-washing with toluene to afford M1 as a solid. Alternatively, the quenched reaction mixture may be extracted with ethyl acetate and brine. The combined organic layers are then dried over MgSO₄, the solvent is removed under reduced pressure, and the residue recrystallized from ethyl acetate. The resulting crude product is heated to reflux in dichloromethane for 2 h, followed by hot filtration and washing of the solid with ethanol to afford M1 as a solid.

### Procedure 4, Two-Fold Nucleophilic Substitution Reaction (P1→M1)

The procedure is analogous to the above-mentioned procedure 3, with the exception that P1 (1.00 equivalents) is used instead of P2, alongside 3.00 equivalents of the donor molecule E3=E4 as well as 3.00 equivalents of sodium hydride.

### Procedures for Synthesis Scheme II

### Procedure 5

Under nitrogen atmosphere, E6 (1.00 equivalents) is dissolved in dry THF, followed by nitrogen sparging for 10 min. Upon cooling to −20° C., isopropylmagnesium chloride-lithium chloride complex (1.10 equivalents, CAS: 745038-86-2) is added, followed by stirring at the same temperature for 1 h. Using a cannula, the cold Grignard solution was slowly transferred into a solution of cyanuric chloride (E5, 1.10 equivalents, CAS: 108-77-0) in dry THF (nitrogen atmosphere, room temperature). The resultant reaction mixture is heated to 70° C. and stirred for 1.5 h (reaction is monitored via GC/MS and TLC) and, after cooling to room temperature, quenched by the addition of water. Extraction with dichloromethane is followed by treating the combined organic layers with charcoal, filtration, and removal of the solvent under reduced pressure. Purification of the crude product by column chromatography using cyclohexane/dichloromethane as eluents affords product P3 as a solid.

### Procedure 6

The procedure is analogous to procedure 4, with the exception that P3 (1.00 equivalent) is used instead of P1 and that E7 (4.00 equivalents) is as reactant, alongside 4.00 equivalents of sodium hydride. Product M2 is obtained as a solid.

### Procedures for Synthesis Scheme III

### Procedure 7

Under nitrogen atmosphere E3 (1.00 eq) is dissolved in THF. At 0° C., n-butyl lithium (1.0 eq, 2.5 M in hexanes) is added dropwise and subsequently stirred at room temperature for 20 min. In a separate flask E1 (1.50 eq) is dissolved in THF under nitrogen atmosphere. To this solution the previously prepared lithium species is added dropwise. Subsequently, the resultant mixture is heated at reflux until full conversion of E1 is reached as judged by GC/MS and TLC. After cooling to room temperature, water is added and the precipitated solid is filtered off and washed with water and ethanol to obtain P4 as a solid. The substance can be further purified by recrystallization.

### Procedure 8

Under nitrogen atmosphere, a mixture of toluene and water is added to the boronic acid E8 (1.20 equivalents), P4 (1.00 equivalents, product of procedure 7), potassium carbonate (2.00 equivalents), and [1,1′-bis(diphenylphosphino)ferrocene]dichloropalladium(II) (0.05 equivalents, CAS 72287-26-4). The resultant reaction mixture is stirred under reflux until full conversion of P4 is reached as judged by GC/MS and TLC. After cooling to room temperature, water is added, followed by extraction with dichloromethane and water. The combined organic layers are dried over MgSO₄ and concentrated under reduced pressure. The resulting crude product is heated to reflux in ethanol for 2 h and, upon hot filtration, washed with ethanol. The hot-filtration procedure is then repeated with a 1:1 mixture of methanol and water and the product P5 is obtained as a solid.

### Procedure 9

The procedure is analogous to procedure 2, with the exception that P5 (1.00 equivalents, product of procedure 8) is used instead of P1 and E4 (1.10 equivalents) is used instead of E3, alongside 2.20 equivalents of tribasic potassium phosphate. M3 is obtained as a solid.

### Procedures for Synthesis Scheme IV

### Procedure 10

Under nitrogen atmosphere E3 (2.00 eq) is dissolved in THF. At 0° C. n-butyl lithium (2.10 eq, 2.5 M in hexanes) is added dropwise and subsequently stirred at room temperature for 20 min. In a separate flask E5 (1.00 eq) is dissolved in THF under nitrogen atmosphere. To this solution the previously prepared lithium species is added dropwise. Subsequently the resultant mixture is heated at reflux until full conversion of E5 is reached as judged by GC/MS and TLC. After cooling to room temperature, water is added and the precipitated solid is filtered off and washed with water and ethanol to obtain P6 as a solid. The substance can be further purified by recrystallization.

### Procedure 11

The procedure is analogous to procedure 8, with the exception that P6 (1.00 equivalents, product of procedure 10) is used instead of P4. Upon completion of the reaction as judged by GC/MS and TLC, the reaction mixture is cooled to room temperature, poured into water and the resulting precipitate is filtered off. Washing with water and ethyl acetate affords P7 as a solid.

### Procedure 12

The procedure is analogous to procedure 2, with the exception that P7 (1.00 equivalents, product of procedure 11) is used instead of P1 and E4 (1.10 equivalents) is used instead of E3, alongside 2.20 equivalents of tribasic potassium phosphate. M4 is obtained as a solid.

### Procedure for Synthesis of E1

### Procedure 13

Under nitrogen atmosphere, E1aa (1.00 equivalents, CAS: 2052-07-5) is dissolved in dry THF, followed by nitrogen sparging for 10 min. The resultant solution is added dropwise to activated magnesium (3.00 equivalents, CAS: 7439-95-4) in dry THF, followed by stirring at the same temperature for 2 h. Using a cannula, the cold Grignard solution was slowly transferred into a solution of cyanuric chloride (E5, 1.50 equivalents, CAS: 108-77-0) in dry toluene (nitrogen atmosphere, room temperature). The resultant reaction mixture is heated to 78° C. and stirred for 8 h (reaction is monitored via GC/MS and TLC) and, after cooling to room temperature, quenched by the addition of water. Extraction with dichloromethane is followed by treating the combined organic layers with charcoal, filtration, and removal of the solvent under reduced pressure. Purification of the crude product by column chromatography using cyclohexane/dichloromethane as eluents affords product E1 as a solid.

### Procedures for Synthesis Scheme V

### Procedure 13 a

Under nitrogen atmosphere, a mixture of toluene and water (ratio of 7:1) is added to the boronic acid E7aa (1.00 equivalents), E6aa (1.50 equivalents), potassium carbonate (2.00 equivalents), and tetrakis(triphenylphosphine)palladium(0) (0.03 equivalents, CAS 14221-01-3), followed by nitrogen-sparging for 10 min. The resultant reaction mixture is stirred at 60° C. until full conversion of the boronic acid E7aa is reached as judged by GC/MS and TLC. After cooling to room temperature, the reaction mixture is extracted with ethyl acetate and brine. The organic extracts are concentrated under reduced pressure. The resulting crude product is purified by column chromatography to afford P4 as a solid.

### Procedure 14

Under nitrogen atmosphere, a mixture of dioxane and water (10:1) is added to the boronic acid ester E8aa (1.30 equivalents), P4 (1.00 equivalents, product of procedure 7), potassium acetate (2.00 equivalents, CAS: 127-08-2), and [1,1′-bis(diphenylphosphino)ferrocene]dichloropalladium(II) (0.05 equivalents, CAS 72287-26-4). The resultant reaction mixture is stirred at 80° C. until full conversion of P4 is reached as judged by GC/MS and TLC. After cooling to room temperature, water is added, followed by extraction with dichloromethane and water. The combined organic layers are dried over MgSO₄ and concentrated under reduced pressure. The resulting crude product is heated to reflux in ethanol for 2 h and, upon hot filtration, washed with ethanol. The hot-filtration procedure is then repeated with a 1:1 mixture of methanol and water and the product P8aa is obtained as a solid.

### Procedure 15

E4 (1.30 equivalents) and subsequently P8aa (1.00 equivalents, product of procedure 1) were added to NaH (1.40 equivalents, CAS: 7646-69-7) under nitrogen atmosphere in dry THF and stirred at reflux until completion of the reaction (reaction monitored via GC/MS and TLC). Subsequently, the resultant reaction mixture is poured into water and ice. Extraction with dichloromethane is followed by treating the combined organic layers with charcoal, filtration, and removal of the solvent under reduced pressure. Purification of the crude product by column chromatography or recrystallization affords product M5 as a solid.

### Procedures for Synthesis Scheme VI

### Procedure 16

E9 (1.00 equivalents) and E1 (1.00 equivalents) were stirred at 105° C. under nitrogen atmosphere in dry dioxane until completion of the reaction (reaction monitored via GC/MS and TLC). After cooling to room temperature, water is added, followed by extraction with dichloromethane and water. The combined organic layers are dried over MgSO₄ and concentrated under reduced pressure. The resulting crude product is heated to reflux in ethanol for 2 h and, upon hot filtration, washed with ethanol. P4 is obtained as a solid.

### Procedure 17

The procedure is analogous to the above-mentioned procedure 14, whereas for the hot-filtration procedure THF was used instead of ethanol.

### Procedure 18

The procedure is analogous to the above-mentioned procedure 15.

### Procedures for Synthesis Scheme VII

### Procedure 19

The procedure is analogous to the above-mentioned procedure 1.

### Procedure 20

P1 (1.00 equivalents), E7 (3.00 equivalents), and tribasic potassium phosphate (4.00 equivalents) are suspended under nitrogen atmosphere in dry DMSO and stirred at 90° C. for 2 h (reaction monitored via GC/MS and TLC). Subsequently, the resultant reaction mixture is poured into a stirred mixture of water and ice. The resulting precipitate is filtered off and washed with water and ethanol. The crude product is purified by additional washing with dichloromethane to obtain M6 as a solid.

### Cyclic Voltammetry

Cyclic voltammograms are measured from solutions having a concentration of 10⁻³ mol/L of respective organic molecules in dichloromethane or a suitable solvent and a suitable supporting electrolyte (e.g., 0.1 mol/L of tetrabutylammonium hexafluorophosphate). The measurements are conducted at room temperature under nitrogen atmosphere with a three-electrode assembly (Working and counter electrodes: Pt wire, reference electrode: Pt wire) and calibrated using FeCp₂/FeCp₂⁺ as internal standard. The HOMO data was corrected using ferrocene as internal standard against a saturated calomel electrode (SCE).

### Density Functional Theory Calculation

Molecular structures are optimized employing the BP86 functional and the resolution of identity approach (RI). Excitation energies are calculated using the (BP86) optimized structures employing Time-Dependent DFT (TD-DFT) methods. Orbital and excited state energies are calculated with the B3LYP functional. Def2-SVP basis sets and a m4-grid for numerical integration were used. The Turbomole program package was used for all calculations.

### Photophysical Measurements

Sample pretreatment: Spin-coating

Apparatus: Spin150, SPS euro.

The sample concentration is 0.2 mg/ml, dissolved in Toluene/DCM as suitable solvent.

Program: 7-30 sec. at 2000 U/min. After coating, the films are tried at 70° C. for 1 min.

### Absorption Measurements

A Thermo Scientific Evolution 201 UV-Visible Spectrophotometer is used to determine wavelength of the absorption maximum of the sample in the wavelength region above 270 nm. This wavelength is used as excitation wavelength for photoluminescence spectral and quantum yield measurements.

Photoluminescence Spectroscopy and Phosphorescence Spectroscopy

For the analysis of Phosphorescence and Photoluminescence spectroscopy a fluorescence spectrometer “Fluoromax 4P” from Horiba is used.

Time-resolved PL spectroscopy in the μs-range and ns-range (FS5)

Time-resolved PL measurements were performed on a FS5 fluorescence spectrometer from Edinburgh Instruments. Compared to measurements on the HORIBA setup, better light gathering allows for an optimized signal to noise ratio, which favors the FS5 system especially for transient PL measurements of delayed fluorescence characteristics. As continuous light source, the spectrometer comprises a 150 W xenon arc lamp and set or specific wavelengths may be selected by a Czerny-Turner monochromator. However, the standard measurements were instead performed using an external VPLED variable pulsed LED with an emission wavelength of 310 nm. The sample emission is directed towards a sensitive R928P photomultiplier tube (PMT), allowing the detection of single photons with a peak quantum efficiency of up to 25% in the spectral range between 200 nm to 870 nm. The detector is a temperature stabilized PMT, providing dark counts below 300 cps (counts per second). Finally, to determine the transient decay lifetime of the delayed fluorescence, a tail fit using three exponential functions is applied. By weighting set or specific lifetimes Ti with their corresponding amplitudes Aᵢ,

$${τ}_{D F} = {\sum }_{i = 1}^{3} \frac{{A}_{i} {τ}_{i}}{{A}_{i}}$$

the delayed fluorescence lifetime τ_{DF} is determined.

### Photoluminescence Quantum Yield Measurements

For photoluminescence quantum yield (PLQY) measurements an Absolute PL Quantum Yield Measurement C9920-03G system (Hamamatsu Photonics) is used. Quantum yields and CIE coordinates are determined using the software U6039-05 version 3.6.0.

Emission maxima are given in nm, quantum yields D in % and CIE coordinates as x,y values.

PLQY is determined using the following protocol:

Quality assurance: Anthracene in ethanol (known concentration) is used as reference

Excitation wavelength: the absorption maximum of the organic molecule is determined and the molecule is excited using this wavelength

Measurement

Quantum yields are measured for sample of films (10% by weight of the emitter in PMMA) under nitrogen atmosphere. The yield is calculated using the equation:

$${Φ}_{P L} = \frac{{n}_{photon} , emitted}{{n}_{p h o t o n} , absorbed} = \frac{\int \frac{λ}{hc} [ {Int}_{e m i t t e d}^{sample} ( λ ) - {Int}_{absorbed}^{sample} ( λ ) ] d λ}{\int \frac{λ}{h c} [ {Int}_{e m i t t e d}^{reference} ( λ ) - {Int}_{absorbed}^{reference} ( λ ) ] d λ}$$

wherein nₚₕₒₜₒₙ denotes the photon count and Int. the intensity. For quality assurance, anthracene in ethanol (known concentration) is used as reference.

### TCSPC (Time-Correlated Single-Photon Counting)

Excited state population dynamics are determined employing Edinburgh Instruments FS5 Spectrofluoremeters, equipped with an emission monochromator, a temperature stabilized photomultiplier as a detector unit and a pulsed LED (310 nm central wavelength, 910 μs pulse width) as excitation source. The samples are placed in a cuvette and flushed with nitrogen during the measurements.

### Full Decay Dynamics

The full excited state population decay dynamics over several orders of magnitude in time and signal intensity is achieved by carrying out TCSPC measurements in 4 time windows: 200 ns, 1 μs, and 20 μs, and a longer measurement spanning greater than 80 μs. The measured time curves are then processed in the following way:

A background correction is applied by determining the average signal level before excitation and subtracting.

The time axes are aligned by taking the initial rise of the main signal as reference.

The curves are scaled onto each other using overlapping measurement time regions.

The processed curves are merged to one curve.

### Data Analysis

Data analysis was done using monoexponential and bi-exponential fitting of prompt fluorescence (PF) and delayed fluorescence (DF) decays separately. The ratio of delayed and prompt fluorescence (n-value) is calculated by the integration of respective photoluminescence decays in time.

$$\frac{\int {I}_{D F} ( t ) d t}{\int {I}_{P F} ( t ) d t} = n$$

The average excited state life time is calculated by taking the average of prompt and delayed fluorescence decay time, weighted with the respective contributions of PF and DF.

### Production and Characterization of Optoelectronic Devices

Via vacuum-deposition methods OLED devices comprising organic molecules according to embodiments of the present disclosure can be produced. If a layer contains more than one compound, the weight-percentage of one or more compounds is given in %. The total weight-percentage values amount to 100%, thus if a value is not given, the fraction of this compound equals to the difference between the given values and 100%.

The not fully optimized OLEDs are characterized using standard methods and measuring electroluminescence spectra, the external quantum efficiency (in %) in dependency on the intensity, calculated using the light detected by the photodiode, and the current. The OLED device lifetime is extracted from the change of the luminance during operation at constant current density. The LT50 value corresponds to the time, where the measured luminance decreased to 50% of the initial luminance, analogously LT80 corresponds to the time point, at which the measured luminance decreased to 80% of the initial luminance, LT97 to the time point, at which the measured luminance decreased to 97% of the initial luminance etc.

Accelerated lifetime measurements are performed (e.g., applying increased current densities). For example, LT80 values at 500 cd/m² are determined using the following equation:

$$LT 80 ( 500 \frac{{cd}^{2}}{{m}^{2}} ) = LT 80 ( {L}_{0} ) {( \frac{{L}_{0}}{500 \frac{{cd}^{2}}{{m}^{2}}} )}^{1.6}$$

wherein L₀ denotes the initial luminance at the applied current density.

The values correspond to the average of several pixels (for example, two to eight), the standard deviation between these pixels is given. The accompanying drawings show the data series for one OLED pixel, respectively.

### HPLC-MS

This analysis is performed on an HPLC-MS by Agilent (HPLC1260 Infinity) with MS-detector

### Single Quadrupole

For example, an example HPLC method is as follows: a reverse phase column 3.0 mm×100 mm, particle size 2.7 μm from Agilent (Poroshell 120EC-C18, 3.0×100 mm, 2.7 μm HPLC column) is used in the HPLC. The HPLC-MS measurements are performed at 45° C. and an example gradient is as follows:

| Flow rate | Time | A | B | C |
|---|---|---|---|---|
| [ml/min] | [min] | [%] | [%] | [%] |
| 2.1 | 0.0 | 40 | 50 | 10 |
| 2.1 | 1.00 | 40 | 50 | 10 |
| 2.1 | 3.50 | 10 | 65 | 25 |
| 2.1 | 6.00 | 10 | 40 | 50 |
| 2.1 | 8.00 | 10 | 10 | 80 |
| 2.1 | 11.50 | 10 | 10 | 80 |
| 2.1 | 11.51 | 40 | 50 | 10 |
| 2.1 | 12.50 | 40 | 50 | 10 |

and the following solvent mixtures (all solvents contain 0.1% (V/v) of formic acid):

| | | |
|---|---|---|
| Solvent A: | H₂O (10%) | MeCN (90%) |
| Solvent B: | H₂O (90%) | MeCN (10%) |
| Solvent C: | THF (50%) | MeCN (50%) |

An injection volume of 2 μL of a solution with a concentration of 0.5 mg/mL of the analyte is used for the measurements.

Ionization of the probe is performed using an atmospheric pressure chemical ionization (APCI) source either in positive (APCI +) or negative (APCI −) ionization mode or an atmospheric pressure photoionization (APPI) source.

### Example 1

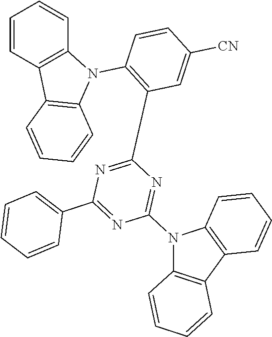 [SMILES: N#Cc1ccc(-n2c3ccccc3c3ccccc32)c(-c2nc(-c3ccccc3)nc(-n3c4ccccc4c4ccccc43)n2)c1]

Example 1 was synthesized according to procedure 1 (yield 64%), and procedure 4 (yield 32%).

MS (HPLC-MS), m/z (retention time): 589.6 (5.53 min).

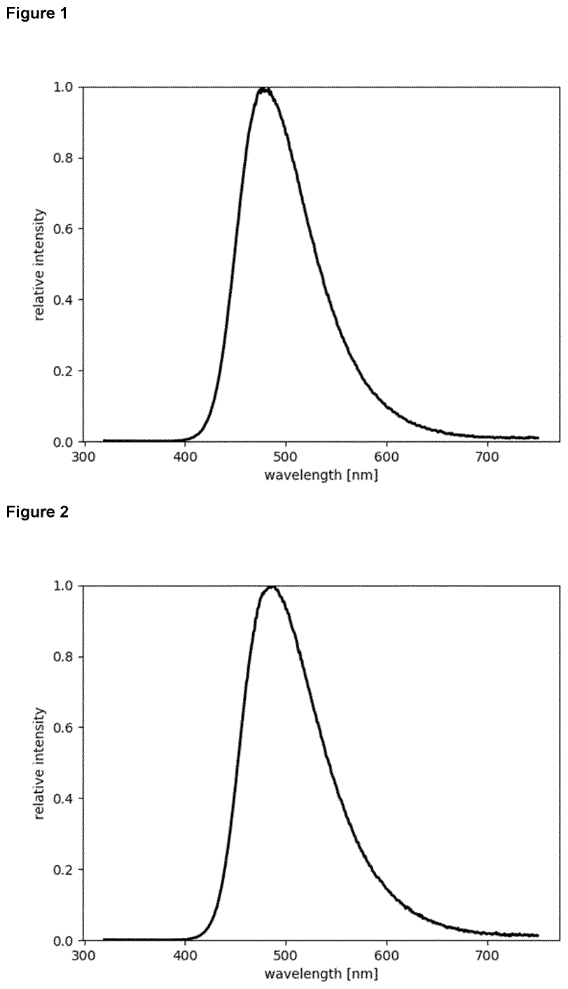

FIG. 1 depicts the emission spectrum of example 1 (10% by weight in PMMA) at room temperature (e.g., approximately 20° C.). The emission maximum (λₘₐₓ) is at 478 nm. The photoluminescence quantum yield (PLQY) is 79%, the full width at half maximum (FWHM) is 0.43 eV, and the emission lifetime is 25.8 μs. The resulting CIEₓ coordinate is determined at 0.18 and the CIE_{y} coordinate at 0.31.

### Example 2

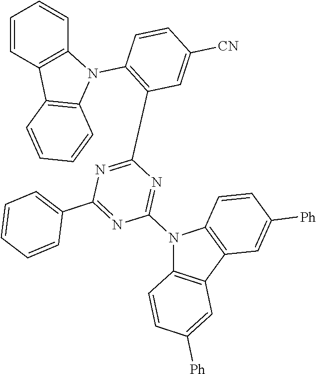 [SMILES: N#Cc1ccc(-n2c3ccccc3c3ccccc32)c(-c2nc(-c3ccccc3)nc(-n3c4ccc(-c5ccccc5)cc4c4cc(-c5ccccc5)ccc43)n2)c1]

Example 2 was synthesized according to procedure 1 (yield 64%), procedure 2 (yield 52%), and procedure 3 (yield 27%).

MS (HPLC-MS), m/z (retention time): 741.7 (5.53 min).

FIG. 2 depicts the emission spectrum of example 2 (10% by weight in PMMA) at room temperature (e.g., approximately 20° C.). The emission maximum (λₘₐₓ) is at 485 nm. The photoluminescence quantum yield (PLQY) is 59%, the full width at half maximum (FWHM) is 0.45 eV, and the emission lifetime is 21.3 μs. The resulting CIEₓ coordinate is determined at 0.21 and the CIE_{y} coordinate at 0.36.

### Example 3

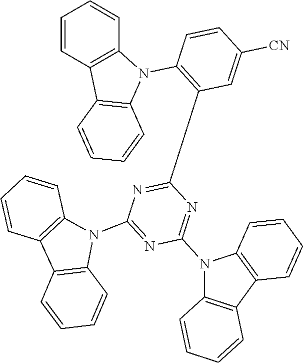 [SMILES: N#Cc1ccc(-n2c3ccccc3c3ccccc32)c(-c2nc(-n3c4ccccc4c4ccccc43)nc(-n3c4ccccc4c4ccccc43)n2)c1]

Example 3 was synthesized according to procedure 5 (yield 32%) and procedure 6 (yield 59%).

MS (HPLC-MS), m/z (retention time): 678.7 (5.00 min).

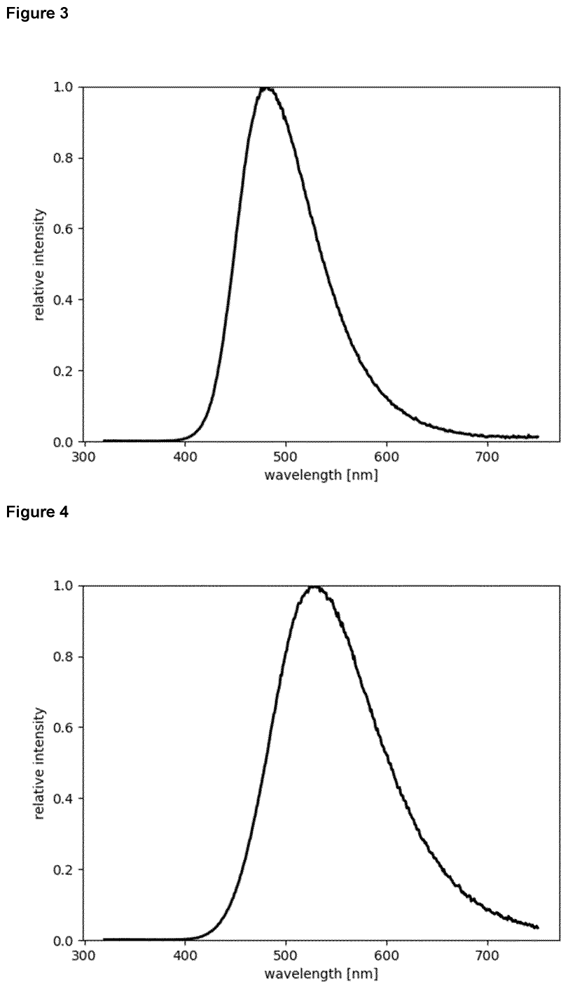

FIG. 3 depicts the emission spectrum of example 3 (10% by weight in PMMA) at room temperature (e.g., approximately 20° C.). The emission maximum (λₘₐₓ) is at 482 nm. The photoluminescence quantum yield (PLQY) is 74%, the full width at half maximum (FWHM) is 0.47 eV, and the emission lifetime is 28.6 μs. The resulting CIEₓ coordinate is determined at 0.19 and the CIE_{y} coordinate at 0.32.

### Example 4

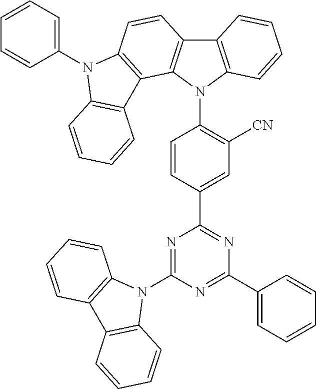 [SMILES: N#Cc1cc(-c2nc(-c3ccccc3)nc(-n3c4ccccc4c4ccccc43)n2)ccc1-n1c2ccccc2c2ccc3c(c4ccccc4n3-c3ccccc3)c21]

Example 4 was synthesized according to procedure 7 (yield 60%), procedure 8 (yield 89%), and procedure 9 (yield 79%).

MS (HPLC-MS), m/z (retention time): 754.8 (5.80 min).

FIG. 4 depicts the emission spectrum of example 4 (10% by weight in PMMA) at room temperature (e.g., approximately 20° C.). The emission maximum (λₘₐₓ) is at 528 nm. The full width at half maximum (FWHM) is 0.52 eV, and the emission lifetime is 10.8 μs. The resulting CIEₓ coordinate is determined at 0.34 and the CIE_{y} coordinate at 0.50.

### Example 5

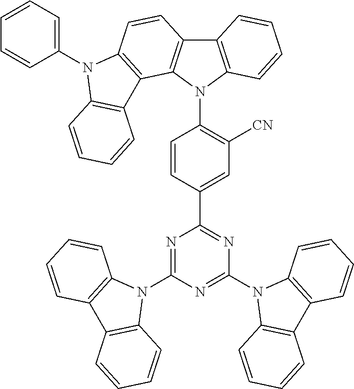 [SMILES: N#Cc1cc(-c2nc(-n3c4ccccc4c4ccccc43)nc(-n3c4ccccc4c4ccccc43)n2)ccc1-n1c2ccccc2c2ccc3c(c4ccccc4n3-c3ccccc3)c21]

Example 5 was synthesized according to procedure 11 (yield 88%, procedure 10 not performed as E8 is commercially available in this case), and procedure 12 (yield 48%).

MS (HPLC-MS), m/z (retention time): 843.9 (6.51 min).

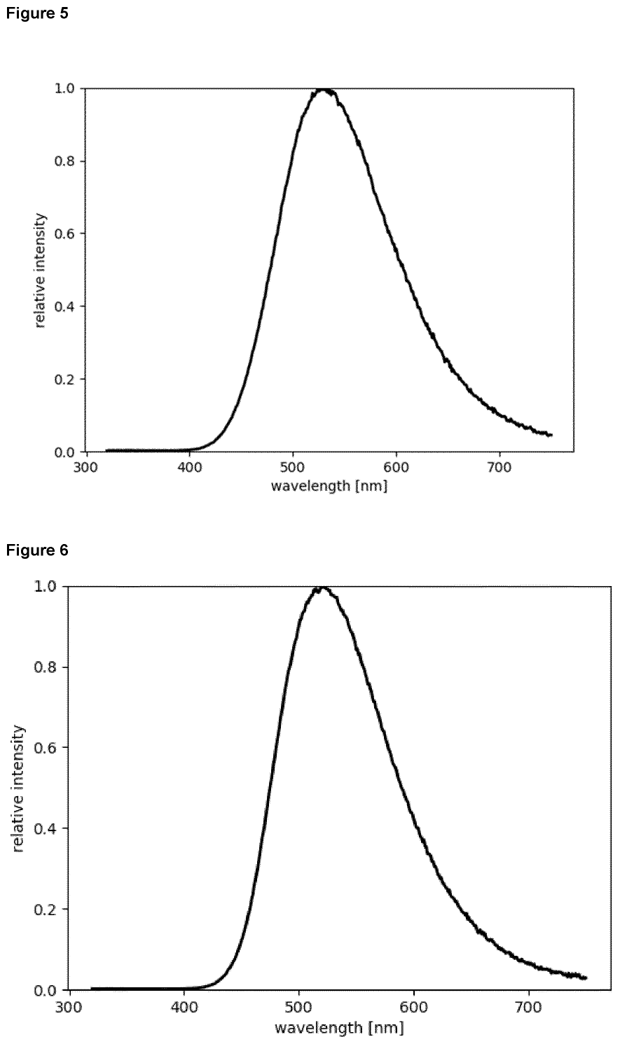

FIG. 5 depicts the emission spectrum of example 5 (10% by weight in PMMA) at room temperature (e.g., approximately 20° C.). The emission maximum (λₘₐₓ) is at 527 nm. The full width at half maximum (FWHM) is 0.56 eV, and the emission lifetime is 8.0 μs. The resulting CIEₓ coordinate is determined at 0.34 and the CIE_{y} coordinate at 0.49.

### Example 6

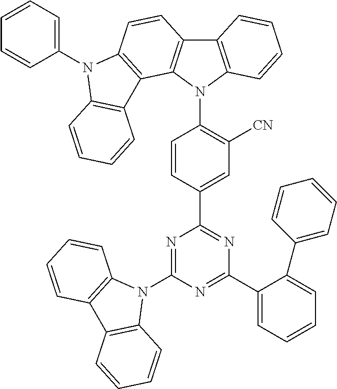 [SMILES: N#Cc1cc(-c2nc(-c3ccccc3-c3ccccc3)nc(-n3c4ccccc4c4ccccc43)n2)ccc1-n1c2ccccc2c2ccc3c(c4ccccc4n3-c3ccccc3)c21]

Example 6 was synthesized according to procedure 13 using 2-bromobiphenyl (CAS: 2052-07-5) as E1aa ((yield 10%), procedure 7 using carbazole (CAS: 86-74-8) as E3 (yield 41%), procedure 8 (yield 17%) using 3-cyano-4-fluorophenylboronic acid (CAS:214210-21-6) as E8, and procedure 9 (yield 30%) using 5,12-dihydro-5-phenyl-Indolo[3,2-a]carbazole (CAS: 1247053-55-9) as E4.

MS (HPLC-MS), m/z (retention time): 830.9 (5.88 min).

FIG. 6 depicts the emission spectrum of example 6 (10% by weight in PMMA) at room temperature (e.g., approximately 20° C.). The emission maximum (λₘₐₓ) is at 520 nm. The full width at half maximum (FWHM) is 0.51 eV, and the emission lifetime is 12.7 μs. The resulting CIEₓ coordinate is determined at 0.31 and the CIE_{y} coordinate at 0.50.

### Example 7

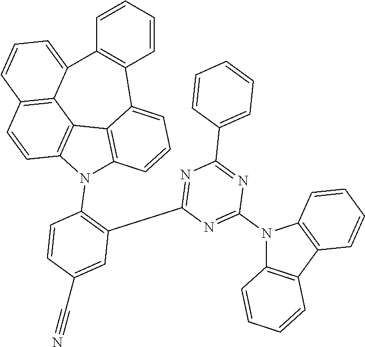 [SMILES: N#Cc1ccc(-n2c3cccc4c3c3c5c(cccc5ccc32)-c2ccccc2-4)c(-c2nc(-c3ccccc3)nc(-n3c4ccccc4c4ccccc43)n2)c1]

Example 7 was synthesized according to procedure 14 (yield 74%) using 9-(4-chloro-6-phenyl-1,3,5-triazin-2-yl)-9H-carbaz (CAS: 1268244-56-9) and 4-fluoro-3-(4,4,5,5-tetramethyl-1,3,2-dioxaborolan-2-yl)benzonitrile (CAS: 863868-29-59) as P4 and E8aa, respectively as and procedure 15 (yield 62%) using 3H-3-azadibenzo[g,ij]naphth[2,1,8-cde]azulene (CAS: 2408302-78-1) as E4.

MS (HPLC-MS), m/z (retention time): 713.6 (4.97 min).

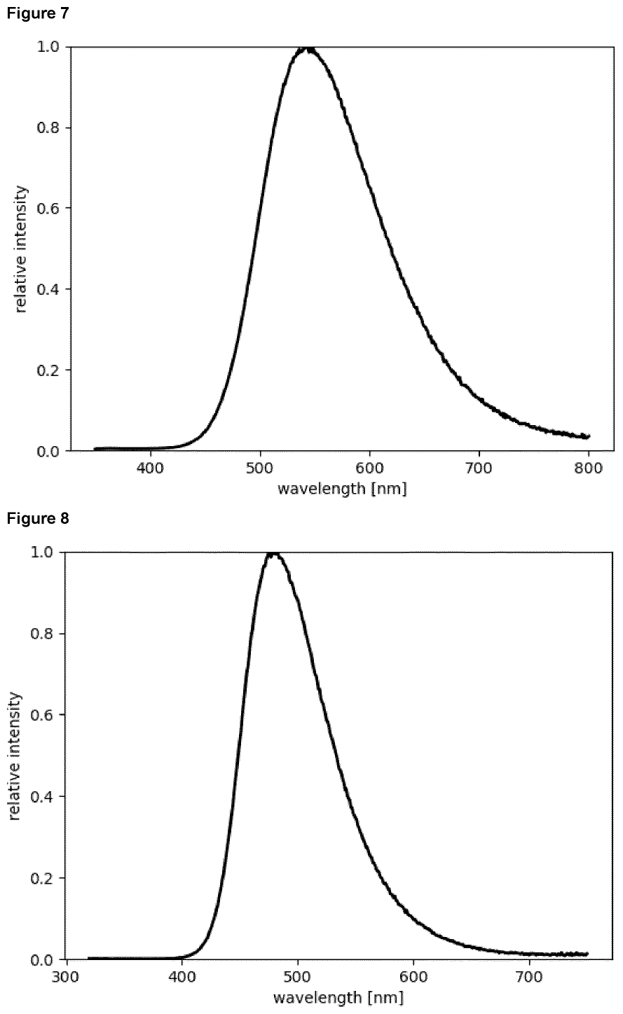

FIG. 7 depicts the emission spectrum of example 7 (10% by weight in PMMA) at room temperature (e.g., approximately 20° C.). The emission maximum (λₘₐₓ) is at 541 nm. The full width at half maximum (FWHM) is 0.51 eV. The resulting CIEₓ coordinate is determined at 0.39 and the CIE_{y} coordinate at 0.53.

### Example 8

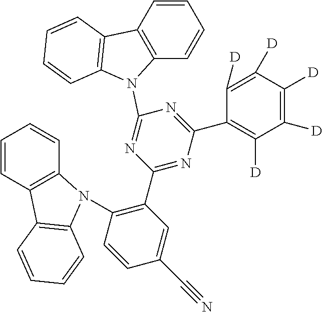 [SMILES: [2H]c1c([2H])c([2H])c(-c2nc(-c3cc(C#N)ccc3-n3c4ccccc4c4ccccc43)nc(-n3c4ccccc4c4ccccc43)n2)c([2H])c1[2H]]

Example 8 was synthesized according to procedure 13a (yield 47%) using

9-(4,6-dichloro-[1,3,5]triazin-2-yl)-carbazole (CAS: 24209-95-8) and phenyl-d5-boronic acid (CAS: 215527-70-1) as E6aa and E7aa, respectively, procedure 14 (yield 69%) using 4-fluoro-3-(4,4,5,5-tetramethyl-1,3,2-dioxaborolan-2-yl)benzonitrile (CAS: 863868-29-59) as E8aa, respectively and procedure 15 (yield 41%) using carbazole (CAS: 86-74-8) as E4.

MS (HPLC-MS), m/z (retention time): 594.6 (4.46 min).

FIG. 8 depicts the emission spectrum of example 8 (10% by weight in PMMA) at room temperature (e.g., approximately 20° C.). The emission maximum (λₘₐₓ) is at 479 nm. The photoluminescence quantum yield (PLQY) is 78%, the full width at half maximum (FWHM) is 0.44 eV, and the emission lifetime is 29.3 μs. The resulting CIEₓ coordinate is determined at 0.18 and the CIE_{y} coordinate at 0.31.

### Example 9

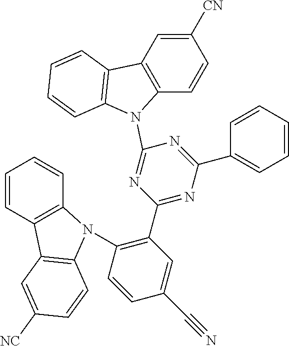 [SMILES: N#Cc1ccc(-n2c3ccccc3c3cc(C#N)ccc32)c(-c2nc(-c3ccccc3)nc(-n3c4ccccc4c4cc(C#N)ccc43)n2)c1]

Example 9 was synthesized according to procedure 1 (yield 42%) using 2,4-dichloro-6-phenyl-1,3,5-triazine (CAS: 1700-02-3) and 4-fluoro-3-(4,4,5,5-tetramethyl-1,3,2-dioxaborolan-2-yl)benzonitrile (CAS: 863868-29-5) as E1 and E2, respectively and procedure 2 (yield 38%) using 9H-carbazole-3-carbonitrile (3.00 equivalents, CAS: 57102-93-9) as E3, while the reaction was performed at 120° C. to yield directly M1 as product.

MS (HPLC-MS), m/z (retention time): 639.6 (3.50 min).

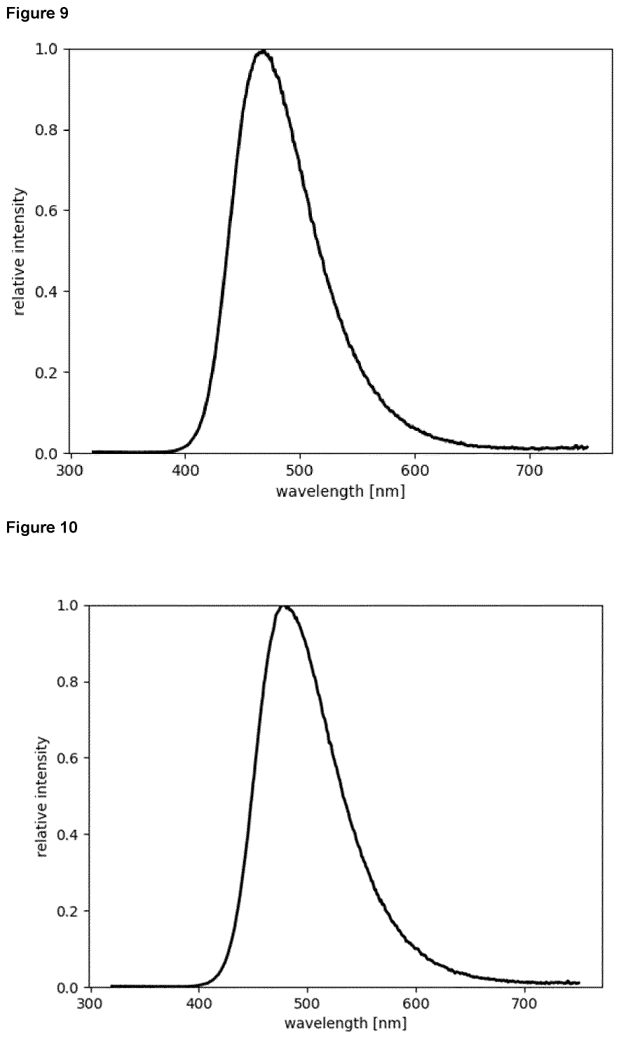

FIG. 9 depicts the emission spectrum of example 9 (10% by weight in PMMA) at room temperature (e.g., approximately 20° C.). The emission maximum (λₘₐₓ) is at 468 nm. The photoluminescence quantum yield (PLQY) is 71%, the full width at half maximum (FWHM) is 0.44 eV, and the emission lifetime is 38.0 μs. The resulting CIEₓ coordinate is determined at 0.17 and the CIE_{y} coordinate at 0.22.

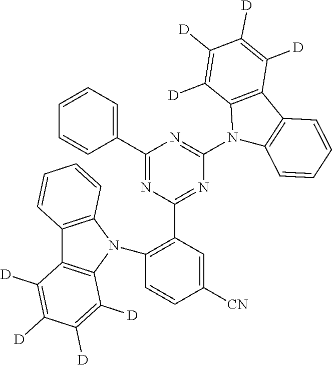 [SMILES: [2H]c1c([2H])c([2H])c2c(c1[2H])c1ccccc1n2-c1nc(-c2ccccc2)nc(-c2cc(C#N)ccc2-n2c3ccccc3c3c([2H])c([2H])c([2H])c([2H])c32)n1]

### Example 10

Example 10 was synthesized according to procedure 19 (yield 38%) using 2,4-dichloro-6-phenyl-1,3,5-triazine (CAS: 1700-02-3) and 5-cyano-2-fluorobenzeneboronic acid (CAS: 468718-30-1) as E1 and E8aa, respectively and procedure 20 (yield 26%) using 9H-carbazole-1,2,3,4-d4 (3.00 equivalents, CAS: 935425-39-1) as E7. M7 is obtained as a solid. MS (HPLC-MS), m/z (retention time) 597.7 (4.341 min).

FIG. 10 depicts the emission spectrum of example 10 (10% by weight in PMMA) at room temperature (e.g., approximately 20° C.). The emission maximum (λₘₐₓ) is at 479 nm. The photoluminescence quantum yield (PLQY) is 76%, the full width at half maximum (FWHM) is 0.43 eV, and the emission lifetime is 28.9 μs. The resulting CIEₓ coordinate is determined at 0.18 and the CIE_{y} coordinate at 0.32.

### Example 11

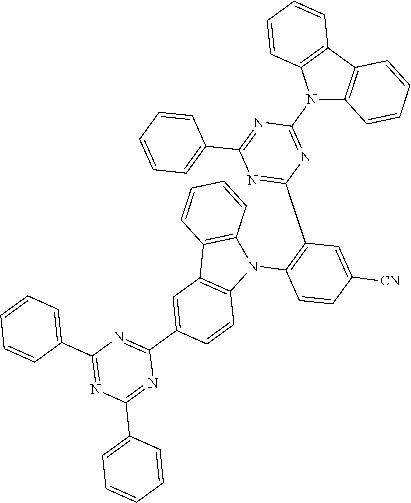 [SMILES: N#Cc1ccc(-n2c3ccccc3c3cc(-c4nc(-c5ccccc5)nc(-c5ccccc5)n4)ccc32)c(-c2nc(-c3ccccc3)nc(-n3c4ccccc4c4ccccc43)n2)c1]

Example 11 was synthesized according to procedure 16 (yield 57%) using carbazole potassium salt (CAS: 6033-87-0) as E9 and 2,4-dichloro-6-phenyl-1,3,5-triazine (CAS: 1700-02-3) as E1, procedure 17 with 4-fluoro-3-(4,4,5,5-tetramethyl-1,3,2-dioxaborolan-2-yl)benzonitrile (CAS: 863868-29-5) as E8aa (yield 58%) and procedure 18 with 3-(4,6-diphenyl-1,3,5-trazin-2-yl)-9H-carbazole (CAS: 1313391-57-9) as E4 (yield 57%). MS (HPLC-MS), m/z (retention time) 820.9 (6.137 min).

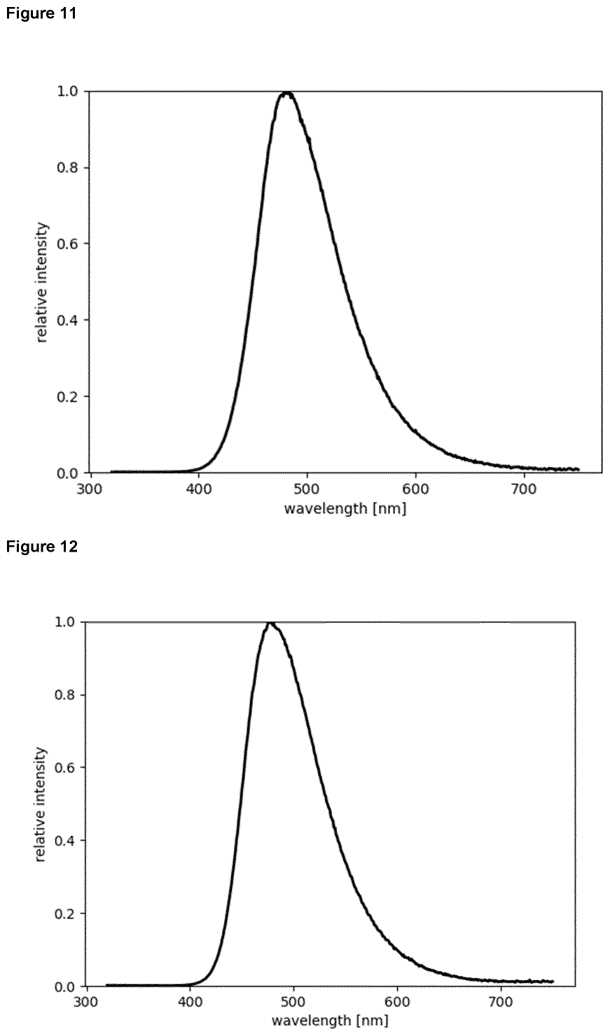

FIG. 11 depicts the emission spectrum of example 10 (10% by weight in PMMA) at room temperature (e.g., approximately 20° C.). The emission maximum (λₘₐₓ) is at 482 nm. The photoluminescence quantum yield (PLQY) is 58%, the full width at half maximum (FWHM) is 0.43 eV, and the emission lifetime is 33.3 μs. The resulting CIEₓ coordinate is determined at 0.19 and the CIE_{y} coordinate at 0.32.

### Example 12

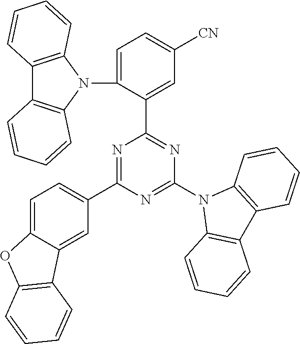 [SMILES: N#Cc1ccc(-n2c3ccccc3c3ccccc32)c(-c2nc(-c3ccc4oc5ccccc5c4c3)nc(-n3c4ccccc4c4ccccc43)n2)c1]

Example 12 was synthesized according to procedure 13 with 9-(4,6-dichloro-1,3,5-trazin-2-yl)-carbazole (CAS: 24209-95-8) as E6aa and dibenzo[b,d]furan-2-ylboronic acid (CAS: 402936-15-6) as E7aa (yield 53.5%), whereas the crude product was purified by two hot-filtration procedures using ethanol and a 1:1 mixture of methanol and water, procedure 14 using 4-fluoro-3-(4,4,5,5-tetramethyl-1,3,2-dioxaborolan-2-yl)benzonitrile (CAS: 863868-29-5) as E8aa (yield 67.1%) and procedure 15 using carbazole (CAS: 86-74-8) as E4 (yield 66.5%).

MS (HPLC-MS), m/z (retention time) 679.8 (5.238 min).

FIG. 12 depicts the emission spectrum of example 10 (10% by weight in PMMA) at room temperature (e.g., approximately 20° C.). The emission maximum (λₘₐₓ) is at 478 nm. The photoluminescence quantum yield (PLQY) is 77%, the full width at half maximum (FWHM) is 0.43 eV, and the emission lifetime is 29.7 μs. The resulting CIEₓ coordinate is determined at 0.18 and the CIE_{y} coordinate at 0.31.

### Example 13

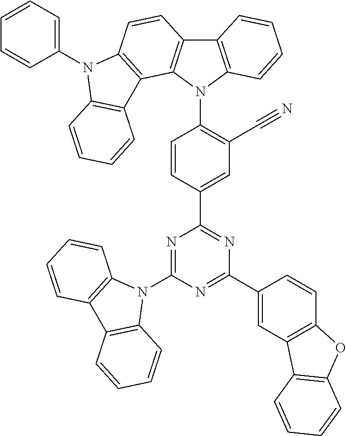 [SMILES: N#Cc1cc(-c2nc(-c3ccc4oc5ccccc5c4c3)nc(-n3c4ccccc4c4ccccc43)n2)ccc1-n1c2ccccc2c2ccc3c(c4ccccc4n3-c3ccccc3)c21]

Example 13 was synthesized according to procedure 13 with 9-(4,6-dichloro-1,3,5-trazin-2-yl)-carbazole (CAS: 24209-95-8) as E6aa and dibenzo[b,d]furan-2-ylboronic acid (CAS: 402936-15-6) as E7aa (yield 53.5%), procedure 14 with 3-cyano-4-fluorophenylboronic acid (CAS: 214210-21-6) as E8aa (yield 77.9%) and procedure 17 using 5,12-dihydro-5-phenyl-Indolo[3,2-a]carbazole (CAS: 1247053-55-9) as E4 (yield 35.2%).

MS (HPLC-MS), m/z (retention time) 845.0 (6.613 min).

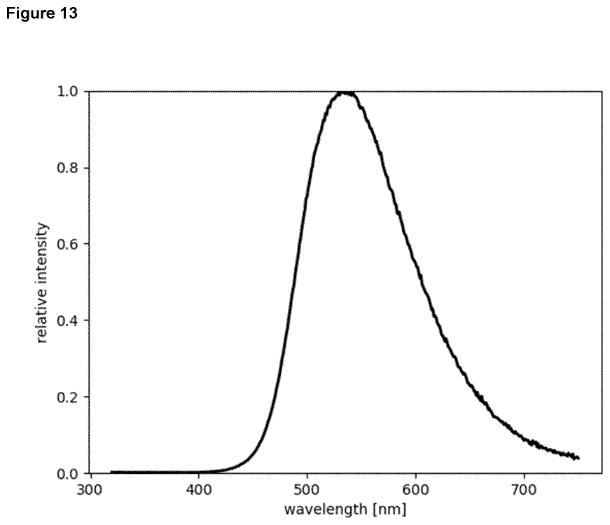

FIG. 13 depicts the emission spectrum of example 10 (10% by weight in PMMA) at room temperature (e.g., approximately 20° C.). The emission maximum (λₘₐₓ) is at 537 nm. The photoluminescence quantum yield (PLQY) is 35%, the full width at half maximum (FWHM) is 0.49 eV, and the emission lifetime is 21.9 μs. The resulting CIEₓ coordinate is determined at 0.36 and the CIE_{y} coordinate at 0.53.

### Device Examples

Stack Materials

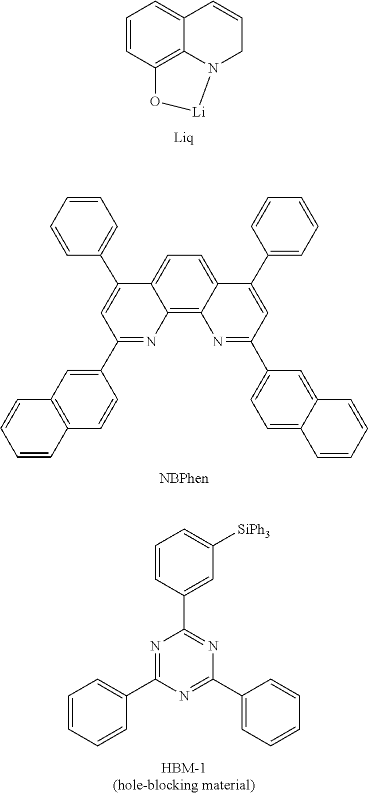

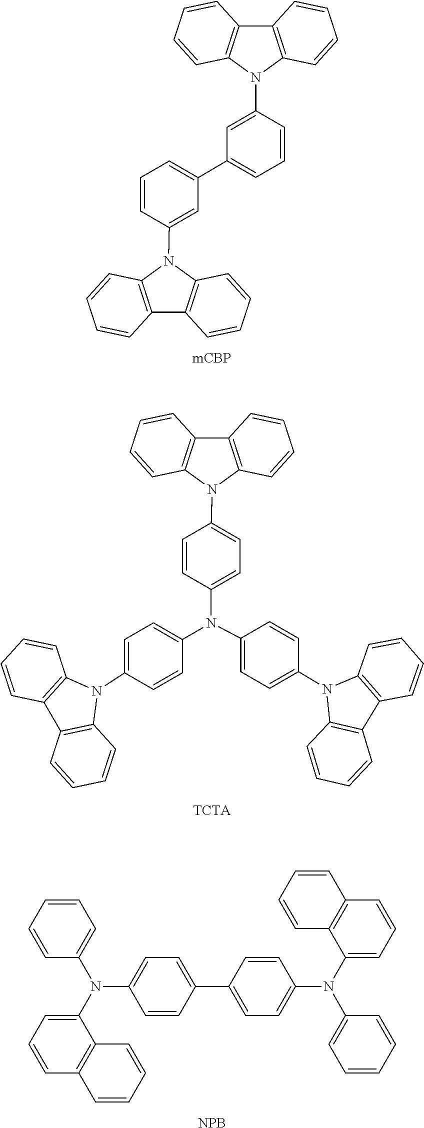 [SMILES: c1cc(-c2cccc(-n3c4ccccc4c4ccccc43)c2)cc(-n2c3ccccc3c3ccccc32)c1 ; c1ccc2c(c1)c1ccccc1n2-c1ccc(N(c2ccc(-n3c4ccccc4c4ccccc43)cc2)c2ccc(-n3c4ccccc4c4ccccc43)cc2)cc1 ; c1ccc(N(c2ccc(-c3ccc(N(c4ccccc4)c4cccc5ccccc45)cc3)cc2)c2cccc3ccccc23)cc1 ; "NPB"]

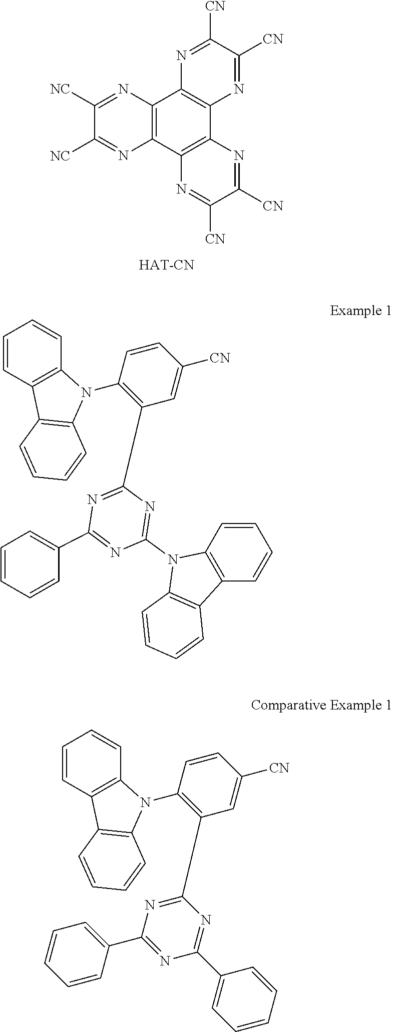 [SMILES: N#Cc1nc2c3nc(C#N)c(C#N)nc3c3nc(C#N)c(C#N)nc3c2nc1C#N ; N#Cc1ccc(-n2c3ccccc3c3ccccc32)c(-c2nc(-c3ccccc3)nc(-n3c4ccccc4c4ccccc43)n2)c1 ; N#Cc1ccc(-n2c3ccccc3c3ccccc32)c(-c2nc(-c3ccccc3)nc(-c3ccccc3)n2)c1]

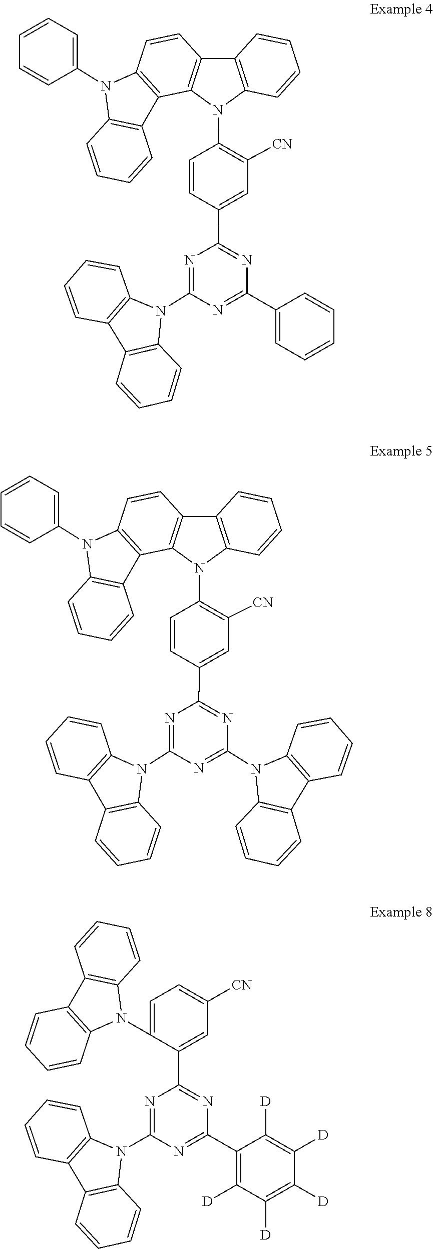 [SMILES: N#Cc1cc(-c2nc(-c3ccccc3)nc(-n3c4ccccc4c4ccccc43)n2)ccc1-n1c2ccccc2c2ccc3c(c4ccccc4n3-c3ccccc3)c21 ; N#Cc1cc(-c2nc(-n3c4ccccc4c4ccccc43)nc(-n3c4ccccc4c4ccccc43)n2)ccc1-n1c2ccccc2c2ccc3c(c4ccccc4n3-c3ccccc3)c21 ; [2H]c1c([2H])c([2H])c(-c2nc(-c3cc(C#N)ccc3-n3c4ccccc4c4ccccc43)nc(-n3c4ccccc4c4ccccc43)n2)c([2H])c1[2H]]

Device Architecture

TABLE 1
Setup A of the example optoelectronic
devices (OLEDs).

| Layer | | | |
|---|---|---|---|
| number | Layer | Thickness | Material(s) |
| 10 | cathode | 100 nm | Al |
| 9 | EIL | 2 nm | Liq |
| 8 | ETL | 20 nm | NBPhen |
| 7 | HBL | 10 nm | HBM1 |
| 6 | EML | 50 nm | mCPB and |
| | | | TADF |
| | | | emitter |
| 5 | EBL | 10 nm | mCBP |
| 4 | HTL-2 | 10 nm | TCTA |
| 3 | HTL-1 | 40 nm | NPB |
| 2 | HIL | 5 nm | HAT-CN |
| 1 | anode | 50 nm | ITO |
| | substrate | | glass |

TABLE 2
Setup B of the example optoelectronic
devices (OLEDs).

| Layer | | | |
|---|---|---|---|
| number | Layer | Thickness | Material(s) |
| 9 | cathode | 100 nm | Al |
| 8 | EIL | 2 nm | Liq |
| 7 | ETL | 30 nm | NBPhen |
| 6 | EML | 50 nm | mCPB and |
| | | | TADF |
| | | | emitter |
| 5 | EBL | 10 nm | mCBP |
| 4 | HTL-2 | 10 nm | TCTA |
| 3 | HTL-1 | 40 nm | NPB |
| 2 | HIL | 5 nm | HAT-CN |
| 1 | anode | 50 nm | ITO |
| | substrate | | glass |

TABLE 3
Setup C of the example optoelectronic
devices (OLEDs).

| Layer | | | |
|---|---|---|---|
| number | Layer | Thickness | Material(s) |
| 10 | cathode | 100 nm | Al |
| 9 | EIL | 2 nm | Liq |
| 8 | ETL | 20 nm | NBPhen |
| 7 | HBL | 10 nm | HBM1 |
| 6 | EML | 50 nm | mCPB and |
| | | | TADF |
| | | | emitter |
| 5 | EBL | 10 nm | mCBP |
| 4 | HTL-2 | 10 nm | TCTA |
| 3 | HTL-1 | 50 nm | NPB |
| 2 | HIL | 5 nm | HAT-CN |
| 1 | anode | 50 nm | ITO |
| | substrate | | glass |

TABLE 4
Example optoelectronic devices (OLEDs).

| | | | Ratio of |
|---|---|---|---|
| | | | mCBP:TADF |
| OLED | | TADF | emitter in the |
| number | Setup | emitter | EML by weight |
| 1 | A | Comparative | 80:20 |
| | | Example 1 | |
| 2 | B | Comparative | 80:20 |
| | | Example 1 | |
| 3 | A | Example 1 | 80:20 |
| 4 | B | Example 1 | 80:20 |
| 5 | A | Example 1 | 70:30 |
| 6 | B | Example 1 | 70:30 |
| 7 | C | Example 4 | 80:20 |
| 8 | C | Example 4 | 70:30 |
| 9 | C | Example 5 | 80:20 |
| 10 | C | Example 5 | 70:30 |
| 11 | A | Example 8 | 80:20 |
| 12 | A | Example 8 | 70:30 |
| 13 | B | Example 8 | 80:20 |
| 14 | B | Example 8 | 70:30 |
| 15 | A | Example 10 | 80:20 |
| 16 | B | Example 10 | 80:20 |
| 17 | A | Example 11 | 70:30 |
| 18 | A | Example 12 | 70:30 |

### Device Results

TABLE 5
Device results for all optoelectronic
devices (OLEDs) listed in Table 4.

| | | | | | | | Relative |
|---|---|---|---|---|---|---|---|
| | | | | | | EQE at | lifetime |
| | | | | | Voltage at | 1000 | LT95 at |
| OLED | FWHM | λₘₐₓ | | | 10 mA/cm² | nits | 1200 |
| number | [eV] | [nm] | CIEx | CIEy | [Volt] | [%] | nits |
| 1 | 0.37 | 482 | 0.17 | 0.35 | 4.98 | 17.0 | 1.00 |
| 2 | 0.36 | 484 | 0.17 | 0.36 | 4.88 | 14.6 | 1.29 |
| 3 | 0.38 | 484 | 0.17 | 0.36 | 5.28 | 15.2 | 1.67 |
| 4 | 0.37 | 485 | 0.18 | 0.37 | 5.18 | 12.1 | 2.91 |
| 5 | 0.37 | 486 | 0.18 | 0.38 | 4.76 | 15.4 | 3.71 |
| 6 | 0.37 | 486 | 0.18 | 0.39 | 4.67 | 13.4 | 5.09 |
| 7 | 0.42 | 517 | 0.28 | 0.54 | 6.23 | 19.1 | 6.73 |
| 8 | 0.42 | 521 | 0.31 | 0.56 | 5.72 | 19.0 | 22.0 |
| 9 | 0.43 | 522 | 0.31 | 0.55 | 6.47 | 14.7 | 6.52 |
| 10 | 0.43 | 528 | 0.33 | 0.56 | 6.04 | 13.8 | 19.1 |
| 11 | 0.39 | 486 | 0.18 | 0.39 | 4.71 | 17.1 | 3.84 |
| 12 | 0.39 | 488 | 0.19 | 0.41 | 5.10 | 18.1 | 2.82 |
| 13 | 0.39 | 488 | 0.18 | 0.39 | 5.03 | 13.8 | 5.07 |
| 14 | 0.38 | 489 | 0.19 | 0.42 | 4.60 | 14.2 | 5.31 |
| 15 | 0.37 | 486 | 0.18 | 0.39 | 4.65 | 19.1 | 5.12 |
| 16 | 0.37 | 486 | 0.18 | 0.39 | 4.57 | 15.7 | 8.96 |
| 17 | 0.38 | 480 | 0.17 | 0.33 | 5.08 | 19.6 | |
| 18 | 0.39 | 488 | 0.19 | 0.40 | 5.14 | 17.0 | 1.32 |

Organic molecules according to embodiments of the present disclosure lead to optoelectronic devices having prolonged lifetime as compared to a similar OLEDs using, for example, Comparative Example 1 as TADF emitter in the emission layer, while exhibiting a high external quantum efficiency (EQE) and a similar color point.

Additional Examples of Organic Molecules of embodiments of the present disclosure

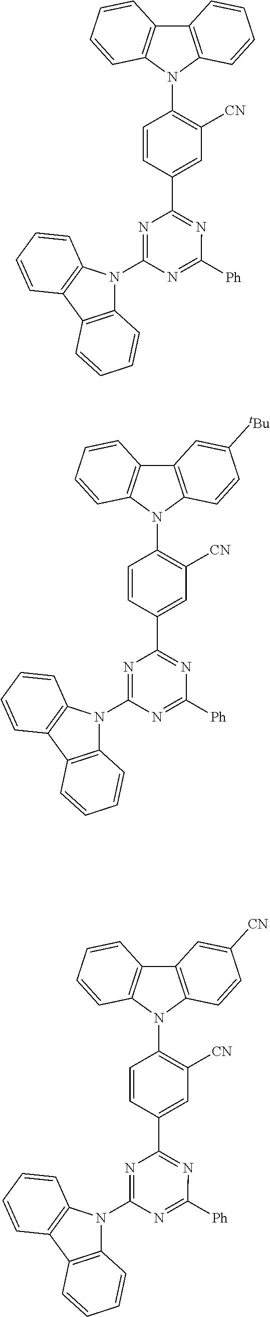 [SMILES: N#Cc1cc(-c2nc(-c3ccccc3)nc(-n3c4ccccc4c4ccccc43)n2)ccc1-n1c2ccccc2c2ccccc21 ; CC(C)(C)c1ccc2c(c1)c1ccccc1n2-c1ccc(-c2nc(-c3ccccc3)nc(-n3c4ccccc4c4ccccc43)n2)cc1C#N ; N#Cc1ccc2c(c1)c1ccccc1n2-c1ccc(-c2nc(-c3ccccc3)nc(-n3c4ccccc4c4ccccc43)n2)cc1C#N]

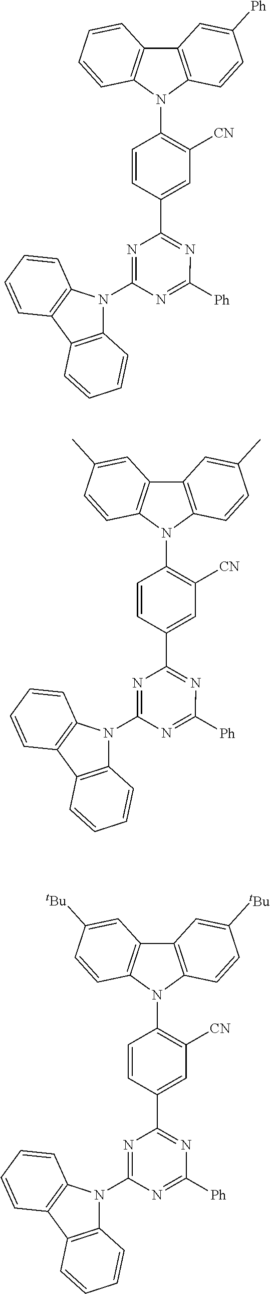

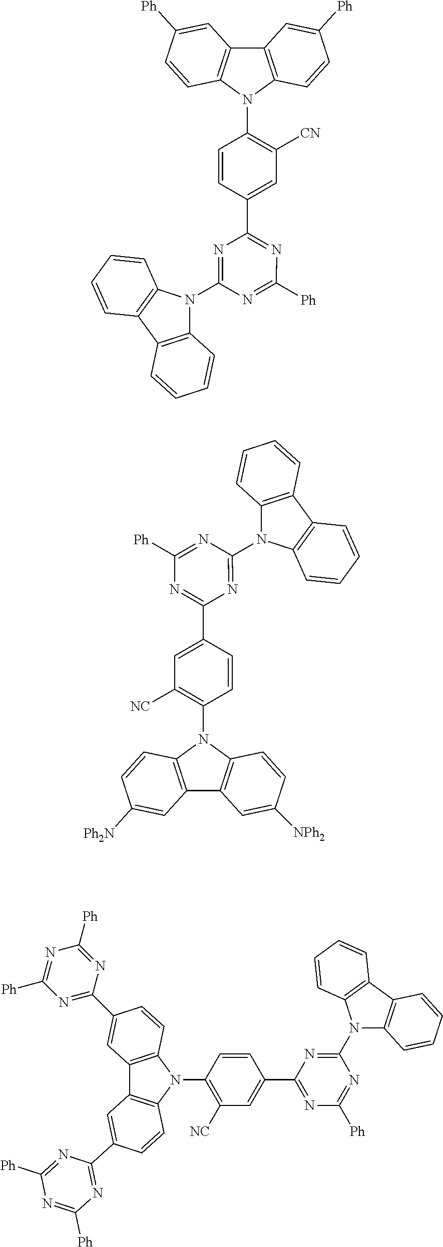

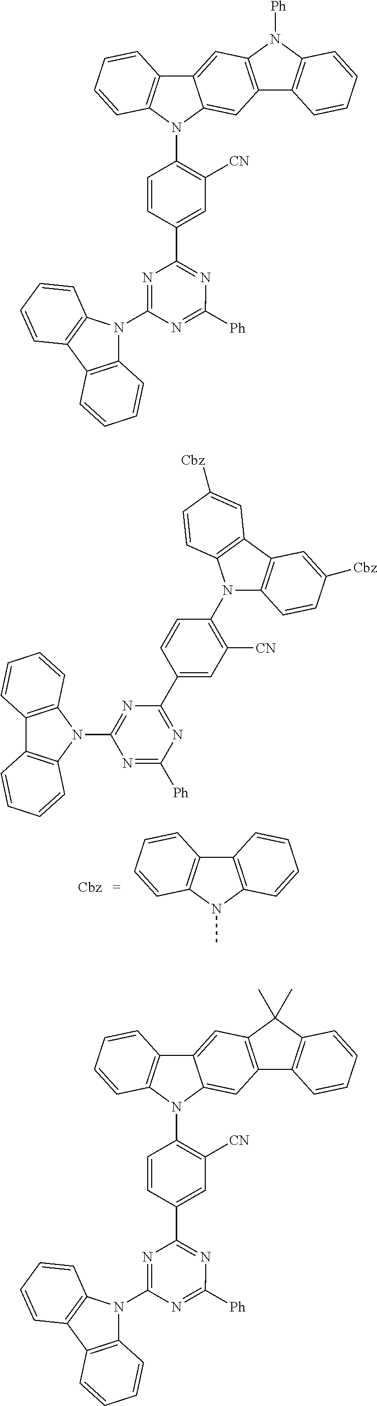

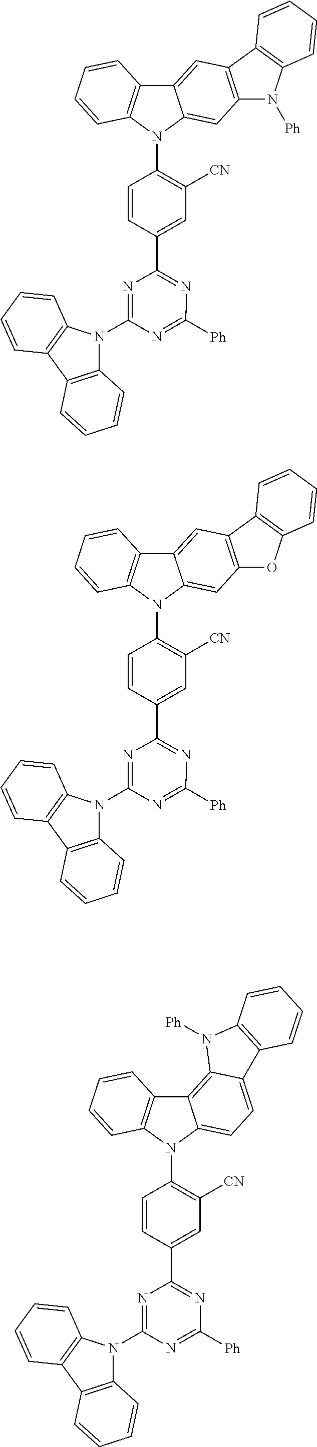 [SMILES: N#Cc1cc(-c2nc(-c3ccccc3)nc(-n3c4ccccc4c4ccccc43)n2)ccc1-n1c2ccccc2c2cc3c4ccccc4n(-c4ccccc4)c3cc21 ; N#Cc1cc(-c2nc(-c3ccccc3)nc(-n3c4ccccc4c4ccccc43)n2)ccc1-n1c2ccccc2c2cc3c(cc21)oc1ccccc13 ; N#Cc1cc(-c2nc(-c3ccccc3)nc(-n3c4ccccc4c4ccccc43)n2)ccc1-n1c2ccccc2c2c1ccc1c3ccccc3n(-c3ccccc3)c12]

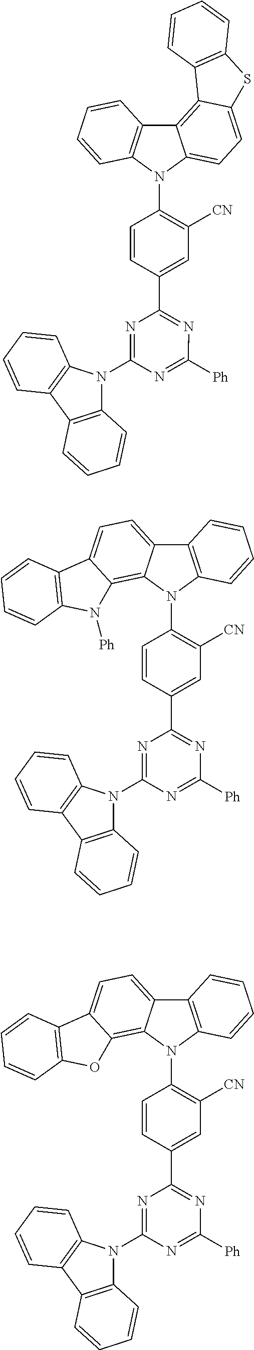 [SMILES: N#Cc1cc(-c2nc(-c3ccccc3)nc(-n3c4ccccc4c4ccccc43)n2)ccc1-n1c2ccccc2c2c3c(ccc21)sc1ccccc13 ; N#Cc1cc(-c2nc(-c3ccccc3)nc(-n3c4ccccc4c4ccccc43)n2)ccc1-n1c2ccccc2c2ccc3c4ccccc4n(-c4ccccc4)c3c21 ; N#Cc1cc(-c2nc(-c3ccccc3)nc(-n3c4ccccc4c4ccccc43)n2)ccc1-n1c2ccccc2c2ccc3c4ccccc4oc3c21]

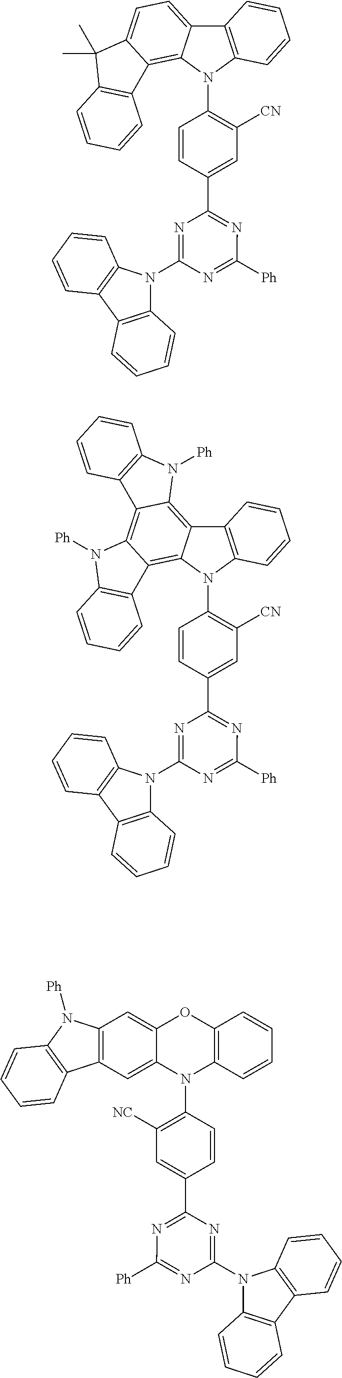 [SMILES: CC1(C)c2ccccc2-c2c1ccc1c3ccccc3n(-c3ccc(-c4nc(-c5ccccc5)nc(-n5c6ccccc6c6ccccc65)n4)cc3C#N)c21 ; N#Cc1cc(-c2nc(-c3ccccc3)nc(-n3c4ccccc4c4ccccc43)n2)ccc1-n1c2ccccc2c2c3c(c4ccccc4n3-c3ccccc3)c3c(c4ccccc4n3-c3ccccc3)c21 ; N#Cc1cc(-c2nc(-c3ccccc3)nc(-n3c4ccccc4c4ccccc43)n2)ccc1N1c2ccccc2Oc2cc3c(cc21)c1ccccc1n3-c1ccccc1]

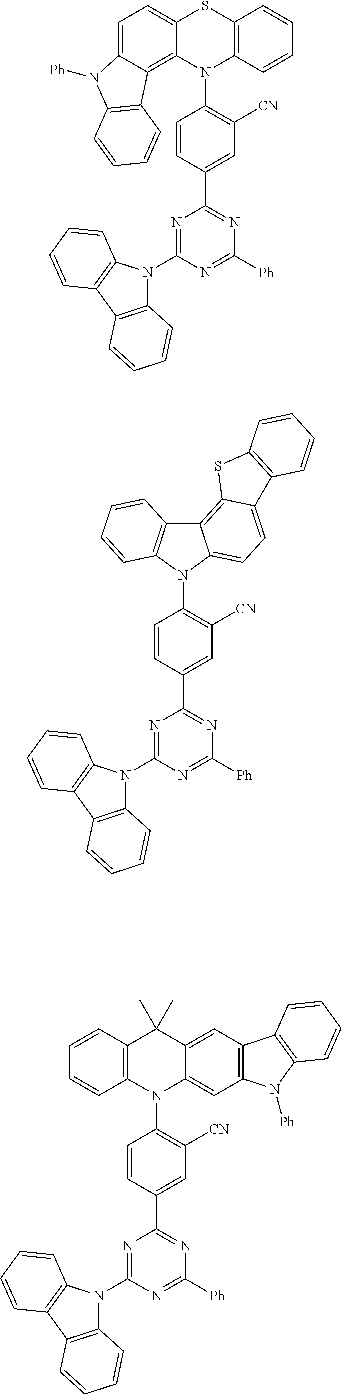 [SMILES: N#Cc1cc(-c2nc(-c3ccccc3)nc(-n3c4ccccc4c4ccccc43)n2)ccc1N1c2ccccc2Sc2ccc3c(c21)c1ccccc1n3-c1ccccc1 ; N#Cc1cc(-c2nc(-c3ccccc3)nc(-n3c4ccccc4c4ccccc43)n2)ccc1-n1c2ccccc2c2c3sc4ccccc4c3ccc21 ; CC1(C)c2ccccc2N(c2ccc(-c3nc(-c4ccccc4)nc(-n4c5ccccc5c5ccccc54)n3)cc2C#N)c2cc3c(cc21)c1ccccc1n3-c1ccccc1]

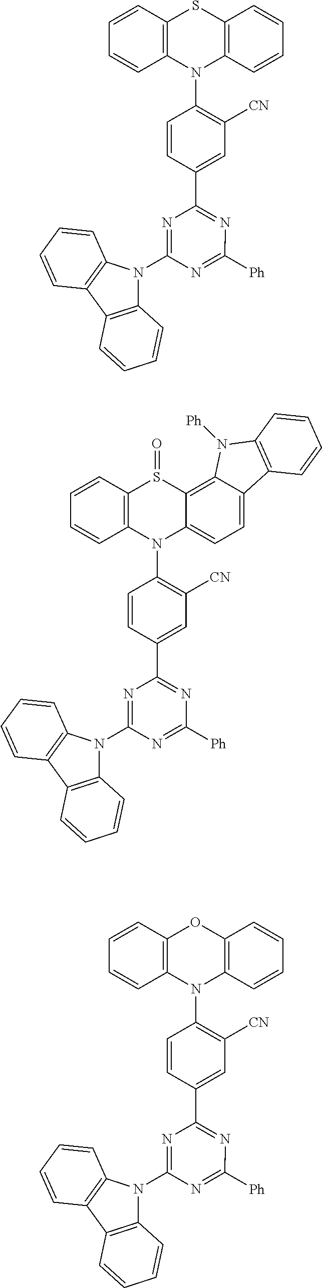 [SMILES: N#Cc1cc(-c2nc(-c3ccccc3)nc(-n3c4ccccc4c4ccccc43)n2)ccc1N1c2ccccc2Sc2ccccc21 ; N#Cc1cc(-c2nc(-c3ccccc3)nc(-n3c4ccccc4c4ccccc43)n2)ccc1N1c2ccccc2S(=O)c2c1ccc1c3ccccc3n(-c3ccccc3)c21 ; N#Cc1cc(-c2nc(-c3ccccc3)nc(-n3c4ccccc4c4ccccc43)n2)ccc1N1c2ccccc2Oc2ccccc21]

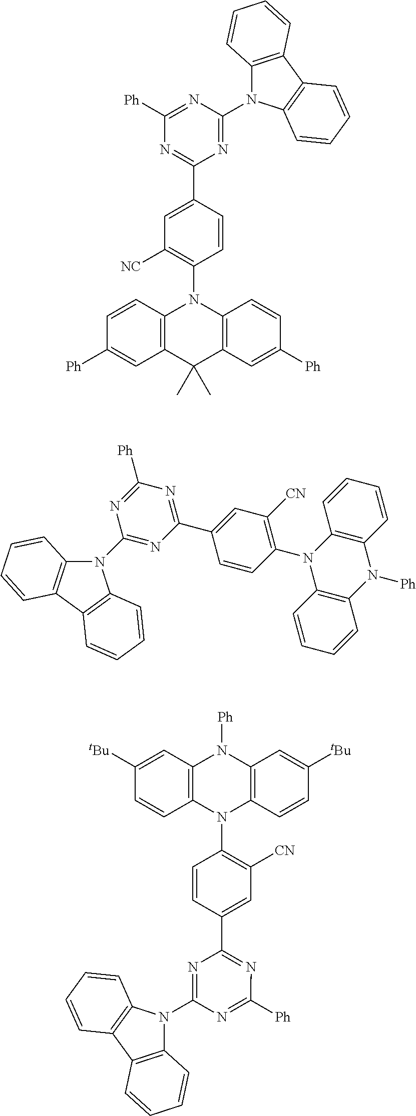 [SMILES: CC1(C)c2cc(-c3ccccc3)ccc2N(c2ccc(-c3nc(-c4ccccc4)nc(-n4c5ccccc5c5ccccc54)n3)cc2C#N)c2ccc(-c3ccccc3)cc21 ; N#Cc1cc(-c2nc(-c3ccccc3)nc(-n3c4ccccc4c4ccccc43)n2)ccc1N1c2ccccc2N(c2ccccc2)c2ccccc21 ; CC(C)(C)c1ccc2c(c1)N(c1ccccc1)c1cc(C(C)(C)C)ccc1N2c1ccc(-c2nc(-c3ccccc3)nc(-n3c4ccccc4c4ccccc43)n2)cc1C#N]

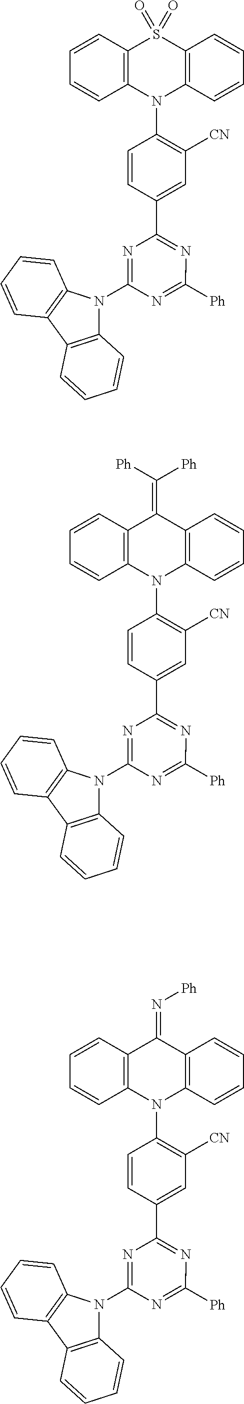 [SMILES: N#Cc1cc(-c2nc(-c3ccccc3)nc(-n3c4ccccc4c4ccccc43)n2)ccc1N1c2ccccc2S(=O)(=O)c2ccccc21 ; N#Cc1cc(-c2nc(-c3ccccc3)nc(-n3c4ccccc4c4ccccc43)n2)ccc1N1c2ccccc2C(=C(c2ccccc2)c2ccccc2)c2ccccc21 ; N#Cc1cc(-c2nc(-c3ccccc3)nc(-n3c4ccccc4c4ccccc43)n2)ccc1-n1c2ccccc2c(=Nc2ccccc2)c2ccccc21]

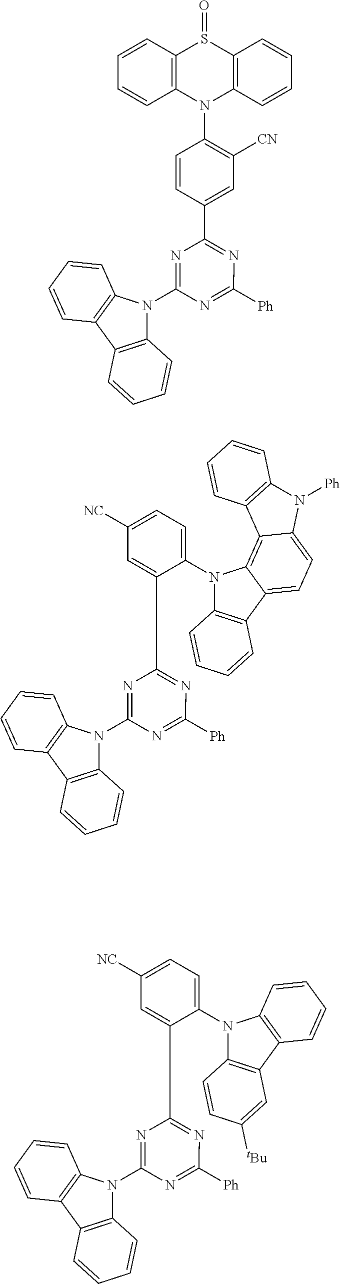 [SMILES: N#Cc1cc(-c2nc(-c3ccccc3)nc(-n3c4ccccc4c4ccccc43)n2)ccc1N1c2ccccc2S(=O)c2ccccc21 ; N#Cc1ccc(-n2c3ccccc3c3ccc4c(c5ccccc5n4-c4ccccc4)c32)c(-c2nc(-c3ccccc3)nc(-n3c4ccccc4c4ccccc43)n2)c1 ; CC(C)(C)c1ccc2c(c1)c1ccccc1n2-c1ccc(C#N)cc1-c1nc(-c2ccccc2)nc(-n2c3ccccc3c3ccccc32)n1]

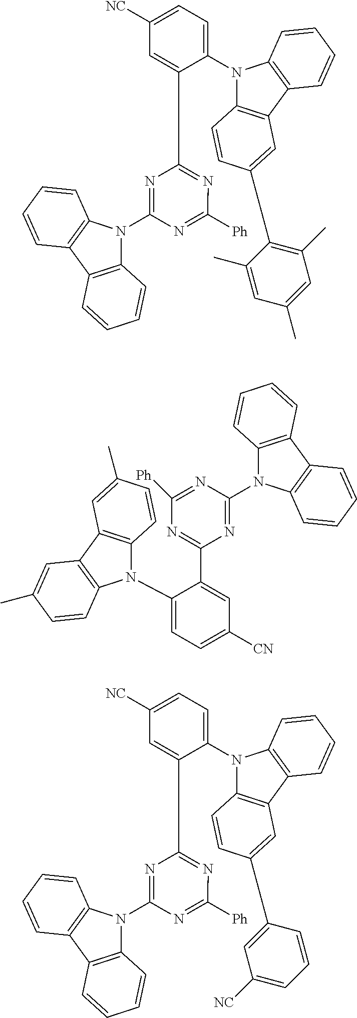 [SMILES: Cc1cc(C)c(-c2ccc3c(c2)c2ccccc2n3-c2ccc(C#N)cc2-c2nc(-c3ccccc3)nc(-n3c4ccccc4c4ccccc43)n2)c(C)c1 ; Cc1ccc2c(c1)c1cc(C)ccc1n2-c1ccc(C#N)cc1-c1nc(-c2ccccc2)nc(-n2c3ccccc3c3ccccc32)n1 ; N#Cc1cccc(-c2ccc3c(c2)c2ccccc2n3-c2ccc(C#N)cc2-c2nc(-c3ccccc3)nc(-n3c4ccccc4c4ccccc43)n2)c1]

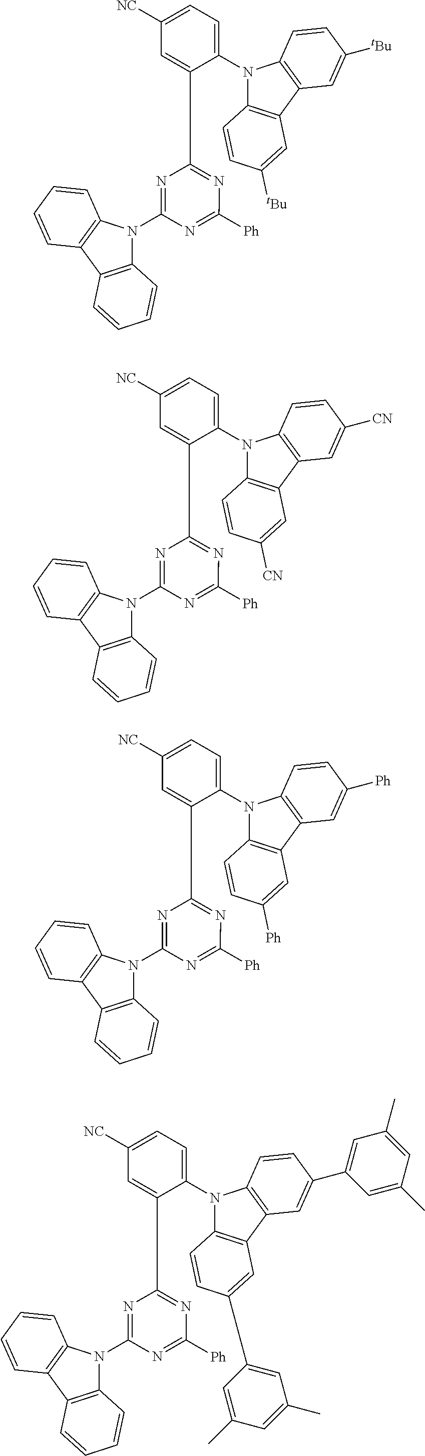

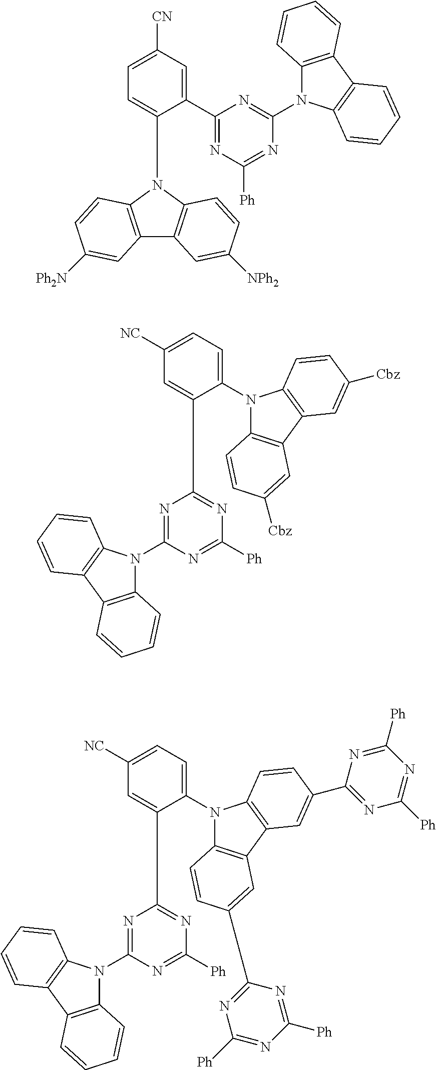

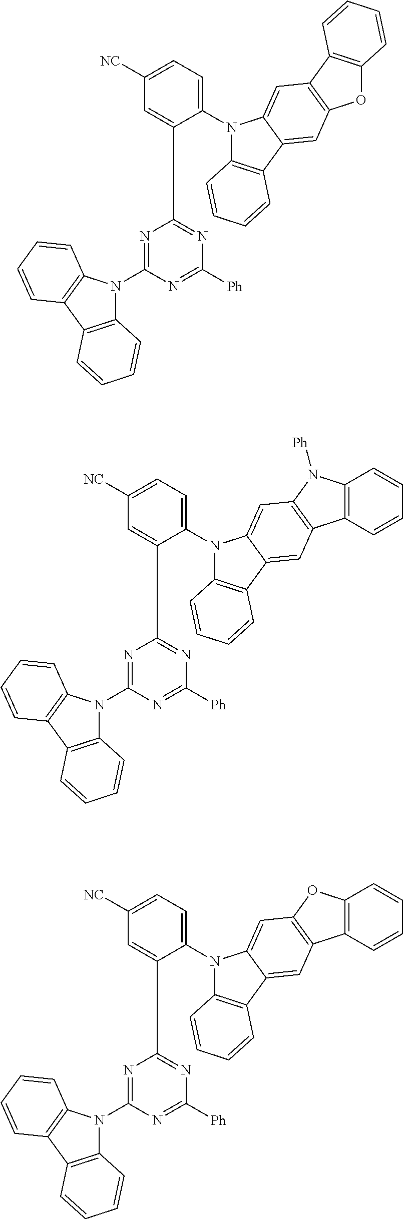 [SMILES: N#Cc1ccc(-n2c3ccccc3c3cc4oc5ccccc5c4cc32)c(-c2nc(-c3ccccc3)nc(-n3c4ccccc4c4ccccc43)n2)c1 ; N#Cc1ccc(-n2c3ccccc3c3cc4c5ccccc5n(-c5ccccc5)c4cc32)c(-c2nc(-c3ccccc3)nc(-n3c4ccccc4c4ccccc43)n2)c1 ; N#Cc1ccc(-n2c3ccccc3c3cc4c(cc32)oc2ccccc24)c(-c2nc(-c3ccccc3)nc(-n3c4ccccc4c4ccccc43)n2)c1]

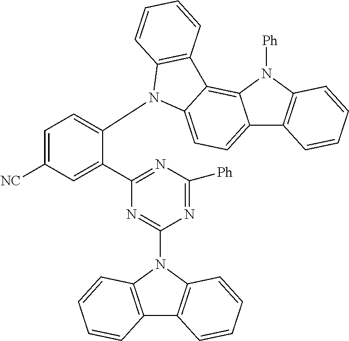 [SMILES: N#Cc1ccc(-n2c3ccccc3c3c2ccc2c4ccccc4n(-c4ccccc4)c23)c(-c2nc(-c3ccccc3)nc(-n3c4ccccc4c4ccccc43)n2)c1]

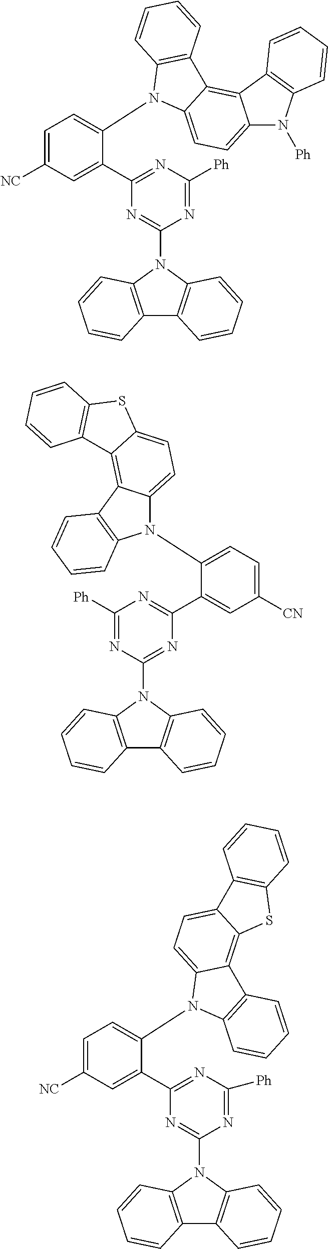 [SMILES: N#Cc1ccc(-n2c3ccccc3c3c4c5ccccc5n(-c5ccccc5)c4ccc32)c(-c2nc(-c3ccccc3)nc(-n3c4ccccc4c4ccccc43)n2)c1 ; N#Cc1ccc(-n2c3ccccc3c3c4c(ccc32)sc2ccccc24)c(-c2nc(-c3ccccc3)nc(-n3c4ccccc4c4ccccc43)n2)c1 ; N#Cc1ccc(-n2c3ccccc3c3c4sc5ccccc5c4ccc32)c(-c2nc(-c3ccccc3)nc(-n3c4ccccc4c4ccccc43)n2)c1]

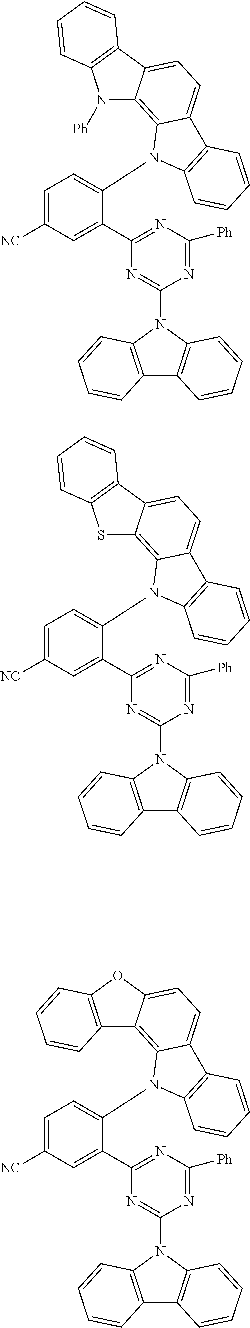

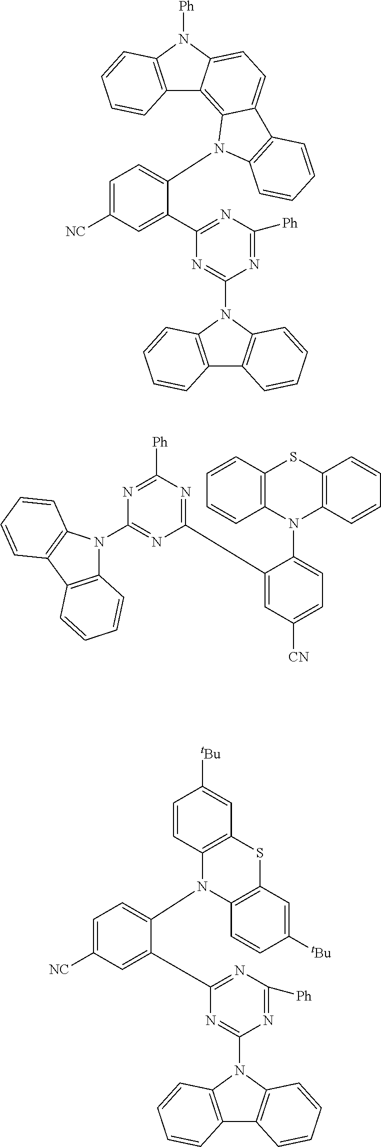 [SMILES: N#Cc1ccc(-n2c3ccccc3c3ccc4c(c5ccccc5n4-c4ccccc4)c32)c(-c2nc(-c3ccccc3)nc(-n3c4ccccc4c4ccccc43)n2)c1 ; N#Cc1ccc(N2c3ccccc3Sc3ccccc32)c(-c2nc(-c3ccccc3)nc(-n3c4ccccc4c4ccccc43)n2)c1 ; CC(C)(C)c1ccc2c(c1)Sc1cc(C(C)(C)C)ccc1N2c1ccc(C#N)cc1-c1nc(-c2ccccc2)nc(-n2c3ccccc3c3ccccc32)n1]

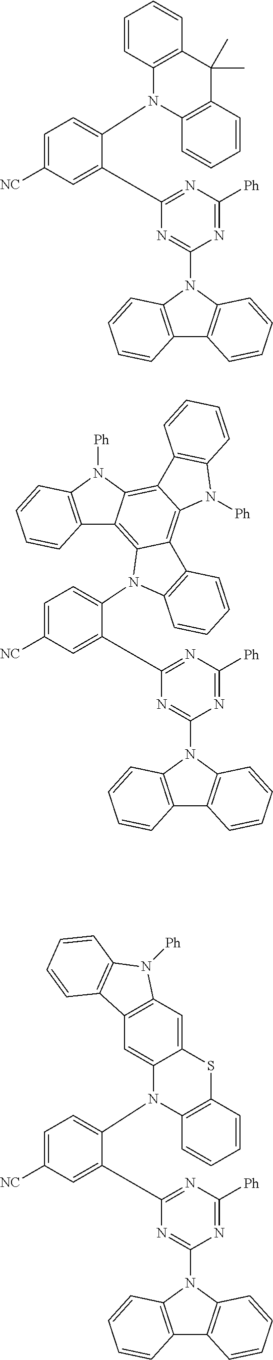 [SMILES: CC1(C)c2ccccc2N(c2ccc(C#N)cc2-c2nc(-c3ccccc3)nc(-n3c4ccccc4c4ccccc43)n2)c2ccccc21 ; N#Cc1ccc(-n2c3ccccc3c3c4c(c5ccccc5n4-c4ccccc4)c4c(c5ccccc5n4-c4ccccc4)c32)c(-c2nc(-c3ccccc3)nc(-n3c4ccccc4c4ccccc43)n2)c1 ; N#Cc1ccc(N2c3ccccc3Sc3cc4c(cc32)c2ccccc2n4-c2ccccc2)c(-c2nc(-c3ccccc3)nc(-n3c4ccccc4c4ccccc43)n2)c1]

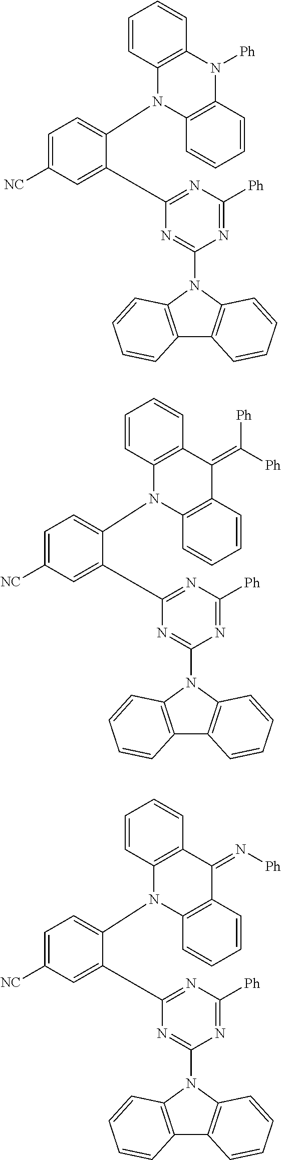

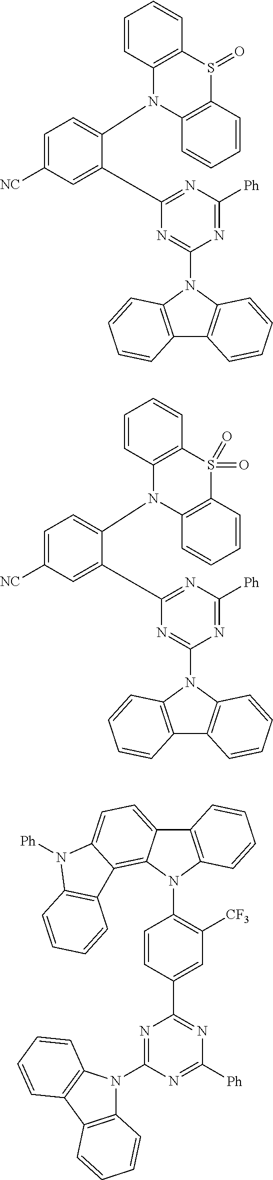

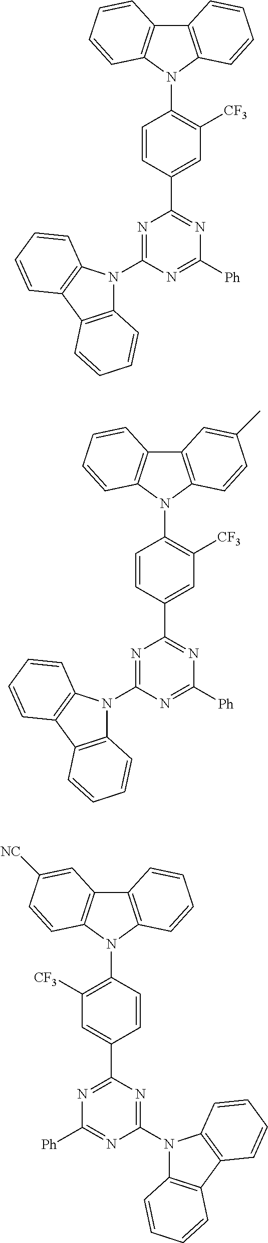 [SMILES: FC(F)(F)c1cc(-c2nc(-c3ccccc3)nc(-n3c4ccccc4c4ccccc43)n2)ccc1-n1c2ccccc2c2ccccc21 ; Cc1ccc2c(c1)c1ccccc1n2-c1ccc(-c2nc(-c3ccccc3)nc(-n3c4ccccc4c4ccccc43)n2)cc1C(F)(F)F ; N#Cc1ccc2c(c1)c1ccccc1n2-c1ccc(-c2nc(-c3ccccc3)nc(-n3c4ccccc4c4ccccc43)n2)cc1C(F)(F)F]

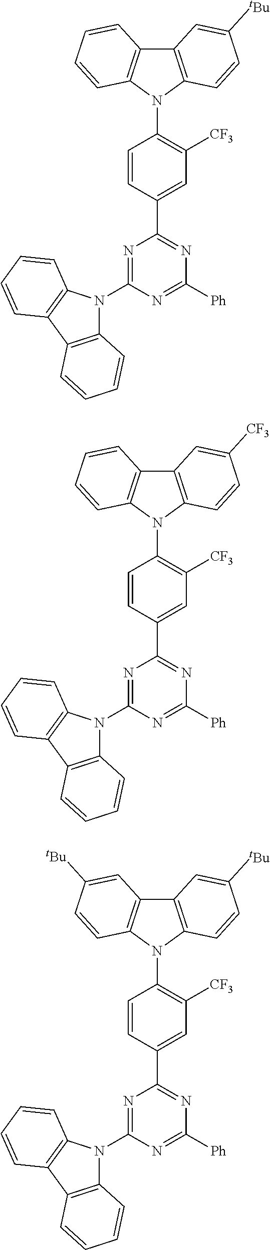

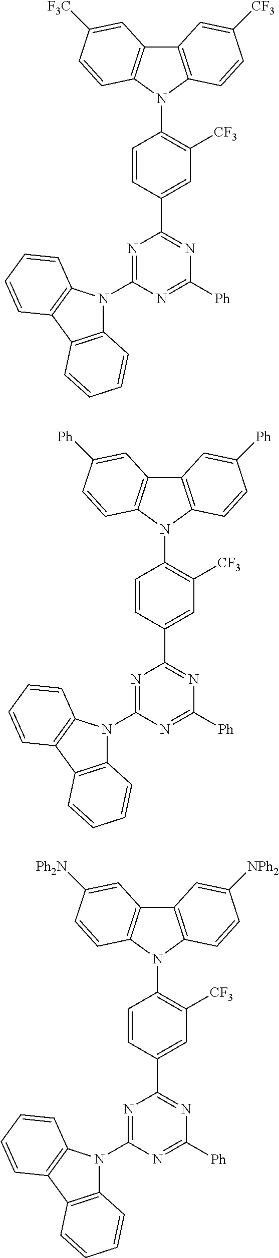

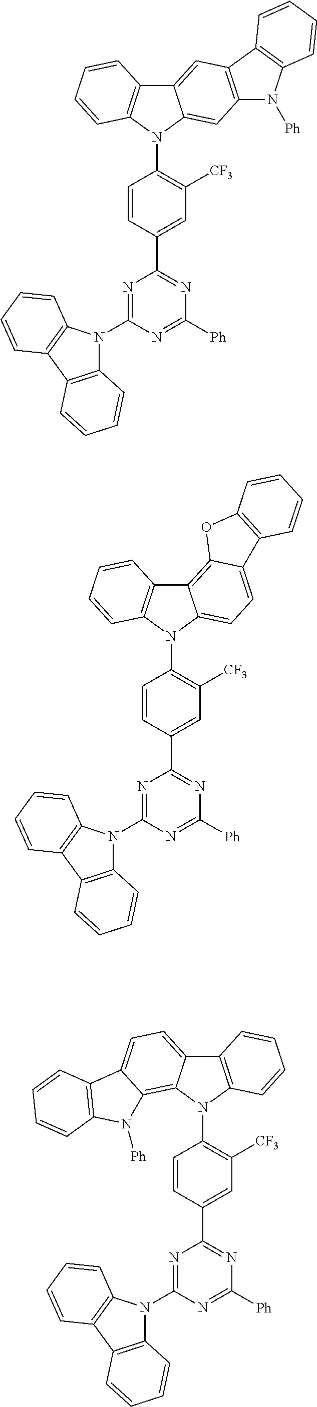 [SMILES: FC(F)(F)c1cc(-c2nc(-c3ccccc3)nc(-n3c4ccccc4c4ccccc43)n2)ccc1-n1c2ccccc2c2cc3c4ccccc4n(-c4ccccc4)c3cc21 ; FC(F)(F)c1cc(-c2nc(-c3ccccc3)nc(-n3c4ccccc4c4ccccc43)n2)ccc1-n1c2ccccc2c2c3oc4ccccc4c3ccc21 ; FC(F)(F)c1cc(-c2nc(-c3ccccc3)nc(-n3c4ccccc4c4ccccc43)n2)ccc1-n1c2ccccc2c2ccc3c4ccccc4n(-c4ccccc4)c3c21]

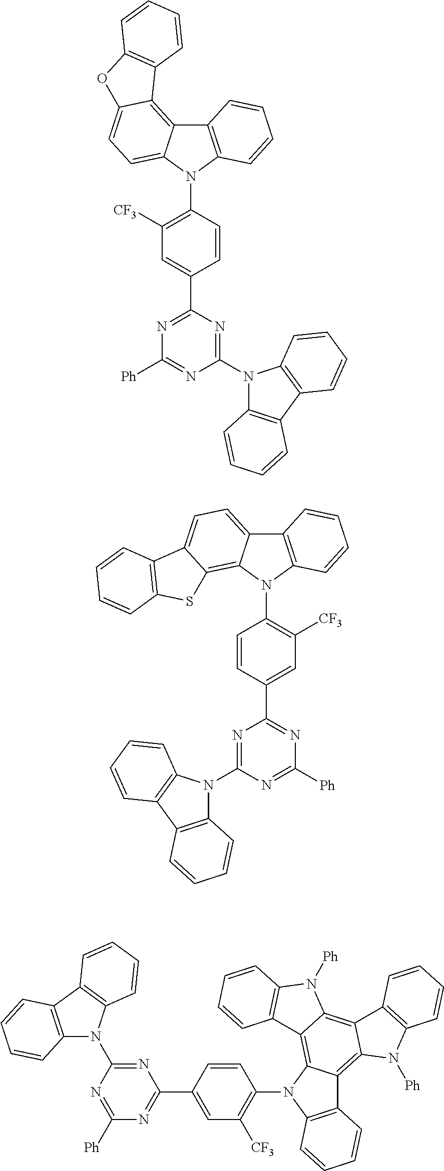

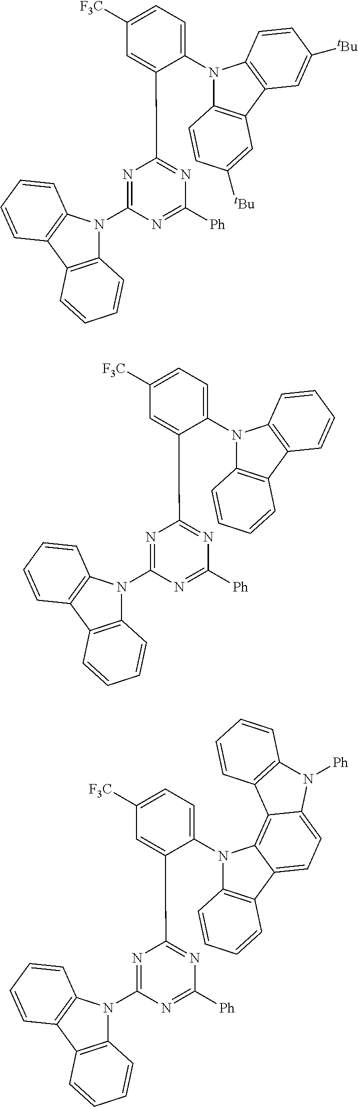 [SMILES: CC(C)(C)c1ccc2c(c1)c1cc(C(C)(C)C)ccc1n2-c1ccc(C(F)(F)F)cc1-c1nc(-c2ccccc2)nc(-n2c3ccccc3c3ccccc32)n1 ; FC(F)(F)c1ccc(-n2c3ccccc3c3ccccc32)c(-c2nc(-c3ccccc3)nc(-n3c4ccccc4c4ccccc43)n2)c1 ; FC(F)(F)c1ccc(-n2c3ccccc3c3ccc4c(c5ccccc5n4-c4ccccc4)c32)c(-c2nc(-c3ccccc3)nc(-n3c4ccccc4c4ccccc43)n2)c1]

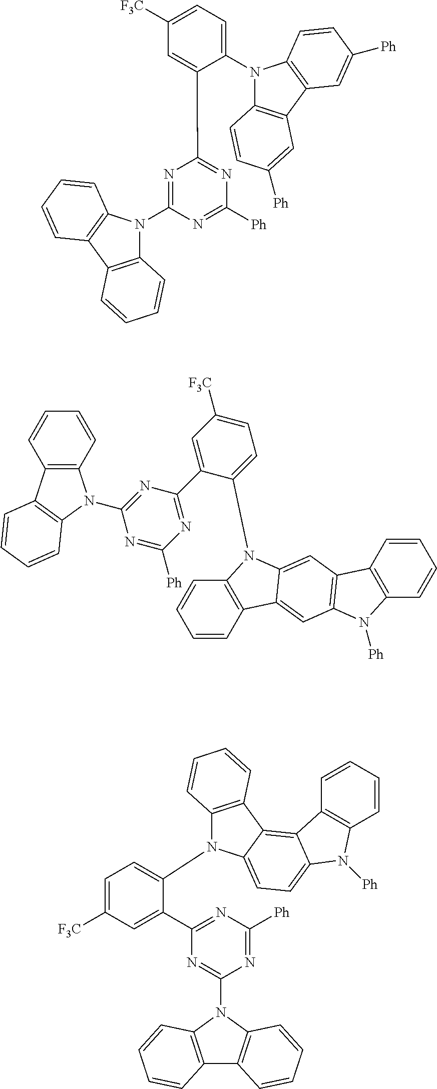 [SMILES: FC(F)(F)c1ccc(-n2c3ccc(-c4ccccc4)cc3c3cc(-c4ccccc4)ccc32)c(-c2nc(-c3ccccc3)nc(-n3c4ccccc4c4ccccc43)n2)c1 ; FC(F)(F)c1ccc(-n2c3ccccc3c3cc4c(cc32)c2ccccc2n4-c2ccccc2)c(-c2nc(-c3ccccc3)nc(-n3c4ccccc4c4ccccc43)n2)c1 ; FC(F)(F)c1ccc(-n2c3ccccc3c3c4c5ccccc5n(-c5ccccc5)c4ccc32)c(-c2nc(-c3ccccc3)nc(-n3c4ccccc4c4ccccc43)n2)c1]

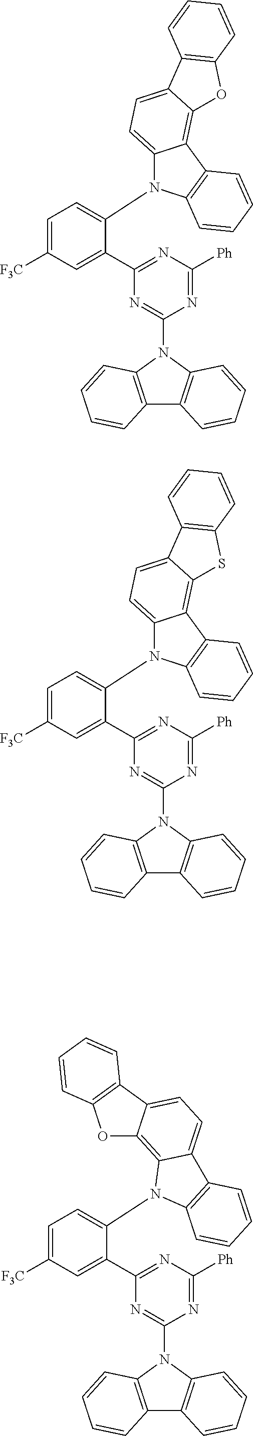

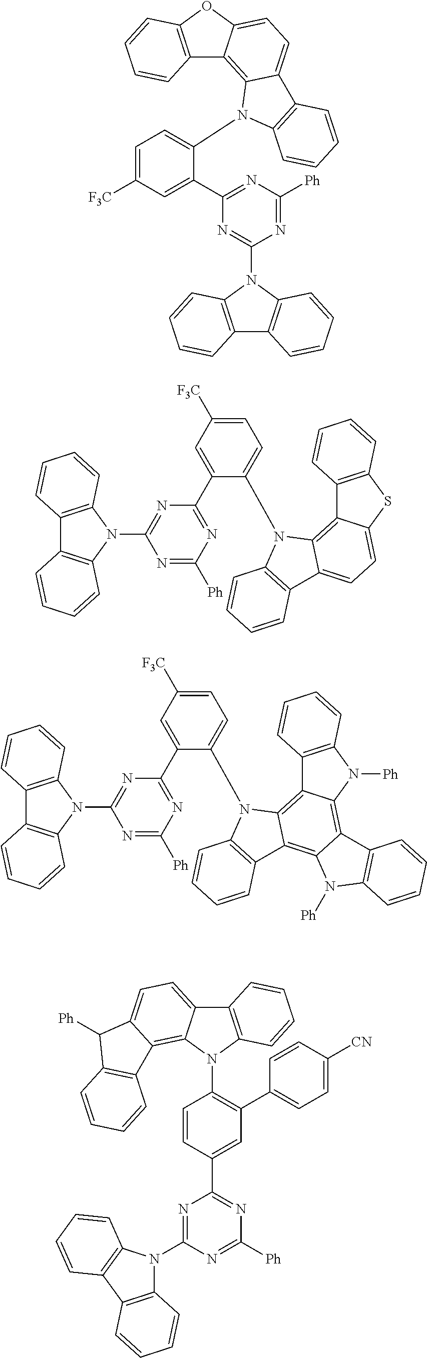

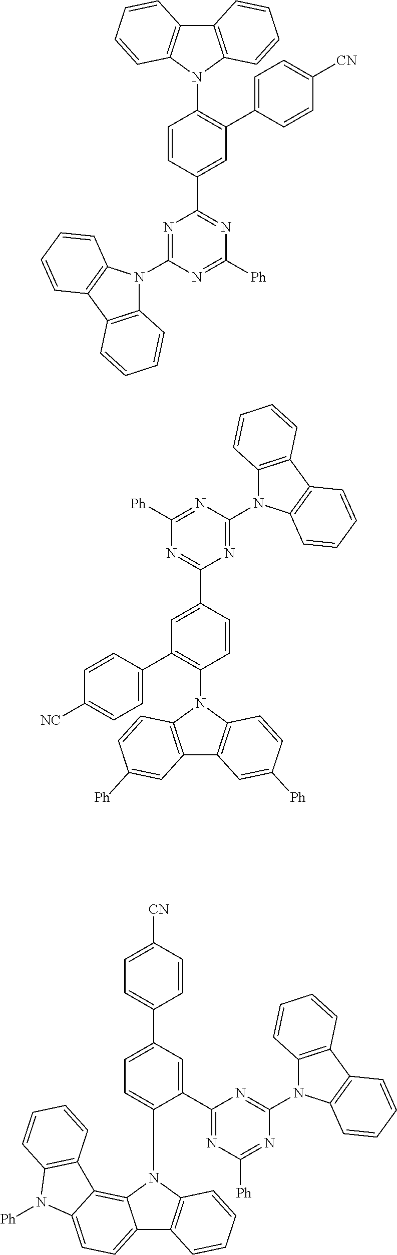 [SMILES: N#Cc1ccc(-c2cc(-c3nc(-c4ccccc4)nc(-n4c5ccccc5c5ccccc54)n3)ccc2-n2c3ccccc3c3ccccc32)cc1 ; N#Cc1ccc(-c2cc(-c3nc(-c4ccccc4)nc(-n4c5ccccc5c5ccccc54)n3)ccc2-n2c3ccc(-c4ccccc4)cc3c3cc(-c4ccccc4)ccc32)cc1 ; N#Cc1ccc(-c2ccc(-n3c4ccccc4c4ccc5c(c6ccccc6n5-c5ccccc5)c43)c(-c3nc(-c4ccccc4)nc(-n4c5ccccc5c5ccccc54)n3)c2)cc1]

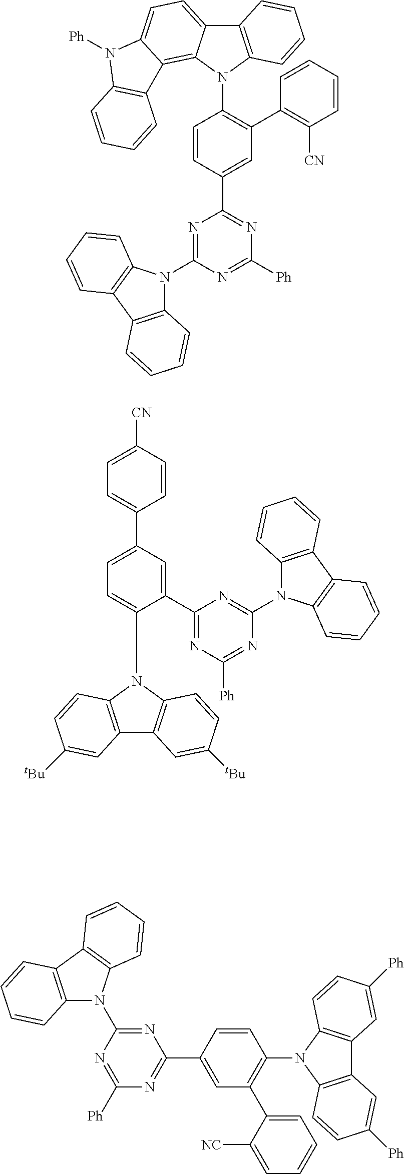

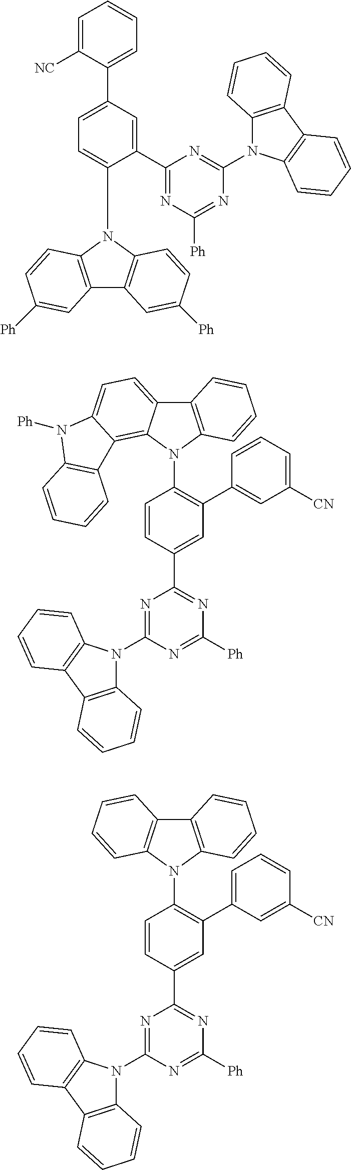 [SMILES: N#Cc1ccccc1-c1ccc(-n2c3ccc(-c4ccccc4)cc3c3cc(-c4ccccc4)ccc32)c(-c2nc(-c3ccccc3)nc(-n3c4ccccc4c4ccccc43)n2)c1 ; N#Cc1cccc(-c2cc(-c3nc(-c4ccccc4)nc(-n4c5ccccc5c5ccccc54)n3)ccc2-n2c3ccccc3c3ccc4c(c5ccccc5n4-c4ccccc4)c32)c1 ; N#Cc1cccc(-c2cc(-c3nc(-c4ccccc4)nc(-n4c5ccccc5c5ccccc54)n3)ccc2-n2c3ccccc3c3ccccc32)c1]

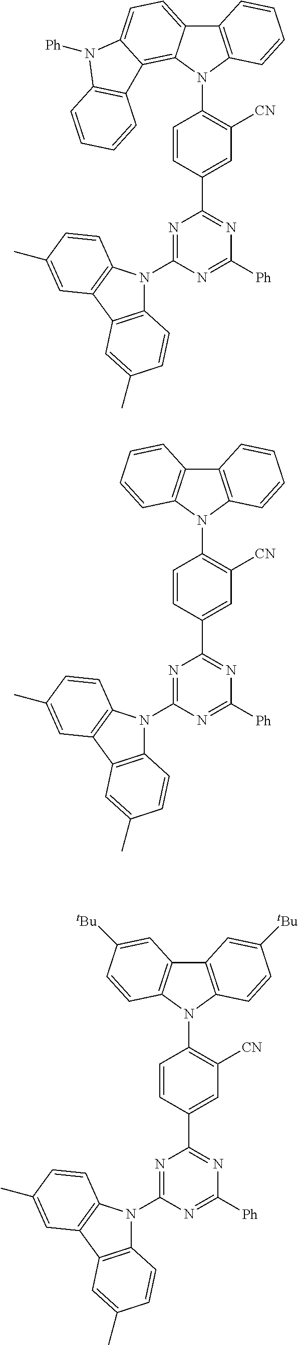

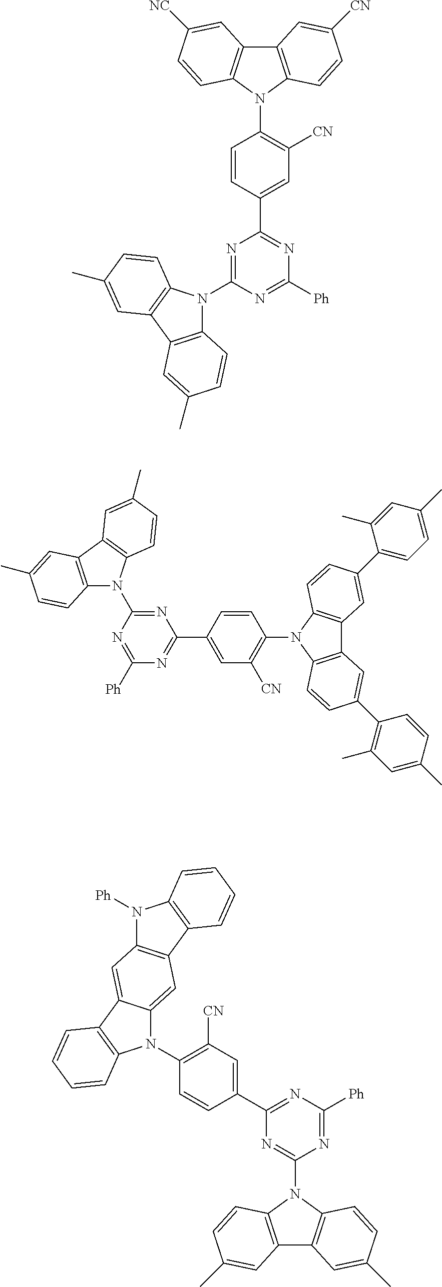

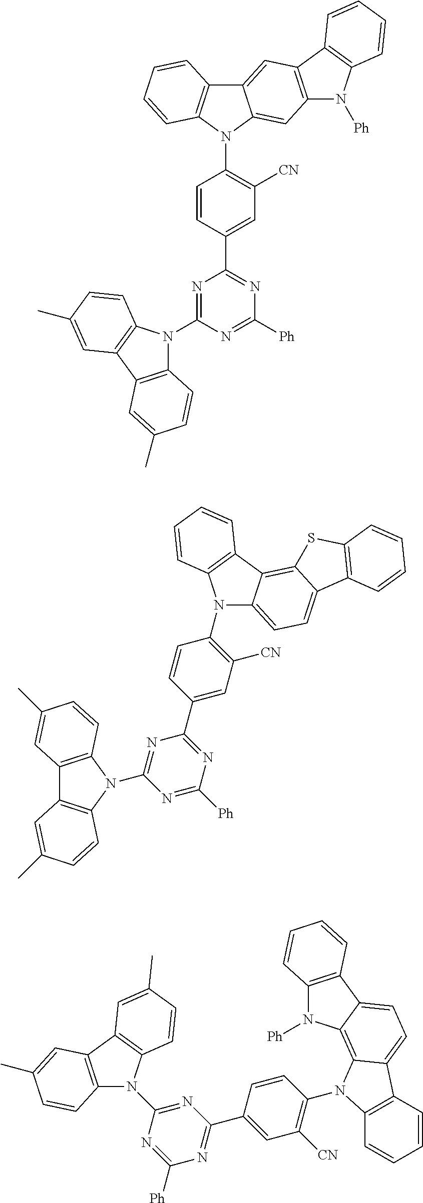 [SMILES: Cc1ccc2c(c1)c1cc(C)ccc1n2-c1nc(-c2ccccc2)nc(-c2ccc(-n3c4ccccc4c4cc5c6ccccc6n(-c6ccccc6)c5cc43)c(C#N)c2)n1 ; Cc1ccc2c(c1)c1cc(C)ccc1n2-c1nc(-c2ccccc2)nc(-c2ccc(-n3c4ccccc4c4c5sc6ccccc6c5ccc43)c(C#N)c2)n1 ; Cc1ccc2c(c1)c1cc(C)ccc1n2-c1nc(-c2ccccc2)nc(-c2ccc(-n3c4ccccc4c4ccc5c6ccccc6n(-c6ccccc6)c5c43)c(C#N)c2)n1]

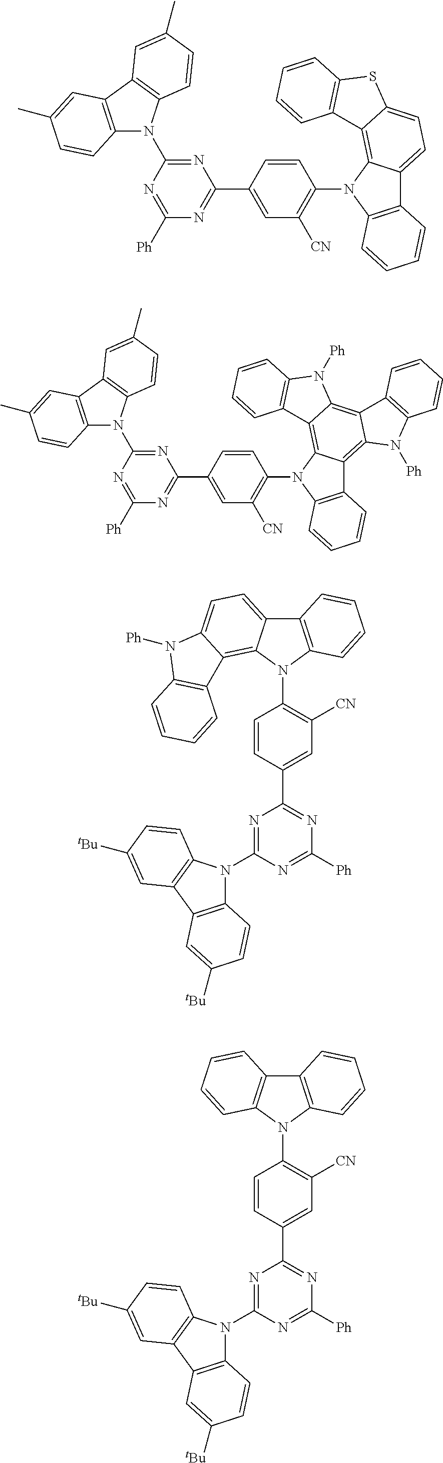

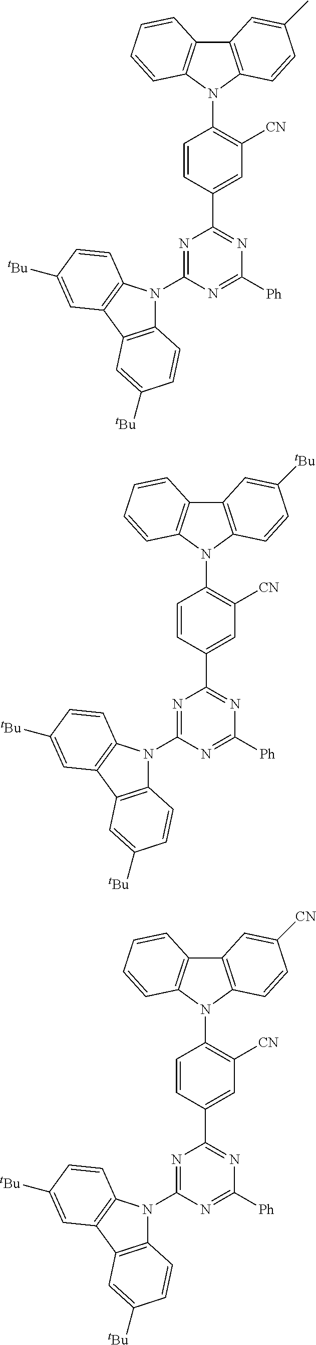 [SMILES: Cc1ccc2c(c1)c1ccccc1n2-c1ccc(-c2nc(-c3ccccc3)nc(-n3c4ccc(C(C)(C)C)cc4c4cc(C(C)(C)C)ccc43)n2)cc1C#N ; CC(C)(C)c1ccc2c(c1)c1cc(C(C)(C)C)ccc1n2-c1nc(-c2ccccc2)nc(-c2ccc(-n3c4ccccc4c4cc(C(C)(C)C)ccc43)c(C#N)c2)n1 ; CC(C)(C)c1ccc2c(c1)c1cc(C(C)(C)C)ccc1n2-c1nc(-c2ccccc2)nc(-c2ccc(-n3c4ccccc4c4cc(C#N)ccc43)c(C#N)c2)n1]

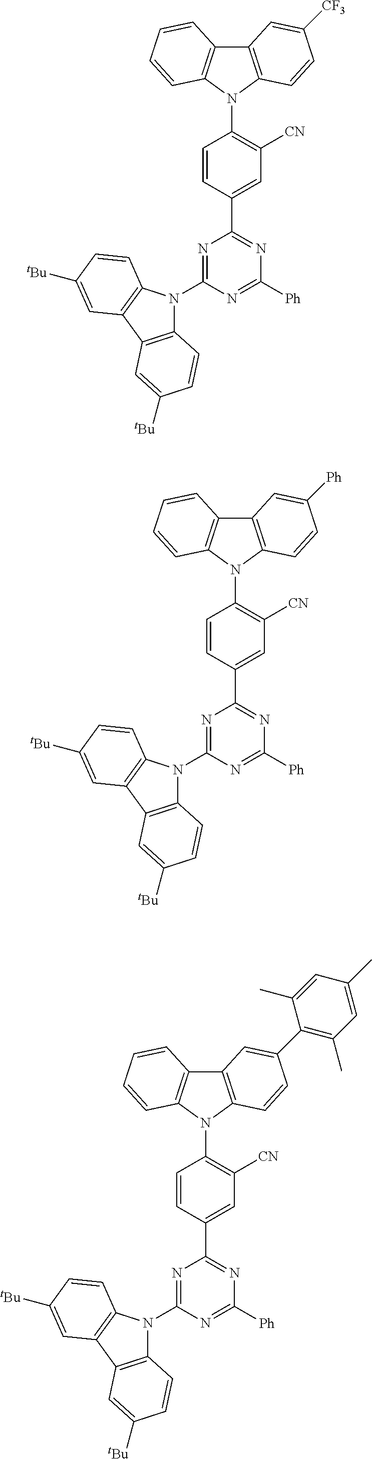 [SMILES: CC(C)(C)c1ccc2c(c1)c1cc(C(C)(C)C)ccc1n2-c1nc(-c2ccccc2)nc(-c2ccc(-n3c4ccccc4c4cc(C(F)(F)F)ccc43)c(C#N)c2)n1 ; CC(C)(C)c1ccc2c(c1)c1cc(C(C)(C)C)ccc1n2-c1nc(-c2ccccc2)nc(-c2ccc(-n3c4ccccc4c4cc(-c5ccccc5)ccc43)c(C#N)c2)n1 ; Cc1cc(C)c(-c2ccc3c(c2)c2ccccc2n3-c2ccc(-c3nc(-c4ccccc4)nc(-n4c5ccc(C(C)(C)C)cc5c5cc(C(C)(C)C)ccc54)n3)cc2C#N)c(C)c1]

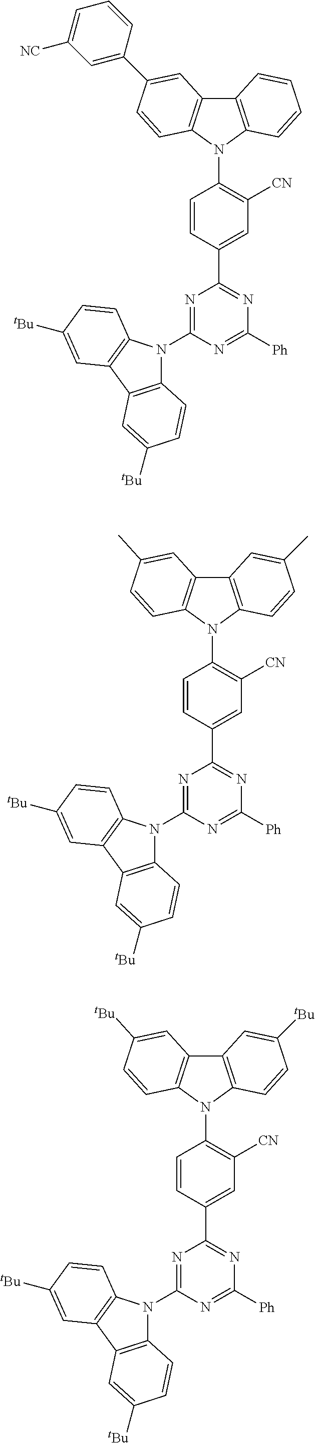 [SMILES: CC(C)(C)c1ccc2c(c1)c1cc(C(C)(C)C)ccc1n2-c1nc(-c2ccccc2)nc(-c2ccc(-n3c4ccccc4c4cc(-c5cccc(C#N)c5)ccc43)c(C#N)c2)n1 ; Cc1ccc2c(c1)c1cc(C)ccc1n2-c1ccc(-c2nc(-c3ccccc3)nc(-n3c4ccc(C(C)(C)C)cc4c4cc(C(C)(C)C)ccc43)n2)cc1C#N ; CC(C)(C)c1ccc2c(c1)c1cc(C(C)(C)C)ccc1n2-c1nc(-c2ccccc2)nc(-c2ccc(-n3c4ccc(C(C)(C)C)cc4c4cc(C(C)(C)C)ccc43)c(C#N)c2)n1]

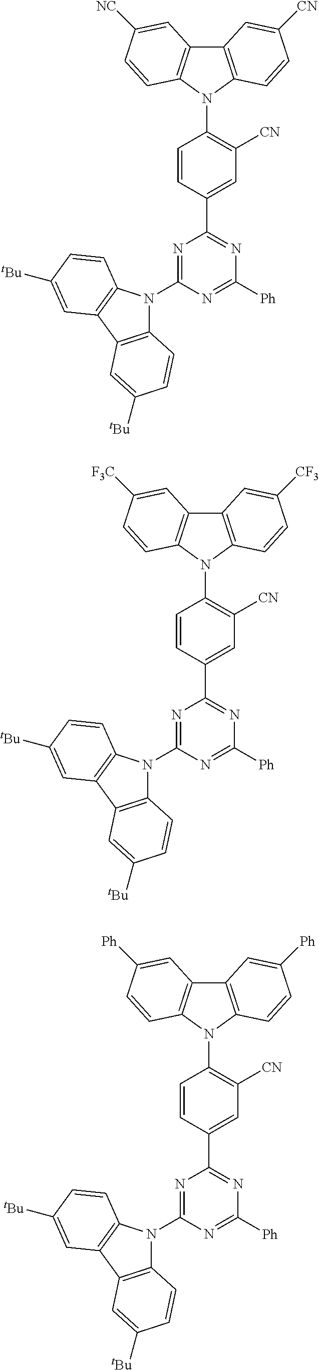

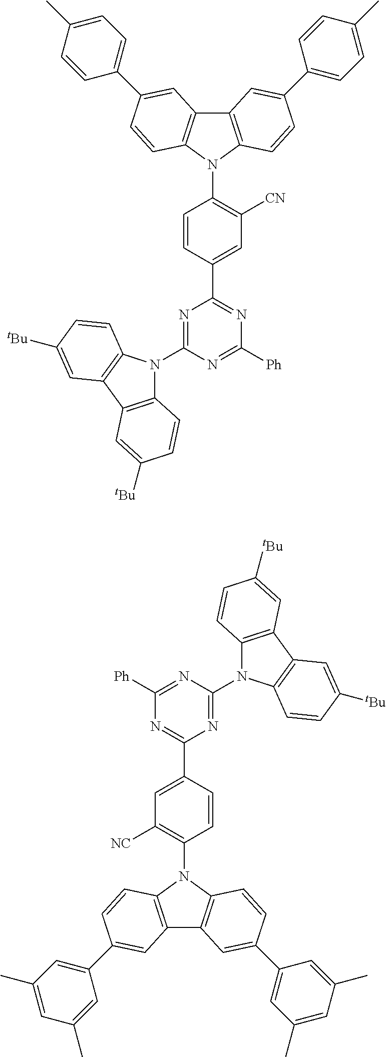

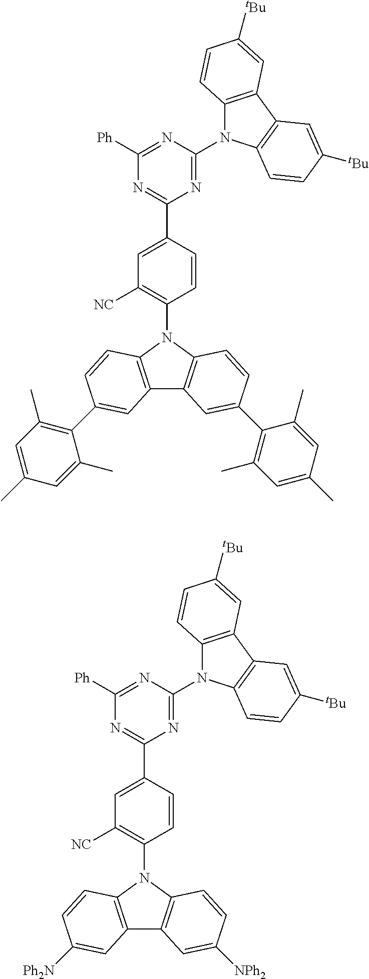

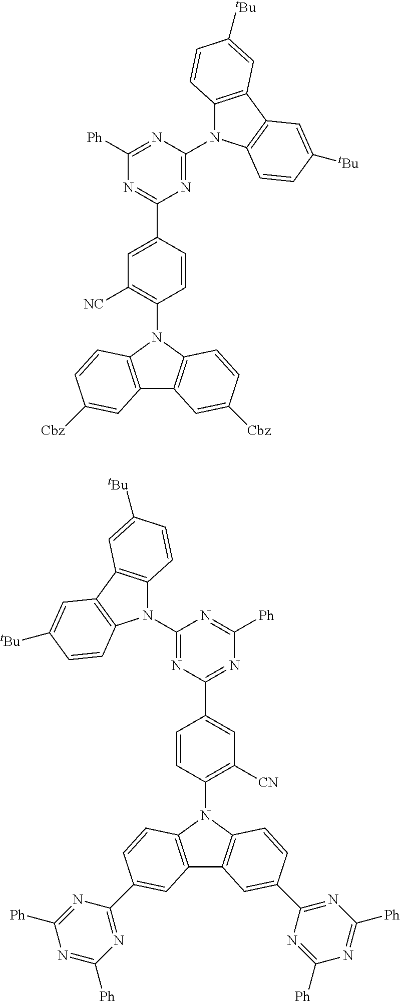 [SMILES: CC(C)(C)c1ccc2c(c1)c1cc(C(C)(C)C)ccc1n2-c1nc(-c2ccccc2)nc(-c2ccc(-n3c4ccc(C(=O)OCc5ccccc5)cc4c4cc(C(=O)OCc5ccccc5)ccc43)c(C#N)c2)n1 ; CC(C)(C)c1ccc2c(c1)c1cc(C(C)(C)C)ccc1n2-c1nc(-c2ccccc2)nc(-c2ccc(-n3c4ccc(-c5nc(-c6ccccc6)nc(-c6ccccc6)n5)cc4c4cc(-c5nc(-c6ccccc6)nc(-c6ccccc6)n5)ccc43)c(C#N)c2)n1]

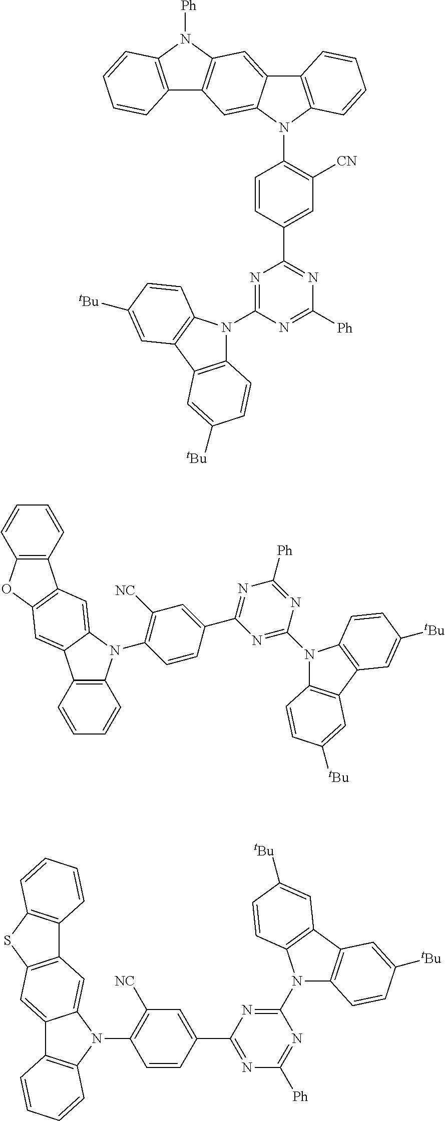 [SMILES: CC(C)(C)c1ccc2c(c1)c1cc(C(C)(C)C)ccc1n2-c1nc(-c2ccccc2)nc(-c2ccc(-n3c4ccccc4c4cc5c(cc43)c3ccccc3n5-c3ccccc3)c(C#N)c2)n1 ; CC(C)(C)c1ccc2c(c1)c1cc(C(C)(C)C)ccc1n2-c1nc(-c2ccccc2)nc(-c2ccc(-n3c4ccccc4c4cc5oc6ccccc6c5cc43)c(C#N)c2)n1 ; CC(C)(C)c1ccc2c(c1)c1cc(C(C)(C)C)ccc1n2-c1nc(-c2ccccc2)nc(-c2ccc(-n3c4ccccc4c4cc5sc6ccccc6c5cc43)c(C#N)c2)n1]

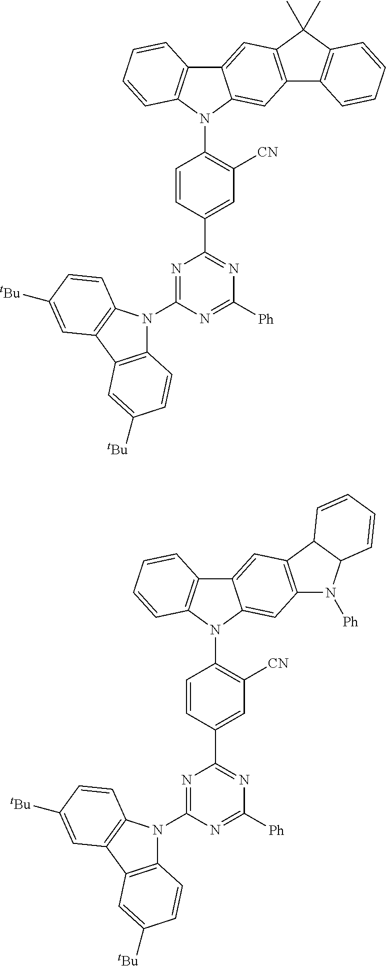 [SMILES: CC(C)(C)c1ccc2c(c1)c1cc(C(C)(C)C)ccc1n2-c1nc(-c2ccccc2)nc(-c2ccc(-n3c4ccccc4c4cc5c(cc43)-c3ccccc3C5(C)C)c(C#N)c2)n1 ; CC(C)(C)c1ccc2c(c1)c1cc(C(C)(C)C)ccc1n2-c1nc(-c2ccccc2)nc(-c2ccc(-n3c4ccccc4c4cc5c(cc43)N(c3ccccc3)C3C=CC=CC53)c(C#N)c2)n1]

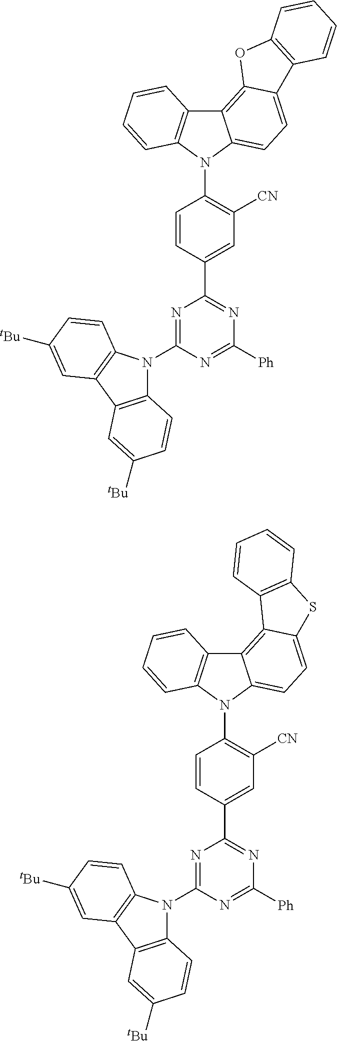 [SMILES: CC(C)(C)c1ccc2c(c1)c1cc(C(C)(C)C)ccc1n2-c1nc(-c2ccccc2)nc(-c2ccc(-n3c4ccccc4c4c5oc6ccccc6c5ccc43)c(C#N)c2)n1 ; CC(C)(C)c1ccc2c(c1)c1cc(C(C)(C)C)ccc1n2-c1nc(-c2ccccc2)nc(-c2ccc(-n3c4ccccc4c4c5c(ccc43)sc3ccccc35)c(C#N)c2)n1]

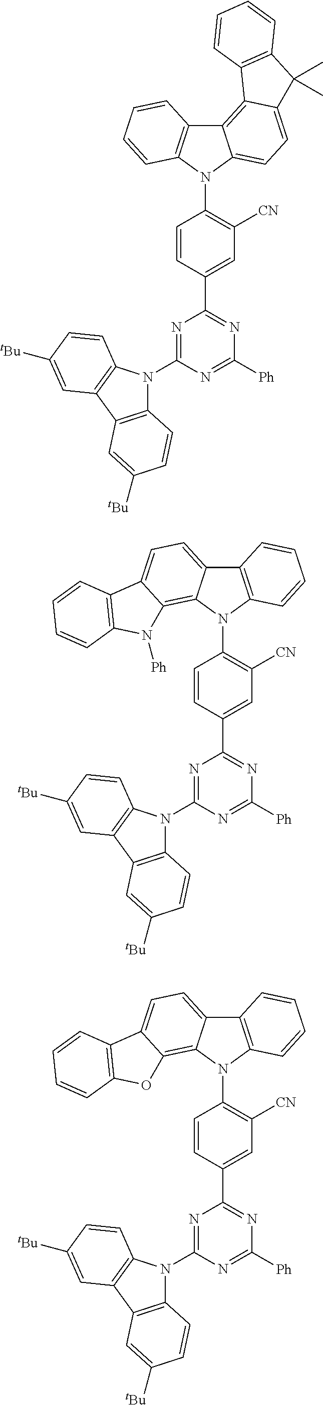 [SMILES: CC(C)(C)c1ccc2c(c1)c1cc(C(C)(C)C)ccc1n2-c1nc(-c2ccccc2)nc(-c2ccc(-n3c4ccccc4c4c5c(ccc43)C(C)(C)c3ccccc3-5)c(C#N)c2)n1 ; CC(C)(C)c1ccc2c(c1)c1cc(C(C)(C)C)ccc1n2-c1nc(-c2ccccc2)nc(-c2ccc(-n3c4ccccc4c4ccc5c6ccccc6n(-c6ccccc6)c5c43)c(C#N)c2)n1 ; CC(C)(C)c1ccc2c(c1)c1cc(C(C)(C)C)ccc1n2-c1nc(-c2ccccc2)nc(-c2ccc(-n3c4ccccc4c4ccc5c6ccccc6oc5c43)c(C#N)c2)n1]

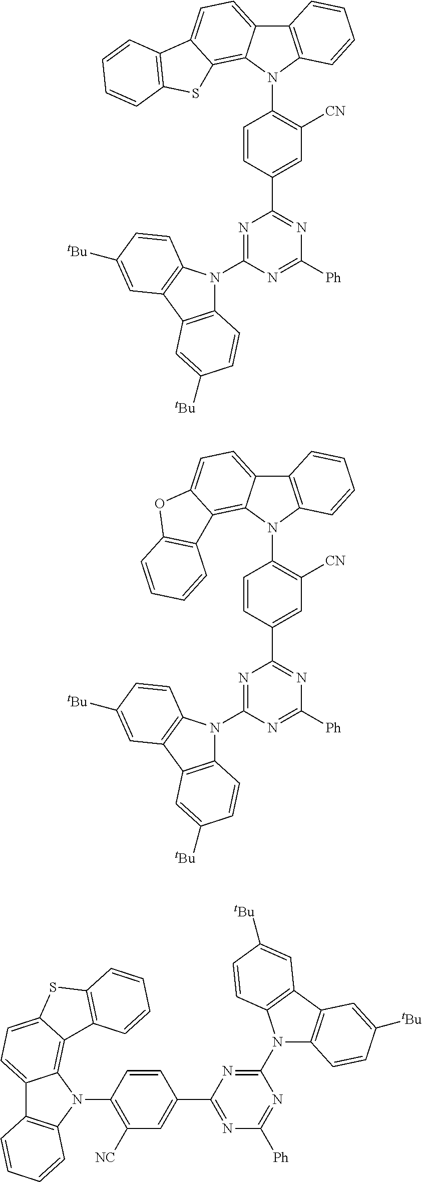 [SMILES: CC(C)(C)c1ccc2c(c1)c1cc(C(C)(C)C)ccc1n2-c1nc(-c2ccccc2)nc(-c2ccc(-n3c4ccccc4c4ccc5c6ccccc6sc5c43)c(C#N)c2)n1 ; CC(C)(C)c1ccc2c(c1)c1cc(C(C)(C)C)ccc1n2-c1nc(-c2ccccc2)nc(-c2ccc(-n3c4ccccc4c4ccc5oc6ccccc6c5c43)c(C#N)c2)n1 ; CC(C)(C)c1ccc2c(c1)c1cc(C(C)(C)C)ccc1n2-c1nc(-c2ccccc2)nc(-c2ccc(-n3c4ccccc4c4ccc5sc6ccccc6c5c43)c(C#N)c2)n1]

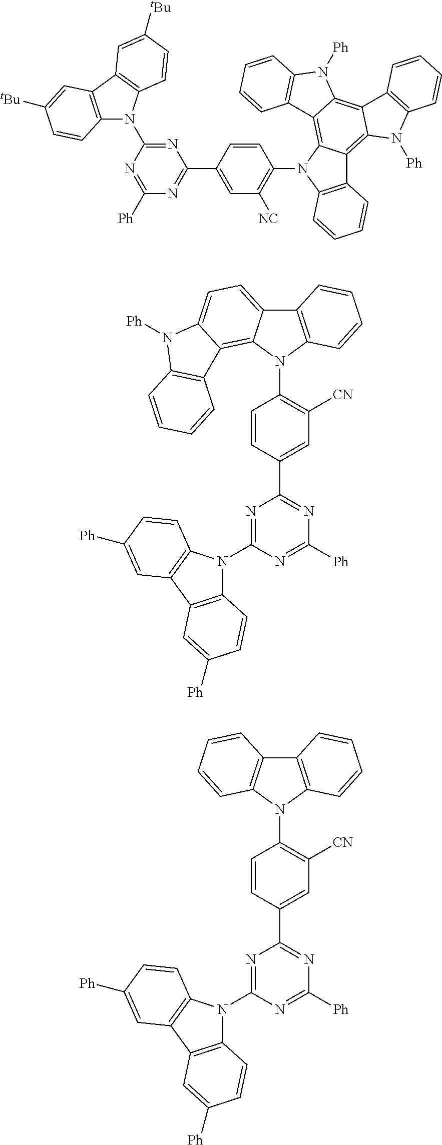 [SMILES: CC(C)(C)c1ccc2c(c1)c1cc(C(C)(C)C)ccc1n2-c1nc(-c2ccccc2)nc(-c2ccc(-n3c4ccccc4c4c5c(c6ccccc6n5-c5ccccc5)c5c(c6ccccc6n5-c5ccccc5)c43)c(C#N)c2)n1 ; N#Cc1cc(-c2nc(-c3ccccc3)nc(-n3c4ccc(-c5ccccc5)cc4c4cc(-c5ccccc5)ccc43)n2)ccc1-n1c2ccccc2c2ccc3c(c4ccccc4n3-c3ccccc3)c21 ; N#Cc1cc(-c2nc(-c3ccccc3)nc(-n3c4ccc(-c5ccccc5)cc4c4cc(-c5ccccc5)ccc43)n2)ccc1-n1c2ccccc2c2ccccc21]

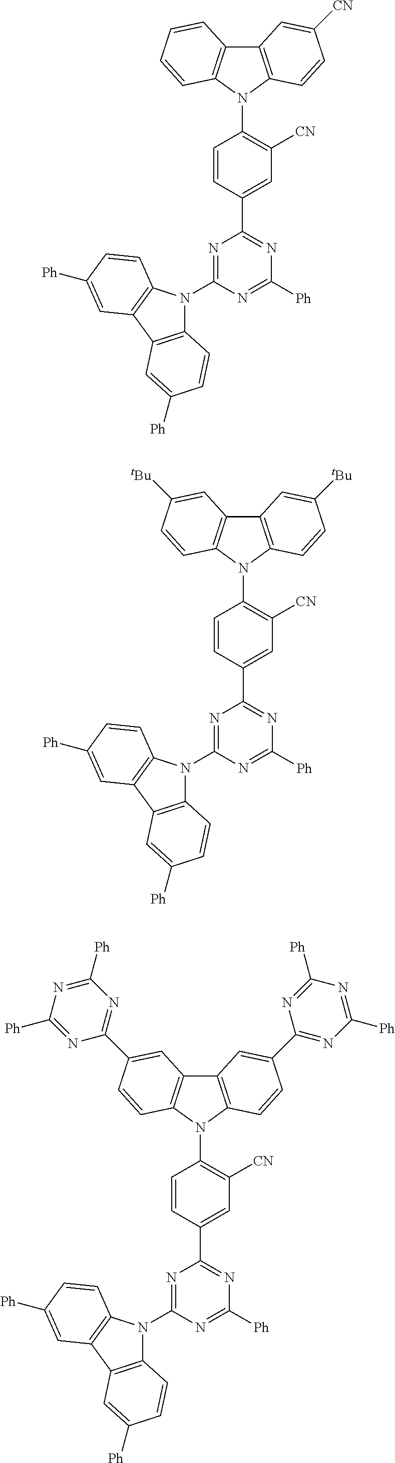 [SMILES: N#Cc1ccc2c(c1)c1ccccc1n2-c1ccc(-c2nc(-c3ccccc3)nc(-n3c4ccc(-c5ccccc5)cc4c4cc(-c5ccccc5)ccc43)n2)cc1C#N ; CC(C)(C)c1ccc2c(c1)c1cc(C(C)(C)C)ccc1n2-c1ccc(-c2nc(-c3ccccc3)nc(-n3c4ccc(-c5ccccc5)cc4c4cc(-c5ccccc5)ccc43)n2)cc1C#N ; N#Cc1cc(-c2nc(-c3ccccc3)nc(-n3c4ccc(-c5ccccc5)cc4c4cc(-c5ccccc5)ccc43)n2)ccc1-n1c2ccc(-c3nc(-c4ccccc4)nc(-c4ccccc4)n3)cc2c2cc(-c3nc(-c4ccccc4)nc(-c4ccccc4)n3)ccc21]

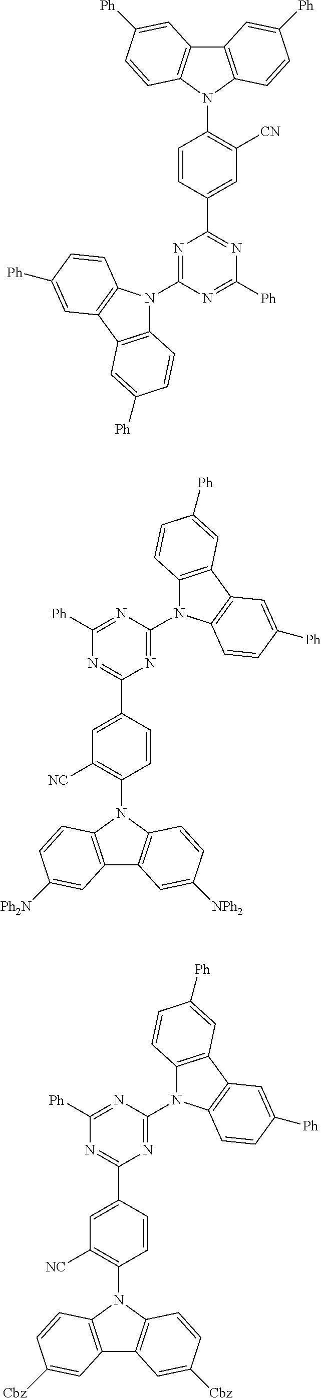

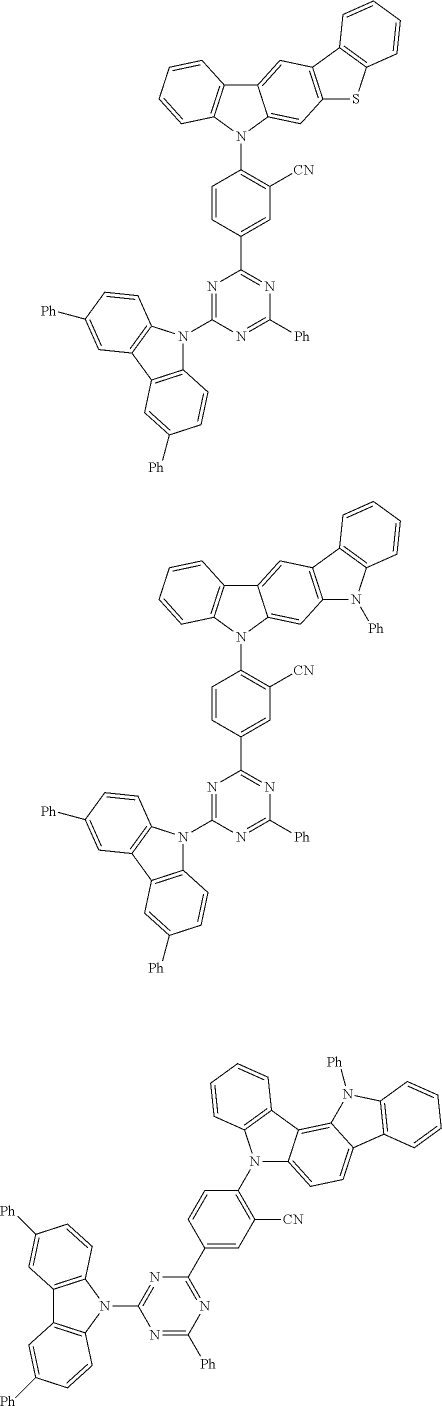 [SMILES: N#Cc1cc(-c2nc(-c3ccccc3)nc(-n3c4ccc(-c5ccccc5)cc4c4cc(-c5ccccc5)ccc43)n2)ccc1-n1c2ccccc2c2cc3c(cc21)sc1ccccc13 ; N#Cc1cc(-c2nc(-c3ccccc3)nc(-n3c4ccc(-c5ccccc5)cc4c4cc(-c5ccccc5)ccc43)n2)ccc1-n1c2ccccc2c2cc3c4ccccc4n(-c4ccccc4)c3cc21 ; N#Cc1cc(-c2nc(-c3ccccc3)nc(-n3c4ccc(-c5ccccc5)cc4c4cc(-c5ccccc5)ccc43)n2)ccc1-n1c2ccccc2c2c1ccc1c3ccccc3n(-c3ccccc3)c12]

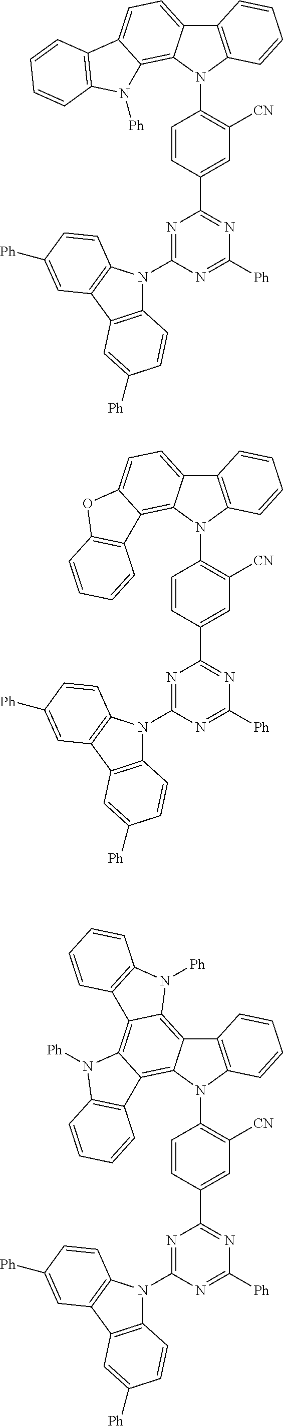 [SMILES: N#Cc1cc(-c2nc(-c3ccccc3)nc(-n3c4ccc(-c5ccccc5)cc4c4cc(-c5ccccc5)ccc43)n2)ccc1-n1c2ccccc2c2ccc3c4ccccc4n(-c4ccccc4)c3c21 ; N#Cc1cc(-c2nc(-c3ccccc3)nc(-n3c4ccc(-c5ccccc5)cc4c4cc(-c5ccccc5)ccc43)n2)ccc1-n1c2ccccc2c2ccc3oc4ccccc4c3c21 ; N#Cc1cc(-c2nc(-c3ccccc3)nc(-n3c4ccc(-c5ccccc5)cc4c4cc(-c5ccccc5)ccc43)n2)ccc1-n1c2ccccc2c2c3c(c4ccccc4n3-c3ccccc3)c3c(c4ccccc4n3-c3ccccc3)c21]

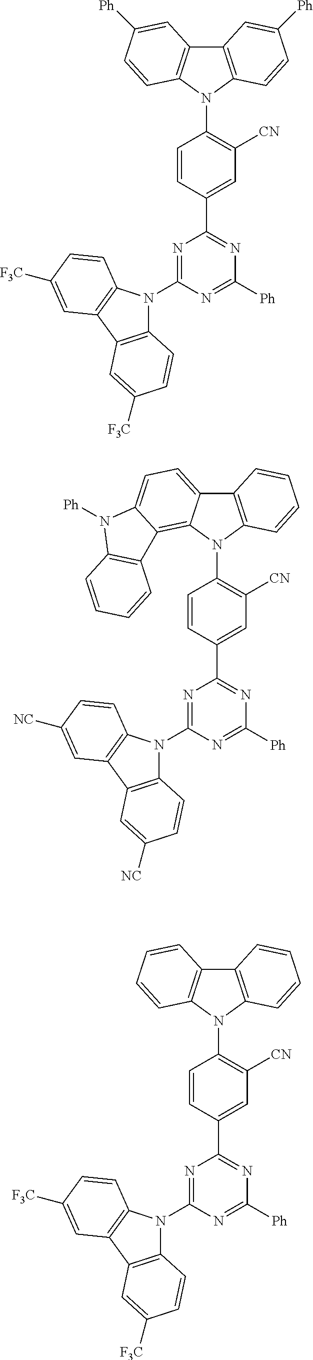 [SMILES: N#Cc1cc(-c2nc(-c3ccccc3)nc(-n3c4ccc(C(F)(F)F)cc4c4cc(C(F)(F)F)ccc43)n2)ccc1-n1c2ccc(-c3ccccc3)cc2c2cc(-c3ccccc3)ccc21 ; N#Cc1ccc2c(c1)c1cc(C#N)ccc1n2-c1nc(-c2ccccc2)nc(-c2ccc(-n3c4ccccc4c4ccc5c(c6ccccc6n5-c5ccccc5)c43)c(C#N)c2)n1 ; N#Cc1cc(-c2nc(-c3ccccc3)nc(-n3c4ccc(C(F)(F)F)cc4c4cc(C(F)(F)F)ccc43)n2)ccc1-n1c2ccccc2c2ccccc21]

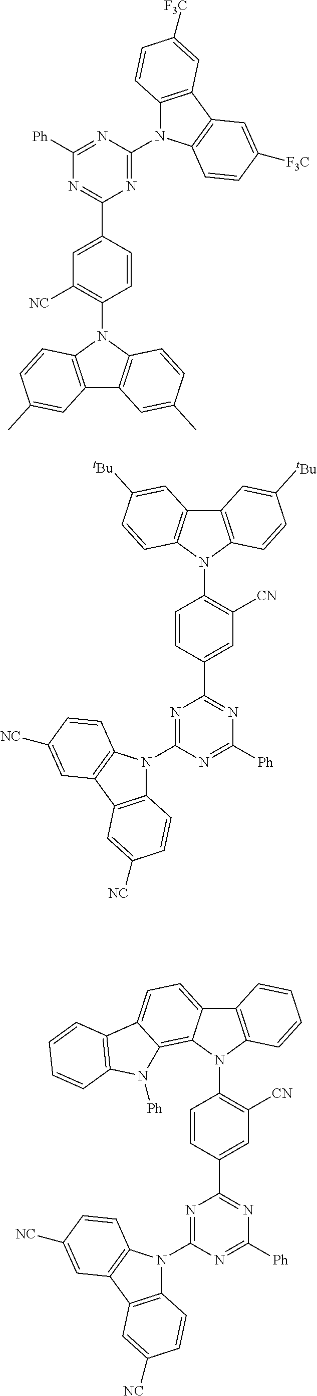 [SMILES: Cc1ccc2c(c1)c1cc(C)ccc1n2-c1ccc(-c2nc(-c3ccccc3)nc(-n3c4ccc(C(F)(F)F)cc4c4cc(C(F)(F)F)ccc43)n2)cc1C#N ; CC(C)(C)c1ccc2c(c1)c1cc(C(C)(C)C)ccc1n2-c1ccc(-c2nc(-c3ccccc3)nc(-n3c4ccc(C#N)cc4c4cc(C#N)ccc43)n2)cc1C#N ; N#Cc1ccc2c(c1)c1cc(C#N)ccc1n2-c1nc(-c2ccccc2)nc(-c2ccc(-n3c4ccccc4c4ccc5c6ccccc6n(-c6ccccc6)c5c43)c(C#N)c2)n1]

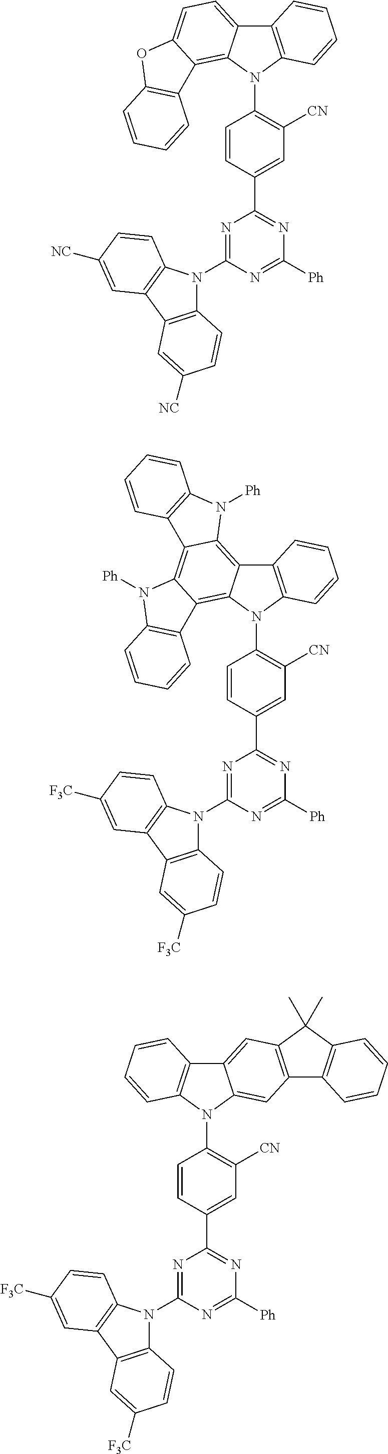 [SMILES: N#Cc1ccc2c(c1)c1cc(C#N)ccc1n2-c1nc(-c2ccccc2)nc(-c2ccc(-n3c4ccccc4c4ccc5oc6ccccc6c5c43)c(C#N)c2)n1 ; N#Cc1cc(-c2nc(-c3ccccc3)nc(-n3c4ccc(C(F)(F)F)cc4c4cc(C(F)(F)F)ccc43)n2)ccc1-n1c2ccccc2c2c3c(c4ccccc4n3-c3ccccc3)c3c(c4ccccc4n3-c3ccccc3)c21 ; CC1(C)c2ccccc2-c2cc3c(cc21)c1ccccc1n3-c1ccc(-c2nc(-c3ccccc3)nc(-n3c4ccc(C(F)(F)F)cc4c4cc(C(F)(F)F)ccc43)n2)cc1C#N]

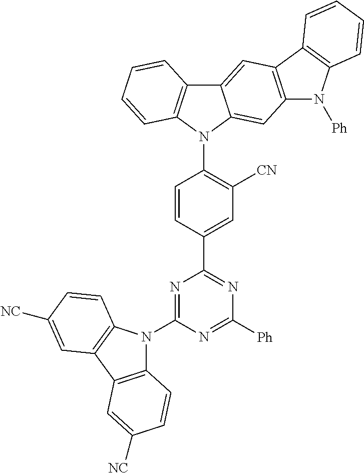 [SMILES: N#Cc1ccc2c(c1)c1cc(C#N)ccc1n2-c1nc(-c2ccccc2)nc(-c2ccc(-n3c4ccccc4c4cc5c6ccccc6n(-c6ccccc6)c5cc43)c(C#N)c2)n1]

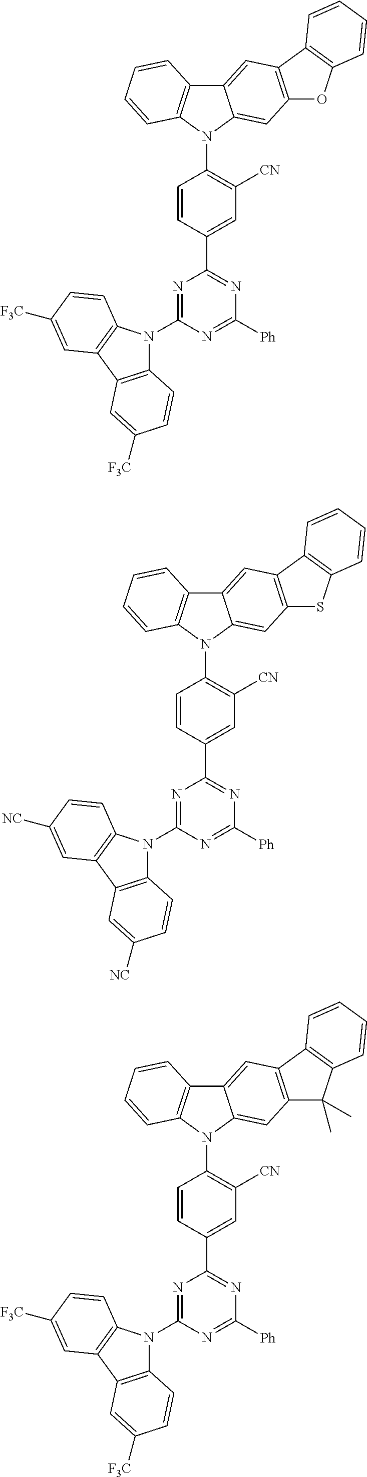 [SMILES: N#Cc1cc(-c2nc(-c3ccccc3)nc(-n3c4ccc(C(F)(F)F)cc4c4cc(C(F)(F)F)ccc43)n2)ccc1-n1c2ccccc2c2cc3c(cc21)oc1ccccc13 ; N#Cc1ccc2c(c1)c1cc(C#N)ccc1n2-c1nc(-c2ccccc2)nc(-c2ccc(-n3c4ccccc4c4cc5c(cc43)sc3ccccc35)c(C#N)c2)n1 ; CC1(C)c2ccccc2-c2cc3c4ccccc4n(-c4ccc(-c5nc(-c6ccccc6)nc(-n6c7ccc(C(F)(F)F)cc7c7cc(C(F)(F)F)ccc76)n5)cc4C#N)c3cc21]

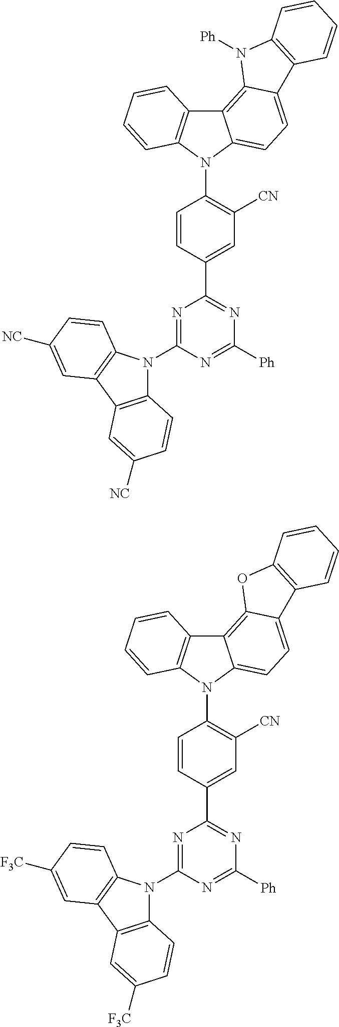 [SMILES: N#Cc1ccc2c(c1)c1cc(C#N)ccc1n2-c1nc(-c2ccccc2)nc(-c2ccc(-n3c4ccccc4c4c3ccc3c5ccccc5n(-c5ccccc5)c34)c(C#N)c2)n1 ; N#Cc1cc(-c2nc(-c3ccccc3)nc(-n3c4ccc(C(F)(F)F)cc4c4cc(C(F)(F)F)ccc43)n2)ccc1-n1c2ccccc2c2c3oc4ccccc4c3ccc21]

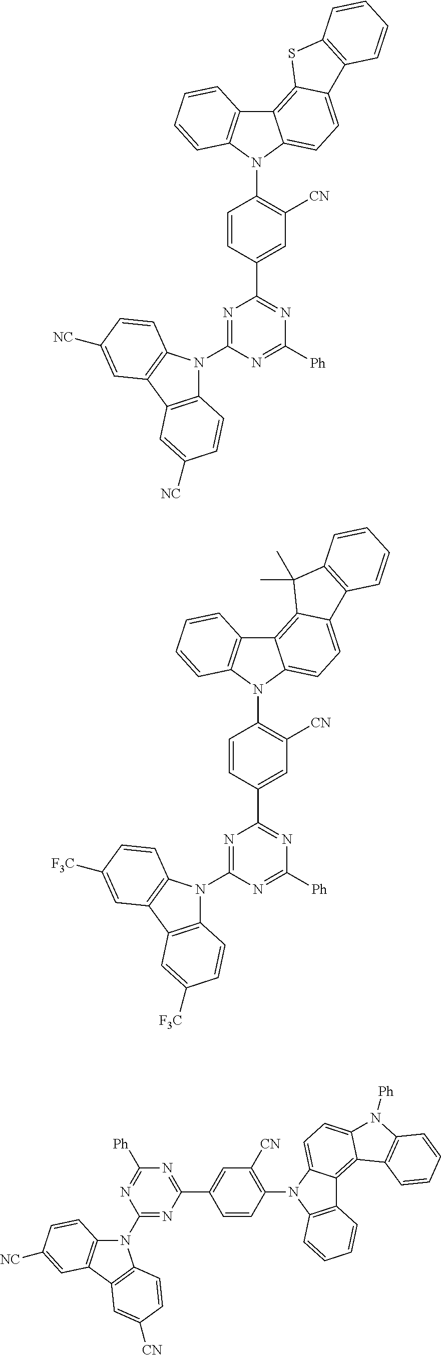 [SMILES: N#Cc1ccc2c(c1)c1cc(C#N)ccc1n2-c1nc(-c2ccccc2)nc(-c2ccc(-n3c4ccccc4c4c5sc6ccccc6c5ccc43)c(C#N)c2)n1 ; CC1(C)c2ccccc2-c2ccc3c(c21)c1ccccc1n3-c1ccc(-c2nc(-c3ccccc3)nc(-n3c4ccc(C(F)(F)F)cc4c4cc(C(F)(F)F)ccc43)n2)cc1C#N ; N#Cc1ccc2c(c1)c1cc(C#N)ccc1n2-c1nc(-c2ccccc2)nc(-c2ccc(-n3c4ccccc4c4c5c6ccccc6n(-c6ccccc6)c5ccc43)c(C#N)c2)n1]

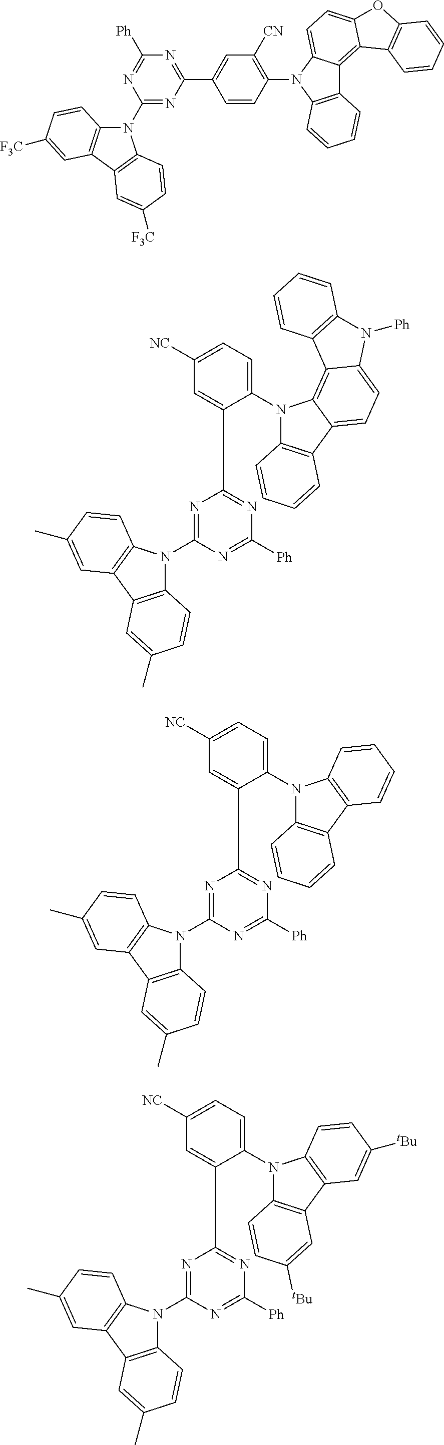

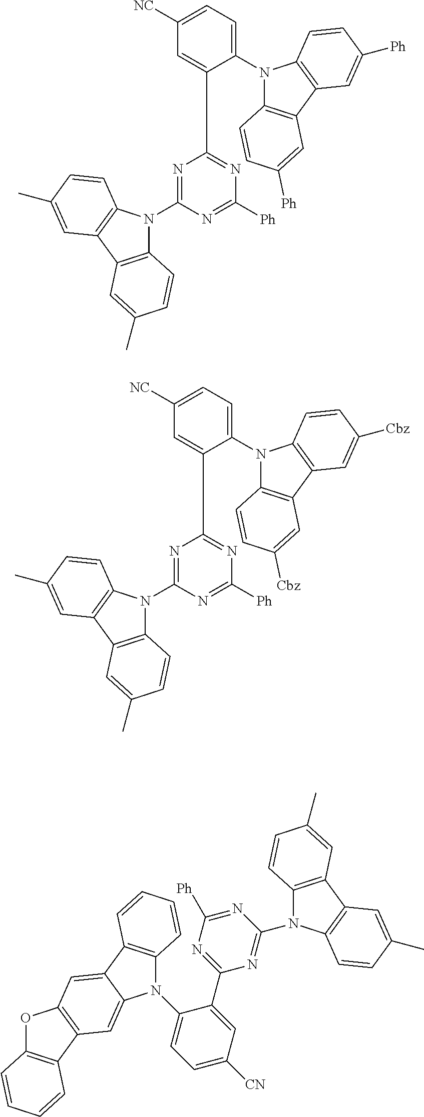

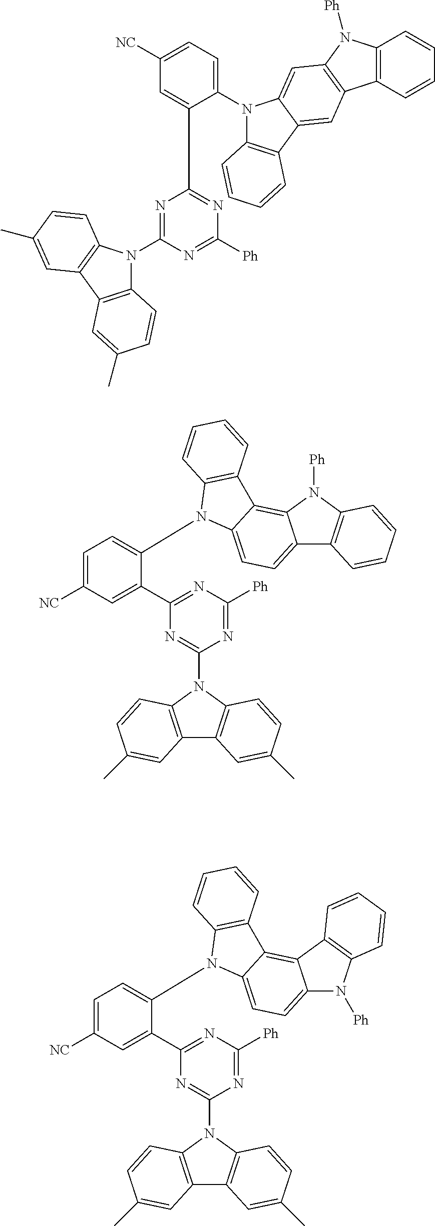 [SMILES: Cc1ccc2c(c1)c1cc(C)ccc1n2-c1nc(-c2ccccc2)nc(-c2cc(C#N)ccc2-n2c3ccccc3c3cc4c5ccccc5n(-c5ccccc5)c4cc32)n1 ; Cc1ccc2c(c1)c1cc(C)ccc1n2-c1nc(-c2ccccc2)nc(-c2cc(C#N)ccc2-n2c3ccccc3c3c2ccc2c4ccccc4n(-c4ccccc4)c23)n1 ; Cc1ccc2c(c1)c1cc(C)ccc1n2-c1nc(-c2ccccc2)nc(-c2cc(C#N)ccc2-n2c3ccccc3c3c4c5ccccc5n(-c5ccccc5)c4ccc32)n1]

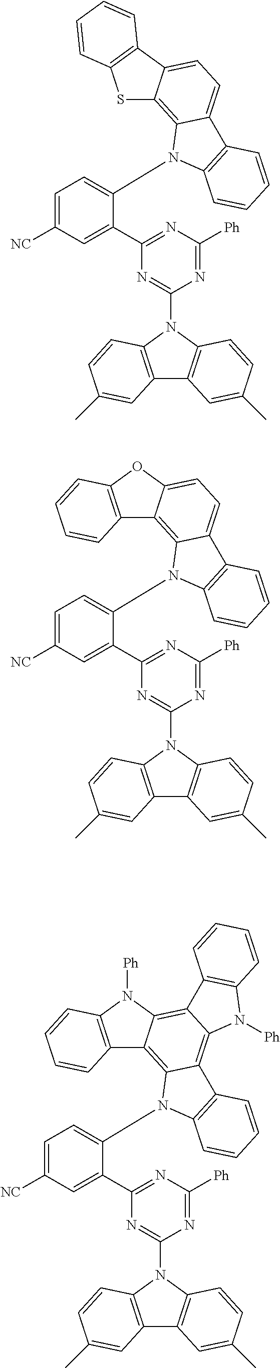 [SMILES: Cc1ccc2c(c1)c1cc(C)ccc1n2-c1nc(-c2ccccc2)nc(-c2cc(C#N)ccc2-n2c3ccccc3c3ccc4c5ccccc5sc4c32)n1 ; Cc1ccc2c(c1)c1cc(C)ccc1n2-c1nc(-c2ccccc2)nc(-c2cc(C#N)ccc2-n2c3ccccc3c3ccc4oc5ccccc5c4c32)n1 ; Cc1ccc2c(c1)c1cc(C)ccc1n2-c1nc(-c2ccccc2)nc(-c2cc(C#N)ccc2-n2c3ccccc3c3c4c(c5ccccc5n4-c4ccccc4)c4c(c5ccccc5n4-c4ccccc4)c32)n1]

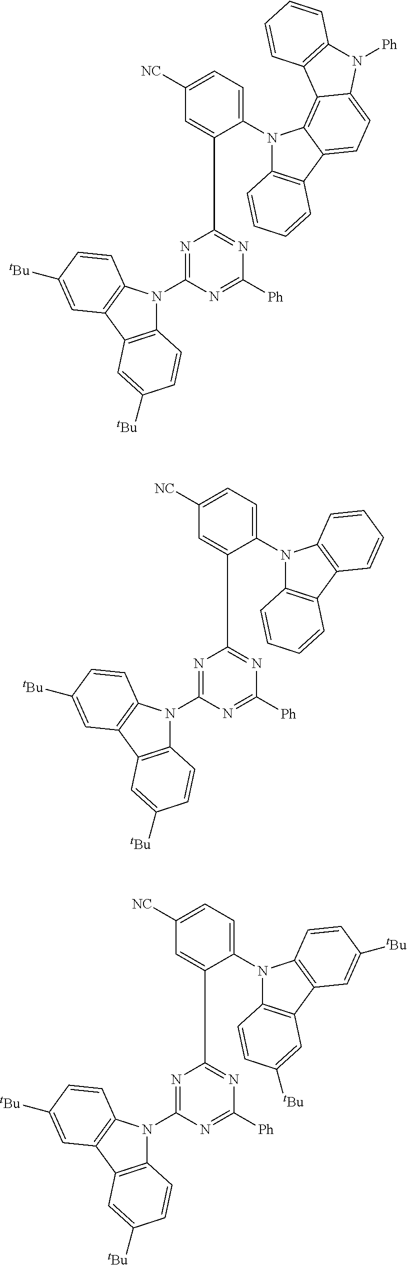 [SMILES: CC(C)(C)c1ccc2c(c1)c1cc(C(C)(C)C)ccc1n2-c1nc(-c2ccccc2)nc(-c2cc(C#N)ccc2-n2c3ccccc3c3ccc4c(c5ccccc5n4-c4ccccc4)c32)n1 ; CC(C)(C)c1ccc2c(c1)c1cc(C(C)(C)C)ccc1n2-c1nc(-c2ccccc2)nc(-c2cc(C#N)ccc2-n2c3ccccc3c3ccccc32)n1 ; CC(C)(C)c1ccc2c(c1)c1cc(C(C)(C)C)ccc1n2-c1nc(-c2ccccc2)nc(-c2cc(C#N)ccc2-n2c3ccc(C(C)(C)C)cc3c3cc(C(C)(C)C)ccc32)n1]

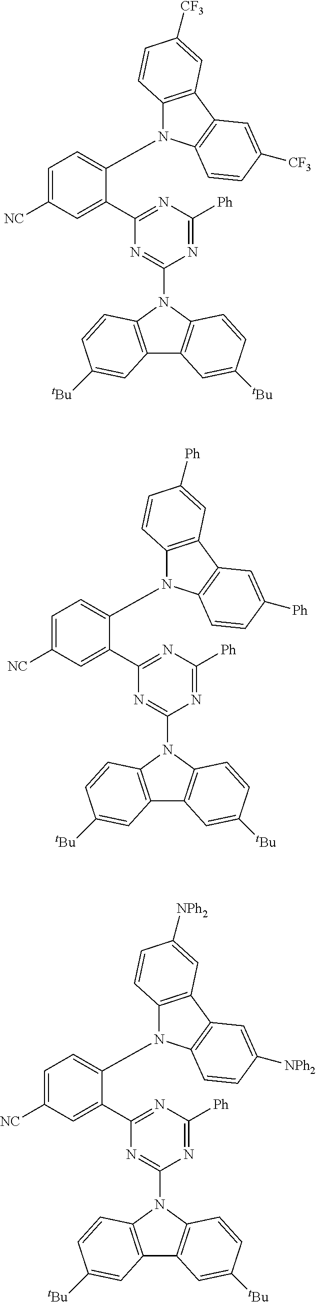

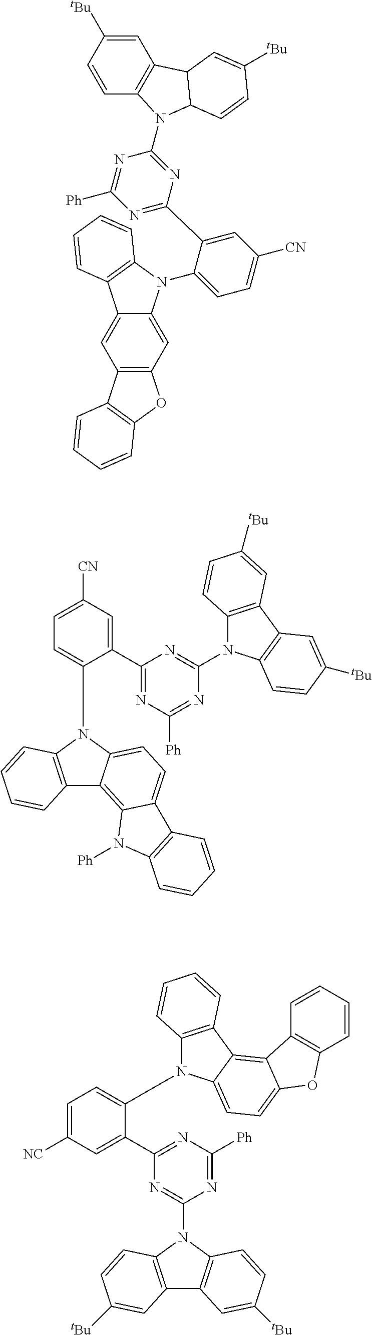 [SMILES: CC(C)(C)C1=CC2c3cc(C(C)(C)C)ccc3N(c3nc(-c4ccccc4)nc(-c4cc(C#N)ccc4-n4c5ccccc5c5cc6c(cc54)oc4ccccc46)n3)C2C=C1 ; CC(C)(C)c1ccc2c(c1)c1cc(C(C)(C)C)ccc1n2-c1nc(-c2ccccc2)nc(-c2cc(C#N)ccc2-n2c3ccccc3c3c2ccc2c4ccccc4n(-c4ccccc4)c23)n1 ; CC(C)(C)c1ccc2c(c1)c1cc(C(C)(C)C)ccc1n2-c1nc(-c2ccccc2)nc(-c2cc(C#N)ccc2-n2c3ccccc3c3c4c(ccc32)oc2ccccc24)n1]

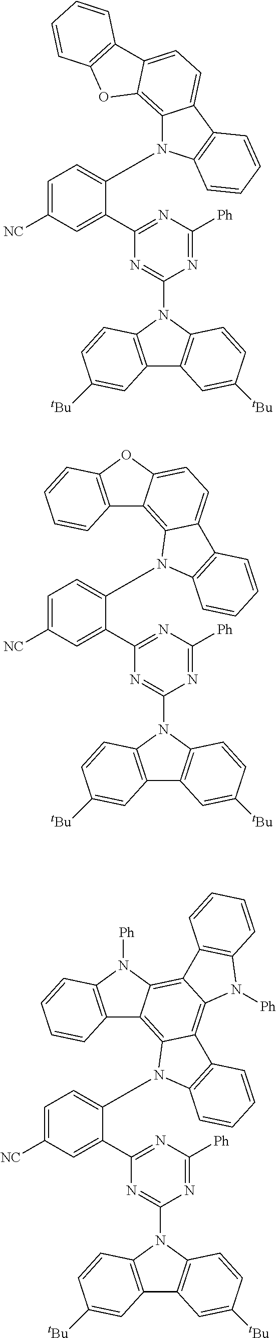 [SMILES: CC(C)(C)c1ccc2c(c1)c1cc(C(C)(C)C)ccc1n2-c1nc(-c2ccccc2)nc(-c2cc(C#N)ccc2-n2c3ccccc3c3ccc4c5ccccc5oc4c32)n1 ; CC(C)(C)c1ccc2c(c1)c1cc(C(C)(C)C)ccc1n2-c1nc(-c2ccccc2)nc(-c2cc(C#N)ccc2-n2c3ccccc3c3ccc4oc5ccccc5c4c32)n1 ; CC(C)(C)c1ccc2c(c1)c1cc(C(C)(C)C)ccc1n2-c1nc(-c2ccccc2)nc(-c2cc(C#N)ccc2-n2c3ccccc3c3c4c(c5ccccc5n4-c4ccccc4)c4c(c5ccccc5n4-c4ccccc4)c32)n1]

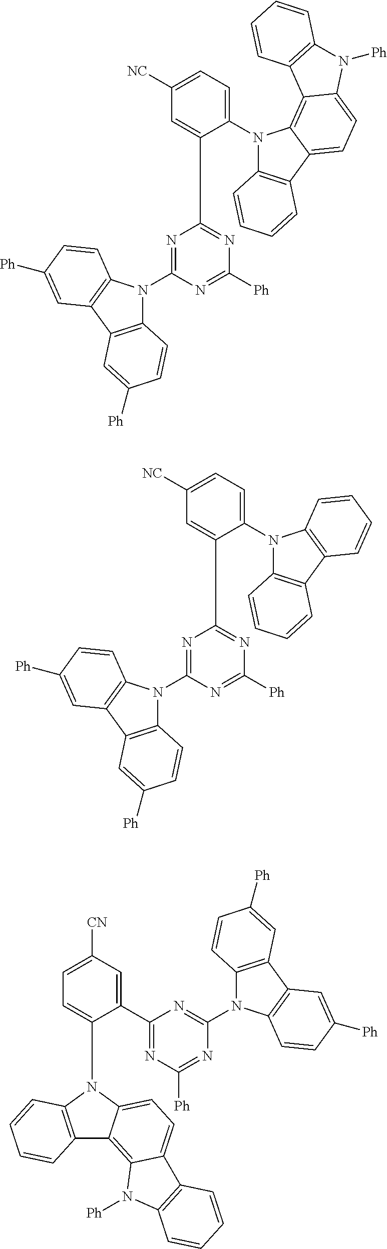 [SMILES: N#Cc1ccc(-n2c3ccccc3c3ccc4c(c5ccccc5n4-c4ccccc4)c32)c(-c2nc(-c3ccccc3)nc(-n3c4ccc(-c5ccccc5)cc4c4cc(-c5ccccc5)ccc43)n2)c1 ; N#Cc1ccc(-n2c3ccccc3c3ccccc32)c(-c2nc(-c3ccccc3)nc(-n3c4ccc(-c5ccccc5)cc4c4cc(-c5ccccc5)ccc43)n2)c1 ; N#Cc1ccc(-n2c3ccccc3c3c2ccc2c4ccccc4n(-c4ccccc4)c23)c(-c2nc(-c3ccccc3)nc(-n3c4ccc(-c5ccccc5)cc4c4cc(-c5ccccc5)ccc43)n2)c1]

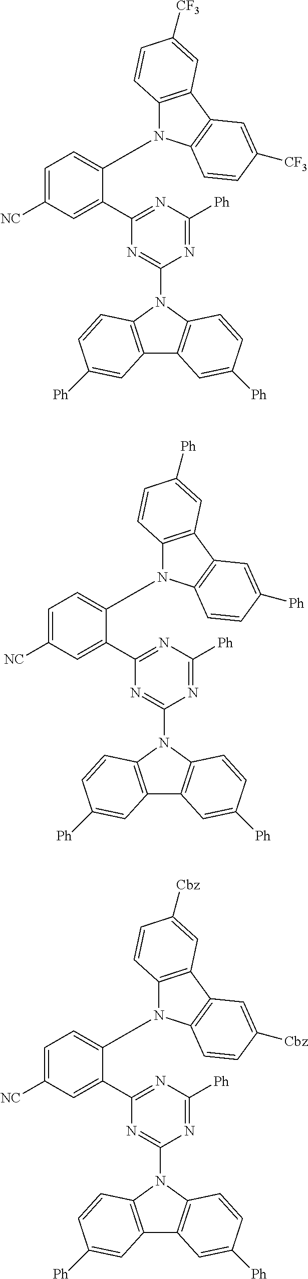 [SMILES: N#Cc1ccc(-n2c3ccc(C(F)(F)F)cc3c3cc(C(F)(F)F)ccc32)c(-c2nc(-c3ccccc3)nc(-n3c4ccc(-c5ccccc5)cc4c4cc(-c5ccccc5)ccc43)n2)c1 ; N#Cc1ccc(-n2c3ccc(-c4ccccc4)cc3c3cc(-c4ccccc4)ccc32)c(-c2nc(-c3ccccc3)nc(-n3c4ccc(-c5ccccc5)cc4c4cc(-c5ccccc5)ccc43)n2)c1 ; N#Cc1ccc(-n2c3ccc(C(=O)OCc4ccccc4)cc3c3cc(C(=O)OCc4ccccc4)ccc32)c(-c2nc(-c3ccccc3)nc(-n3c4ccc(-c5ccccc5)cc4c4cc(-c5ccccc5)ccc43)n2)c1]

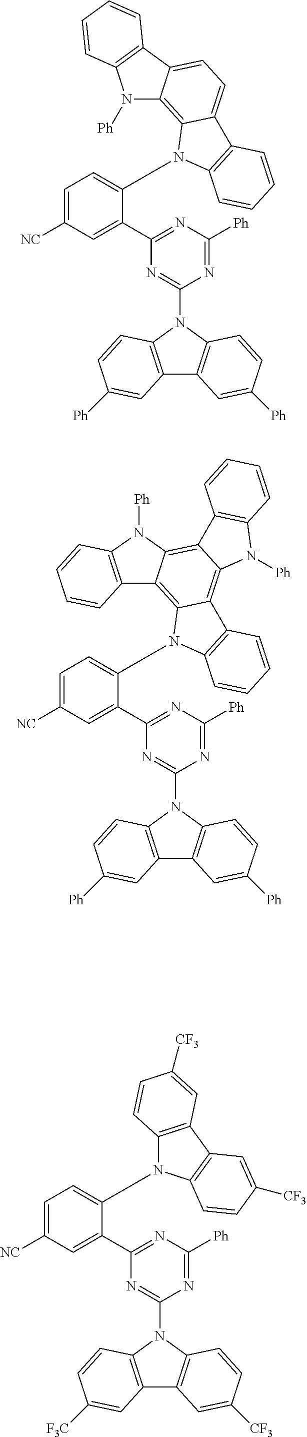 [SMILES: N#Cc1ccc(-n2c3ccccc3c3ccc4c5ccccc5n(-c5ccccc5)c4c32)c(-c2nc(-c3ccccc3)nc(-n3c4ccc(-c5ccccc5)cc4c4cc(-c5ccccc5)ccc43)n2)c1 ; N#Cc1ccc(-n2c3ccccc3c3c4c(c5ccccc5n4-c4ccccc4)c4c(c5ccccc5n4-c4ccccc4)c32)c(-c2nc(-c3ccccc3)nc(-n3c4ccc(-c5ccccc5)cc4c4cc(-c5ccccc5)ccc43)n2)c1 ; N#Cc1ccc(-n2c3ccc(C(F)(F)F)cc3c3cc(C(F)(F)F)ccc32)c(-c2nc(-c3ccccc3)nc(-n3c4ccc(C(F)(F)F)cc4c4cc(C(F)(F)F)ccc43)n2)c1]

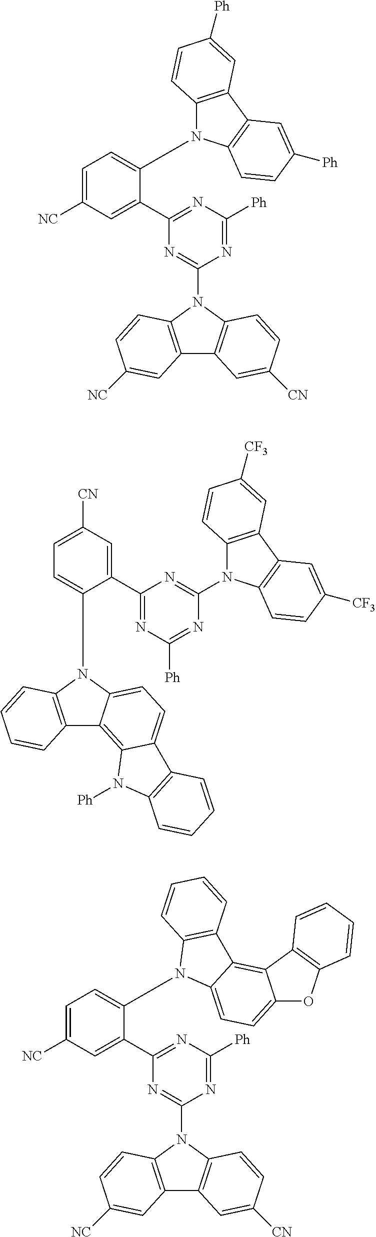

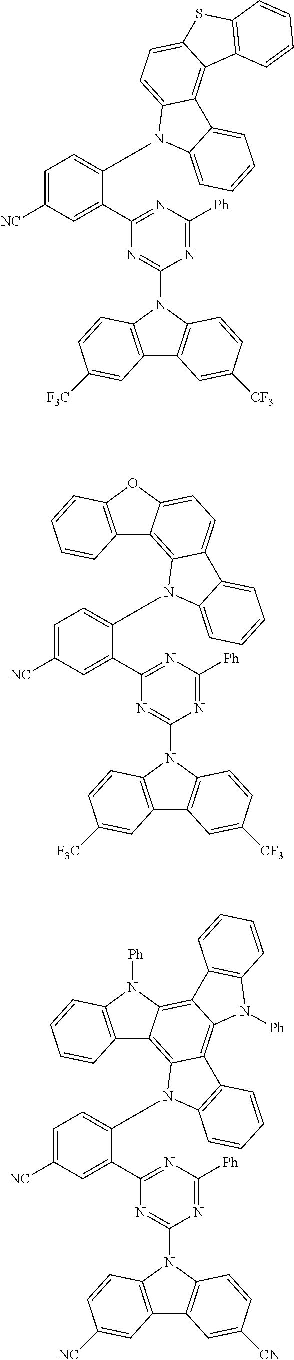 [SMILES: N#Cc1ccc(-n2c3ccccc3c3c4c(ccc32)sc2ccccc24)c(-c2nc(-c3ccccc3)nc(-n3c4ccc(C(F)(F)F)cc4c4cc(C(F)(F)F)ccc43)n2)c1 ; N#Cc1ccc(-n2c3ccccc3c3ccc4oc5ccccc5c4c32)c(-c2nc(-c3ccccc3)nc(-n3c4ccc(C(F)(F)F)cc4c4cc(C(F)(F)F)ccc43)n2)c1 ; N#Cc1ccc(-n2c3ccccc3c3c4c(c5ccccc5n4-c4ccccc4)c4c(c5ccccc5n4-c4ccccc4)c32)c(-c2nc(-c3ccccc3)nc(-n3c4ccc(C#N)cc4c4cc(C#N)ccc43)n2)c1]

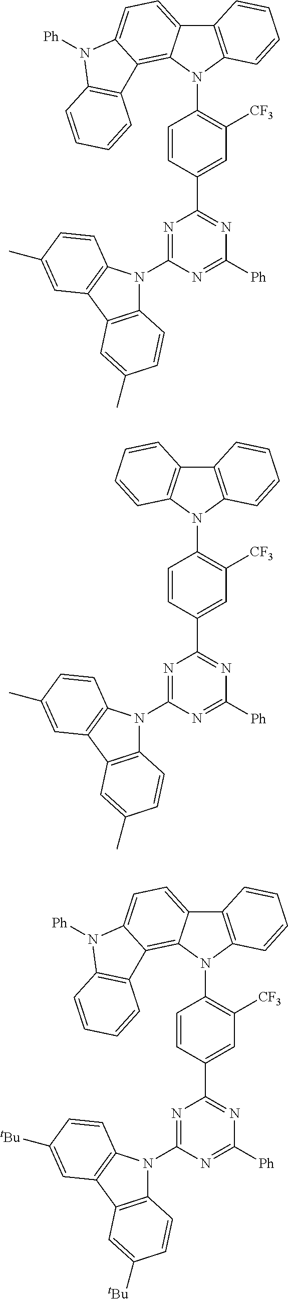

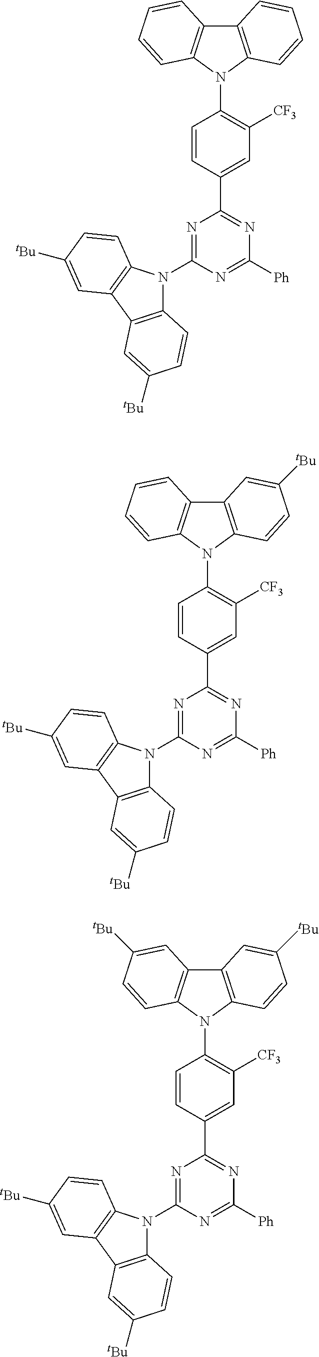 [SMILES: CC(C)(C)c1ccc2c(c1)c1cc(C(C)(C)C)ccc1n2-c1nc(-c2ccccc2)nc(-c2ccc(-n3c4ccccc4c4ccccc43)c(C(F)(F)F)c2)n1 ; CC(C)(C)c1ccc2c(c1)c1cc(C(C)(C)C)ccc1n2-c1nc(-c2ccccc2)nc(-c2ccc(-n3c4ccccc4c4cc(C(C)(C)C)ccc43)c(C(F)(F)F)c2)n1 ; CC(C)(C)c1ccc2c(c1)c1cc(C(C)(C)C)ccc1n2-c1nc(-c2ccccc2)nc(-c2ccc(-n3c4ccc(C(C)(C)C)cc4c4cc(C(C)(C)C)ccc43)c(C(F)(F)F)c2)n1]

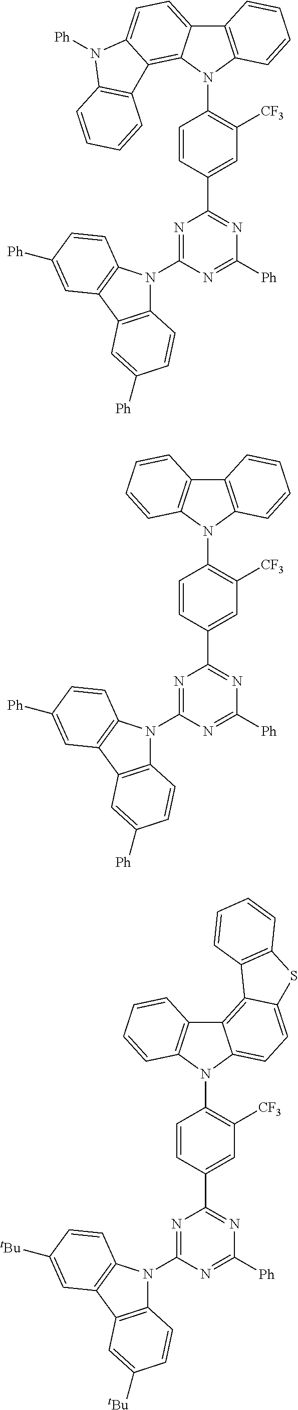 [SMILES: FC(F)(F)c1cc(-c2nc(-c3ccccc3)nc(-n3c4ccc(-c5ccccc5)cc4c4cc(-c5ccccc5)ccc43)n2)ccc1-n1c2ccccc2c2ccc3c(c4ccccc4n3-c3ccccc3)c21 ; FC(F)(F)c1cc(-c2nc(-c3ccccc3)nc(-n3c4ccc(-c5ccccc5)cc4c4cc(-c5ccccc5)ccc43)n2)ccc1-n1c2ccccc2c2ccccc21 ; CC(C)(C)c1ccc2c(c1)c1cc(C(C)(C)C)ccc1n2-c1nc(-c2ccccc2)nc(-c2ccc(-n3c4ccccc4c4c5c(ccc43)sc3ccccc35)c(C(F)(F)F)c2)n1]

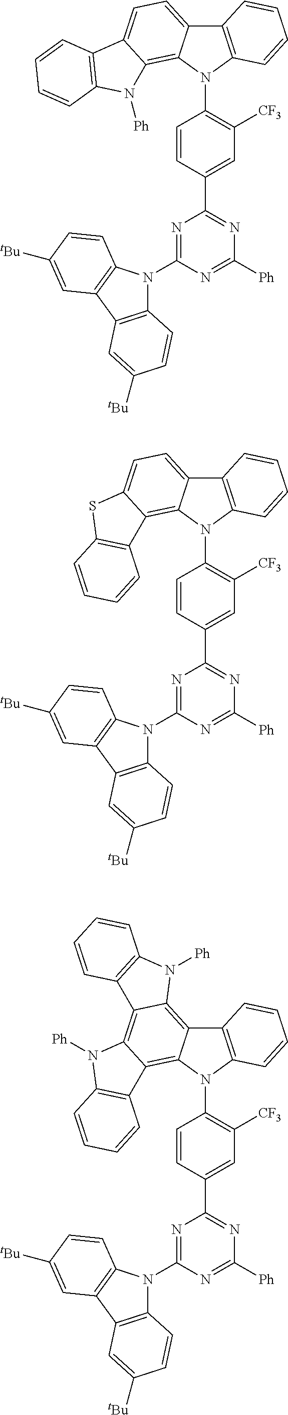 [SMILES: CC(C)(C)c1ccc2c(c1)c1cc(C(C)(C)C)ccc1n2-c1nc(-c2ccccc2)nc(-c2ccc(-n3c4ccccc4c4ccc5c6ccccc6n(-c6ccccc6)c5c43)c(C(F)(F)F)c2)n1 ; CC(C)(C)c1ccc2c(c1)c1cc(C(C)(C)C)ccc1n2-c1nc(-c2ccccc2)nc(-c2ccc(-n3c4ccccc4c4ccc5sc6ccccc6c5c43)c(C(F)(F)F)c2)n1 ; CC(C)(C)c1ccc2c(c1)c1cc(C(C)(C)C)ccc1n2-c1nc(-c2ccccc2)nc(-c2ccc(-n3c4ccccc4c4c5c(c6ccccc6n5-c5ccccc5)c5c(c6ccccc6n5-c5ccccc5)c43)c(C(F)(F)F)c2)n1]

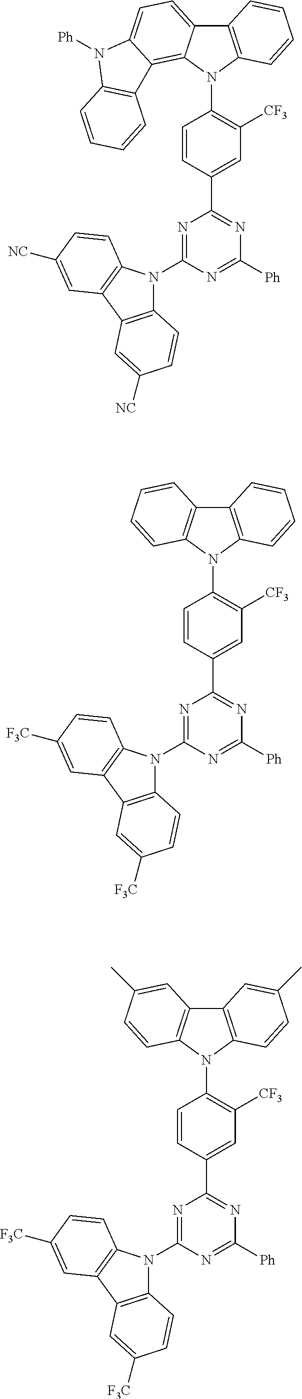 [SMILES: N#Cc1ccc2c(c1)c1cc(C#N)ccc1n2-c1nc(-c2ccccc2)nc(-c2ccc(-n3c4ccccc4c4ccc5c(c6ccccc6n5-c5ccccc5)c43)c(C(F)(F)F)c2)n1 ; FC(F)(F)c1ccc2c(c1)c1cc(C(F)(F)F)ccc1n2-c1nc(-c2ccccc2)nc(-c2ccc(-n3c4ccccc4c4ccccc43)c(C(F)(F)F)c2)n1 ; Cc1ccc2c(c1)c1cc(C)ccc1n2-c1ccc(-c2nc(-c3ccccc3)nc(-n3c4ccc(C(F)(F)F)cc4c4cc(C(F)(F)F)ccc43)n2)cc1C(F)(F)F]

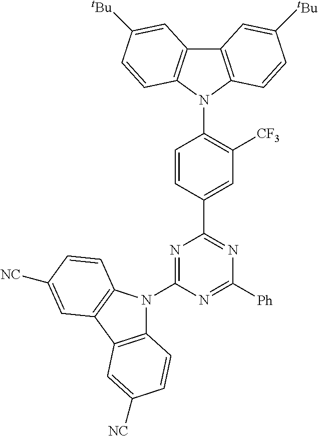 [SMILES: CC(C)(C)c1ccc2c(c1)c1cc(C(C)(C)C)ccc1n2-c1ccc(-c2nc(-c3ccccc3)nc(-n3c4ccc(C#N)cc4c4cc(C#N)ccc43)n2)cc1C(F)(F)F]

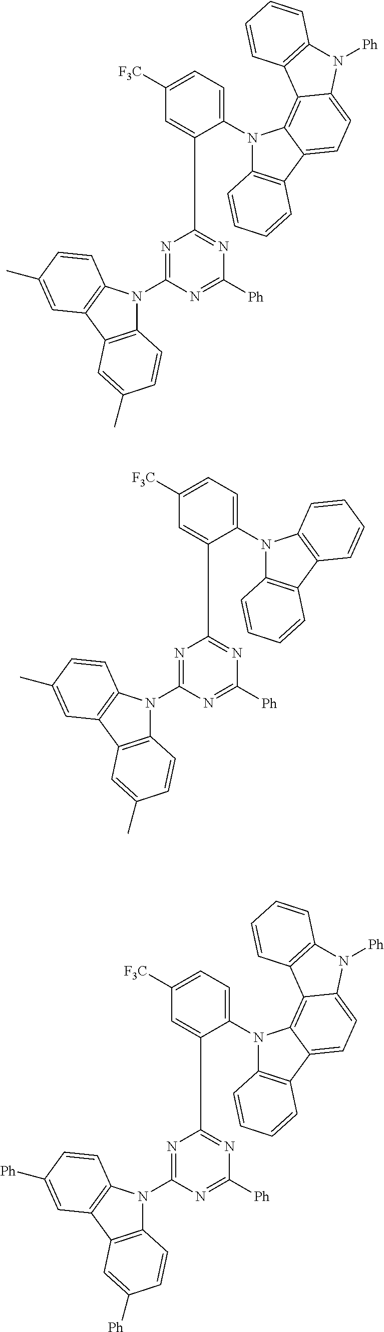 [SMILES: Cc1ccc2c(c1)c1cc(C)ccc1n2-c1nc(-c2ccccc2)nc(-c2cc(C(F)(F)F)ccc2-n2c3ccccc3c3ccc4c(c5ccccc5n4-c4ccccc4)c32)n1 ; Cc1ccc2c(c1)c1cc(C)ccc1n2-c1nc(-c2ccccc2)nc(-c2cc(C(F)(F)F)ccc2-n2c3ccccc3c3ccccc32)n1 ; FC(F)(F)c1ccc(-n2c3ccccc3c3ccc4c(c5ccccc5n4-c4ccccc4)c32)c(-c2nc(-c3ccccc3)nc(-n3c4ccc(-c5ccccc5)cc4c4cc(-c5ccccc5)ccc43)n2)c1]

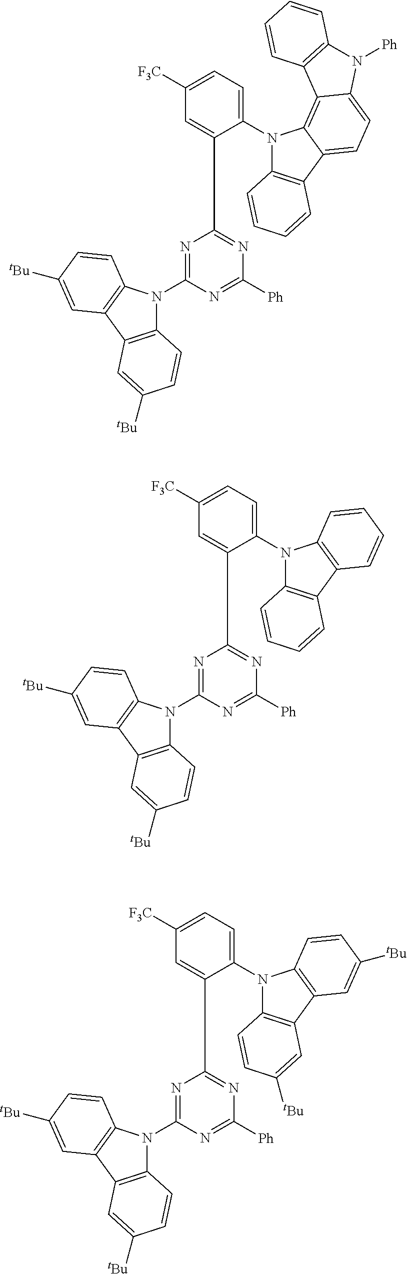 [SMILES: CC(C)(C)c1ccc2c(c1)c1cc(C(C)(C)C)ccc1n2-c1nc(-c2ccccc2)nc(-c2cc(C(F)(F)F)ccc2-n2c3ccccc3c3ccc4c(c5ccccc5n4-c4ccccc4)c32)n1 ; CC(C)(C)c1ccc2c(c1)c1cc(C(C)(C)C)ccc1n2-c1nc(-c2ccccc2)nc(-c2cc(C(F)(F)F)ccc2-n2c3ccccc3c3ccccc32)n1 ; CC(C)(C)c1ccc2c(c1)c1cc(C(C)(C)C)ccc1n2-c1nc(-c2ccccc2)nc(-c2cc(C(F)(F)F)ccc2-n2c3ccc(C(C)(C)C)cc3c3cc(C(C)(C)C)ccc32)n1]

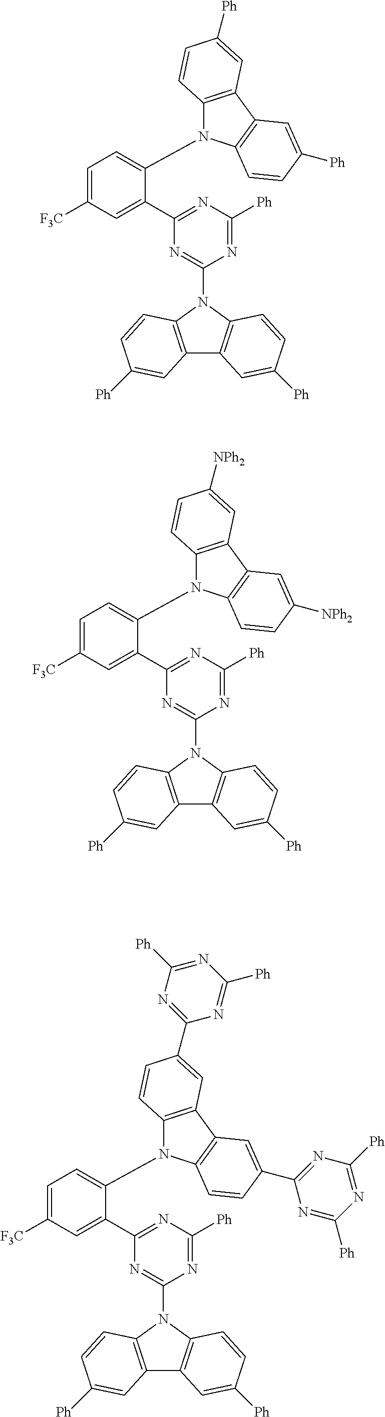

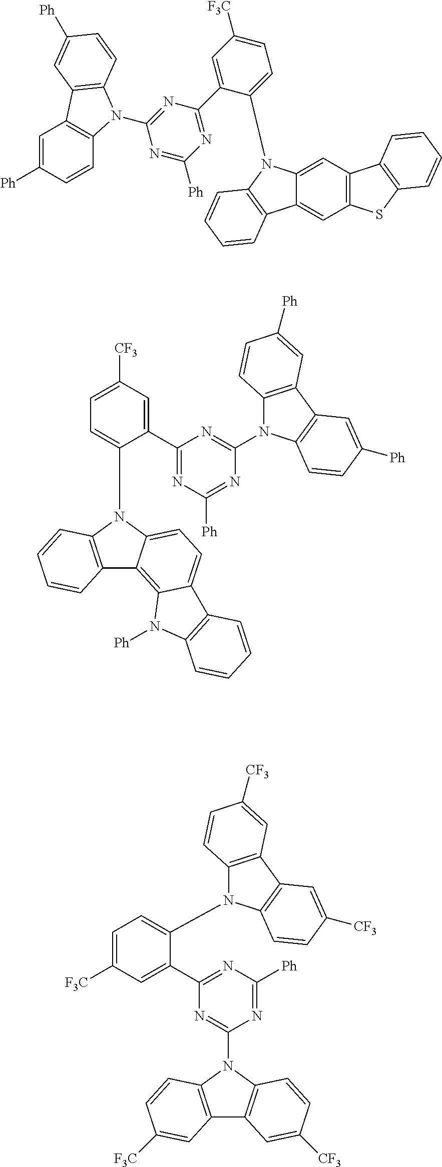 [SMILES: FC(F)(F)c1ccc(-n2c3ccccc3c3cc4sc5ccccc5c4cc32)c(-c2nc(-c3ccccc3)nc(-n3c4ccc(-c5ccccc5)cc4c4cc(-c5ccccc5)ccc43)n2)c1 ; FC(F)(F)c1ccc(-n2c3ccccc3c3c2ccc2c4ccccc4n(-c4ccccc4)c23)c(-c2nc(-c3ccccc3)nc(-n3c4ccc(-c5ccccc5)cc4c4cc(-c5ccccc5)ccc43)n2)c1 ; FC(F)(F)c1ccc(-n2c3ccc(C(F)(F)F)cc3c3cc(C(F)(F)F)ccc32)c(-c2nc(-c3ccccc3)nc(-n3c4ccc(C(F)(F)F)cc4c4cc(C(F)(F)F)ccc43)n2)c1]

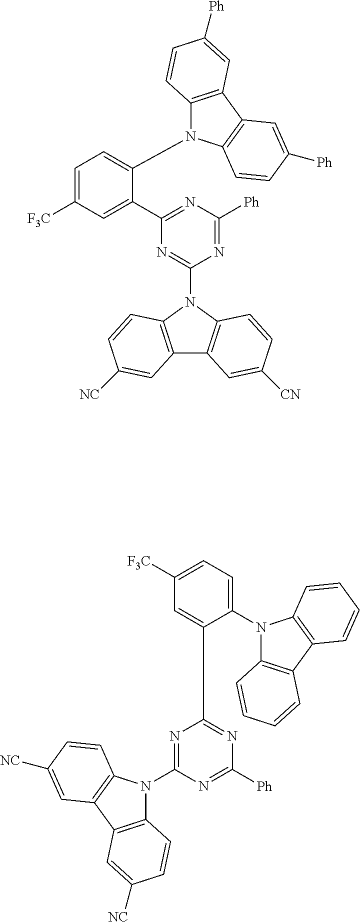 [SMILES: N#Cc1ccc2c(c1)c1cc(C#N)ccc1n2-c1nc(-c2ccccc2)nc(-c2cc(C(F)(F)F)ccc2-n2c3ccc(-c4ccccc4)cc3c3cc(-c4ccccc4)ccc32)n1 ; N#Cc1ccc2c(c1)c1cc(C#N)ccc1n2-c1nc(-c2ccccc2)nc(-c2cc(C(F)(F)F)ccc2-n2c3ccccc3c3ccccc32)n1]

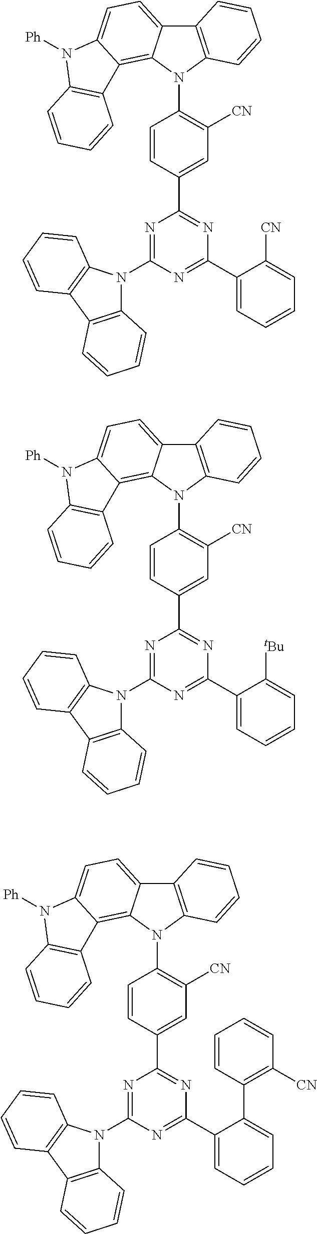 [SMILES: N#Cc1ccccc1-c1nc(-c2ccc(-n3c4ccccc4c4ccc5c(c6ccccc6n5-c5ccccc5)c43)c(C#N)c2)nc(-n2c3ccccc3c3ccccc32)n1 ; CC(C)(C)c1ccccc1-c1nc(-c2ccc(-n3c4ccccc4c4ccc5c(c6ccccc6n5-c5ccccc5)c43)c(C#N)c2)nc(-n2c3ccccc3c3ccccc32)n1 ; N#Cc1ccccc1-c1ccccc1-c1nc(-c2ccc(-n3c4ccccc4c4ccc5c(c6ccccc6n5-c5ccccc5)c43)c(C#N)c2)nc(-n2c3ccccc3c3ccccc32)n1]

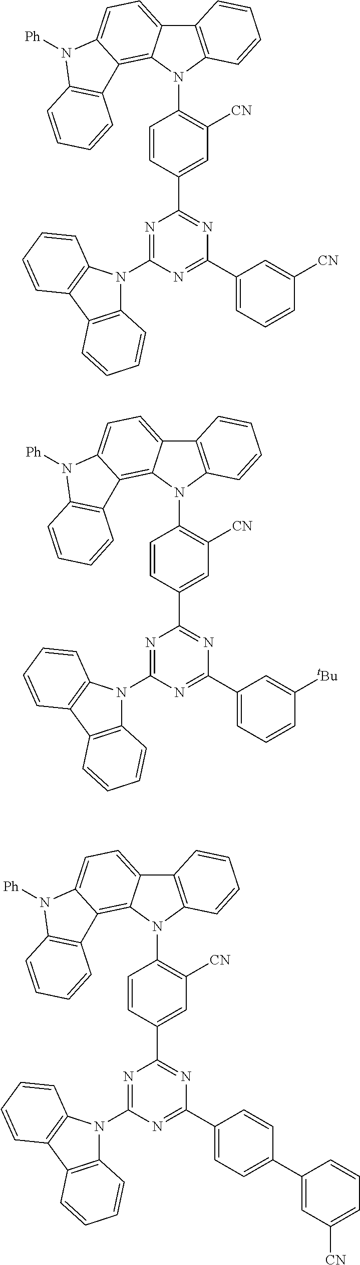 [SMILES: N#Cc1cccc(-c2nc(-c3ccc(-n4c5ccccc5c5ccc6c(c7ccccc7n6-c6ccccc6)c54)c(C#N)c3)nc(-n3c4ccccc4c4ccccc43)n2)c1 ; CC(C)(C)c1cccc(-c2nc(-c3ccc(-n4c5ccccc5c5ccc6c(c7ccccc7n6-c6ccccc6)c54)c(C#N)c3)nc(-n3c4ccccc4c4ccccc43)n2)c1 ; N#Cc1cccc(-c2ccc(-c3nc(-c4ccc(-n5c6ccccc6c6ccc7c(c8ccccc8n7-c7ccccc7)c65)c(C#N)c4)nc(-n4c5ccccc5c5ccccc54)n3)cc2)c1]

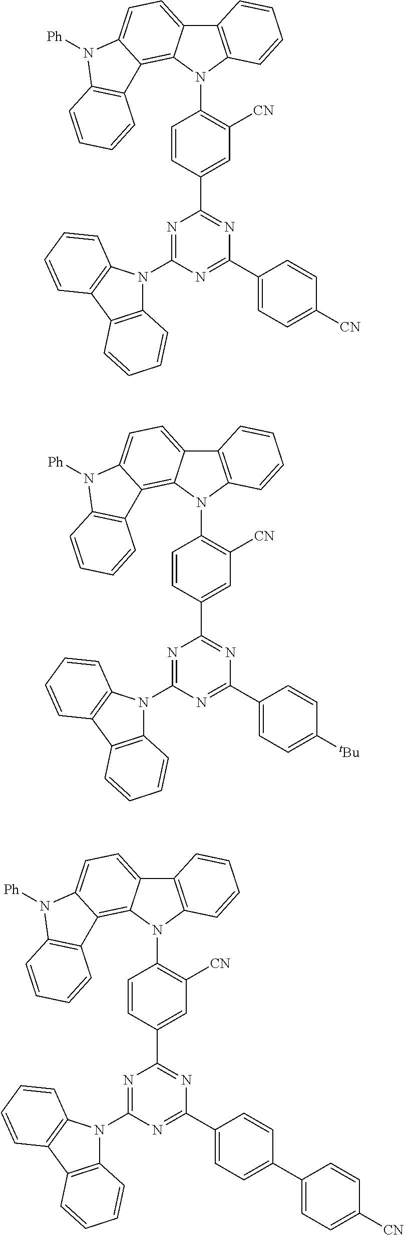 [SMILES: N#Cc1ccc(-c2nc(-c3ccc(-n4c5ccccc5c5ccc6c(c7ccccc7n6-c6ccccc6)c54)c(C#N)c3)nc(-n3c4ccccc4c4ccccc43)n2)cc1 ; CC(C)(C)c1ccc(-c2nc(-c3ccc(-n4c5ccccc5c5ccc6c(c7ccccc7n6-c6ccccc6)c54)c(C#N)c3)nc(-n3c4ccccc4c4ccccc43)n2)cc1 ; N#Cc1ccc(-c2ccc(-c3nc(-c4ccc(-n5c6ccccc6c6ccc7c(c8ccccc8n7-c7ccccc7)c65)c(C#N)c4)nc(-n4c5ccccc5c5ccccc54)n3)cc2)cc1]

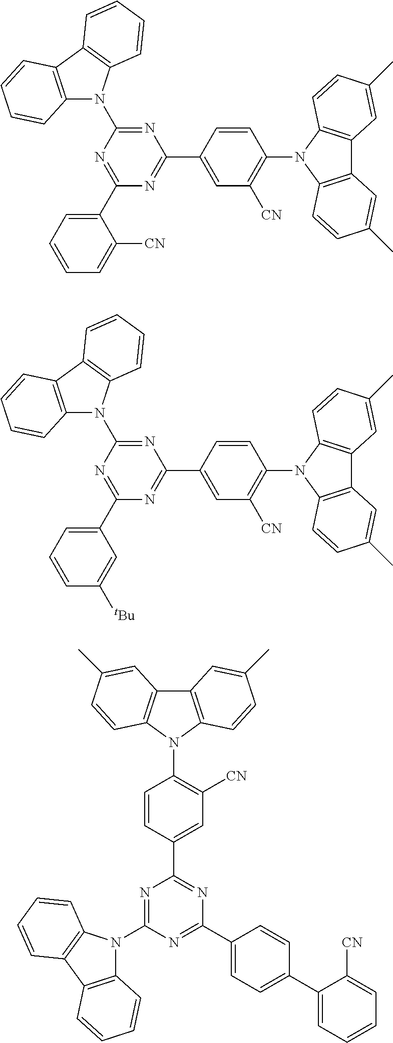

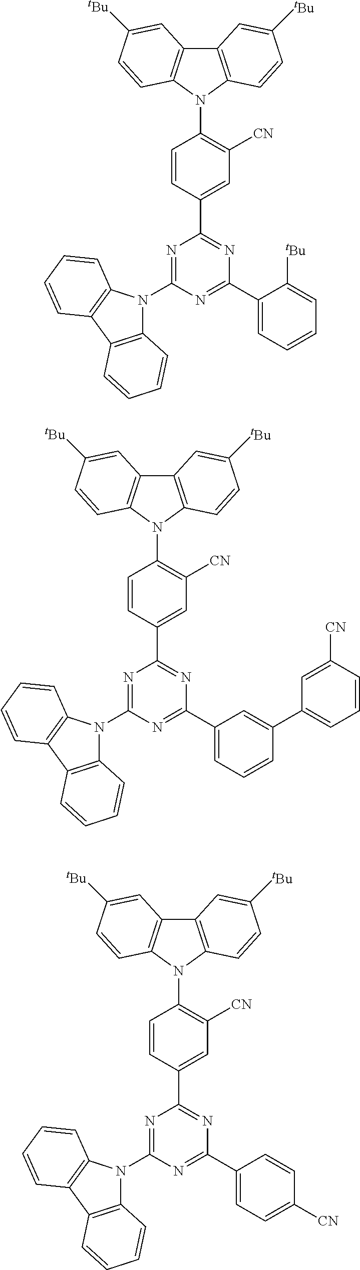 [SMILES: CC(C)(C)c1ccc2c(c1)c1cc(C(C)(C)C)ccc1n2-c1ccc(-c2nc(-c3ccccc3C(C)(C)C)nc(-n3c4ccccc4c4ccccc43)n2)cc1C#N ; CC(C)(C)c1ccc2c(c1)c1cc(C(C)(C)C)ccc1n2-c1ccc(-c2nc(-c3cccc(-c4cccc(C#N)c4)c3)nc(-n3c4ccccc4c4ccccc43)n2)cc1C#N ; CC(C)(C)c1ccc2c(c1)c1cc(C(C)(C)C)ccc1n2-c1ccc(-c2nc(-c3ccc(C#N)cc3)nc(-n3c4ccccc4c4ccccc43)n2)cc1C#N]

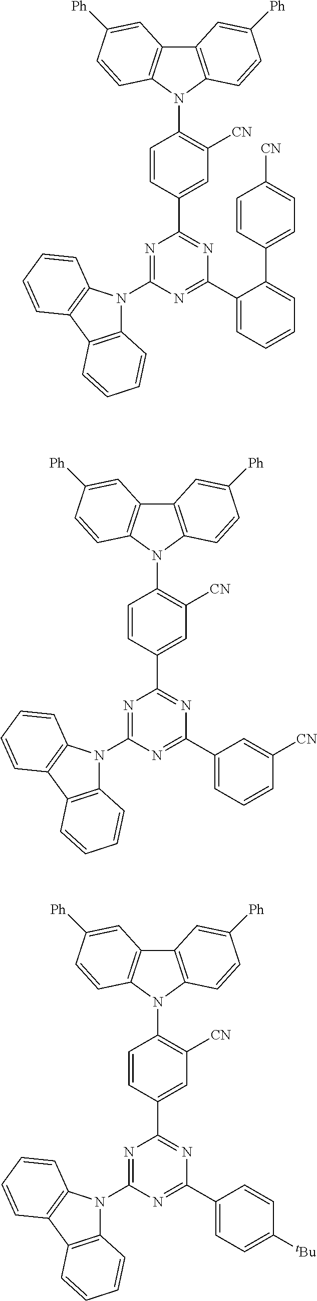 [SMILES: N#Cc1ccc(-c2ccccc2-c2nc(-c3ccc(-n4c5ccc(-c6ccccc6)cc5c5cc(-c6ccccc6)ccc54)c(C#N)c3)nc(-n3c4ccccc4c4ccccc43)n2)cc1 ; N#Cc1cccc(-c2nc(-c3ccc(-n4c5ccc(-c6ccccc6)cc5c5cc(-c6ccccc6)ccc54)c(C#N)c3)nc(-n3c4ccccc4c4ccccc43)n2)c1 ; CC(C)(C)c1ccc(-c2nc(-c3ccc(-n4c5ccc(-c6ccccc6)cc5c5cc(-c6ccccc6)ccc54)c(C#N)c3)nc(-n3c4ccccc4c4ccccc43)n2)cc1]

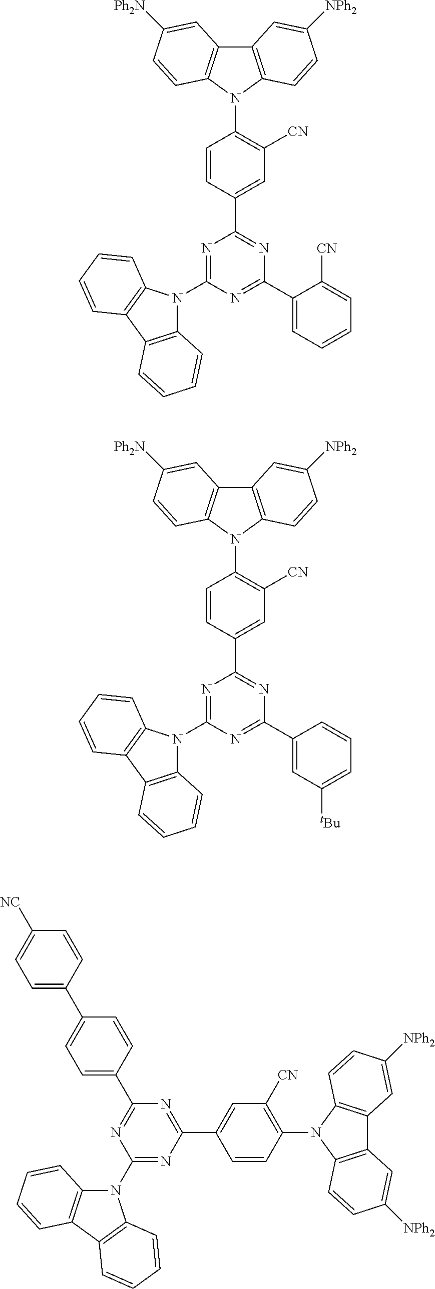

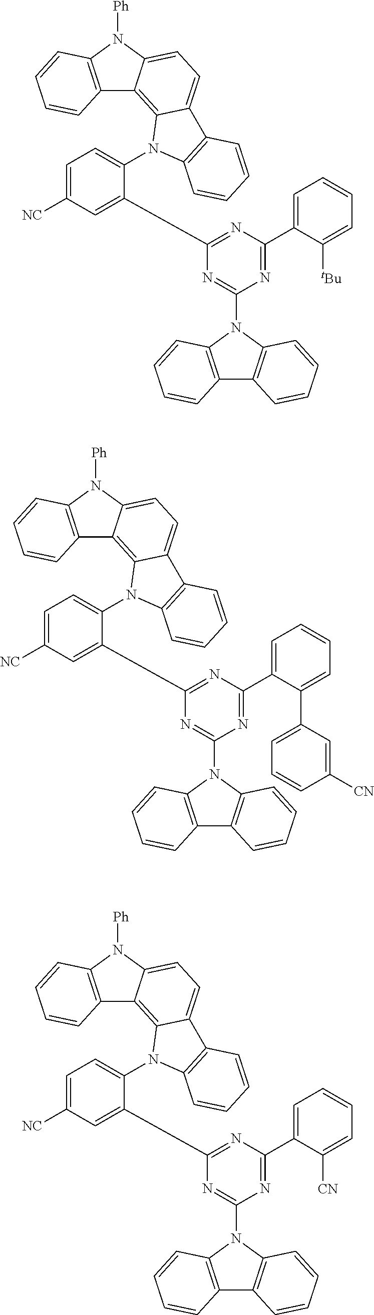 [SMILES: CC(C)(C)c1ccccc1-c1nc(-c2cc(C#N)ccc2-n2c3ccccc3c3ccc4c(c5ccccc5n4-c4ccccc4)c32)nc(-n2c3ccccc3c3ccccc32)n1 ; N#Cc1cccc(-c2ccccc2-c2nc(-c3cc(C#N)ccc3-n3c4ccccc4c4ccc5c(c6ccccc6n5-c5ccccc5)c43)nc(-n3c4ccccc4c4ccccc43)n2)c1 ; N#Cc1ccc(-n2c3ccccc3c3ccc4c(c5ccccc5n4-c4ccccc4)c32)c(-c2nc(-c3ccccc3C#N)nc(-n3c4ccccc4c4ccccc43)n2)c1]

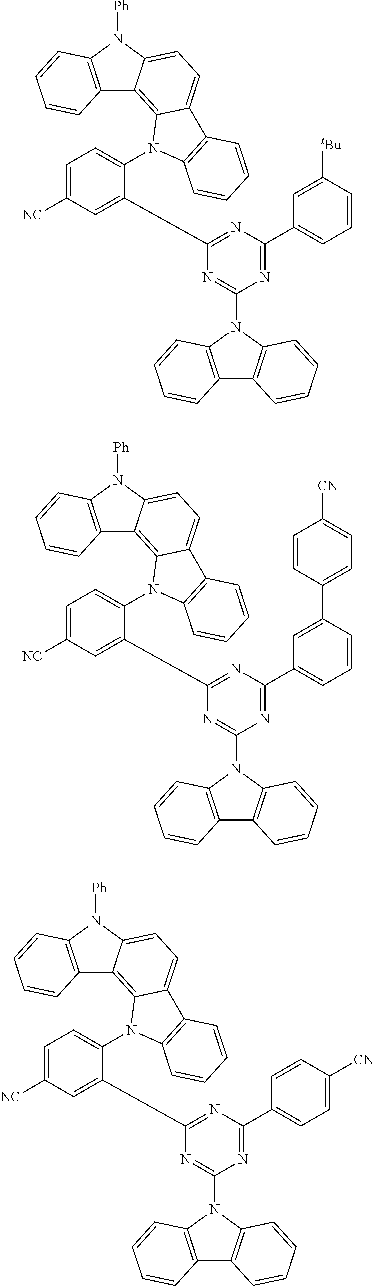 [SMILES: CC(C)(C)c1cccc(-c2nc(-c3cc(C#N)ccc3-n3c4ccccc4c4ccc5c(c6ccccc6n5-c5ccccc5)c43)nc(-n3c4ccccc4c4ccccc43)n2)c1 ; N#Cc1ccc(-c2cccc(-c3nc(-c4cc(C#N)ccc4-n4c5ccccc5c5ccc6c(c7ccccc7n6-c6ccccc6)c54)nc(-n4c5ccccc5c5ccccc54)n3)c2)cc1 ; N#Cc1ccc(-c2nc(-c3cc(C#N)ccc3-n3c4ccccc4c4ccc5c(c6ccccc6n5-c5ccccc5)c43)nc(-n3c4ccccc4c4ccccc43)n2)cc1]

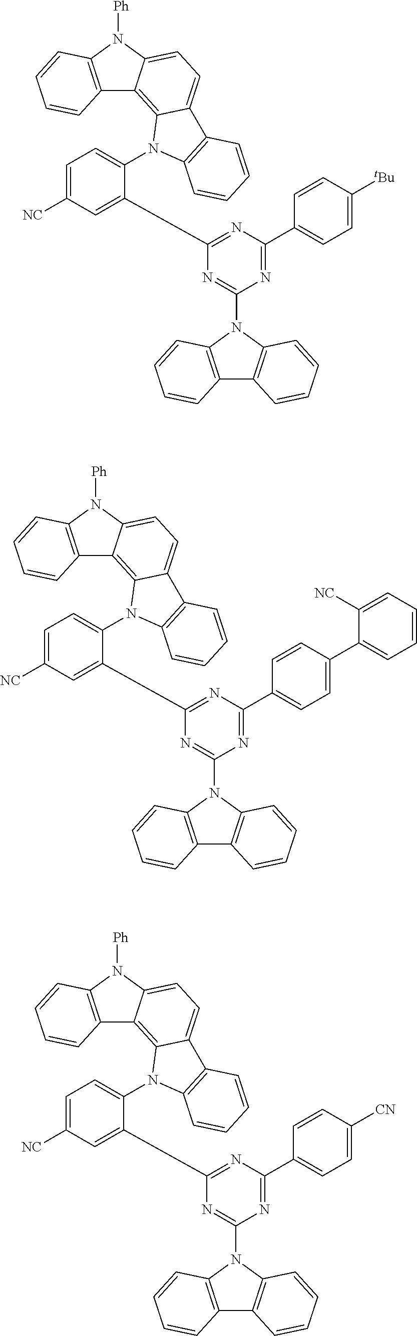 [SMILES: CC(C)(C)c1ccc(-c2nc(-c3cc(C#N)ccc3-n3c4ccccc4c4ccc5c(c6ccccc6n5-c5ccccc5)c43)nc(-n3c4ccccc4c4ccccc43)n2)cc1 ; N#Cc1ccc(-n2c3ccccc3c3ccc4c(c5ccccc5n4-c4ccccc4)c32)c(-c2nc(-c3ccc(-c4ccccc4C#N)cc3)nc(-n3c4ccccc4c4ccccc43)n2)c1 ; N#Cc1ccc(-c2nc(-c3cc(C#N)ccc3-n3c4ccccc4c4ccc5c(c6ccccc6n5-c5ccccc5)c43)nc(-n3c4ccccc4c4ccccc43)n2)cc1]

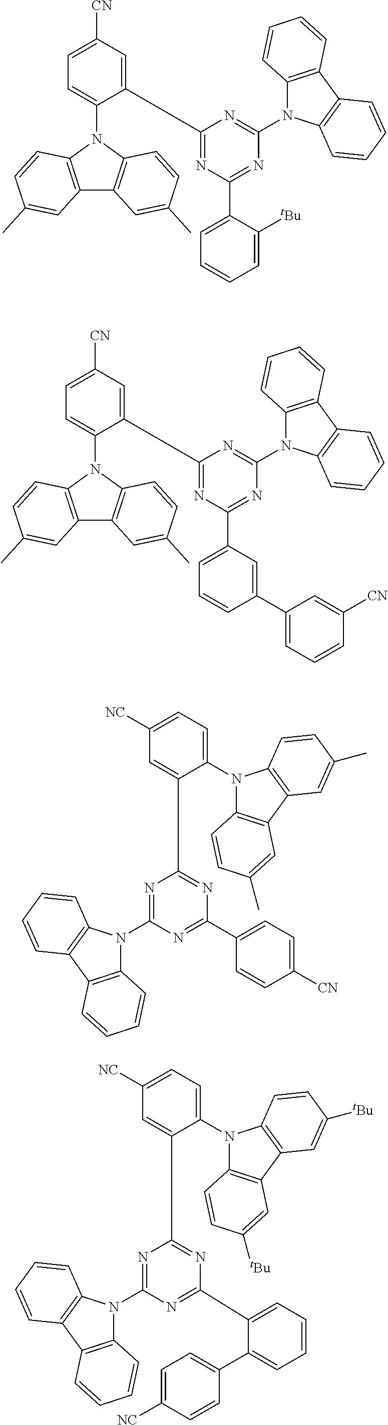

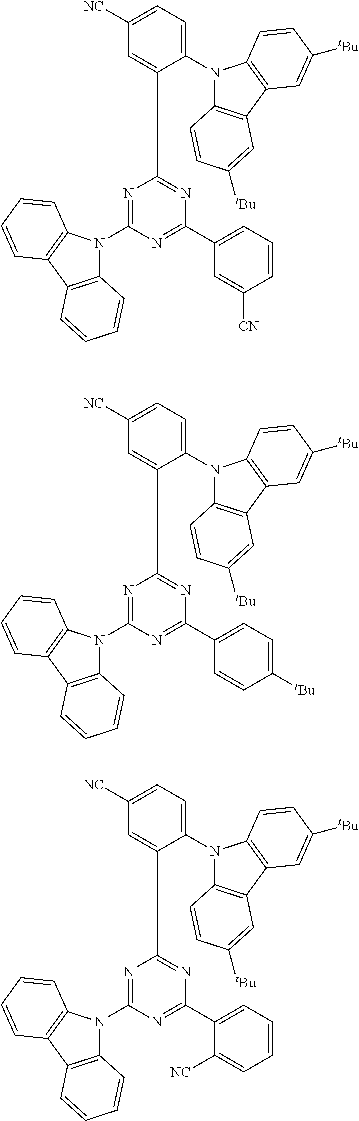 [SMILES: CC(C)(C)c1ccc2c(c1)c1cc(C(C)(C)C)ccc1n2-c1ccc(C#N)cc1-c1nc(-c2cccc(C#N)c2)nc(-n2c3ccccc3c3ccccc32)n1 ; CC(C)(C)c1ccc(-c2nc(-c3cc(C#N)ccc3-n3c4ccc(C(C)(C)C)cc4c4cc(C(C)(C)C)ccc43)nc(-n3c4ccccc4c4ccccc43)n2)cc1 ; CC(C)(C)c1ccc2c(c1)c1cc(C(C)(C)C)ccc1n2-c1ccc(C#N)cc1-c1nc(-c2ccccc2C#N)nc(-n2c3ccccc3c3ccccc32)n1]

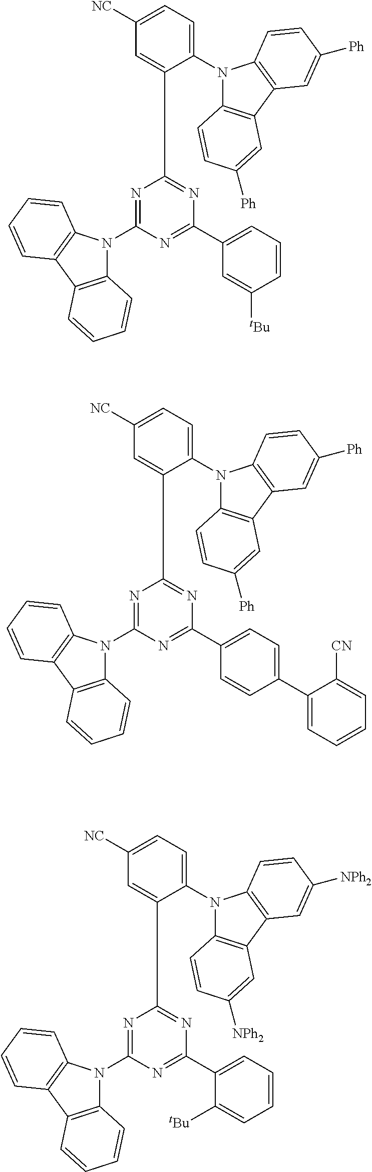

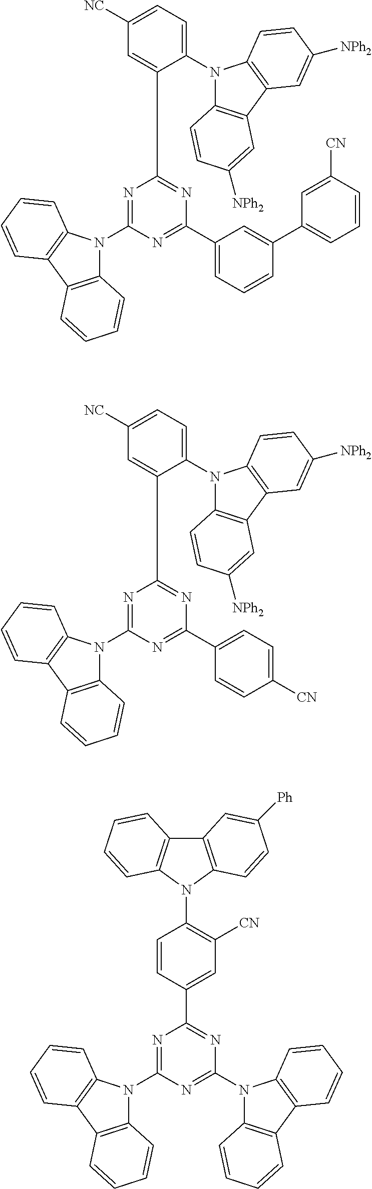

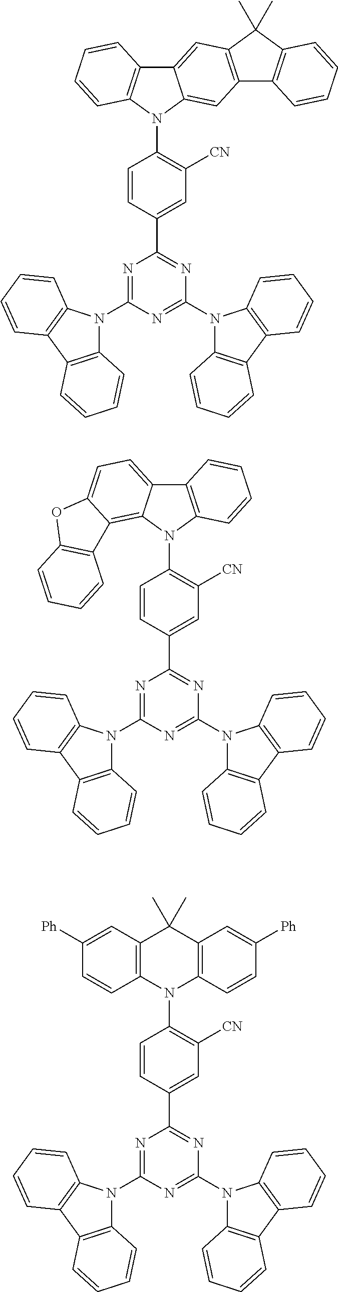 [SMILES: CC1(C)c2ccccc2-c2cc3c(cc21)c1ccccc1n3-c1ccc(-c2nc(-n3c4ccccc4c4ccccc43)nc(-n3c4ccccc4c4ccccc43)n2)cc1C#N ; N#Cc1cc(-c2nc(-n3c4ccccc4c4ccccc43)nc(-n3c4ccccc4c4ccccc43)n2)ccc1-n1c2ccccc2c2ccc3oc4ccccc4c3c21 ; CC1(C)c2cc(-c3ccccc3)ccc2N(c2ccc(-c3nc(-n4c5ccccc5c5ccccc54)nc(-n4c5ccccc5c5ccccc54)n3)cc2C#N)c2ccc(-c3ccccc3)cc21]

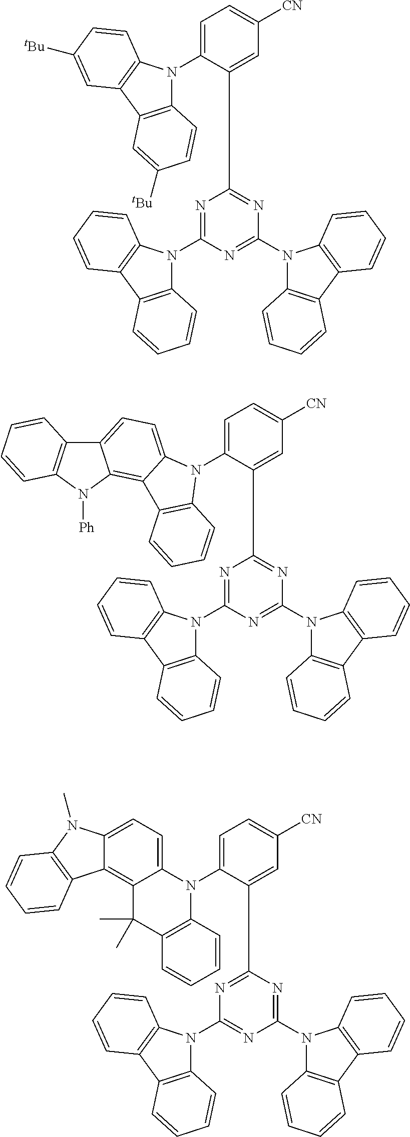 [SMILES: CC(C)(C)c1ccc2c(c1)c1cc(C(C)(C)C)ccc1n2-c1ccc(C#N)cc1-c1nc(-n2c3ccccc3c3ccccc32)nc(-n2c3ccccc3c3ccccc32)n1 ; N#Cc1ccc(-n2c3ccccc3c3c2ccc2c4ccccc4n(-c4ccccc4)c23)c(-c2nc(-n3c4ccccc4c4ccccc43)nc(-n3c4ccccc4c4ccccc43)n2)c1 ; Cn1c2ccccc2c2c3c(ccc21)N(c1ccc(C#N)cc1-c1nc(-n2c4ccccc4c4ccccc42)nc(-n2c4ccccc4c4ccccc42)n1)c1ccccc1C3(C)C]

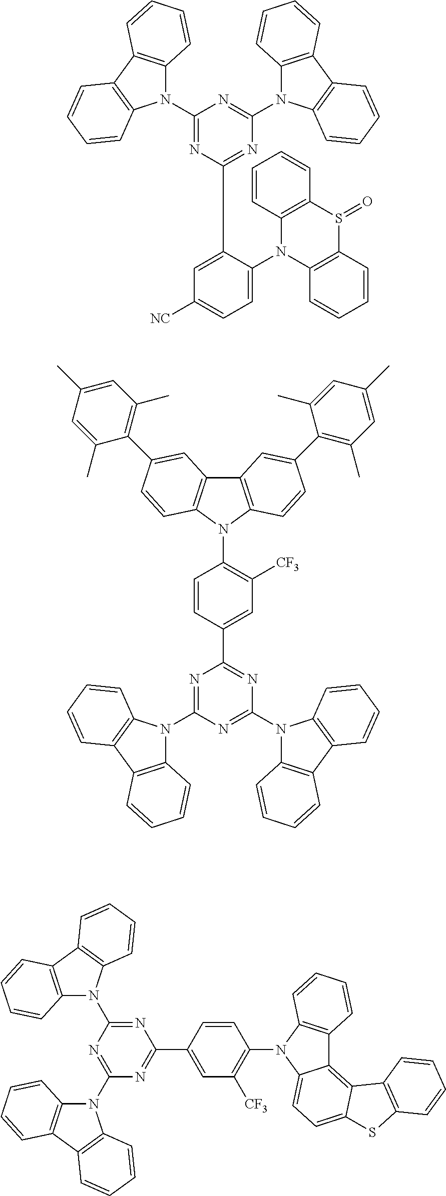

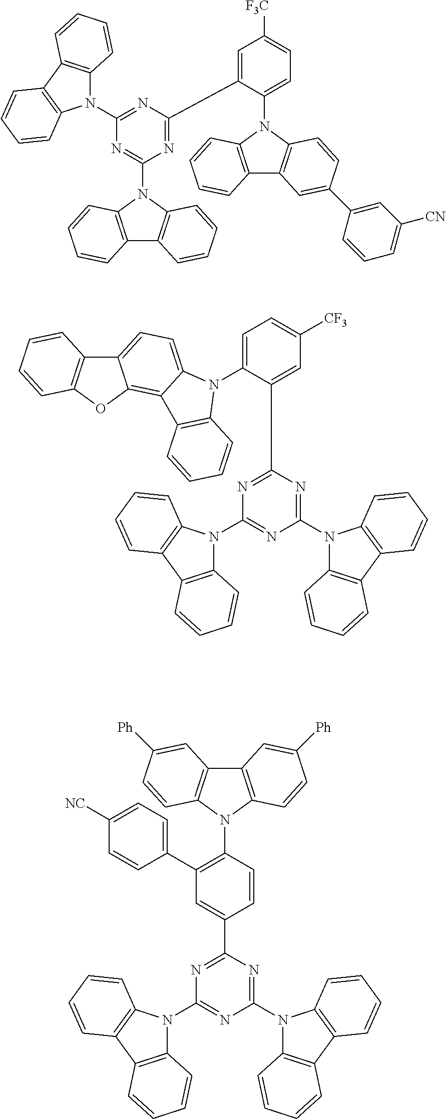 [SMILES: N#Cc1cccc(-c2ccc3c(c2)c2ccccc2n3-c2ccc(C(F)(F)F)cc2-c2nc(-n3c4ccccc4c4ccccc43)nc(-n3c4ccccc4c4ccccc43)n2)c1 ; FC(F)(F)c1ccc(-n2c3ccccc3c3c4oc5ccccc5c4ccc32)c(-c2nc(-n3c4ccccc4c4ccccc43)nc(-n3c4ccccc4c4ccccc43)n2)c1 ; N#Cc1ccc(-c2cc(-c3nc(-n4c5ccccc5c5ccccc54)nc(-n4c5ccccc5c5ccccc54)n3)ccc2-n2c3ccc(-c4ccccc4)cc3c3cc(-c4ccccc4)ccc32)cc1]

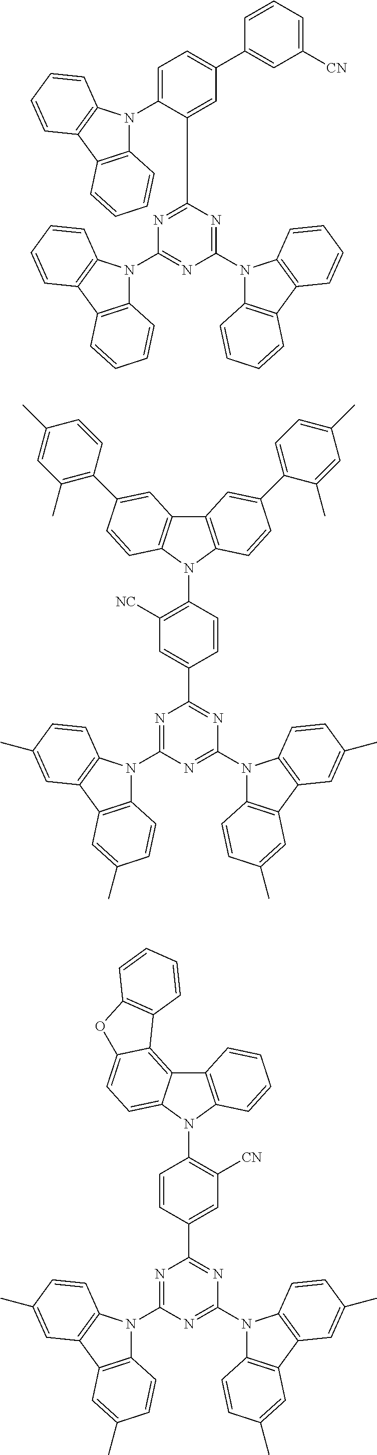

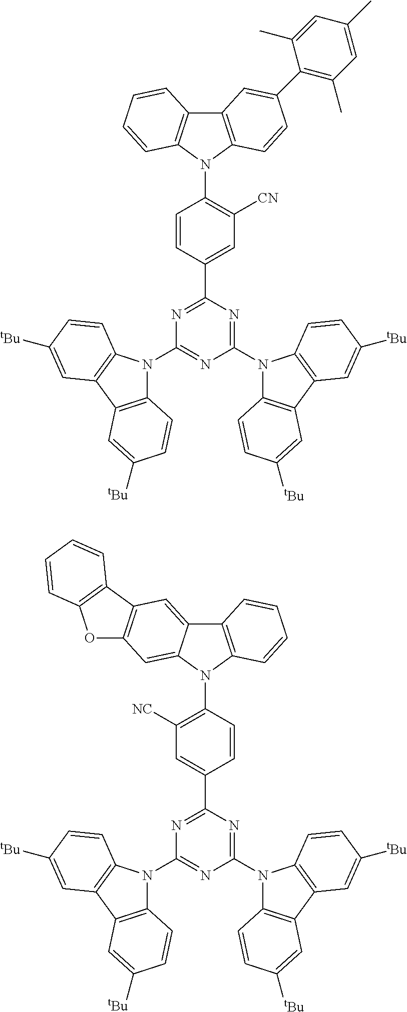 [SMILES: Cc1cc(C)c(-c2ccc3c(c2)c2ccccc2n3-c2ccc(-c3nc(-n4c5ccc(C(C)(C)C)cc5c5cc(C(C)(C)C)ccc54)nc(-n4c5ccc(C(C)(C)C)cc5c5cc(C(C)(C)C)ccc54)n3)cc2C#N)c(C)c1 ; CC(C)(C)c1ccc2c(c1)c1cc(C(C)(C)C)ccc1n2-c1nc(-c2ccc(-n3c4ccccc4c4cc5c(cc43)oc3ccccc35)c(C#N)c2)nc(-n2c3ccc(C(C)(C)C)cc3c3cc(C(C)(C)C)ccc32)n1]

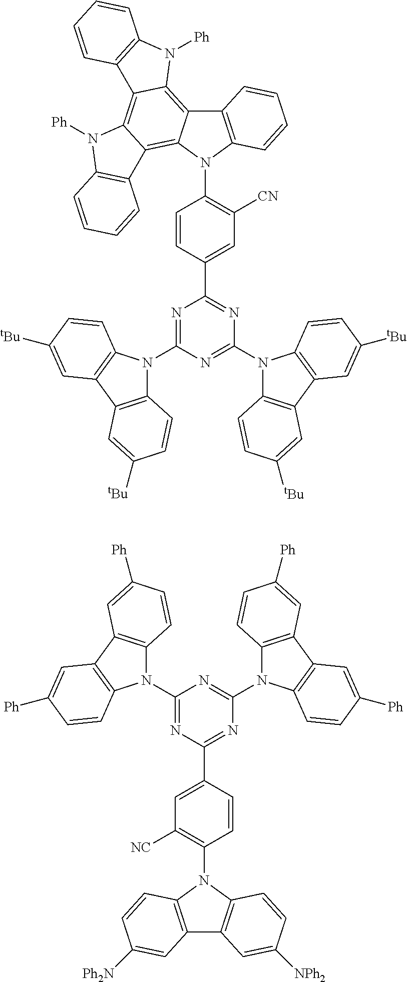

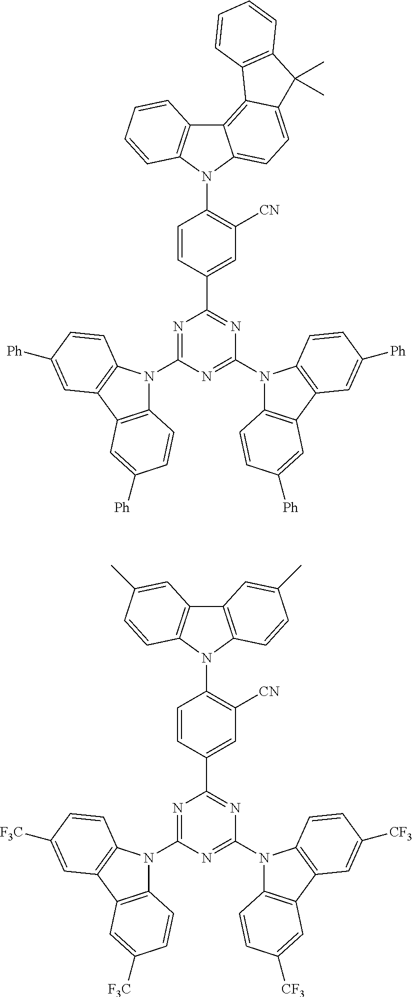 [SMILES: CC1(C)c2ccccc2-c2c1ccc1c2c2ccccc2n1-c1ccc(-c2nc(-n3c4ccc(-c5ccccc5)cc4c4cc(-c5ccccc5)ccc43)nc(-n3c4ccc(-c5ccccc5)cc4c4cc(-c5ccccc5)ccc43)n2)cc1C#N ; Cc1ccc2c(c1)c1cc(C)ccc1n2-c1ccc(-c2nc(-n3c4ccc(C(F)(F)F)cc4c4cc(C(F)(F)F)ccc43)nc(-n3c4ccc(C(F)(F)F)cc4c4cc(C(F)(F)F)ccc43)n2)cc1C#N]

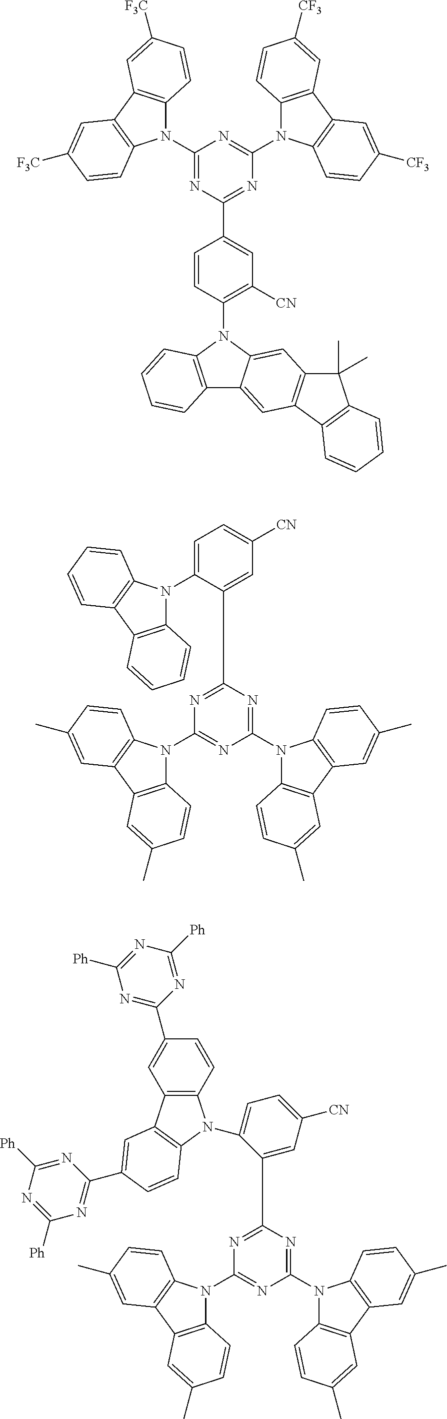

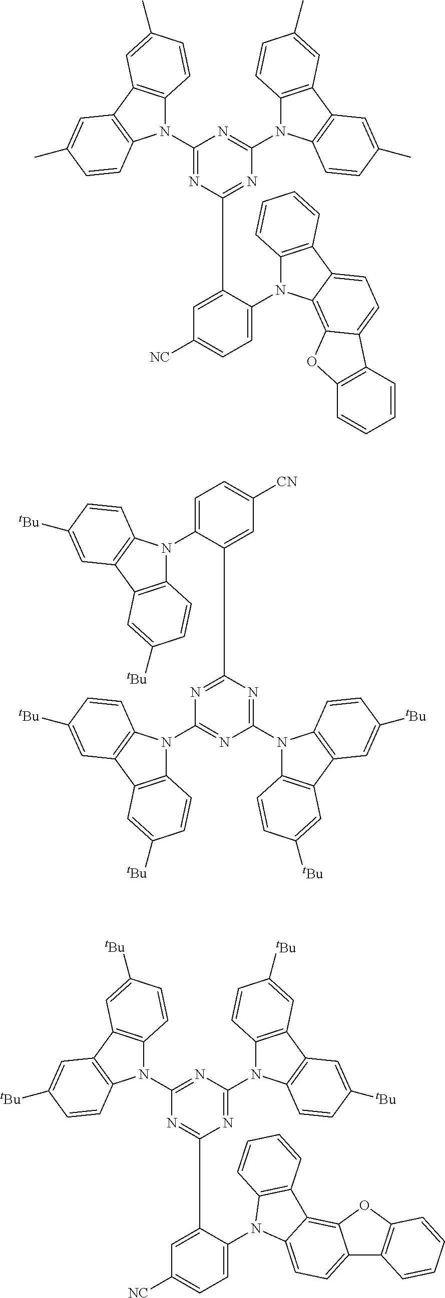

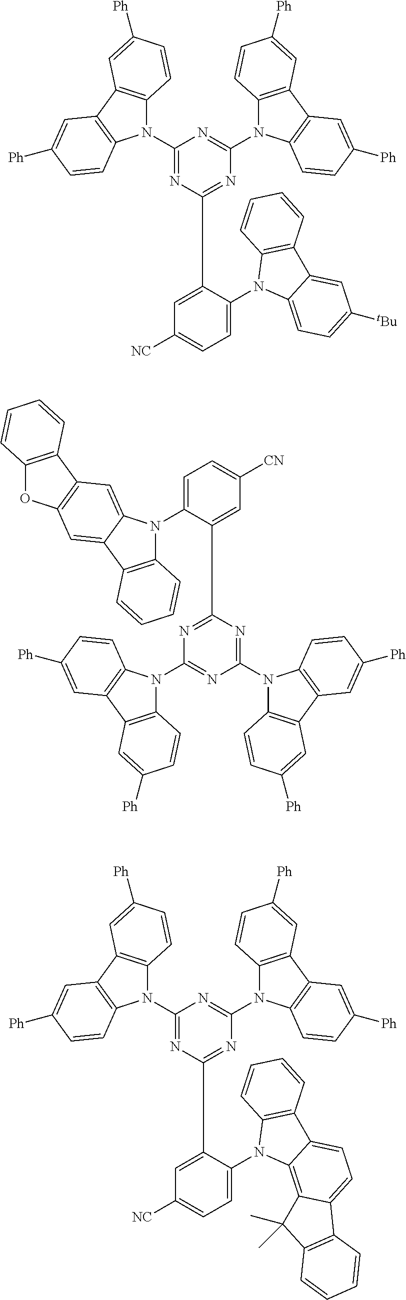 [SMILES: CC(C)(C)c1ccc2c(c1)c1ccccc1n2-c1ccc(C#N)cc1-c1nc(-n2c3ccc(-c4ccccc4)cc3c3cc(-c4ccccc4)ccc32)nc(-n2c3ccc(-c4ccccc4)cc3c3cc(-c4ccccc4)ccc32)n1 ; N#Cc1ccc(-n2c3ccccc3c3cc4oc5ccccc5c4cc32)c(-c2nc(-n3c4ccc(-c5ccccc5)cc4c4cc(-c5ccccc5)ccc43)nc(-n3c4ccc(-c5ccccc5)cc4c4cc(-c5ccccc5)ccc43)n2)c1 ; CC1(C)c2ccccc2-c2ccc3c4ccccc4n(-c4ccc(C#N)cc4-c4nc(-n5c6ccc(-c7ccccc7)cc6c6cc(-c7ccccc7)ccc65)nc(-n5c6ccc(-c7ccccc7)cc6c6cc(-c7ccccc7)ccc65)n4)c3c21]

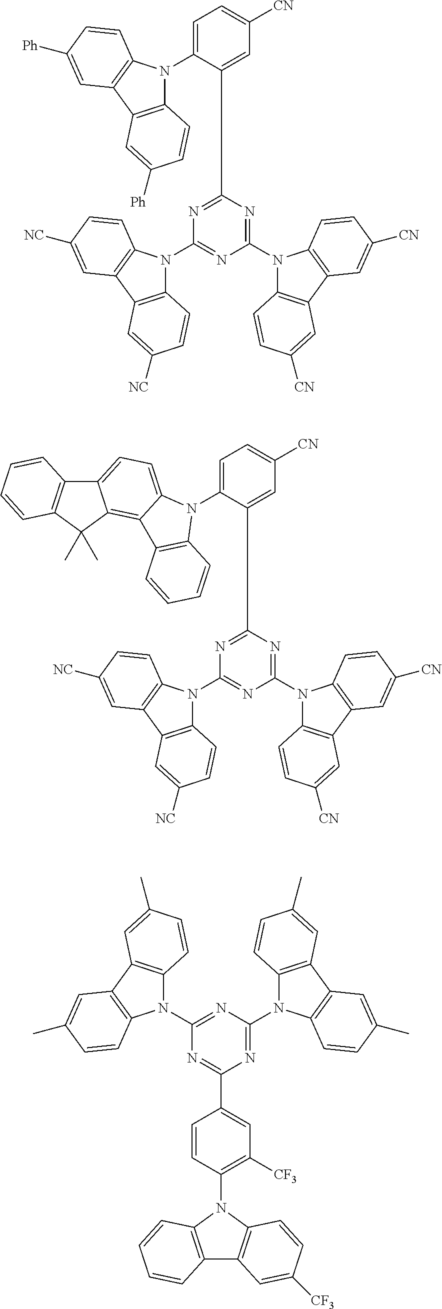

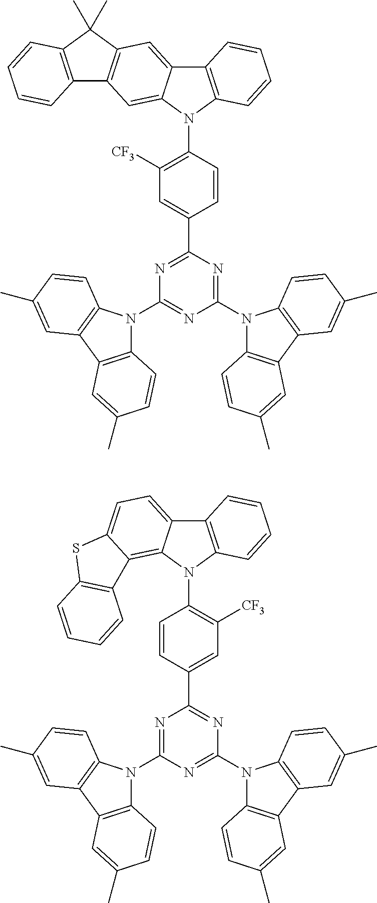

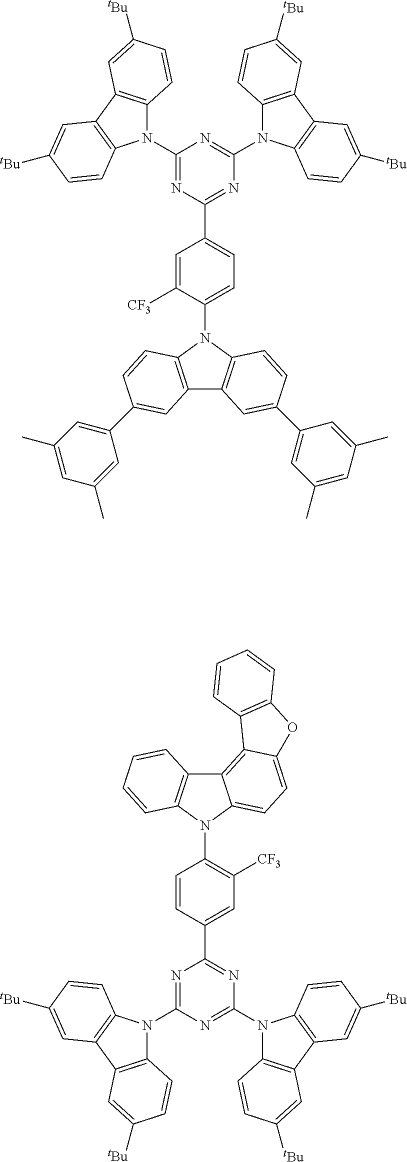 [SMILES: Cc1cc(C)cc(-c2ccc3c(c2)c2cc(-c4cc(C)cc(C)c4)ccc2n3-c2ccc(-c3nc(-n4c5ccc(C(C)(C)C)cc5c5cc(C(C)(C)C)ccc54)nc(-n4c5ccc(C(C)(C)C)cc5c5cc(C(C)(C)C)ccc54)n3)cc2C(F)(F)F)c1 ; CC(C)(C)c1ccc2c(c1)c1cc(C(C)(C)C)ccc1n2-c1nc(-c2ccc(-n3c4ccccc4c4c5c(ccc43)oc3ccccc35)c(C(F)(F)F)c2)nc(-n2c3ccc(C(C)(C)C)cc3c3cc(C(C)(C)C)ccc32)n1]

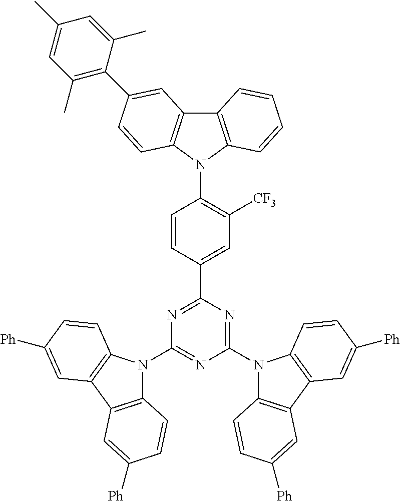 [SMILES: Cc1cc(C)c(-c2ccc3c(c2)c2ccccc2n3-c2ccc(-c3nc(-n4c5ccc(-c6ccccc6)cc5c5cc(-c6ccccc6)ccc54)nc(-n4c5ccc(-c6ccccc6)cc5c5cc(-c6ccccc6)ccc54)n3)cc2C(F)(F)F)c(C)c1]

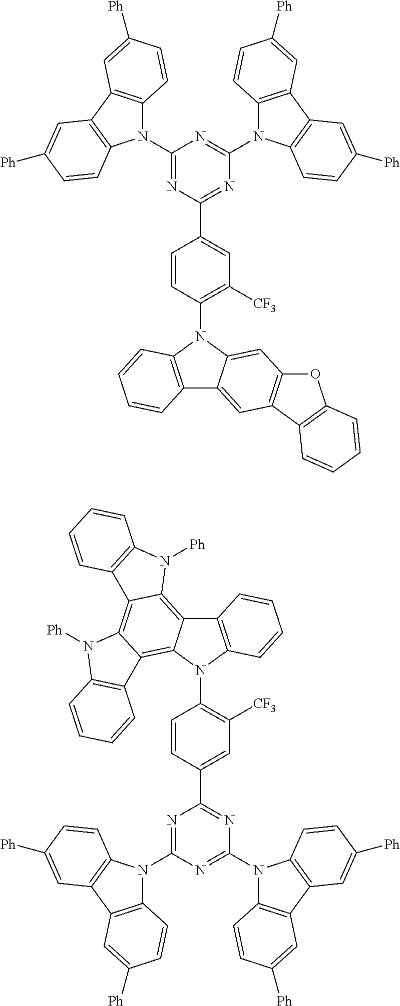 [SMILES: FC(F)(F)c1cc(-c2nc(-n3c4ccc(-c5ccccc5)cc4c4cc(-c5ccccc5)ccc43)nc(-n3c4ccc(-c5ccccc5)cc4c4cc(-c5ccccc5)ccc43)n2)ccc1-n1c2ccccc2c2cc3c(cc21)oc1ccccc13 ; FC(F)(F)c1cc(-c2nc(-n3c4ccc(-c5ccccc5)cc4c4cc(-c5ccccc5)ccc43)nc(-n3c4ccc(-c5ccccc5)cc4c4cc(-c5ccccc5)ccc43)n2)ccc1-n1c2ccccc2c2c3c(c4ccccc4n3-c3ccccc3)c3c(c4ccccc4n3-c3ccccc3)c21]

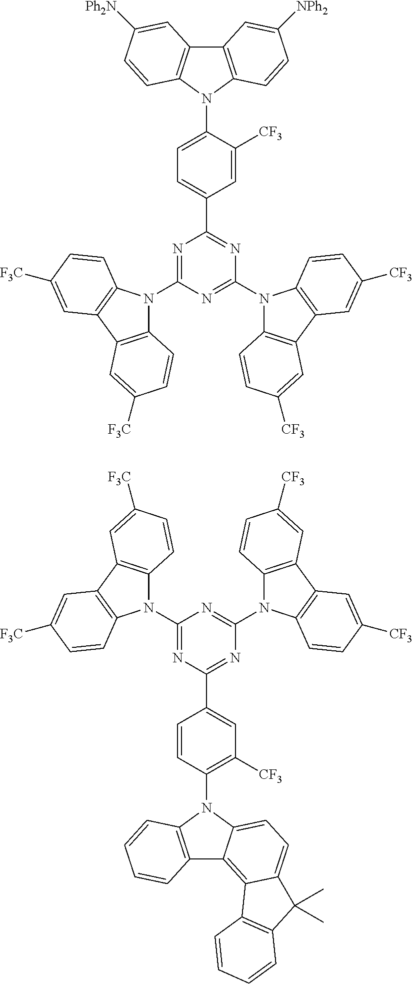

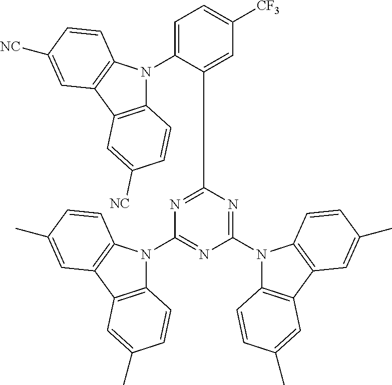

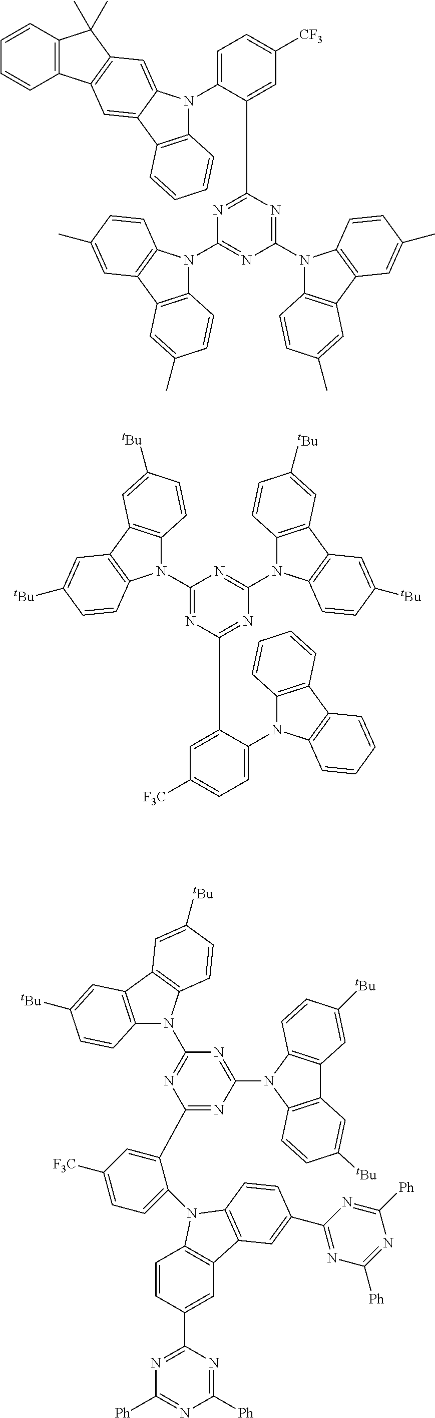 [SMILES: Cc1ccc2c(c1)c1cc(C)ccc1n2-c1nc(-c2cc(C(F)(F)F)ccc2-n2c3ccccc3c3cc4c(cc32)C(C)(C)c2ccccc2-4)nc(-n2c3ccc(C)cc3c3cc(C)ccc32)n1 ; CC(C)(C)c1ccc2c(c1)c1cc(C(C)(C)C)ccc1n2-c1nc(-c2cc(C(F)(F)F)ccc2-n2c3ccccc3c3ccccc32)nc(-n2c3ccc(C(C)(C)C)cc3c3cc(C(C)(C)C)ccc32)n1 ; CC(C)(C)c1ccc2c(c1)c1cc(C(C)(C)C)ccc1n2-c1nc(-c2cc(C(F)(F)F)ccc2-n2c3ccc(-c4nc(-c5ccccc5)nc(-c5ccccc5)n4)cc3c3cc(-c4nc(-c5ccccc5)nc(-c5ccccc5)n4)ccc32)nc(-n2c3ccc(C(C)(C)C)cc3c3cc(C(C)(C)C)ccc32)n1]

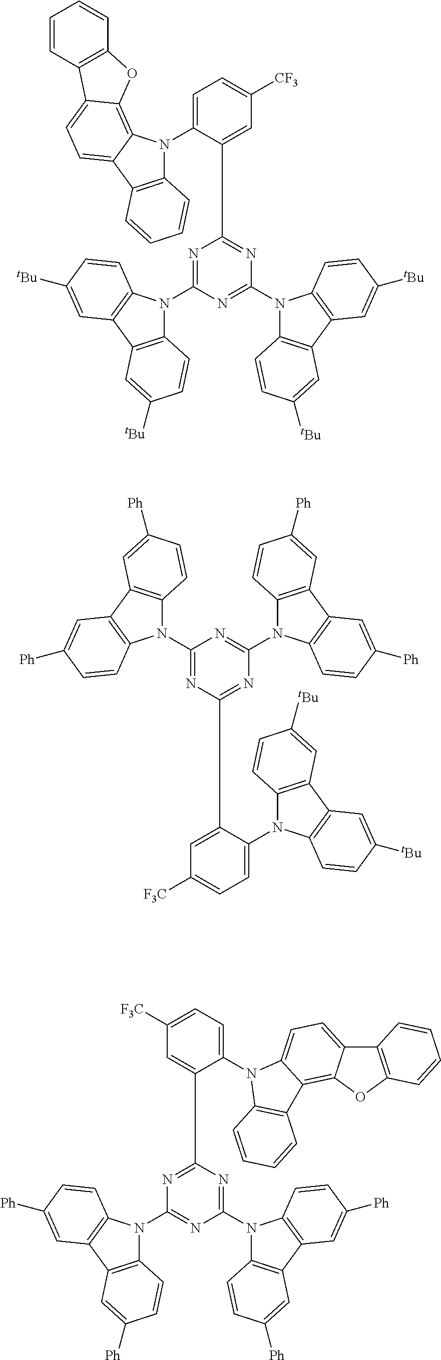 [SMILES: CC(C)(C)c1ccc2c(c1)c1cc(C(C)(C)C)ccc1n2-c1nc(-c2cc(C(F)(F)F)ccc2-n2c3ccccc3c3ccc4c5ccccc5oc4c32)nc(-n2c3ccc(C(C)(C)C)cc3c3cc(C(C)(C)C)ccc32)n1 ; CC(C)(C)c1ccc2c(c1)c1cc(C(C)(C)C)ccc1n2-c1ccc(C(F)(F)F)cc1-c1nc(-n2c3ccc(-c4ccccc4)cc3c3cc(-c4ccccc4)ccc32)nc(-n2c3ccc(-c4ccccc4)cc3c3cc(-c4ccccc4)ccc32)n1 ; FC(F)(F)c1ccc(-n2c3ccccc3c3c4oc5ccccc5c4ccc32)c(-c2nc(-n3c4ccc(-c5ccccc5)cc4c4cc(-c5ccccc5)ccc43)nc(-n3c4ccc(-c5ccccc5)cc4c4cc(-c5ccccc5)ccc43)n2)c1]

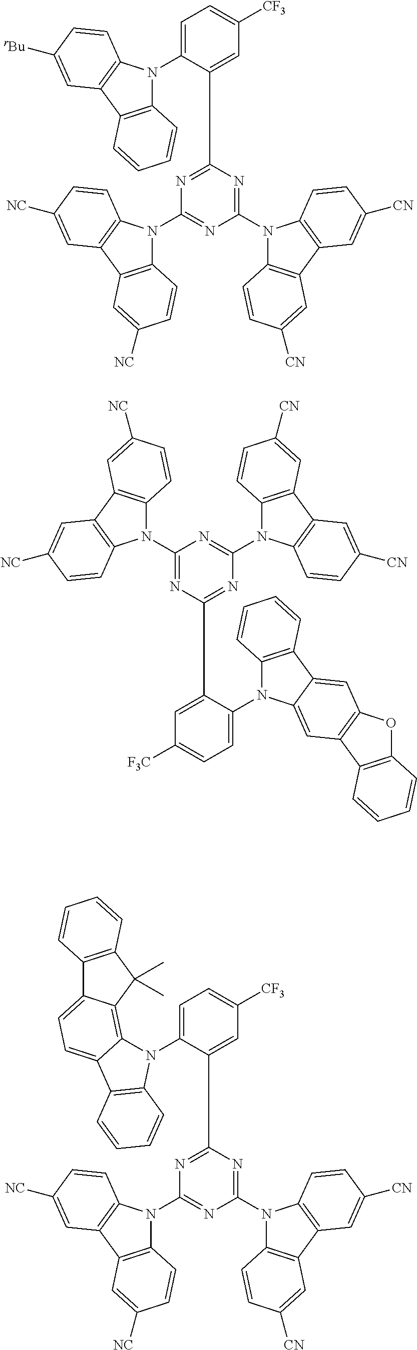

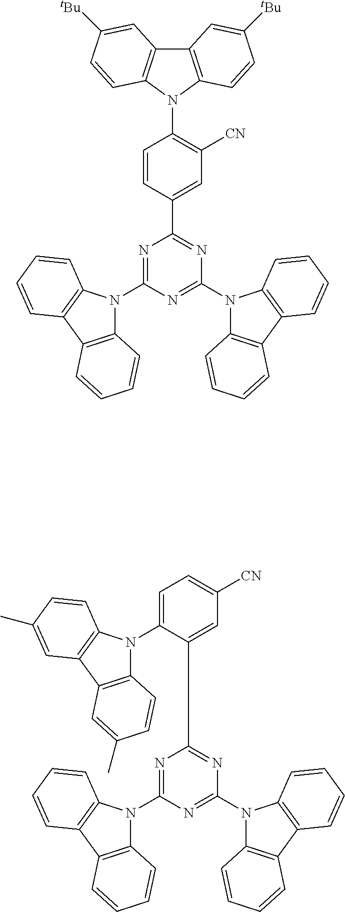 [SMILES: CC(C)(C)c1ccc2c(c1)c1cc(C(C)(C)C)ccc1n2-c1ccc(-c2nc(-n3c4ccccc4c4ccccc43)nc(-n3c4ccccc4c4ccccc43)n2)cc1C#N ; Cc1ccc2c(c1)c1cc(C)ccc1n2-c1ccc(C#N)cc1-c1nc(-n2c3ccccc3c3ccccc32)nc(-n2c3ccccc3c3ccccc32)n1]

1. An organic molecule, comprising:
a first chemical moiety comprising a structure of formula I-a: 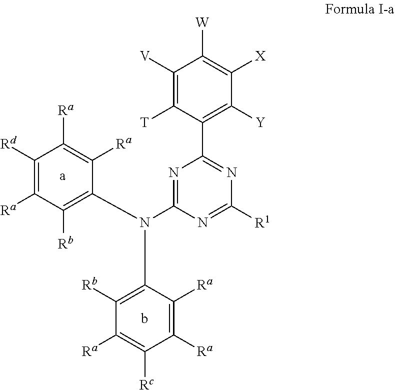
and
a second chemical moiety, comprising a structure of formula II: 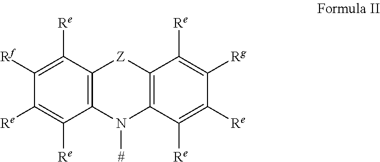
wherein:
the first chemical moiety is linked to the second chemical moiety via a single bond;
T is the binding site of a single bond linking the first chemical moiety to the second chemical moiety, or is selected from the group consisting of R² and R^{X};
V is the binding site of a single bond linking the first chemical moiety to the second chemical moiety, or is hydrogen (H);
W is the binding site of a single bond linking the first chemical moiety to the second chemical moiety, or is selected from the group consisting of R² and R^{X};
X is selected from the group consisting of R² and R^{X};
Y is selected from the group consisting of R² and R^{X};
R^{X} is selected from the group consisting of CN and CF₃ or R^{X} comprises a structure of formula BN-I, 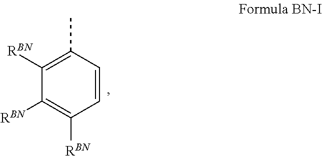
which is bonded to the structure of formula I-a via a single bond indicated by the dashed line and wherein exactly one group R^{BN} is CN while the other two groups R^{BN} are both hydrogen (H);
R¹ is selected from the group consisting of: 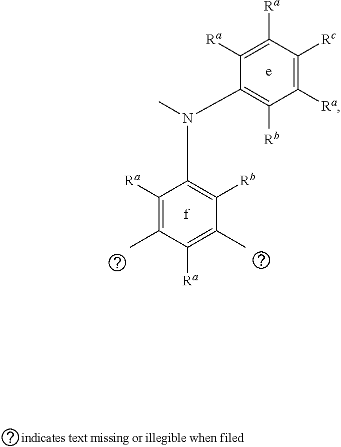
hydrogen, deuterium, OR³, Si(R³)₃, B(OR³)₂, OSO₂R³, CF₃, CN, F, Cl, Br, I, C₁-C₄₀-alkyl,
which is optionally substituted with one or more substituents R³ and
wherein one or more non-adjacent CH₂-groups are optionally substituted by R³C═CR³, C≡C, Si(R³)₂, Ge(R³)₂, Sn(R³)₂, C═O, C═S, C═Se, C═NR³, P(═O)(R³), SO, SO₂, NR³, O, S or CONR³;
C₁-C₄₀-alkoxy,
which is optionally substituted with one or more substituents R³ and
wherein one or more non-adjacent CH₂-groups are optionally substituted by R³C═CR³, C≡C, Si(R³)₂, Ge(R³)₂, Sn(R³)₂, C═O, C═S, C═Se, C═NR³, P(═O)(R³), SO, SO₂, NR³, O, S or CONR³;
C₁-C₄₀-thioalkoxy,
which is optionally substituted with one or more substituents R³ and
wherein one or more non-adjacent CH₂-groups are optionally substituted by R³C═CR³, C≡C, Si(R³)₂, Ge(R³)₂, Sn(R³)₂, C═O, C═S, C═Se, C═NR³, P(═O)(R³), SO, SO₂, NR³, O, S or CONR³;
C₂-C₄₀-alkenyl,
which is optionally substituted with one or more substituents R³ and
wherein one or more non-adjacent CH₂-groups are optionally substituted by R³C═CR³, C≡C, Si(R³)₂, Ge(R³)₂, Sn(R³)₂, C═O, C═S, C═Se, C═NR³, P(═O)(R³), SO, SO₂, NR³, O, S or CONR³;
C₂-C₄₀-alkynyl,
which is optionally substituted with one or more substituents R³ and
wherein one or more non-adjacent CH₂-groups are optionally substituted by R³C═CR³, C≡C, Si(R³)₂, Ge(R³)₂, Sn(R³)₂, C═O, C═S, C═Se, C═NR⁷, P(═O)(R³), SO, SO₂, NR³, O, S or CONR³;
C₆-C₆₀-aryl,
which is optionally substituted with one or more substituents R³;
R² is at each occurrence independently selected from the group consisting of:
hydrogen, deuterium,
C₁-C₁₀-alkyl,
wherein one or more hydrogen atoms are optionally substituted by deuterium;
C₂-C₁₀-alkenyl,
wherein one or more hydrogen atoms are optionally substituted by deuterium;
C₂-C₁₀-alkynyl,
wherein one or more hydrogen atoms are optionally substituted by deuterium;
C₅-C₁₀-aryl;
wherein one or more hydrogen atoms are optionally substituted by a group R⁵;
R³ is at each occurrence independently selected from the group consisting of: hydrogen, deuterium, N(R⁴)₂, OR⁴, Si(R⁴)₃, B(OR⁴)₂, OSO₂R⁴, CF₃, CN, F, Cl, Br, I, C₁-C₄₀-alkyl,
which is optionally substituted with one or more substituents R⁴ and
wherein one or more non-adjacent CH₂-groups are optionally substituted by R⁴C═CR⁴, C≡C, Si(R⁴)₂, Ge(R⁴)₂, Sn(R⁴)₂, C═O, C═S, C═Se, C═NR⁴, P(═O)(R⁴), SO, SO₂, NR⁴, O, S or CONR⁴;
C₁-C₄₀-alkoxy,
which is optionally substituted with one or more substituents R⁴ and
wherein one or more non-adjacent CH₂-groups are optionally substituted by R⁴C═CR⁴, C≡C, Si(R⁴)₂, Ge(R⁴)₂, Sn(R⁴)₂, C═O, C═S, C═Se, C═NR⁴, P(═O)(R⁴), SO, SO₂, NR⁴, O, S or CONR⁴;
C₁-C₄₀-thioalkoxy,
which is optionally substituted with one or more substituents R⁴ and
wherein one or more non-adjacent CH₂-groups are optionally substituted by R⁴C═CR⁴, C≡C, Si(R⁴)₂, Ge(R⁴)₂, Sn(R⁴)₂, C═O, C═S, C═Se, C═NR⁴, P(═O)(R⁴), SO, SO₂, NR⁴, O, S or CONR⁴;
C₂-C₄₀-alkenyl,
which is optionally substituted with one or more substituents R⁴ and
wherein one or more non-adjacent CH₂-groups are optionally substituted by R⁴C═CR⁴, C≡C, Si(R⁴)₂, Ge(R⁴)₂, Sn(R⁴)₂, C═O, C═S, C═Se, C═NR⁴, P(═O)(R⁴), SO, SO₂, NR⁴, O, S or CONR⁴;
C₂-C₄₀-alkynyl,
which is optionally substituted with one or more substituents R⁴ and
wherein one or more non-adjacent CH₂-groups are optionally substituted by R⁴C═CR⁴, C≡C, Si(R⁴)₂, Ge(R⁴)₂, Sn(R⁴)₂, C═O, C═S, C═Se, C═NR⁴, P(═O)(R⁴), SO, SO₂, NR⁴, O, S or CONR⁴;
C₆-C₆₀-aryl,
which is optionally substituted with one or more substituents R⁴; and
C₃-C₆₀-heteroaryl,
which is optionally substituted with one or more substituents R⁴;
R^{a}, R^{b}, R^{c}, R^{d}, R⁶, and R⁷ are at each occurrence independently selected from the group consisting of: hydrogen, deuterium, N(R⁸)₂, OR⁸, Si(R⁸)₃, B(OR⁸)₂, OSO₂R⁸, CF₃, CN, F, Cl, Br, I,
C₁-C₄₀-alkyl,
which is optionally substituted with one or more substituents R⁸ and
wherein one or more non-adjacent CH₂-groups are optionally substituted by R⁸C═CR⁸, C≡C, Si(R⁸)₂, Ge(R⁸)₂, Sn(R⁸)₂, C═O, C═S, C═Se, C═NR⁸, P(═O)(R⁸), SO, SO₂, NR⁸, O, S or CONR⁸;
C₁-C₄₀-alkoxy,
which is optionally substituted with one or more substituents R⁸ and
wherein one or more non-adjacent CH₂-groups are optionally substituted by R⁸C═CR⁸, C≡C, Si(R⁸)₂, Ge(R⁸)₂, Sn(R⁸)₂, C═O, C═S, C═Se, C═NR⁸, P(═O)(R⁸), SO, SO₂, NR⁸, O, S or CONR⁸;
C₁-C₄₀-thioalkoxy,
which is optionally substituted with one or more substituents R⁸ and
wherein one or more non-adjacent CH₂-groups are optionally substituted by R⁸C═CR⁸, C≡C, Si(R⁸)₂, Ge(R⁸)₂, Sn(R⁸)₂, C═O, C═S, C═Se, C═NR⁸, P(═O)(R⁸), SO, SO₂, NR⁸, O, S or CONR⁸;
C₂-C₄₀-alkenyl,
which is optionally substituted with one or more substituents R⁸ and
wherein one or more non-adjacent CH₂-groups are optionally substituted by R⁸C═CR⁸, C≡C, Si(R⁸)₂, Ge(R⁸)₂, Sn(R⁸)₂, C═O, C═S, C═Se, C═NR⁸, P(═O)(R⁸), SO, SO₂, NR⁸, O, S or CONR⁸;
C₂-C₄₀-alkynyl,
which is optionally substituted with one or more substituents R⁸ and
wherein one or more non-adjacent CH₂-groups are optionally substituted by R⁸C═CR⁸, C≡C, Si(R⁸)₂, Ge(R⁸)₂, Sn(R⁸)₂, C═O, C═S, C═Se, C═NR⁸, P(═O)(R⁸), SO, SO₂, NR⁸, O, S or CONR⁸;
C₆-C₆₀-aryl,
which is optionally substituted with one or more substituents R⁸; and
C₃-C₆₀-heteroaryl,
which is optionally substituted with one or more substituents R⁸;
wherein, optionally, two moieties R^{b} form a group Y, which is selected from the group consisting of a direct bond, CR⁶R⁷, C═CR⁶R⁷, C═O, C═NR⁶, NR⁶, O, SiR⁶R⁷, S, S(O) and S(O)₂;
R⁸ is at each occurrence independently selected from the group consisting of: hydrogen, deuterium, N(R⁹)₂, OR⁹, Si(R⁹)₃, B(OR⁹)₂, OSO₂R⁹, CF₃, CN, F, Cl, Br, I, C₁-C₄₀-alkyl,
which is optionally substituted with one or more substituents R⁹ and
wherein one or more non-adjacent CH₂-groups are optionally substituted by R⁹C═CR⁹, C≡C, Si(R⁹)₂, Ge(R⁹)₂, Sn(R⁹)₂, C═O, C═S, C═Se, C═NR⁹, P(═O)(R⁹), SO, SO₂, NR⁹, O, S or CONR⁹;
C₁-C₄₀-alkoxy,
which is optionally substituted with one or more substituents R⁹ and
wherein one or more non-adjacent CH₂-groups are optionally substituted by R⁹C═CR⁹, C≡C, Si(R⁹)₂, Ge(R⁹)₂, Sn(R⁹)₂, C═O, C═S, C═Se, C═NR⁹, P(═O)(R⁹), SO, SO₂, NR⁹, O, S or CONR⁹;
C₁-C₄₀-thioalkoxy,
which is optionally substituted with one or more substituents R⁹ and
wherein one or more non-adjacent CH₂-groups are optionally substituted by R⁹C═CR⁹, C≡C, Si(R⁹)₂, Ge(R⁹)₂, Sn(R⁹)₂, C═O, C═S, C═Se, C═NR⁹, P(═O)(R⁹), SO, SO₂, NR⁹, O, S or CONR⁹;
C₂-C₄₀-alkenyl,
which is optionally substituted with one or more substituents R⁹ and
wherein one or more non-adjacent CH₂-groups are optionally substituted by R⁹C═CR⁹, C≡C, Si(R⁹)₂, Ge(R⁹)₂, Sn(R⁹)₂, C═O, C═S, C═Se, C═NR⁹, P(═O)(R⁹), SO, SO₂, NR⁹, O, S or CONR⁹;
C₂-C₄₀-alkynyl,
which is optionally substituted with one or more substituents R⁹ and
wherein one or more non-adjacent CH₂-groups are optionally substituted by R⁹C═CR⁹, C≡C, Si(R⁹)₂, Ge(R⁹)₂, Sn(R⁹)₂, C═O, C═S, C═Se, C═NR⁹, P(═O)(R⁹), SO, SO₂, NR⁹, O, S or CONR⁹;
C₆-C₆₀-aryl,
which is optionally substituted with one or more substituents R⁹; and
C₃-C₅₇-heteroaryl,
which is optionally substituted with one or more substituents R⁹;
wherein, optionally, any of the substituents selected from the group consisting of R^{a}, R^{b}, R^{c}, R^{d}, R⁶, R⁷, and R⁸ independently form a mono- or polycyclic, aliphatic or aromatic, carbo- or heterocyclic and/or benzo-fused ring or ring system with one or more adjacent substituents selected from the group consisting of R^{a}, R^{b}, R^{c}, R^{d}, R⁶, R⁷, and R⁸; wherein the formed additional ring or rings are optionally substituted with one or more substituents R¹⁰;
#represents the binding site of the first chemical moiety to the second chemical moiety;
Z is selected from the group consisting of a direct bond, CR¹¹R¹², C═CR¹¹R¹², C═O, C═NR¹¹, NR¹¹, O, SiR¹¹R¹², S, S(O), and S(O)₂;
R^{e}, R^{f}, R^{g}, R¹¹, and R¹² are at each occurrence independently selected from the group consisting of: hydrogen, deuterium, N(R¹³)₂, OR¹³, Si(R¹³)₃, B(OR¹³)₂, OSO₂R¹³, CF₃, CN, F, Cl, Br, I,
C₁-C₄₀-alkyl,
which is optionally substituted with one or more substituents R¹³ and
wherein one or more non-adjacent CH₂-groups are optionally substituted by R¹³C═CR¹³, C≡C, Si(R¹³)₂, Ge(R¹³)₂, Sn(R¹³)₂, C═O, C═S, C═Se, C═NR¹³, P(═O)(R¹³), SO, SO₂, NR¹³, O, S or CONR¹³;
C₁-C₄₀-alkoxy,
which is optionally substituted with one or more substituents R¹³ and
wherein one or more non-adjacent CH₂-groups are optionally substituted by R¹³C═CR¹³, C≡C, Si(R¹³)₂, Ge(R¹³)₂, Sn(R¹³)₂, C═O, C═S, C═Se, C═NR¹³, P(═O)(R¹³), SO, SO₂, NR¹³, O, S or CONR¹³;
C₁-C₄₀-thioalkoxy,
which is optionally substituted with one or more substituents R¹³ and
wherein one or more non-adjacent CH₂-groups are optionally substituted by R¹³C═CR¹³, C≡C, Si(R¹³)₂, Ge(R¹³)₂, Sn(R¹³)₂, C═O, C═S, C═Se, C═NR¹³, P(═O)(R¹³), SO, SO₂, NR¹³, O, S or CONR¹³;
C₂-C₄₀-alkenyl,
which is optionally substituted with one or more substituents R¹³ and
wherein one or more non-adjacent CH₂-groups are optionally substituted by R¹³C═CR¹³, C≡C, Si(R¹³)₂, Ge(R¹³)₂, Sn(R¹³)₂, C═O, C═S, C═Se, C═NR¹³, P(═O)(R¹³), SO, SO₂, NR¹³, O, S or CONR¹³;
C₂-C₄₀-alkynyl,
which is optionally substituted with one or more substituents R¹³ and
wherein one or more non-adjacent CH₂-groups are optionally substituted by R¹³C═CR¹³, C≡C, Si(R¹³)₂, Ge(R¹³)₂, Sn(R¹³)₂, C═O, C═S, C═Se, C═NR¹³, P(═O)(R¹³), SO, SO₂, NR¹³, O, S or CONR¹³;
C₆-C₆₀-aryl,
which is optionally substituted with one or more substituents R¹³; and
C₃-C₆₀-heteroaryl,
which is optionally substituted with one or more substituents R¹³;
R¹³ is at each occurrence independently selected from the group consisting of: hydrogen, deuterium, N(R¹⁴)₂, OR¹⁴, Si(R¹⁴)₃, B(OR¹⁴)₂, OSO₂R¹⁴, CF₃, CN, F, Br, I, C₁-C₄₀-alkyl,
which is optionally substituted with one or more substituents R¹⁴ and
wherein one or more non-adjacent CH₂-groups are optionally substituted by R¹⁴C═CR¹⁴, C≡C, Si(R¹⁴)₂, Ge(R¹⁴)₂, Sn(R¹⁴)₂, C═O, C═S, C═Se, C═NR¹⁴, P(═O)(R¹⁴), SO, SO₂, NR¹⁴, O, S or CONR¹⁴;
C₁-C₄₀-alkoxy,
which is optionally substituted with one or more substituents R¹⁴ and
wherein one or more non-adjacent CH₂-groups are optionally substituted by R¹⁴C═CR¹⁴, C≡C, Si(R¹⁴)₂, Ge(R¹⁴)₂, Sn(R¹⁴)₂, C═O, C═S, C═Se, C═NR¹⁴, P(═O)(R¹⁴), SO, SO₂, NR¹⁴, O, S or CONR¹⁴;
C₁-C₄₀-thioalkoxy,
which is optionally substituted with one or more substituents R¹⁴ and
wherein one or more non-adjacent CH₂-groups are optionally substituted by R¹⁴C═CR¹⁴, C≡C, Si(R¹⁴)₂, Ge(R¹⁴)₂, Sn(R¹⁴)₂, C═O, C═S, C═Se, C═NR¹⁴, P(═O)(R¹⁴), SO, SO₂, NR¹⁴, O, S or CONR¹⁴;
C₂-C₄₀-alkenyl,
which is optionally substituted with one or more substituents R¹⁴ and
wherein one or more non-adjacent CH₂-groups are optionally substituted by R¹⁴C═CR¹⁴, C≡C, Si(R¹⁴)₂, Ge(R¹⁴)₂, Sn(R¹⁴)₂, C═O, C═S, C═Se, C═NR¹⁴, P(═O)(R¹⁴), SO, SO₂, NR¹⁴, O, S or CONR¹⁴;
C₂-C₄₀-alkynyl,
which is optionally substituted with one or more substituents R¹⁴ and
wherein one or more non-adjacent CH₂-groups are optionally substituted by R¹⁴C═CR¹⁴, C≡C, Si(R¹⁴)₂, Ge(R¹⁴)₂, Sn(R¹⁴)₂, C═O, C═S, C═Se, C═NR¹⁴, P(═O)(R¹⁴), SO, SO₂, NR¹⁴, O, S or CONR¹⁴;
C₆-C₆₀-aryl,
which is optionally substituted with one or more substituents R¹⁴; and
C₃-C₅₇-heteroaryl,
which is optionally substituted with one or more substituents R¹⁴;
wherein, optionally, any of the substituents selected from the group consisting of R^{e}, R^{f}, R^{g}, R¹¹, R¹², and R¹³ independently form a mono- or polycyclic, aliphatic or aromatic, carbo- or heterocyclic and/or benzo-fused ring or ring system with one or more adjacent substituents selected from selected from the group consisting of R^{e}, R^{f}, R^{g}, R¹¹, R¹², and R¹³; wherein the formed additional ring or rings are optionally substituted with one or more substituents R¹⁵;
R⁴, R⁹, R¹⁰, R¹⁴, and R¹⁵ are at each occurrence independently selected from the group consisting of: hydrogen, deuterium, OPh, CF₃, CN, F,
C₁-C₅-alkyl,
wherein one or more hydrogen atoms are optionally, independently substituted by deuterium, CN, CF₃, or F;
C₁-C₅-alkoxy,
wherein one or more hydrogen atoms are optionally, independently substituted by deuterium, CN, CF₃, or F;
C₁-C₅-thioalkoxy,
wherein one or more hydrogen atoms are optionally, independently substituted by deuterium, CN, CF₃, or F;
C₂-C₅-alkenyl,
wherein one or more hydrogen atoms are optionally, independently substituted by deuterium, CN, CF₃, or F;
C₂-C₅-alkynyl,
wherein one or more hydrogen atoms are optionally, independently substituted by deuterium, CN, CF₃, or F;
C₃-C₁₈-aryl,
wherein one or more hydrogen atoms are optionally, independently substituted by deuterium, C₁-C₅-alkyl, Ph or CN;
C₃-C₁₅-heteroaryl,
wherein one or more hydrogen atoms are optionally, independently substituted by deuterium, Ph or C₁-C₅-alkyl;
N(C₆-C₁₈-aryl)₂;
N(C₃-C₁₇-heteroaryl)₂, and
N(C₃-C₁₇-heteroaryl)(C₆-C₁₈-aryl);
R⁵ is at each occurrence independently selected from the group consisting of: hydrogen, deuterium, OPh, CF₃, F,
C₁-C₅-alkyl,
wherein one or more hydrogen atoms are optionally, independently substituted by deuterium, CN, CF₃, or F;
C₁-C₅-alkoxy,
wherein one or more hydrogen atoms are optionally, independently substituted by deuterium, CN, CF₃, or F;
C₁-C₅-thioalkoxy,
wherein one or more hydrogen atoms are optionally, independently substituted by deuterium, CN, CF₃, or F;
C₂-C₅-alkenyl,
wherein one or more hydrogen atoms are optionally, independently substituted by deuterium, CN, CF₃, or F;
C₂-C₅-alkynyl,
wherein one or more hydrogen atoms are optionally, independently substituted by deuterium, CN, CF₃, or F;
C₆-C₁₈-aryl,
wherein one or more hydrogen atoms are optionally, independently substituted by deuterium, C₁-C₅-alkyl, Ph or CN;
C₃-C₁₅-heteroaryl, [symbol]
wherein one or more hydrogen atoms are optionally, independently substituted by deuterium, Ph or C₁-C₅-alkyl;
N(C₆-C₁₈-aryl)₂;
N(C₃-C₁₇-heteroaryl)₂, and
N(C₃-C₁₇-heteroaryl)(C₆-C₁₈-aryl);
and wherein:
exactly one substituent selected from the group consisting of T, W, X, and Y is R^{X}, and exactly one substituent selected from the group consisting of T, V, and W represents the binding site of a single bond linking the first chemical moiety to the second chemical moiety; and
wherein W is hydrogen (H), if T is R^{X} and V is the binding site of a single bond linking the first chemical moiety and the second chemical moiety.

2. The organic molecule according to claim 1, wherein R¹ is selected from the group consisting of: 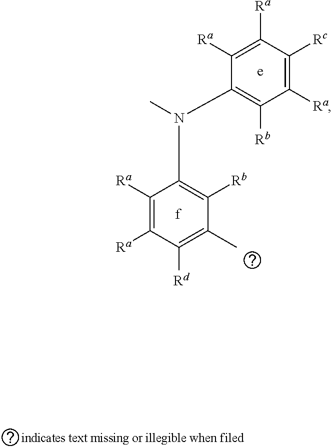
hydrogen, deuterium,
C₁-C₅-alkyl,
which is optionally substituted with one or more substituents R³;
C₆-C₁₈-aryl,
which is optionally substituted with one or more substituents R³;
R³ is at each occurrence independently selected from the group consisting of: hydrogen, deuterium, N(Ph)₂, OPh, Si(Me)₃, Si(Ph)₃, CF₃, CN, F, C₁-C₅-alkyl,
which is optionally substituted with one or more substituents R⁴;
C₆-C₁₈-aryl,
which is optionally substituted with one or more substituents R⁴; and
C₃-C₁₅-heteroaryl,
which is optionally substituted with one or more substituents R⁴;
R² is at each occurrence independently selected from the group consisting of:
hydrogen, deuterium,
C₁-C₅-alkyl,
wherein one or more hydrogen atoms are optionally substituted by deuterium;
C₆-C₁₈-aryl;
wherein one or more hydrogen atoms are optionally substituted by a group R⁵;
wherein the two moieties R^{b} optionally form a group Y, which is selected from the group consisting of a direct bond, C═O, NR⁶, O, SiR⁶R⁷, S, S(O) and S(O)₂;
R^{a}, R^{b}, R^{c}, R^{d}, R⁶, and R⁷ are at each occurrence independently selected from the group consisting of: hydrogen, deuterium, Oph, Si(Me)₃, Si(Ph)₃, N(Ph)₂, CF₃, CN,
C₁-C₅-alkyl,
which is optionally substituted with one or more substituents R⁸ and C₆-C₁₈-aryl,
which is optionally substituted with one or more substituents R⁸; and
C₃-C₁₅-heteroaryl,
which is optionally substituted with one or more substituents R⁸;
R⁸ is at each occurrence independently selected from the group consisting of: hydrogen, deuterium, Oph, Si(Me)₃, Si(Ph)₃, CF₃, CN, F,
C₁-C₅-alkyl,
which is optionally substituted with one or more substituents R⁹ and
C₆-C₁₈-aryl,
which is optionally substituted with one or more substituents R⁹; and
C₃-C₁₅-heteroaryl,
which is optionally substituted with one or more substituents R⁹;
wherein, optionally, any of the substituents selected from the group consisting of R^{a}, R^{b}, R^{c}, R^{d}, R⁶, R⁷, and R⁸ independently form a mono- or polycyclic, aliphatic or aromatic, carbo- or heterocyclic and/or benzo-fused ring or ring system with one or more adjacent substituents selected from selected from the group consisting of R^{a}, R^{b}, R^{c}, R^{d}, R⁶, R⁷, and R⁸; wherein the formed fused ring system of the respective benzene ring a, b, c, d, e or f and the additional rings formed by adjacent substituents has 9 to 30 ring atoms, of which 1 to 3 atoms may be heteroatoms independently selected from the group consisting of N, O and S and wherein the formed additional ring or rings are optionally substituted with one or more substituents R¹⁰;
R⁴, R⁹, and R¹⁰ are at each occurrence independently selected from the group consisting of: hydrogen, deuterium, Oph, CF₃, CN, F, N(Ph)₂, C₁-C₅-alkyl,
wherein one or more hydrogen atoms are optionally, independently substituted by deuterium;
C₆-C₁₈-aryl, [symbol]
wherein one or more hydrogen atoms are optionally, independently substituted by deuterium, C₁-C₅-alkyl, Ph or CN;
C₃-C₁₅-heteroaryl,
wherein one or more hydrogen atoms are optionally, independently substituted by deuterium, C₁-C₅-alkyl, Ph or CN;
R⁵ is at each occurrence independently selected from the group consisting of: hydrogen, deuterium, Oph, CF₃, F, N(Ph)₂,
C₁-C₅-alkyl,
wherein one or more hydrogen atoms are optionally, independently substituted by deuterium;
C₆-C₁₈-aryl,
wherein one or more hydrogen atoms are optionally, independently substituted by deuterium, Me, ⁱPr, ^{t}Bu, Ph or CN;
C₃-C₁₅-heteroaryl,
wherein one or more hydrogen atoms are optionally, independently substituted by deuterium, Me, ⁱPr, ^{t}Bu, Ph or CN.

3. The organic molecule according to claim 1, wherein R¹ is selected from the group consisting of: 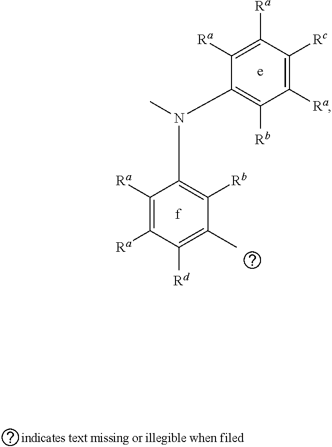
hydrogen, deuterium, Me, ⁱPr, ^{t}Bu, and
Ph, wherein one or more hydrogen atoms are optionally, independently substituted by deuterium, Me, ⁱPr, ^{t}Bu, Ph or CN;
R² is at each occurrence independently selected from the group consisting of:
hydrogen, deuterium, Me, ⁱPr, ^{t}Bu, and
Ph, wherein one or more hydrogen atoms are optionally substituted by deuterium, Me, ⁱPr, ^{t}Bu or Ph;
wherein the two moieties R^{b} optionally form a group Y, which is at each occurrence a direct bond;
R^{a}, R^{b}, R^{c}, and R^{d} are at each occurrence independently selected from the group consisting of: hydrogen, deuterium, CN, Me, ⁱPr, ^{t}Bu,
Ph, wherein one or more hydrogen atoms are optionally, independently substituted by deuterium, Me, ⁱPr, ^{t}Bu, CN or Ph;
wherein, optionally, any of the substituents selected from the group consisting of R^{a}, R^{b}, R^{c}, and R^{d} independently form a mono- or polycyclic, aliphatic or aromatic, carbo- or heterocyclic and/or benzo-fused ring or ring system with one or more adjacent substituents selected from the group consisting of R^{a}, R^{b}, R^{c}, and R^{d}; wherein the formed fused ring system of the respective benzene ring a, b, c, d, e or f and the additional rings formed by adjacent substituents has 9 to 30 ring atoms, of which 1 to 3 atoms may be heteroatoms independently selected from the group consisting of N, O and S and wherein the formed additional ring or rings are optionally substituted with one or more substituents R¹⁰; and
R¹⁰ is at each occurrence independently selected from the group consisting of: hydrogen, deuterium, CN, Me, ⁱPr, ^{t}Bu,
Ph, wherein one or more hydrogen atoms are optionally, independently substituted by deuterium, Me, ⁱPr, ^{t}Bu or Ph.

4. The organic molecule according to claim 1, wherein the first chemical moiety comprises a structure according to any of formulas I-a-1, I-b-1, I-a-2, and I-b-2: 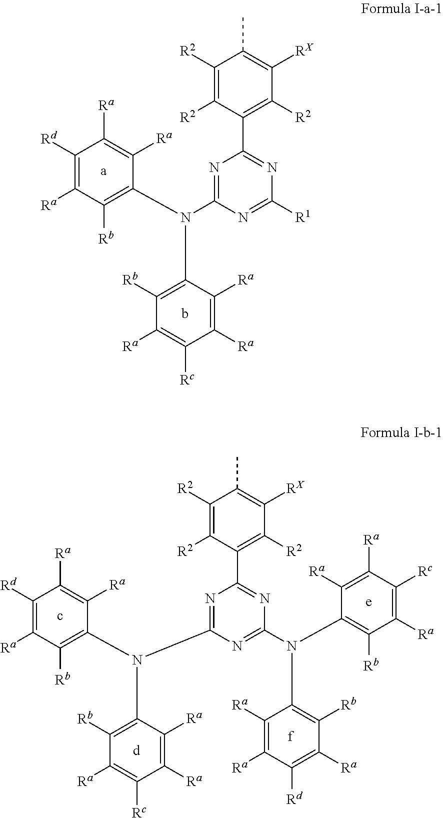 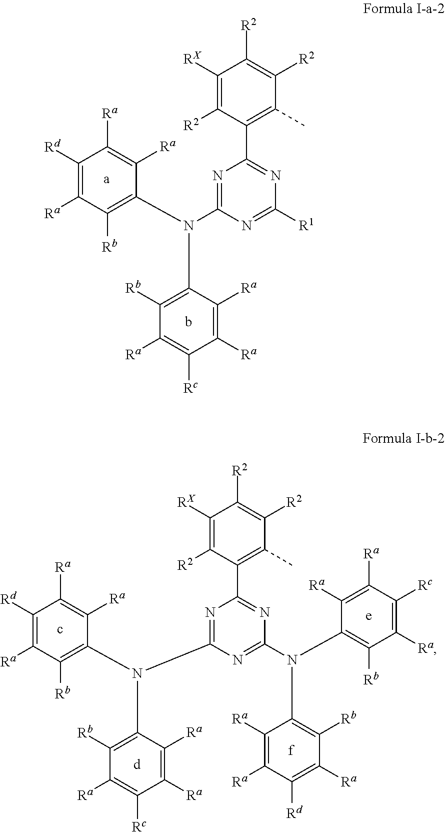
wherein the dashed line indicates the single bond linking the first chemical moiety to the second chemical moiety.

5. The organic molecule according to claim 1, wherein R^{e}, R^{f}, R^{g}, R¹¹, and R¹² are at each occurrence independently selected from the group consisting of: hydrogen, deuterium, N(Ph)₂, OPh, Si(Me)₃, Si(Ph)₃, F, CF₃, CN,
C₆-C₁₈-aryl,
wherein one or more hydrogen atoms are optionally, independently substituted by deuterium, Me, ⁱPr, ^{t}Bu, CF₃, CN or Ph;
C₃-C₁₅-heteroaryl,
wherein one or more hydrogen atoms are optionally, independently substituted by deuterium, Me, ⁱPr, ^{t}Bu, CF₃, CN or Ph;
wherein, optionally, any of the substituents selected from the group consisting of R^{e}, R^{f}, R^{g}, R¹¹ and R¹² independently form a mono- or polycyclic, aliphatic or aromatic, carbo- or heterocyclic and/or benzo-fused ring or ring system with one or more adjacent substituents selected from the group consisting of R^{e}, R^{f}, R^{g}, R¹¹, and R¹²; wherein the formed fused ring system of the structure according to formula II and the additional rings formed by adjacent substituents has 16 to 30 ring atoms, of which 1 to 3 atoms may be heteroatoms independently selected from N, O, and S; and wherein the formed additional ring or rings is optionally substituted with one or more substituents R¹⁵;
R¹⁵ is at each occurrence independently selected from the group consisting of: hydrogen, deuterium, CN, Me, ⁱPr, ^{t}Bu, and
Ph, wherein one or more hydrogen atoms are optionally, independently substituted by deuterium, Me, ⁱPr, ^{t}Bu, Ph or CN.

6. The organic molecule according to claim 1, wherein the second chemical moiety comprises a structure of formula II-a:

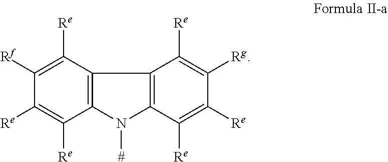

7. The organic molecule according to claim 1, wherein the second chemical moiety comprises a structure according to any of formulas II-a-1, II-a-5, II-a-9, II-a-10, II-a-11, II-a-12, II-a-13, II-a-14, and II-a-15: 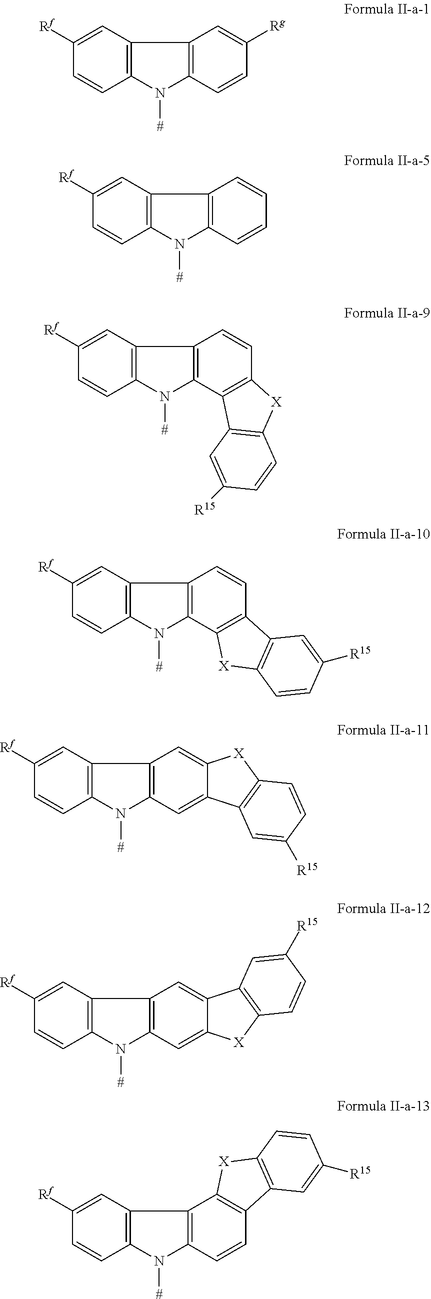 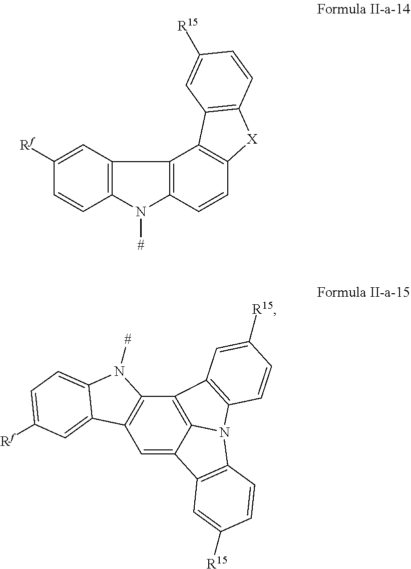
wherein:
X is selected from the group consisting of C(R¹⁶)₂, NR¹⁶, O, and S; and
R¹⁶ is at each occurrence independently selected from the group consisting of: hydrogen, deuterium, Me, ⁱPr, ^{t}Bu, and
Ph, wherein one or more hydrogen atoms are optionally substituted by deuterium, Me, ⁱPr, ^{t}Bu, and Ph.

8. The organic molecule according to claim 1, wherein R^{X} is CN at each occurrence.

9. (canceled)

10. (canceled)

11. A composition, comprising:
(a) an organic molecule according to claim 1, in the form of an emitter and/or a host, and
(b) an emitter and/or a host material, which differs from the organic molecule, and
(c) optionally, one or more dyes and/or one or more solvents.

12. The composition according to claim 11, comprising:
(i) 1-50% by weight of the organic molecule;
(ii) 5-98% by weight of a first host compound;
(iii) 1-30% by weight of at least one further emitter molecule having a structure differing from the structure of the organic molecule; and
(iv) optionally, 0-94% by weight of at least a second host compound having a structure differing from the structure of the organic molecule; and
(v) optionally, 0-94% by weight of a solvent,
wherein the sum of the components of the composition is 100% by weight.

13. An optoelectronic device, comprising an organic molecule according to claim 1, wherein the optoelectronic device is selected from the group consisting of an organic light-emitting diode (OLED), a light-emitting electrochemical cell, an OLED-sensor, an organic diode, an organic solar cell, an organic transistor, an organic field-effect transistor, an organic laser, and a down-conversion element.

14. The optoelectronic device according to claim 13, comprising:
a substrate,
an anode, and
a cathode, wherein the anode or the cathode are on the substrate, and
a light-emitting layer, which is arranged between the anode and the cathode and which comprises the organic molecule or the composition.

15. A method for producing an optoelectronic device, the method comprising processing the organic molecule according to claim 1 using a vacuum evaporation method or from a solution.
